(19) United States
(12) Patent Application Publication
(10) Pub. No.: US 2025/0197481 A1
(43) Pub. Date: Jun. 19, 2025

(54) Antigen Binding Polypeptides, Antigen Binding Polypeptide Complexes and Methods of Use Thereof in HIV

(71) Applicant: ModeX Therapeutics, Inc., Natick, MA (US)

(72) Inventors: Ronnie Rong WEI, Weston, MA (US); Ling XU, Newton Center, MA (US); Zhi-Yong YANG, Newton Center, MA (US); Gary J. NABEL, Delray Beach, FL (US)

(21) Appl. No.: 18/696,493

(22) PCT Filed: Sep. 28, 2022
(86) PCT No.: PCT/US2022/077203

§ 371 Date: Mar. 28, 2024

Related U.S. Application Data
(60) Provisional application No. 63/249,722, filed on Sep. 29, 2021.

(51) Int. Cl. C07K 16/10; A61K 40/31

(52) U.S. Cl. CPC C07K 16/1045; A61K 40/31; C07K 2317/14; C07K 2317/31; C07K 2317/522; C07K 2317/524; C07K 2317/526

(57) ABSTRACT

Disclosed are antigen binding polypeptides and antigen binding polypeptide complexes (e.g., antibodies and antigen binding fragments thereof) that bind to HIV proteins and have certain structural features. Also disclosed are polynucleotides and vectors encoding such polypeptides and polypeptide complexes; host cells; chimeric antigen receptors (CARs); immune cells; pharmaceutical compositions and kits containing such polypeptides and polypeptide complexes; and methods of using such polypeptides and polypeptide complexes in HIV.

### CROSS REFERENCE TO RELATED APPLICATIONS

This application claims the priority benefit of U.S. Provisional Application No. 63/249,722, filed Sep. 29, 2021, which is incorporated herein by reference in its entirety.

### REFERENCE TO SEQUENCE LISTING SUBMITTED ELECTRONICALLY

The content of the electronically submitted sequence listing (Name: 4850_006PC01_Seqlisting_ST26; Size: 196,015 bytes; and Date of Creation: Sep. 26, 2022) is herein incorporated by reference in its entirety.

### FIELD

The present disclosure relates to antigen binding polypeptides and antigen binding polypeptide complexes (e.g., antibodies and antigen binding fragments thereof) that specifically bind to HIV proteins and have certain structural features. The present disclosure also relates to polynucleotides and vectors encoding such polypeptides and polypeptide complexes, host cells, chimeric antigen receptors (CARs), immune cells, pharmaceutical compositions and kits containing such polypeptides and polypeptide complexes, and methods of using such polypeptides and polypeptide complexes.

### BACKGROUND

Human immunodeficiency virus (HIV) poses a major infectious disease burden with immense medical and economic impact around the world. Globally, ˜38 million people have been infected with HIV, and more than 30 million individuals have succumbed to acquired immunodeficiency syndrome (AIDS), a chronic condition of weakened immune system caused by HIV infection. “Global Health Sector Strategy On HIV—2016-2021—Towards Ending AIDS,” World Health Organization, June 2016. There are two major forms of HIV: HIV-1 and HIV-2. HIV-1 is the more prevalent form worldwide, while HIV-2 is less pathogenic and mostly confined to West Africa.

The major structural proteins of HIV are Gag, Pol and Env. Gag (group specific antigen) is the structural protein for the viral core. Pol is a polyprotein containing the enzymes critical for viral replication: protease (PR), reverse transcriptase (RT), and integrase (IN). Env (envelope) encodes glycoproteins that form the virus's exterior envelope. Env is synthesized as a precursor glycoprotein, gp160, and is then processed into gp120 and gp41. Env interacts with the primary receptor CD4 and a coreceptor (such as chemokine receptor CCR5) to fuse viral and target-cell membranes.

The genetic heterogeneity and glycan shielding of Env have resisted the development of natural immunity to HIV and posed challenges to traditional vaccine development. It has also prompted a search for alternative approaches to HIV prevention, one of the highest priorities in global health.

Despite a significant collection of anti-HIV/AIDS drugs available, HIV patients still face daily challenges in taking multiple medicines with strict regimens. Inevitably, most patients will bear the consequences of emergence of drug-resistant viral variants, and develop other health issues from the toxicities of taking anti-HIV medicines long term, such as cardiovascular disease, kidney disease, diabetes, bone disease, liver disease, cognitive disorders, etc. Alternative treatment options are urgently needed for HIV/AIDS patients.

Broadly neutralizing HIV-1 antibodies (bnAbs) are antibodies that neutralize multiple HIV-1 viral strains. bnAbs target conserved epitopes of the virus, meaning that the targeted epitopes may be more likely to remain even if the virus mutates. As such, bnAbs have been investigated recently for HIV/AIDS treatment and prevention. Human clinical studies have revealed two factors critical for efficacy of bnAbs. First, there is the need to exceed a minimally effective dose, or trough level of circulating bnAbs to prevent infection. Second, there is a need to prevent the emergence of viral escape through resistance mutations.

Early human clinical studies using bnAbs demonstrated the feasibility and safety of this approach with transient reductions of viral load and acceptable tolerability and immunogenicity. Burton et al., Annu. Rev. Immunol. 34:635-659 (2016); Mascola et al., Immunol. Rev. 254:225-244 (2013); Wu et al., Science. 329:856-861 (2010). However, resistant HIV strains emerged rapidly following treatment with individual bnAbs in vitro and in vivo. More recently, a phase II clinical trial with the VRC01 bnAb highlighted the importance of maintaining adequate circulating antibody levels to reduce acquisition rates, suggesting that combination antibody therapy which enhances potency and minimizes escape mutations will be required for effective prevention. Corey et al., N. Engl. J. Med. 384:1003-1014 (2021).

Multispecific antibodies address the limitations of bnAbs by providing a single antibody type that recognizes multiple independent binding sites on HIV-1 envelope protein. Xu et al., Science. 358(6359):85-90 (2017). Treatment with multispecific antibodies also ensures that independent binding specificities are maintained with the same pharmacokinetics, while treatment with multiple single-target antibodies results in different antibody half-lives that wane at different rates. Furthermore, multispecific antibodies simplify manufacturing and regulatory processes by using one product for clinical development instead of a combination of multiple products.

Accordingly, multispecific anti-HIV antibodies provide an important technological platform for developing neutralizing antibody-based therapeutics for treating HIV/AIDS, offering a class of medicines with low long-term toxicities and significantly less frequent treatment regimen. Multispecific antibodies also use completely different targets on HIV from the current standard of care HIV/AIDS medicine, complementing to the existing medicines by providing patients alternatives for their disease control and health management. Multispecific antibodies may also offer a meaningful way for HIV prevention in the current absence of an effective HIV vaccine.

In addition, the development of therapeutic antibodies can be challenging, especially manufacturing and late stage development. For example, the production of multispecific antibodies often requires multiple genes or plasmids for cell line development. These multiple genes or plasmids must be delivered into the same cell to make the correct molecules. Furthermore, multispecific antibodies can have mispairing between the heavy and light chains, which can reduce product yield, increase cell line colony screen workload, and create product heterogeneity.

As such, there is a need for multispecific and multifunctional antibodies, antigen binding polypeptides and antigen binding polypeptide complexes that can bind to HIV proteins for selectivity or breadth/neutralization, bring together two or more cell types, bring together targets and deliver activation signals, modify the HIV microenvironment, and enhance avidity of binding for improved potency.

### BRIEF DESCRIPTION OF THE DRAWINGS

Some aspects of the invention are herein described, by way of example only, with reference to the accompanying drawings. With specific reference now to the drawings in detail, it is stressed that the particulars shown are by way of example and for purposes of illustrative discussion of aspects of the invention.

FIG. 1 shows non-limiting examples of different configurations of tetraspecific antibody molecules.

FIG. 2A shows a non-limiting example of a trispecific antibody configuration, called MX894 (VRC01scFv/PGT121×10e8v4L1IgG1LS). MX894 was analyzed for binding to 10e8 fusion peptide (FIG. 2B), and CD4 site-dependent (FIG. 2C) and CD4 site-independent (FIG. 2D) HIV spike protein by biolayer interferometry (BLI).

FIG. 3A shows a further non-limiting example of a tetraspecific antibody configuration, called MX873 (VRC26.25×10-1074L9/VRC01×PGT121L1 IgG1LS). MX873 was analyzed for binding to CD4 site-dependent (FIG. 3B) and CD4 site-independent (FIG. 3C) HIV spike protein by biolayer interferometry (BLI).

FIG. 4A shows a further non-limiting example of a tetraspecific antibody configuration, called MX875 (10-1074×VRC26.25L9/VRC01×PGT121L1 IgG1LS). MX875 was analyzed for binding to CD4 site-dependent (FIG. 4B) and CD4 site-independent (FIG. 4C) HIV spike protein by biolayer interferometry (BLI).

FIG. 5A shows a further non-limiting example of a tetraspecific antibody configuration, called MX877 (STAR_VRC26.25×PGT128L9/STAR_VRC01×PGT121L1 IgG1LS). MX877 was analyzed for binding to CD4 site-dependent (FIG. 5B) and CD4 site-independent (FIG. 5C) HIV spike protein by biolayer interferometry (BLI).

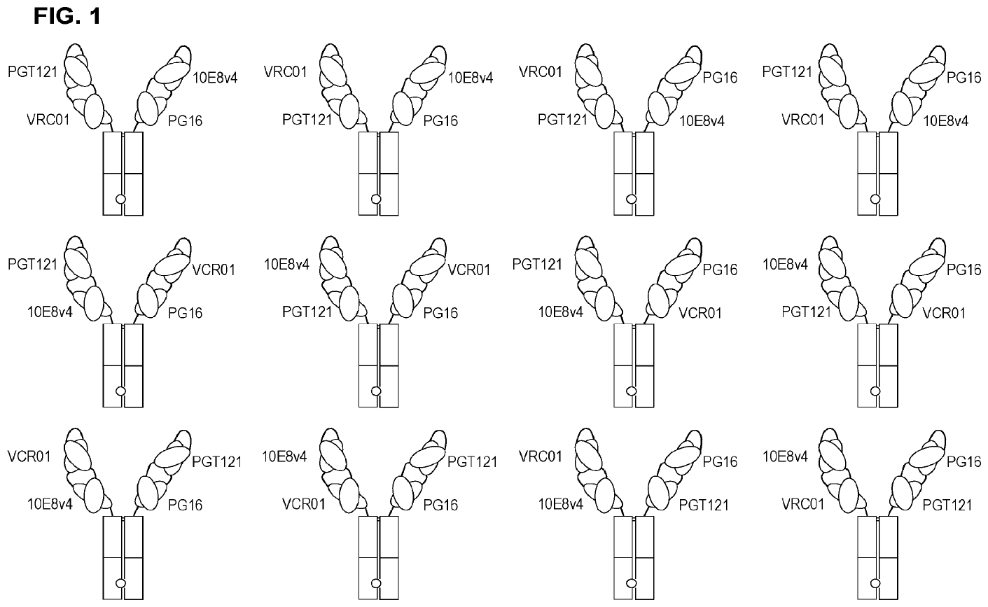

### BRIEF SUMMARY

Provided herein is an antigen binding polypeptide having a structure represented by VL1-VL2-VH2-VH1; VH1-VH2-VL2-VL1; VL1-L1-VL2-L2-VH2-L3-VH1; or VH1-L1-VH2-L2-VL2-L3-VL1; wherein VL1 is a first immunoglobulin light chain variable region that specifically binds to an HIV protein; VL2 is a second immunoglobulin light chain variable region that specifically binds to an HIV protein; VH1 is a first immunoglobulin heavy chain variable region that specifically binds to an HIV protein; VH2 is a second immunoglobulin heavy chain variable region that specifically binds to an HIV protein; and L1, L2 and L3 are amino acid linkers.

Provided herein is an antigen binding polypeptide complex comprising a first polypeptide and a second polypeptide; wherein the first polypeptide has a structure represented by VL1-VL2-VH2-VH1; VH1-VH2-VL2-VL1; VL1-L1-VL2-L2-VH2-L3-VH1; or VH1-L1-VH2-L2-VL2-L3-VL1; wherein the second polypeptide has a structure represented by VL1-VL2-VH2-VH1; VH1-VH2-VL2-VL1; VL1-L1-VL2-L2-VH2-L3-VH1; or VH1-L1-VH2-L2-VL2-L3-VL1; wherein VL1 is a first immunoglobulin light chain variable region that specifically binds to an HIV protein; VL2 is a second immunoglobulin light chain variable region that specifically binds to an HIV protein; VH1 is a first immunoglobulin heavy chain variable region that specifically binds to an HIV protein; VH2 is a second immunoglobulin heavy chain variable region that specifically binds to an HIV protein; and L1, L2 and L3 are amino acid linkers.

Provided herein is an antigen binding polypeptide having a structure represented by VL1-VL2-VH2-VH1-Fc; VH1-VH2-VL2-VL1-Fc; VL1-L1-VL2-L2-VH2-L3-VH1-Fc; VH1-L1-VH2-L2-VL2-L3-VL1-Fc; VL1-L1-VL2-L2-VH2-L3-VH1-L4-Fc; or VH1-L1-VH2-L2-VL2-L3-VL1-L4-Fc; wherein VL1 is a first immunoglobulin light chain variable region that specifically binds to an HIV protein; VL2 is a second immunoglobulin light chain variable region that specifically binds to an HIV protein; VH1 is a first immunoglobulin heavy chain variable region that specifically binds to an HIV protein; VH2 is a second immunoglobulin heavy chain variable region that specifically binds to an HIV protein; Fc is a region comprising an immunoglobulin heavy chain constant region 2 (CH2), an immunoglobulin heavy chain constant region 3 (CH3), and optionally, an immunoglobulin hinge; and L1, L2, L3 and L4 are amino acid linkers.

Provided herein is an antigen binding polypeptide complex comprising a first polypeptide and a second polypeptide; wherein the first polypeptide has a structure represented by VL1-VL2-VH2-VH1-Fc; VH1-VH2-VL2-VL1-Fc; VL1-L1-VL2-L2-VH2-L3-VH1-Fc; VH1-L1-VH2-L2-VL2-L3-VL1-Fc; VL1-L1-VL2-L2-VH2-L3-VH1-L4-Fc; or VH1-L1-VH2-L2-VL2-L3-VL1-L4-Fc; wherein the second polypeptide has a structure represented by Fc; VL1-VL2-VH2-VH1-Fc; VH1-VH2-VL2-VL1-Fc; VL1-L1-VL2-L2-VH2-L3-VH1-Fc; VH1-L1-VH2-L2-VL2-L3-VL1-Fc; VL1-L1-VL2-L2-VH2-L3-VH1-L4-Fc; or VH1-L1-VH2-L2-VL2-L3-VL1-L4-Fc; wherein VL1 is a first immunoglobulin light chain variable region that specifically binds to an HIV protein; VL2 is a second immunoglobulin light chain variable region that specifically binds to an HIV protein; VH1 is a first immunoglobulin heavy chain variable region that specifically binds to an HIV protein; VH2 is a second immunoglobulin heavy chain variable region that specifically binds to an HIV protein; Fc is a region comprising an immunoglobulin heavy chain constant region 2 (CH2), an immunoglobulin heavy chain constant region 3 (CH3), and optionally, an immunoglobulin hinge; and L1, L2, L3 and L4 are amino acid linkers.

Provided herein is an antigen binding polypeptide having a structure represented by VL1-VL2-VH2-VH1-CH1-CL; VH1-VH2-VL2-VL1-CH1-CL; VL1-VL2-VH2-VH1-CL-CH1; VH1-VH2-VL2-VL1-CL-CH1; VL1-L1-VL2-L2-VH2-L3-VH1-L4-CH1-L5-CL; VH1-L1-VH2-L2-VL2-L3-VL1-L4-CH1-L5-CL; VL1-L1-VL2-L2-VH2-L3-VH1-L4-CL-L5-CH1; or VH1-L1-VH2-L2-VL2-L3-VL1-L4-CL-L5-CH1; wherein VL1 is a first immunoglobulin light chain variable region that specifically binds to an HIV protein; VL2 is a second immunoglobulin light chain variable region that specifically binds to an HIV protein; VH1 is a first immunoglobulin heavy chain variable region that specifically binds to an HIV protein; VH2 is a second immunoglobulin heavy chain variable region that specifically binds to an HIV protein; CH1 is an immunoglobulin heavy chain constant region 1; CL is an immunoglobulin light chain constant region; and L1, L2, L3, L4 and L5 are amino acid linkers.

Provided herein is an antigen binding polypeptide complex comprising a first polypeptide and a second polypeptide; wherein the first polypeptide has a structure represented by VL1-VL2-VH2-VH1-CH1; VH1-VH2-VL2-VL1-CH1; VL1-VL2-VH2-VH1-CL; VH1-VH2-VL2-VL1-CL; VL1-VL2-VH2-VH1-CH1-CL; VH1-VH2-VL2-VL1-CH1-CL; VL1-VL2-VH2-VH1-CL-CH1; VH1-VH2-VL2-VL1-CL-CH1; VL1-L1-VL2-L2-VH2-L3-VH1-L4-CH1; VH1-L1-VH2-L2-VL2-L3-VL1-L4-CH1; VL1-L1-VL2-L2-VH2-L3-VH1-L4-CL; VH1-L1-VH2-L2-VL2-L3-VL1-L4-CL; VL1-L1-VL2-L2-VH2-L3-VH1-L4-CH1-L5-CL; VH1-L1-VH2-L2-VL2-L3-VL1-L4-CH1-L5-CL; VL1-L1-VL2-L2-VH2-L3-VH1-L4-CL-L5-CH1; or VH1-L1-VH2-L2-VL2-L3-VL1-L4-CL-L5-CH1; wherein the second polypeptide has a structure represented by VL1-VL2-VH2-VH1-CH1; VL1-VL2-VH2-VH1-CH1; VH1-VH2-VL2-VL1-CH1; VL1-VL2-VH2-VH1-CL; VH1-VH2-VL2-VL1-CL; VL1-VL2-VH2-VH1-CH1-CL; VH1-VH2-VL2-VL1-CH1-CL; VL1-VL2-VH2-VH1-CL-CH1; VH1-VH2-VL2-VL1-CL-CH1; VL1-L1-VL2-L2-VH2-L3-VH1-L4-CH1; VH1-L1-VH2-L2-VL2-L3-VL1-L4-CH1; VL1-L1-VL2-L2-VH2-L3-VH1-L4-CL; VH1-L1-VH2-L2-VL2-L3-VL1-L4-CL; VL1-L1-VL2-L2-VH2-L3-VH1-L4-CH1-L5-CL; VH1-L1-VH2-L2-VL2-L3-VL1-L4-CH1-L5-CL; VL1-L1-VL2-L2-VH2-L3-VH1-L4-CL-L5-CH1; or VH1-L1-VH2-L2-VL2-L3-VL1-L4-CL-L5-CH1; wherein VL1 is a first immunoglobulin light chain variable region that specifically binds to an HIV protein; VL2 is a second immunoglobulin light chain variable region that specifically binds to an HIV protein; VH1 is a first immunoglobulin heavy chain variable region that specifically binds to an HIV protein; VH2 is a second immunoglobulin heavy chain variable region that specifically binds to an HIV protein; CH1 is an immunoglobulin heavy chain constant region 1; CL is an immunoglobulin light chain constant region; and L1, L2, L3, L4 and L5 are amino acid linkers.

Provided herein is an antigen binding polypeptide having a structure represented by VL1-VL2-VH2-VH1-CH1-CL-Fc; VH1-VH2-VL2-VL1-CH1-CL-Fc; VL1-VL2-VH2-VH1-CL-CH1-Fc; VH1-VH2-VL2-VL1-CL-CH1-Fc; VL1-L1-VL2-L2-VH2-L3-VH1-L4-CH1-L5-CL-Fc; VH1-L1-VH2-L2-VL2-L3-VL1-L4-CH1-L5-CL-Fc; VL1-L1-VL2-L2-VH2-L3-VH1-L4-CL-L5-CH1-Fc; VH1-L1-VH2-L2-VL2-L3-VL1-L4-CL-L5-CH1-Fc; VL1-L1-VL2-L2-VH2-L3-VH1-L4-CH1-L5-CL-L6-Fc; VH1-L1-VH2-L2-VL2-L3-VL1-L4-CH1-L5-CL-L6-Fc; VL1-L1-VL2-L2-VH2-L3-VH1-L4-CL-L5-CH1-L6-Fc; or VH1-L1-VH2-L2-VL2-L3-VL1-L4-CL-L5-CH1-L6-Fc; wherein VL1 is a first immunoglobulin light chain variable region that specifically binds to an HIV protein; VL2 is a second immunoglobulin light chain variable region that specifically binds to an HIV protein; VH1 is a first immunoglobulin heavy chain variable region that specifically binds to an HIV protein; VH2 is a second immunoglobulin heavy chain variable region that specifically binds to an HIV protein; CH1 is an immunoglobulin heavy chain constant region 1; CL is an immunoglobulin light chain constant region; Fc is a region comprising an immunoglobulin heavy chain constant region 2 (CH2), an immunoglobulin heavy chain constant region 3 (CH3), and optionally, an immunoglobulin hinge; and L1, L2, L3, L4, L5 and L6 are amino acid linkers.

Provided herein is an antigen binding polypeptide complex comprising a first polypeptide and a second polypeptide; wherein the first polypeptide has a structure represented by VL1-VL2-VH2-VH1-CH1-Fc; VH1-VH2-VL2-VL1-CH1-Fc; VL1-VL2-VH2-VH1-CL-Fc; VH1-VH2-VL2-VL1-CL-Fc; VL1-VL2-VH2-VH1-CH1-CL-Fc; VH1-VH2-VL2-VL1-CH1-CL-Fc; VL1-VL2-VH2-VH1-CL-CH1-Fc; VH1-VH2-VL2-VL1-CL-CH1-Fc; VL1-L1-VL2-L2-VH2-L3-VH1-L4-CH1-Fc; VH1-L1-VH2-L2-VL2-L3-VL1-L4-CH1-Fc; VL1-L1-VL2-L2-VH2-L3-VH1-L4-CL-Fc; VH1-L1-VH2-L2-VL2-L3-VL1-L4-CL-Fc; VL1-L1-VL2-L2-VH2-L3-VH1-L4-CH1-L5-CL-Fc; VH1-L1-VH2-L2-VL2-L3-VL1-L4-CH1-L5-CL-Fc; VL1-L1-VL2-L2-VH2-L3-VH1-L4-CL-L5-CH1-Fc; VH1-L1-VH2-L2-VL2-L3-VL1-L4-CL-L5-CH1-Fc; VL1-L1-VL2-L2-VH2-L3-VH1-L4-CH1-L5-Fc; VH1-L1-VH2-L2-VL2-L3-VL1-L4-CH1-L5-Fc; VL1-L1-VL2-L2-VH2-L3-VH1-L4-CL-L5-Fc; VH1-L1-VH2-L2-VL2-L3-VL1-L4-CL-L5-Fc; VL1-L1-VL2-L2-VH2-L3-VH1-L4-CH1-L5-CL-L6-Fc; VH1-L1-VH2-L2-VL2-L3-VL1-L4-CH1-L5-CL-L6-Fc; VL1-L1-VL2-L2-VH2-L3-VH1-L4-CL-L5-CH1-L6-Fc; or VH1-L1-VH2-L2-VL2-L3-VL1-L4-CL-L5-CH1-L6-Fc; wherein the second polypeptide has a structure represented by Fc; VL1-VL2-VH2-VH1-CH1-Fc; VH1-VH2-VL2-VL1-CH1-Fc; VL1-VL2-VH2-VH1-CL-Fc; VH1-VH2-VL2-VL1-CL-Fc; VL1-VL2-VH2-VH1-CH1-CL-Fc; VH1-VH2-VL2-VL1-CH1-CL-Fc; VL1-VL2-VH2-VH1-CL-CH1-Fc; VH1-VH2-VL2-VL1-CL-CH1-Fc; VL1-L1-VL2-L2-VH2-L3-VH1-L4-CH1-Fc; VH1-L1-VH2-L2-VL2-L3-VL1-L4-CH1-Fc; VL1-L1-VL2-L2-VH2-L3-VH1-L4-CL-Fc; VH1-L1-VH2-L2-VL2-L3-VL1-L4-CL-Fc; VL1-L1-VL2-L2-VH2-L3-VH1-L4-CH1-L5-CL-Fc; VH1-L1-VH2-L2-VL2-L3-VL1-L4-CH1-L5-CL-Fc; VL1-L1-VL2-L2-VH2-L3-VH1-L4-CL-L5-CH1-Fc; VH1-L1-VH2-L2-VL2-L3-VL1-L4-CL-L5-CH1-Fc; VL1-L1-VL2-L2-VH2-L3-VH1-L4-CH1-L5-Fc; VH1-L1-VH2-L2-VL2-L3-VL1-L4-CH1-L5-Fc; VL1-L1-VL2-L2-VH2-L3-VH1-L4-CL-L5-Fc; VH1-L1-VH2-L2-VL2-L3-VL1-L4-CL-L5-Fc; VL1-L1-VL2-L2-VH2-L3-VH1-L4-CH1-L5-CL-L6-Fc; VH1-L1-VH2-L2-VL2-L3-VL1-L4-CH1-L5-CL-L6-Fc; VL1-L1-VL2-L2-VH2-L3-VH1-L4-CL-L5-CH1-L6-Fc; or VH1-L1-VH2-L2-VL2-L3-VL1-L4-CL-L5-CH1-L6-Fc; wherein VL1 is a first immunoglobulin light chain variable region that specifically binds to an HIV protein; VL2 is a second immunoglobulin light chain variable region that specifically binds to an HIV protein; VH1 is a first immunoglobulin heavy chain variable region that specifically binds to an HIV protein; VH2 is a second immunoglobulin heavy chain variable region that specifically binds to an HIV protein; CH1 is an immunoglobulin heavy chain constant region 1; CL is an immunoglobulin light chain constant region; Fc is a region comprising an immunoglobulin heavy chain constant region 2 (CH2), an immunoglobulin heavy chain constant region 3 (CH3), and optionally, an immunoglobulin hinge; and L1, L2, L3, L4, L5 and L6 are amino acid linkers.

Provided herein is an antigen binding polypeptide complex comprising a first polypeptide and a second polypeptide; wherein the first polypeptide has a structure represented by VL1-VL2-VH2-VH1; VH1-VH2-VL2-VL1; VL1-L1-VL2-L2-VH2-L3-VH1; VH1-L1-VH2-L2-VL2-L3-VL1; VL1-VL2-VH2-VH1-Fc; VH1-VH2-VL2-VL1-Fc; VL1-L1-VL2-L2-VH2-L3-VH1-Fc; VH1-L1-VH2-L2-VL2-L3-VL1-Fc; VL1-L1-VL2-L2-VH2-L3-VH1-L4-Fc; VH1-L1-VH2-L2-VL2-L3-VL1-L4-Fc; VL1-VL2-VH2-VH1-CH1; VH1-VH2-VL2-VL1-CH1; VL1-VL2-VH2-VH1-CL; VH1-VH2-VL2-VL1-CL; VL1-VL2-VH2-VH1-CH1-CL; VH1-VH2-VL2-VL1-CH1-CL; VL1-VL2-VH2-VH1-CL-CH1; VH1-VH2-VL2-VL1-CL-CH1; VL1-L1-VL2-L2-VH2-L3-VH1-L4-CH1; VH1-L1-VH2-L2-VL2-L3-VL1-L4-CH1; VL1-L1-VL2-L2-VH2-L3-VH1-L4-CL; VH1-L1-VH2-L2-VL2-L3-VL1-L4-CL; VL1-L1-VL2-L2-VH2-L3-VH1-L4-CH1-L5-CL; VH1-L1-VH2-L2-VL2-L3-VL1-L4-CH1-L5-CL; VL1-L1-VL2-L2-VH2-L3-VH1-L4-CL-L5-CH1; VH1-L1-VH2-L2-VL2-L3-VL1-L4-CL-L5-CH1; VL1-VL2-VH2-VH1-CH1-Fc; VH1-VH2-VL2-VL1-CH1-Fc; VL1-VL2-VH2-VH1-CL-Fc; VH1-VH2-VL2-VL1-CL-Fc; VL1-VL2-VH2-VH1-CH1-CL-Fc; VH1-VH2-VL2-VL1-CH1-CL-Fc; VL1-VL2-VH2-VH1-CL-CH1-Fc; VH1-VH2-VL2-VL1-CL-CH1-Fc; VL1-L1-VL2-L2-VH2-L3-VH1-L4-CH1-Fc; VH1-L1-VH2-L2-VL2-L3-VL1-L4-CH1-Fc; VL1-L1-VL2-L2-VH2-L3-VH1-L4-CL-Fc; VH1-L1-VH2-L2-VL2-L3-VL1-L4-CL-Fc; VL1-L1-VL2-L2-VH2-L3-VH1-L4-CH1-L5-CL-Fc; VH1-L1-VH2-L2-VL2-L3-VL1-L4-CH1-L5-CL-Fc; VL1-L1-VL2-L2-VH2-L3-VH1-L4-CL-L5-CH1-Fc; VH1-L1-VH2-L2-VL2-L3-VL1-L4-CL-L5-CH1-Fc; VL1-L1-VL2-L2-VH2-L3-VH1-L4-CH1-L5-Fc; VH1-L1-VH2-L2-VL2-L3-VL1-L4-CH1-L5-Fc; VL1-L1-VL2-L2-VH2-L3-VH1-L4-CL-L5-Fc; VH1-L1-VH2-L2-VL2-L3-VL1-L4-CL-L5-Fc; VL1-L1-VL2-L2-VH2-L3-VH1-L4-CH1-L5-CL-L6-Fc; VH1-L1-VH2-L2-VL2-L3-VL1-L4-CH1-L5-CL-L6-Fc; VL1-L1-VL2-L2-VH2-L3-VH1-L4-CL-L5-CH1-L6-Fc; or VH1-L1-VH2-L2-VL2-L3-VL1-L4-CL-L5-CH1-L6-Fc; wherein the second polypeptide has a structure represented by Fc; VL1-VL2-VH2-VH1; VH1-VH2-VL2-VL1; VL1-L1-VL2-L2-VH2-L3-VH1; VH1-L1-VH2-L2-VL2-L3-VL1; VL1-VL2-VH2-VH1-Fc; VH1-VH2-VL2-VL1-Fc; VL1-L1-VL2-L2-VH2-L3-VH1-Fc; VH1-L1-VH2-L2-VL2-L3-VL1-Fc; VL1-L1-VL2-L2-VH2-L3-VH1-L4-Fc; VH1-L1-VH2-L2-VL2-L3-VL1-L4-Fc; VL1-VL2-VH2-VH1-CH1; VH1-VH2-VL2-VL1-CH1; VL1-VL2-VH2-VH1-CL; VH1-VH2-VL2-VL1-CL; VL1-VL2-VH2-VH1-CH1-CL; VH1-VH2-VL2-VL1-CH1-CL; VL1-VL2-VH2-VH1-CL-CH1; VH1-VH2-VL2-VL1-CL-CH1; VL1-L1-VL2-L2-VH2-L3-VH1-L4-CH1; VH1-L1-VH2-L2-VL2-L3-VL1-L4-CH1; VL1-L1-VL2-L2-VH2-L3-VH1-L4-CL; VH1-L1-VH2-L2-VL2-L3-VL1-L4-CL; VL1-L1-VL2-L2-VH2-L3-VH1-L4-CH1-L5-CL; VH1-L1-VH2-L2-VL2-L3-VL1-L4-CH1-L5-CL; VL1-L1-VL2-L2-VH2-L3-VH1-L4-CL-L5-CH1; VH1-L1-VH2-L2-VL2-L3-VL1-L4-CL-L5-CH1; VL1-VL2-VH2-VH1-CH1-Fc; VH1-VH2-VL2-VL1-CH1-Fc; VL1-VL2-VH2-VH1-CL-Fc; VH1-VH2-VL2-VL1-CL-Fc; VL1-VL2-VH2-VH1-CH1-CL-Fc; VH1-VH2-VL2-VL1-CH1-CL-Fc; VL1-VL2-VH2-VH1-CL-CH1-Fc; VH1-VH2-VL2-VL1-CL-CH1-Fc; VL1-L1-VL2-L2-VH2-L3-VH1-L4-CH1-Fc; VH1-L1-VH2-L2-VL2-L3-VL1-L4-CH1-Fc; VL1-L1-VL2-L2-VH2-L3-VH1-L4-CL-Fc; VH1-L1-VH2-L2-VL2-L3-VL1-L4-CL-Fc; VL1-L1-VL2-L2-VH2-L3-VH1-L4-CH1-L5-CL-Fc; VH1-L1-VH2-L2-VL2-L3-VL1-L4-CH1-L5-CL-Fc; VL1-L1-VL2-L2-VH2-L3-VH1-L4-CL-L5-CH1-Fc; VH1-L1-VH2-L2-VL2-L3-VL1-L4-CL-L5-CH1-Fc; VL1-L1-VL2-L2-VH2-L3-VH1-L4-CH1-L5-Fc; VH1-L1-VH2-L2-VL2-L3-VL1-L4-CH1-L5-Fc; VL1-L1-VL2-L2-VH2-L3-VH1-L4-CL-L5-Fc; VH1-L1-VH2-L2-VL2-L3-VL1-L4-CL-L5-Fc; VL1-L1-VL2-L2-VH2-L3-VH1-L4-CH1-L5-CL-L6-Fc; VH1-L1-VH2-L2-VL2-L3-VL1-L4-CH1-L5-CL-L6-Fc; VL1-L1-VL2-L2-VH2-L3-VH1-L4-CL-L5-CH1-L6-Fc; or VH1-L1-VH2-L2-VL2-L3-VL1-L4-CL-L5-CH1-L6-Fc; wherein VL1 is a first immunoglobulin light chain variable region that specifically binds to an HIV protein; VL2 is a second immunoglobulin light chain variable region that specifically binds to an HIV protein; VH1 is a first immunoglobulin heavy chain variable region that specifically binds to an HIV protein; VH2 is a second immunoglobulin heavy chain variable region that specifically binds to an HIV protein; CH1 is an immunoglobulin heavy chain constant region 1; CL is an immunoglobulin light chain constant region; Fc is a region comprising an immunoglobulin heavy chain constant region 2 (CH2), an immunoglobulin heavy chain constant region 3 (CH3), and optionally, an immunoglobulin hinge; and L1, L2, L3, L4, L5 and L6 are amino acid linkers.

Provided herein is an antigen binding polypeptide or antigen binding polypeptide complex comprising a polypeptide having a structure represented by VL1-VL2-VH2-VH1-Fc-Fc; VH1-VH2-VL2-VL1-Fc-Fc; VL1-L1-VL2-L2-VH2-L3-VH1-Fc-Fc; VH1-L1-VH2-L2-VL2-L3-VL1-Fc-Fc; VL1-L1-VL2-L2-VH2-L3-VH1-L4-Fc-Fc; VH1-L1-VH2-L2-VL2-L3-VL1-L4-Fc-Fc; VL1-L1-VL2-L2-VH2-L3-VH1-L4-Fc-L5-Fc; or VH1-L1-VH2-L2-VL2-L3-VL1-L4-Fc-L5-Fc; wherein VL1 is a first immunoglobulin light chain variable region that specifically binds to an HIV protein; VL2 is a second immunoglobulin light chain variable region that specifically binds to an HIV protein; VH1 is a first immunoglobulin heavy chain variable region that specifically binds to an HIV protein; VH2 is a second immunoglobulin heavy chain variable region that specifically binds to an HIV protein; Fc is a region comprising an immunoglobulin heavy chain constant region 2 (CH2), an immunoglobulin heavy chain constant region 3 (CH3), and optionally, an immunoglobulin hinge; and L1, L2, L3, L4 and L5 are amino acid linkers.

Provided herein is an antigen binding polypeptide or antigen binding polypeptide complex comprising a polypeptide having a structure represented by VL1-VL2-VH2-VH1-CH3; VH1-VH2-VL2-VL1-CH3; VL1-L1-VL2-L2-VH2-L3-VH1-CH3; VH1-L1-VH2-L2-VL2-L3-VL1-CH3; VL1-L1-VL2-L2-VH2-L3-VH1-L4-CH3; VH1-L1-VH2-L2-VL2-L3-VL1-L4-CH3; VL1-VL2-VH2-VH1-CH3-CH3; VH1-VH2-VL2-VL1-CH3-CH3; VL1-L1-VL2-L2-VH2-L3-VH1-CH3-CH3; VH1-L1-VH2-L2-VL2-L3-VL1-CH3-CH3; VL1-L1-VL2-L2-VH2-L3-VH1-L4-CH3-CH3; VH1-L1-VH2-L2-VL2-L3-VL1-L4-CH3-CH3; VL1-L1-VL2-L2-VH2-L3-VH1-L4-CH3-L5-CH3; or VH1-L1-VH2-L2-VL2-L3-VL1-L4-CH3-L5-CH3; wherein VL1 is a first immunoglobulin light chain variable region that specifically binds to an HIV protein; VL2 is a second immunoglobulin light chain variable region that specifically binds to an HIV protein; VH1 is a first immunoglobulin heavy chain variable region that specifically binds to an HIV protein; VH2 is a second immunoglobulin heavy chain variable region that specifically binds to an HIV protein; CH3 is an immunoglobulin heavy chain constant region 3; and L1, L2, L3, L4 and L5 are amino acid linkers.

Provided herein is an antigen binding polypeptide complex comprising a first polypeptide and a second polypeptide; wherein the first polypeptide has a structure represented by VL1-VL2-VH2-VH1; VH1-VH2-VL2-VL1; VL1-L1-VL2-L2-VH2-L3-VH1; or VH1-L1-VH2-L2-VL2-L3-VL1; wherein the second polypeptide has a structure represented by VL3-VL4-VH4-VH3; VH3-VH4-VL4-VL3; VL3-L4-VL4-L5-VH4-L6-VH3; or VH3-L4-VH4-L5-VL4-L6-VL3; wherein VL1 is a first immunoglobulin light chain variable region that specifically binds to an HIV protein; VL2 is a second immunoglobulin light chain variable region that specifically binds to an HIV protein; VL3 is a third immunoglobulin light chain variable region that specifically binds to an HIV protein; VL4 is a fourth immunoglobulin light chain variable region that specifically binds to an HIV protein; VH1 is a first immunoglobulin heavy chain variable region that specifically binds to an HIV protein; VH2 is a second immunoglobulin heavy chain variable region that specifically binds to an HIV protein; VH3 is a third immunoglobulin heavy chain variable region that specifically binds to an HIV protein; VH4 is a fourth immunoglobulin heavy chain variable region that specifically binds to an HIV protein; and L1, L2, L3, L4, L5 and L6 are amino acid linkers.

Provided herein is an antigen binding polypeptide complex comprising a first polypeptide and a second polypeptide; wherein the first polypeptide has a structure represented by VL1-VL2-VH2-VH1-Fc; VH1-VH2-VL2-VL1-Fc; VL1-L1-VL2-L2-VH2-L3-VH1-Fc; VH1-L1-VH2-L2-VL2-L3-VL1-Fc; VL1-L1-VL2-L2-VH2-L3-VH1-L4-Fc; or VH1-L1-VH2-L2-VL2-L3-VL1-L4-Fc; wherein the second polypeptide has a structure represented by VL3-VL4-VH4-VH3-Fc; VH3-VH4-VL4-VL3-Fc; VL3-L5-VL4-L6-VH4-L7-VH3-Fc; VH3-L5-VH4-L6-VL4-L7-VL3-Fc; VL3-L5-VL4-L6-VH4-L7-VH3-L8-Fc; or VH3-L5-VH4-L6-VL4-L7-VL3-L8-Fc; wherein VL1 is a first immunoglobulin light chain variable region that specifically binds to an HIV protein; VL2 is a second immunoglobulin light chain variable region that specifically binds to an HIV protein; VL3 is a third immunoglobulin light chain variable region that specifically binds to an HIV protein; VL4 is a fourth immunoglobulin light chain variable region that specifically binds to an HIV protein; VH1 is a first immunoglobulin heavy chain variable region that specifically binds to an HIV protein; VH2 is a second immunoglobulin heavy chain variable region that specifically binds to an HIV protein; VH3 is a third immunoglobulin heavy chain variable region that specifically binds to an HIV protein; VH4 is a fourth immunoglobulin heavy chain variable region that specifically binds to an HIV protein; Fc is a region comprising an immunoglobulin heavy chain constant region 2 (CH2), an immunoglobulin heavy chain constant region 3 (CH3), and optionally, an immunoglobulin hinge; and L1, L2, L3, L4, L5, L6, L7 and L8 are amino acid linkers.

Provided herein is an antigen binding polypeptide complex comprising a first polypeptide and a second polypeptide; wherein the first polypeptide has a structure represented by VL1-VL2-VH2-VH1-CH1; VH1-VH2-VL2-VL1-CH1; VL1-VL2-VH2-VH1-CL; VH1-VH2-VL2-VL1-CL; VL1-VL2-VH2-VH1-CH1-CL; VH1-VH2-VL2-VL1-CH1-CL; VL1-VL2-VH2-VH1-CL-CH1; VH1-VH2-VL2-VL1-CL-CH1; VL1-L1-VL2-L2-VH2-L3-VH1-L4-CH1; VH1-L1-VH2-L2-VL2-L3-VL1-L4-CH1; VL1-L1-VL2-L2-VH2-L3-VH1-L4-CL; VH1-L1-VH2-L2-VL2-L3-VL1-L4-CL; VL1-L1-VL2-L2-VH2-L3-VH1-L4-CH1-L5-CL; VH1-L1-VH2-L2-VL2-L3-VL1-L4-CH1-L5-CL; VL1-L1-VL2-L2-VH2-L3-VH1-L4-CL-L5-CH1; or VH1-L1-VH2-L2-VL2-L3-VL1-L4-CL-L5-CH1; wherein the second polypeptide has a structure represented by VL3-VL4-VH4-VH3-CH1; VH3-VH4-VL4-VL3-CH1; VL3-VL4-VH4-VH3-CL; VH3-VH4-VL4-VL3-CL; VL3-VL4-VH4-VH3-CH1-CL; VH3-VH4-VL4-VL3-CH1-CL; VL3-VL4-VH4-VH3-CL-CH1; VH3-VH4-VL4-VL3-CL-CH1; VL3-L6-VL4-L7-VH4-L8-VH3-L9-CH1; VH3-L6-VH4-L7-VL4-L8-VL3-L9-CH1; VL3-L6-VL4-L7-VH4-L8-VH3-L9-CL; VH3-L6-VH4-L7-VL4-L8-VL3-L9-CL; VL3-L6-VL4-L7-VH4-L8-VH3-L9-CH1-L10-CL; VH3-L6-VH4-L7-VL4-L8-VL3-L9-CH1-L10-CL; VL3-L6-VL4-L7-VH4-L8-VH3-L9-CL-L10-CH1; or VH3-L6-VH4-L7-VL4-L8-VL3-L9-CL-L10-CH1; wherein VL1 is a first immunoglobulin light chain variable region that specifically binds to an HIV protein; VL2 is a second immunoglobulin light chain variable region that specifically binds to an HIV protein; VL3 is a third immunoglobulin light chain variable region that specifically binds to an HIV protein; VL4 is a fourth immunoglobulin light chain variable region that specifically binds to an HIV protein; VH1 is a first immunoglobulin heavy chain variable region that specifically binds to an HIV protein; VH2 is a second immunoglobulin heavy chain variable region that specifically binds to an HIV protein; VH3 is a third immunoglobulin heavy chain variable region that specifically binds to an HIV protein; VH4 is a fourth immunoglobulin heavy chain variable region that specifically binds to an HIV protein; CH1 is an immunoglobulin heavy chain constant region 1; CL is an immunoglobulin light chain constant region; and L1, L2, L3, L4, L5, L6, L7, L8, L9 and L10 are amino acid linkers.

Provided herein is an antigen binding polypeptide complex comprising a first polypeptide and a second polypeptide; wherein the first polypeptide has a structure represented by VL1-VL2-VH2-VH1-CH1-Fc; VH1-VH2-VL2-VL1-CH1-Fc; VL1-VL2-VH2-VH1-CL-Fc; VH1-VH2-VL2-VL1-CL-Fc; VL1-VL2-VH2-VH1-CH1-CL-Fc; VH1-VH2-VL2-VL1-CH1-CL-Fc; VL1-VL2-VH2-VH1-CL-CH1-Fc; VH1-VH2-VL2-VL1-CL-CH1-Fc; VL1-L1-VL2-L2-VH2-L3-VH1-L4-CH1-Fc; VH1-L1-VH2-L2-VL2-L3-VL1-L4-CH1-Fc; VL1-L1-VL2-L2-VH2-L3-VH1-L4-CL-Fc; VH1-L1-VH2-L2-VL2-L3-VL1-L4-CL-Fc; VL1-L1-VL2-L2-VH2-L3-VH1-L4-CH1-L5-CL-Fc; VH1-L1-VH2-L2-VL2-L3-VL1-L4-CH1-L5-CL-Fc; VL1-L1-VL2-L2-VH2-L3-VH1-L4-CL-L5-CH1-Fc; VH1-L1-VH2-L2-VL2-L3-VL1-L4-CL-L5-CH1-Fc; VL1-L1-VL2-L2-VH2-L3-VH1-L4-CH1-L5-Fc; VH1-L1-VH2-L2-VL2-L3-VL1-L4-CH1-L5-Fc; VL1-L1-VL2-L2-VH2-L3-VH1-L4-CL-L5-Fc; VH1-L1-VH2-L2-VL2-L3-VL1-L4-CL-L5-Fc; VL1-L1-VL2-L2-VH2-L3-VH1-L4-CH1-L5-CL-L6-Fc; VH1-L1-VH2-L2-VL2-L3-VL1-L4-CH1-L5-CL-L6-Fc; VL1-L1-VL2-L2-VH2-L3-VH1-L4-CL-L5-CH1-L6-Fc; or VH1-L1-VH2-L2-VL2-L3-VL1-L4-CL-L5-CH1-L6-Fc; wherein the second polypeptide has a structure represented by VL3-VL4-VH4-VH3-CH1-Fc; VH3-VH4-VL4-VL3-CH1-Fc; VL3-VL4-VH4-VH3-CL-Fc; VH3-VH4-VL4-VL3-CL-Fc; VL3-VL4-VH4-VH3-CH1-CL-Fc; VH3-VH4-VL4-VL3-CH1-CL-Fc; VL3-VL4-VH4-VH3-CL-CH1-Fc; VH3-VH4-VL4-VL3-CL-CH1-Fc; VL3-L7-VL4-L8-VH4-L9-VH3-L10-CH1-Fc; VH3-L7-VH4-L8-VL4-L9-VL3-L10-CH1-Fc; VL3-L7-VL4-L8-VH4-L9-VH3-L10-CL-Fc; VH3-L7-VH4-L8-VL4-L9-VL3-L10-CL-Fc; VL3-L7-VL4-L8-VH4-L9-VH3-L10-CH1-L11-CL-Fc; VH3-L7-VH4-L8-VL4-L9-VL3-L10-CH1-L11-CL-Fc; VL3-L7-VL4-L8-VH4-L9-VH3-L10-CL-L11-CH1-Fc; VH3-L7-VH4-L8-VL4-L9-VL3-L10-CL-L11-CH1-Fc; VL3-L7-VL4-L8-VH4-L9-VH3-L10-CH1-L11-Fc; VH3-L7-VH4-L8-VL4-L9-VL3-L10-CH1-L11-Fc; VL3-L7-VL4-L8-VH4-L9-VH3-L10-CL-L11-Fc; VH3-L7-VH4-L8-VL4-L9-VL3-L10-CL-L11-Fc; VL3-L7-VL4-L8-VH4-L9-VH3-L10-CH1-L11-CL-L12-Fc; VH3-L7-VH4-L8-VL4-L9-VL3-L10-CH1-L11-CL-L12-Fc; VL3-L7-VL4-L8-VH4-L9-VH3-L10-CL-L11-CH1-L12-Fc; or VH3-L7-VH4-L8-VL4-L9-VL3-L10-CL-L11-CH1-L12-Fc; wherein VL1 is a first immunoglobulin light chain variable region that specifically binds to an HIV protein; VL2 is a second immunoglobulin light chain variable region that specifically binds to an HIV protein; VL3 is a third immunoglobulin light chain variable region that specifically binds to an HIV protein; VL4 is a fourth immunoglobulin light chain variable region that specifically binds to an HIV protein; VH1 is a first immunoglobulin heavy chain variable region that specifically binds to an HIV protein; VH2 is a second immunoglobulin heavy chain variable region that specifically binds to an HIV protein; VH3 is a third immunoglobulin heavy chain variable region that specifically binds to an HIV protein; VH4 is a fourth immunoglobulin heavy chain variable region that specifically binds to an HIV protein; Fc is a region comprising an immunoglobulin heavy chain constant region 2 (CH2), an immunoglobulin heavy chain constant region 3 (CH3), and optionally, an immunoglobulin hinge; CH1 is an immunoglobulin heavy chain constant region 1; CL is an immunoglobulin light chain constant region; and L1, L2, L3, L4, L5, L6, L7, L8, L9, L10, L11 and L12 are amino acid linkers.

Provided herein is an antigen binding polypeptide complex comprising a first polypeptide and a second polypeptide; wherein the first polypeptide has a structure represented by VL1-VL2-VH2-VH1; VH1-VH2-VL2-VL1; VL1-VL2-VH2-VH1-Fc; VH1-VH2-VL2-VL1-Fc; VL1-VL2-VH2-VH1-CH1; VH1-VH2-VL2-VL1-CH1; VL1-VL2-VH2-VH1-CL; VH1-VH2-VL2-VL1-CL; VL1-VL2-VH2-VH1-CH1-CL; VH1-VH2-VL2-VL1-CH1-CL; VL1-VL2-VH2-VH1-CL-CH1; VH1-VH2-VL2-VL1-CL-CH1; VL1-VL2-VH2-VH1-CH1-Fc; VH1-VH2-VL2-VL1-CH1-Fc; VL1-VL2-VH2-VH1-CL-Fc; VH1-VH2-VL2-VL1-CL-Fc; VL1-VL2-VH2-VH1-CH1-CL-Fc; VH1-VH2-VL2-VL1-CH1-CL-Fc; VL1-VL2-VH2-VH1-CL-CH1-Fc; VH1-VH2-VL2-VL1-CL-CH1-Fc; VL1-L1-VL2-L2-VH2-L3-VH1; VH1-L1-VH2-L2-VL2-L3-VL1; VL1-L1-VL2-L2-VH2-L3-VH1-Fc; VH1-L1-VH2-L2-VL2-L3-VL1-Fc; VL1-L1-VL2-L2-VH2-L3-VH1-L4-Fc; VH1-L1-VH2-L2-VL2-L3-VL1-L4-Fc; VL1-L1-VL2-L2-VH2-L3-VH1-L4-CH1; VH1-L1-VH2-L2-VL2-L3-VL1-L4-CH1; VL1-L1-VL2-L2-VH2-L3-VH1-L4-CL; VH1-L1-VH2-L2-VL2-L3-VL1-L4-CL; VL1-L1-VL2-L2-VH2-L3-VH1-L4-CH1-L5-CL; VH1-L1-VH2-L2-VL2-L3-VL1-L4-CH1-L5-CL; VL1-L1-VL2-L2-VH2-L3-VH1-L4-CL-L5-CH1; VH1-L1-VH2-L2-VL2-L3-VL1-L4-CL-L5-CH1; VL1-L1-VL2-L2-VH2-L3-VH1-L4-CH1-Fc; VH1-L1-VH2-L2-VL2-L3-VL1-L4-CH1-Fc; VL1-L1-VL2-L2-VH2-L3-VH1-L4-CL-Fc; VH1-L1-VH2-L2-VL2-L3-VL1-L4-CL-Fc; VL1-L1-VL2-L2-VH2-L3-VH1-L4-CH1-L5-CL-Fc; VH1-L1-VH2-L2-VL2-L3-VL1-L4-CH1-L5-CL-Fc; VL1-L1-VL2-L2-VH2-L3-VH1-L4-CL-L5-CH1-Fc; VH1-L1-VH2-L2-VL2-L3-VL1-L4-CL-L5-CH1-Fc; VL1-L1-VL2-L2-VH2-L3-VH1-L4-CH1-L5-Fc; VH1-L1-VH2-L2-VL2-L3-VL1-L4-CH1-L5-Fc; VL1-L1-VL2-L2-VH2-L3-VH1-L4-CL-L5-Fc; VH1-L1-VH2-L2-VL2-L3-VL1-L4-CL-L5-Fc; VL1-L1-VL2-L2-VH2-L3-VH1-L4-CH1-L5-CL-L6-Fc; VH1-L1-VH2-L2-VL2-L3-VL1-L4-CH1-L5-CL-L6-Fc; VL1-L1-VL2-L2-VH2-L3-VH1-L4-CL-L5-CH1-L6-Fc; or VH1-L1-VH2-L2-VL2-L3-VL1-L4-CL-L5-CH1-L6-Fc; wherein the second polypeptide has a structure represented by VL3-VL4-VH4-VH3; VH3-VH4-VL4-VL3; VL3-VL4-VH4-VH3-Fc; VH3-VH4-VL4-VL3-Fc; VL3-VL4-VH4-VH3-CH1; VH3-VH4-VL4-VL3-CH1; VL3-VL4-VH4-VH3-CL; VH3-VH4-VL4-VL3-CL; VL3-VL4-VH4-VH3-CH1-CL; VH3-VH4-VL4-VL3-CH1-CL; VL3-VL4-VH4-VH3-CL-CH1; VH3-VH4-VL4-VL3-CL-CH1; VL3-VL4-VH4-VH3-CH1-Fc; VH3-VH4-VL4-VL3-CH1-Fc; VL3-VL4-VH4-VH3-CL-Fc; VH3-VH4-VL4-VL3-CL-Fc; VL3-VL4-VH4-VH3-CH1-CL-Fc; VH3-VH4-VL4-VL3-CH1-CL-Fc; VL3-VL4-VH4-VH3-CL-CH1-Fc; VH3-VH4-VL4-VL3-CL-CH1-Fc; VL3-L7-VL4-L8-VH4-L9-VH3; VH3-L7-VH4-L8-VL4-L9-VL3; VL3-L7-VL4-L8-VH4-L9-VH3-Fc; VH3-L7-VH4-L8-VL4-L9-VL3-Fc; VL3-L7-VL4-L8-VH4-L9-VH3-L10-Fc; VH3-L7-VH4-L8-VL4-L9-VL3-L10-Fc; VL3-L7-VL4-L8-VH4-L9-VH3-L10-CH1; VH3-L7-VH4-L8-VL4-L9-VL3-L10-CH1; VL3-L7-VL4-L8-VH4-L9-VH3-L10-CL; VH3-L7-VH4-L8-VL4-L9-VL3-L10-CL; VL3-L7-VL4-L8-VH4-L9-VH3-L10-CH1-L11-CL; VH3-L7-VH4-L8-VL4-L9-VL3-L10-CH1-L11-CL; VL3-L7-VL4-L8-VH4-L9-VH3-L10-CL-L11-CH1; VH3-L7-VH4-L8-VL4-L9-VL3-L10-CL-L11-CH1; VL3-L7-VL4-L8-VH4-L9-VH3-L10-CH1-Fc; VH3-L7-VH4-L8-VL4-L9-VL3-L10-CH1-Fc; VL3-L7-VL4-L8-VH4-L9-VH3-L10-CL-Fc; VH3-L7-VH4-L8-VL4-L9-VL3-L10-CL-Fc; VL3-L7-VL4-L8-VH4-L9-VH3-L10-CH1-L11-CL-Fc; VH3-L7-VH4-L8-VL4-L9-VL3-L10-CH1-L11-CL-Fc; VL3-L7-VL4-L8-VH4-L9-VH3-L10-CL-L11-CH1-Fc; VH3-L7-VH4-L8-VL4-L9-VL3-L10-CL-L11-CH1-Fc; VL3-L7-VL4-L8-VH4-L9-VH3-L10-CH1-L11-Fc; VH3-L7-VH4-L8-VL4-L9-VL3-L10-CH1-L11-Fc; VL3-L7-VL4-L8-VH4-L9-VH3-L10-CL-L11-Fc; VH3-L7-VH4-L8-VL4-L9-VL3-L10-CL-L11-Fc; VL3-L7-VL4-L8-VH4-L9-VH3-L10-CH1-L11-CL-L12-Fc; VH3-L7-VH4-L8-VL4-L9-VL3-L10-CH1-L11-CL-L12-Fc; VL3-L7-VL4-L8-VH4-L9-VH3-L10-CL-L11-CH1-L12-Fc; or VH3-L7-VH4-L8-VL4-L9-VL3-L10-CL-L11-CH1-L12-Fc; wherein VL1 is a first immunoglobulin light chain variable region that specifically binds to an HIV protein; VL2 is a second immunoglobulin light chain variable region that specifically binds to an HIV protein; VL3 is a third immunoglobulin light chain variable region that specifically binds to an HIV protein; VL4 is a fourth immunoglobulin light chain variable region that specifically binds to an HIV protein; VH1 is a first immunoglobulin heavy chain variable region that specifically binds to an HIV protein; VH2 is a second immunoglobulin heavy chain variable region that specifically binds to an HIV protein; VH3 is a third immunoglobulin heavy chain variable region that specifically binds to an HIV protein; VH4 is a fourth immunoglobulin heavy chain variable region that specifically binds to an HIV protein; Fc is a region comprising an immunoglobulin heavy chain constant region 2 (CH2), an immunoglobulin heavy chain constant region 3 (CH3), and optionally, an immunoglobulin hinge; CH1 is an immunoglobulin heavy chain constant region 1; CL is an immunoglobulin light chain constant region; and L1, L2, L3, L4, L5, L6, L7, L8, L9, L10, L11 and L12 are amino acid linkers.

Provided herein is an antigen binding polypeptide complex comprising a first polypeptide and a second polypeptide; wherein the first polypeptide has a structure represented by VL1-VL2-VH2-VH1; VH1-VH2-VL2-VL1; VL1-L1-VL2-L2-VH2-L3-VH1; or VH1-L1-VH2-L2-VL2-L3-VL1; wherein the second polypeptide has a structure represented by VL3-VH3; VH3-VL3; VL3-L4-VH3; or VH3-L4-VL3; wherein VL1 is a first immunoglobulin light chain variable region that specifically binds to an HIV protein; VL2 is a second immunoglobulin light chain variable region that specifically binds to an HIV protein; VL3 is a third immunoglobulin light chain variable region that specifically binds to an HIV protein; VH1 is a first immunoglobulin heavy chain variable region that specifically binds to an HIV protein; VH2 is a second immunoglobulin heavy chain variable region that specifically binds to an HIV protein; VH3 is a third immunoglobulin heavy chain variable region that specifically binds to an HIV protein; and L1, L2, L3 and L4 are amino acid linkers.

Provided herein is an antigen binding polypeptide complex comprising a first polypeptide and a second polypeptide; wherein the first polypeptide has a structure represented by VL1-VL2-VH2-VH1-Fc; VH1-VH2-VL2-VL1-Fc; VL1-L1-VL2-L2-VH2-L3-VH1-Fc; VH1-L1-VH2-L2-VL2-L3-VL1-Fc; VL1-L1-VL2-L2-VH2-L3-VH1-L4-Fc; or VH1-L1-VH2-L2-VL2-L3-VL1-L4-Fc; wherein the second polypeptide has a structure represented by VL3-VH3-Fc; VH3-VL3-Fc; VL3-L5-VH3-Fc; VH3-L5-VL3-Fc; VL3-L5-VH3-L6-Fc; or VH3-L5-VL3-L6-Fc; wherein VL1 is a first immunoglobulin light chain variable region that specifically binds to an HIV protein; VL2 is a second immunoglobulin light chain variable region that specifically binds to an HIV protein; VL3 is a third immunoglobulin light chain variable region that specifically binds to an HIV protein; VH1 is a first immunoglobulin heavy chain variable region that specifically binds to an HIV protein; VH2 is a second immunoglobulin heavy chain variable region that specifically binds to an HIV protein; VH3 is a third immunoglobulin heavy chain variable region that specifically binds to an HIV protein; Fc is a region comprising an immunoglobulin heavy chain constant region 2 (CH2), an immunoglobulin heavy chain constant region 3 (CH3), and optionally, an immunoglobulin hinge; and L1, L2, L3, L4, L5 and L6 are amino acid linkers.

Provided herein is an antigen binding polypeptide complex comprising a first polypeptide and a second polypeptide; wherein the first polypeptide has a structure represented by VL1-VL2-VH2-VH1-CH1; VH1-VH2-VL2-VL1-CH1; VL1-VL2-VH2-VH1-CL; VH1-VH2-VL2-VL1-CL; VL1-VL2-VH2-VH1-CH1-CL; VH1-VH2-VL2-VL1-CH1-CL; VL1-VL2-VH2-VH1-CL-CH1; VH1-VH2-VL2-VL1-CL-CH1; VL1-L1-VL2-L2-VH2-L3-VH1-L4-CH1; VH1-L1-VH2-L2-VL2-L3-VL1-L4-CH1; VL1-L1-VL2-L2-VH2-L3-VH1-L4-CL; VH1-L1-VH2-L2-VL2-L3-VL1-L4-CL; VL1-L1-VL2-L2-VH2-L3-VH1-L4-CH1-L5-CL; VH1-L1-VH2-L2-VL2-L3-VL1-L4-CH1-L5-CL; VL1-L1-VL2-L2-VH2-L3-VH1-L4-CL-L5-CH1; or VH1-L1-VH2-L2-VL2-L3-VL1-L4-CL-L5-CH1; wherein the second polypeptide has a structure represented by VL3-VH3-CH1; VH3-VL3-CH1; VL3-VH3-CL; VH3-VL3-CL; VL3-VH3-CH1-CL; VH3-VL3-CH1-CL; VL3-VH3-CL-CH1; VH3-VL3-CL-CH1; VL3-CL-VH3-CH1; VL3-CH1-VH3-CL; VH3-CH1-VL3-CL; VH3-CL-VL3-CH1; VL3-L6-VH3-L7-CH1; VH3-L6-VL3-L7-CH1; VL3-L6-VH3-L7-CL; VH3-L6-VL3-L7-CL; VL3-L6-VH3-L7-CH1-L8-CL; VH3-L6-VL3-L7-CH1-L8-CL; VL3-L6-VH3-L7-CL-L8-CH1; VH3-L6-VL3-L7-CL-L8-CH1; VL3-L6-CL-L7-VH3-L8-CH1; VL3-L6-CH1-L7-VH3-L8-CL; VH3-L6-CH1-L7-VL3-L8-CL; VH3-L6-CL-L7-VL3-L8-CH1; VL3-VH3-L6-CH1-CL; VH3-VL3-L6-CH1-CL; VL3-VH3-L6-CL-CH1; VH3-VL3-L6-CL-CH1; VL3-CL-L6-VH3-CH1; VL3-CH1-L6-VH3-CL; VH3-CH1-L6-VL3-CL; or VH3-CL-L6-VL3-CH1; wherein VL1 is a first immunoglobulin light chain variable region that specifically binds to an HIV protein; VL2 is a second immunoglobulin light chain variable region that specifically binds to an HIV protein; VL3 is a third immunoglobulin light chain variable region that specifically binds to an HIV protein; VH1 is a first immunoglobulin heavy chain variable region that specifically binds to an HIV protein; VH2 is a second immunoglobulin heavy chain variable region that specifically binds to an HIV protein; VH3 is a third immunoglobulin heavy chain variable region that specifically binds to an HIV protein; CH1 is an immunoglobulin heavy chain constant region 1; CL is an immunoglobulin light chain constant region; and L1, L2, L3, L4, L5, L6, L7 and L8 are amino acid linkers.

Provided herein is an antigen binding polypeptide complex comprising a first polypeptide and a second polypeptide; wherein the first polypeptide has a structure represented by VL1-VL2-VH2-VH1-CH1-Fc; VH1-VH2-VL2-VL1-CH1-Fc; VL1-VL2-VH2-VH1-CL-Fc; VH1-VH2-VL2-VL1-CL-Fc; VL1-VL2-VH2-VH1-CH1-CL-Fc; VH1-VH2-VL2-VL1-CH1-CL-Fc; VL1-VL2-VH2-VH1-CL-CH1-Fc; VH1-VH2-VL2-VL1-CL-CH1-Fc; VL1-L1-VL2-L2-VH2-L3-VH1-L4-CH1-Fc; VH1-L1-VH2-L2-VL2-L3-VL1-L4-CH1-Fc; VL1-L1-VL2-L2-VH2-L3-VH1-L4-CL-Fc; VH1-L1-VH2-L2-VL2-L3-VL1-L4-CL-Fc; VL1-L1-VL2-L2-VH2-L3-VH1-L4-CH1-L5-CL-Fc; VH1-L1-VH2-L2-VL2-L3-VL1-L4-CH1-L5-CL-Fc; VL1-L1-VL2-L2-VH2-L3-VH1-L4-CL-L5-CH1-Fc; VH1-L1-VH2-L2-VL2-L3-VL1-L4-CL-L5-CH1-Fc; VL1-L1-VL2-L2-VH2-L3-VH1-L4-CH1-L5-Fc; VH1-L1-VH2-L2-VL2-L3-VL1-L4-CH1-L5-Fc; VL1-L1-VL2-L2-VH2-L3-VH1-L4-CL-L5-Fc; VH1-L1-VH2-L2-VL2-L3-VL1-L4-CL-L5-Fc; VL1-L1-VL2-L2-VH2-L3-VH1-L4-CH1-L5-CL-L6-Fc; VH1-L1-VH2-L2-VL2-L3-VL1-L4-CH1-L5-CL-L6-Fc; VL1-L1-VL2-L2-VH2-L3-VH1-L4-CL-L5-CH1-L6-Fc; or VH1-L1-VH2-L2-VL2-L3-VL1-L4-CL-L5-CH1-L6-Fc; wherein the second polypeptide has a structure represented by VL3-VH3-CH1-Fc; VH3-VL3-CH1-Fc; VL3-VH3-CL-Fc; VH3-VL3-CL-Fc; VL3-VH3-CH1-CL-Fc; VH3-VL3-CH1-CL-Fc; VL3-VH3-CL-CH1-Fc; VH3-VL3-CL-CH1-Fc; VL3-CL-VH3-CH1-Fc; VL3-CH1-VH3-CL-Fc; VH3-CH1-VL3-CL-Fc; VH3-CL-VL3-CH1-Fc; VL3-L7-VH3-L8-CH1-Fc; VH3-L7-VL3-L8-CH1-Fc; VL3-L7-VH3-L8-CL-Fc; VH3-L7-VL3-L8-CL-Fc; VL3-L7-VH3-L8-CH1-L9-CL-Fc; VH3-L7-VL3-L8-CH1-L9-CL-Fc; VL3-L7-VH3-L8-CL-L9-CH1-Fc; VH3-L7-VL3-L8-CL-L9-CH1-Fc; VL3-L7-CL-L8-VH3-L9-CH1-Fc; VL3-L7-CH1-L8-VH3-L9-CL-Fc; VH3-L7-CH1-L8-VL3-L9-CL-Fc; VH3-L7-CL-L8-VL3-L9-CH1-Fc; VL3-L7-VH3-L8-CH1-L9-CL-L10-Fc; VH3-L7-VL3-L8-CH1-L9-CL-L10-Fc; VL3-L7-VH3-L8-CL-L9-CH1-L10-Fc; VH3-L7-VL3-L8-CL-L9-CH1-L10-Fc; VL3-L7-CL-L8-VH3-L9-CH1-L10-Fc; VL3-L7-CH1-L8-VH3-L9-CL-L10-Fc; VH3-L7-CH1-L8-VL3-L9-CL-L10-Fc; VH3-L7-CL-L8-VL3-L9-CH1-L10-Fc; VL3-VH3-L7-CH1-CL-Fc; VH3-VL3-L7-CH1-CL-Fc; VL3-VH3-L7-CL-CH1-Fc; VH3-VL3-L7-CL-CH1-Fc; VL3-CL-L7-VH3-CH1-Fc; VL3-CH1-L7-VH3-CL-Fc; VH3-CH1-L7-VL3-CL-Fc; or VH3-CL-L7-VL3-CH1-Fc; wherein VL1 is a first immunoglobulin light chain variable region that specifically binds to an HIV protein; VL2 is a second immunoglobulin light chain variable region that specifically binds to an HIV protein; VL3 is a third immunoglobulin light chain variable region that specifically binds to an HIV protein; VH1 is a first immunoglobulin heavy chain variable region that specifically binds to an HIV protein; VH2 is a second immunoglobulin heavy chain variable region that specifically binds to an HIV protein; VH3 is a third immunoglobulin heavy chain variable region that specifically binds to an HIV protein; Fc is a region comprising an immunoglobulin heavy chain constant region 2 (CH2), an immunoglobulin heavy chain constant region 3 (CH3), and optionally, an immunoglobulin hinge; CH1 is an immunoglobulin heavy chain constant region 1; CL is an immunoglobulin light chain constant region; and L1, L2, L3, L4, L5, L6, L7, L8, L9 and L10 are amino acid linkers.

Provided herein is an antigen binding polypeptide complex comprising a first polypeptide and a second polypeptide; wherein the first polypeptide has a structure represented by VL1-VL2-VH2-VH1; VH1-VH2-VL2-VL1; VL1-L1-VL2-L2-VH2-L3-VH1; VH1-L1-VH2-L2-VL2-L3-VL1; VL1-VL2-VH2-VH1-Fc; VH1-VH2-VL2-VL1-Fc; VL1-L1-VL2-L2-VH2-L3-VH1-Fc; VH1-L1-VH2-L2-VL2-L3-VL1-Fc; VL1-L1-VL2-L2-VH2-L3-VH1-L4-Fc; VH1-L1-VH2-L2-VL2-L3-VL1-L4-Fc; VL1-VL2-VH2-VH1-CH1; VH1-VH2-VL2-VL1-CH1; VL1-VL2-VH2-VH1-CL; VH1-VH2-VL2-VL1-CL; VL1-VL2-VH2-VH1-CH1-CL; VH1-VH2-VL2-VL1-CH1-CL; VL1-VL2-VH2-VH1-CL-CH1; VH1-VH2-VL2-VL1-CL-CH1; VL1-L1-VL2-L2-VH2-L3-VH1-L4-CH1; VH1-L1-VH2-L2-VL2-L3-VL1-L4-CH1; VL1-L1-VL2-L2-VH2-L3-VH1-L4-CL; VH1-L1-VH2-L2-VL2-L3-VL1-L4-CL; VL1-L1-VL2-L2-VH2-L3-VH1-L4-CH1-L5-CL; VH1-L1-VH2-L2-VL2-L3-VL1-L4-CH1-L5-CL; VL1-L1-VL2-L2-VH2-L3-VH1-L4-CL-L5-CH1; VH1-L1-VH2-L2-VL2-L3-VL1-L4-CL-L5-CH1; VL1-VL2-VH2-VH1-CH1-Fc; VH1-VH2-VL2-VL1-CH1-Fc; VL1-VL2-VH2-VH1-CL-Fc; VH1-VH2-VL2-VL1-CL-Fc; VL1-VL2-VH2-VH1-CH1-CL-Fc; VH1-VH2-VL2-VL1-CH1-CL-Fc; VL1-VL2-VH2-VH1-CL-CH1-Fc; VH1-VH2-VL2-VL1-CL-CH1-Fc; VL1-L1-VL2-L2-VH2-L3-VH1-L4-CH1-Fc; VH1-L1-VH2-L2-VL2-L3-VL1-L4-CH1-Fc; VL1-L1-VL2-L2-VH2-L3-VH1-L4-CL-Fc; VH1-L1-VH2-L2-VL2-L3-VL1-L4-CL-Fc; VL1-L1-VL2-L2-VH2-L3-VH1-L4-CH1-L5-CL-Fc; VH1-L1-VH2-L2-VL2-L3-VL1-L4-CH1-L5-CL-Fc; VL1-L1-VL2-L2-VH2-L3-VH1-L4-CL-L5-CH1-Fc; VH1-L1-VH2-L2-VL2-L3-VL1-L4-CL-L5-CH1-Fc; VL1-L1-VL2-L2-VH2-L3-VH1-L4-CH1-L5-Fc; VH1-L1-VH2-L2-VL2-L3-VL1-L4-CH1-L5-Fc; VL1-L1-VL2-L2-VH2-L3-VH1-L4-CL-L5-Fc; VH1-L1-VH2-L2-VL2-L3-VL1-L4-CL-L5-Fc; VL1-L1-VL2-L2-VH2-L3-VH1-L4-CH1-L5-CL-L6-Fc; VH1-L1-VH2-L2-VL2-L3-VL1-L4-CH1-L5-CL-L6-Fc; VL1-L1-VL2-L2-VH2-L3-VH1-L4-CL-L5-CH1-L6-Fc; or VH1-L1-VH2-L2-VL2-L3-VL1-L4-CL-L5-CH1-L6-Fc; wherein the second polypeptide has a structure represented by VL3-VH3; VH3-VL3; VL3-L4-VH3; VH3-L4-VL3; VL3-VH3-Fc; VH3-VL3-Fc; VL3-L4-VH3-Fc; VH3-L4-VL3-Fc; VL3-VH3-CH1; VH3-VL3-CH1; VL3-VH3-CL; VH3-VL3-CL; VL3-VH3-CH1-CL; VH3-VL3-CH1-CL; VL3-VH3-CL-CH1; VH3-VL3-CL-CH1; VL3-CL-VH3-CH1; VL3-CH1-VH3-CL; VH3-CH1-VL3-CL; VH3-CL-VL3-CH1; VL3-L7-VH3-L8-CH1; VH3-L7-VL3-L8-CH1; VL3-L7-VH3-L8-CL; VH3-L7-VL3-L8-CL; VL3-L7-VH3-L8-CH1-L9-CL; VH3-L7-VL3-L8-CH1-L9-CL; VL3-L7-VH3-L8-CL-L9-CH1; VH3-L7-VL3-L8-CL-L9-CH1; VL3-L7-CL-L8-VH3-L9-CH1; VL3-L7-CH1-L8-VH3-L9-CL; VH3-L7-CH1-L8-VL3-L9-CL; VH3-L7-CL-L8-VL3-L9-CH1; VL3-VH3-L7-CH1-CL; VH3-VL3-L7-CH1-CL; VL3-VH3-L7-CL-CH1; VH3-VL3-L7-CL-CH1; VL3-CL-L7-VH3-CH1; VL3-CH1-L7-VH3-CL; VH3-CH1-L7-VL3-CL; VH3-CL-L7-VL3-CH1; VL3-VH3-CH1-Fc; VH3-VL3-CH1-Fc; VL3-VH3-CL-Fc; VH3-VL3-CL-Fc; VL3-VH3-CH1-CL-Fc; VH3-VL3-CH1-CL-Fc; VL3-VH3-CL-CH1-Fc; VH3-VL3-CL-CH1-Fc; VL3-CL-VH3-CH1-Fc; VL3-CH1-VH3-CL-Fc; VH3-CH1-VL3-CL-Fc; VH3-CL-VL3-CH1-Fc; VL3-L7-VH3-L8-CH1-Fc; VH3-L7-VL3-L8-CH1-Fc; VL3-L7-VH3-L8-CL-Fc; VH3-L7-VL3-L8-CL-Fc; VL3-L7-VH3-L8-CH1-L9-CL-Fc; VH3-L7-VL3-L8-CH1-L9-CL-Fc; VL3-L7-VH3-L8-CL-L9-CH1-Fc; VH3-L7-VL3-L8-CL-L9-CH1-Fc; VL3-L7-CL-L8-VH3-L9-CH1-Fc; VL3-L7-CH1-L8-VH3-L9-CL-Fc; VH3-L7-CH1-L8-VL3-L9-CL-Fc; VH3-L7-CL-L8-VL3-L9-CH1-Fc; VL3-L7-VH3-L8-CH1-L9-Fc; VH3-L7-VL3-L8-CH1-L9-Fc; VL3-L7-VH3-L8-CL-L9-Fc; VH3-L7-VL3-L8-CL-L9-Fc; VL3-L7-VH3-L8-CH1-L9-CL-L10-Fc; VH3-L7-VL3-L8-CH1-L9-CL-L10-Fc; VL3-L7-VH3-L8-CL-L9-CH1-L10-Fc; VH3-L7-VL3-L8-CL-L9-CH1-L10-Fc; VL3-L7-CL-L8-VH3-L9-CH1-L10-Fc; VL3-L7-CH1-L8-VH3-L9-CL-L10-Fc; VH3-L7-CH1-L8-VL3-L9-CL-L10-Fc; VH3-L7-CL-L8-VL3-L9-CH1-L10-Fc; VL3-VH3-L7-CH1-CL-Fc; VH3-VL3-L7-CH1-CL-Fc; VL3-VH3-L7-CL-CH1-Fc; VH3-VL3-L7-CL-CH1-Fc; VL3-CL-L7-VH3-CH1-Fc; VL3-CH1-L7-VH3-CL-Fc; VH3-CH1-L7-VL3-CL-Fc; or VH3-CL-L7-VL3-CH1-Fc; wherein VL1 is a first immunoglobulin light chain variable region that specifically binds to an HIV protein; VL2 is a second immunoglobulin light chain variable region that specifically binds to an HIV protein; VL3 is a third immunoglobulin light chain variable region that specifically binds to an HIV protein; VH1 is a first immunoglobulin heavy chain variable region that specifically binds to an HIV protein; VH2 is a second immunoglobulin heavy chain variable region that specifically binds to an HIV protein; VH3 is a third immunoglobulin heavy chain variable region that specifically binds to an HIV protein; Fc is a region comprising an immunoglobulin heavy chain constant region 2 (CH2), an immunoglobulin heavy chain constant region 3 (CH3), and optionally, an immunoglobulin hinge; CH1 is an immunoglobulin heavy chain constant region 1; CL is an immunoglobulin light chain constant region; and L1, L2, L3, L4, L5, L6, L7, L8, L9 and L10 are amino acid linkers.

Provided herein is an antigen binding polypeptide complex comprising a first polypeptide, a second polypeptide, and a third polypeptide; wherein the first polypeptide has a structure represented by VL1-VL2-VH2-VH1; VH1-VH2-VL2-VL1; VL1-L1-VL2-L2-VH2-L3-VH1; or VH1-L1-VH2-L2-VL2-L3-VL1; wherein the second polypeptide has a structure represented by VL3; wherein the third polypeptide has a structure represented by VH3; wherein VL1 is a first immunoglobulin light chain variable region that specifically binds to an HIV protein; VL2 is a second immunoglobulin light chain variable region that specifically binds to an HIV protein; VL3 is a third immunoglobulin light chain variable region that specifically binds to an HIV protein; VH1 is a first immunoglobulin heavy chain variable region that specifically binds to an HIV protein; VH2 is a second immunoglobulin heavy chain variable region that specifically binds to an HIV protein; VH3 is a third immunoglobulin heavy chain variable region that specifically binds to an HIV protein; and L1, L2 and L3 are amino acid linkers.

Provided herein is an antigen binding polypeptide complex comprising a first polypeptide, a second polypeptide, and a third polypeptide; wherein the first polypeptide has a structure represented by VL1-VL2-VH2-VH1-Fc; VH1-VH2-VL2-VL1-Fc; VL1-L1-VL2-L2-VH2-L3-VH1-Fc; VH1-L1-VH2-L2-VL2-L3-VL1-Fc; VL1-L1-VL2-L2-VH2-L3-VH1-L4-Fc; or VH1-L1-VH2-L2-VL2-L3-VL1-L4-Fc; wherein the second polypeptide has a structure represented by VL3; or VL3-L5; wherein the third polypeptide has a structure represented by VH3-Fc; or VH3-L6-Fc; wherein VL1 is a first immunoglobulin light chain variable region that specifically binds to an HIV protein; VL2 is a second immunoglobulin light chain variable region that specifically binds to an HIV protein; VL3 is a third immunoglobulin light chain variable region that specifically binds to an HIV protein; VH1 is a first immunoglobulin heavy chain variable region that specifically binds to an HIV protein; VH2 is a second immunoglobulin heavy chain variable region that specifically binds to an HIV protein; VH3 is a third immunoglobulin heavy chain variable region that specifically binds to an HIV protein; Fc is a region comprising an immunoglobulin heavy chain constant region 2 (CH2), an immunoglobulin heavy chain constant region 3 (CH3), and optionally, an immunoglobulin hinge; and L1, L2, L3, L4, L5 or L6 are amino acid linkers.

Provided herein is an antigen binding polypeptide complex comprising a first polypeptide, a second polypeptide, and a third polypeptide; wherein the first polypeptide has a structure represented by VL1-VL2-VH2-VH1-Fc; VH1-VH2-VL2-VL1-Fc; VL1-L1-VL2-L2-VH2-L3-VH1-Fc; VH1-L1-VH2-L2-VL2-L3-VL1-Fc; VL1-L1-VL2-L2-VH2-L3-VH1-L4-Fc; or VH1-L1-VH2-L2-VL2-L3-VL1-L4-Fc; wherein the second polypeptide has a structure represented by VL3-Fc; or VL3-L5-Fc; wherein the third polypeptide has a structure represented by VH3; or VH3-L6; wherein VL1 is a first immunoglobulin light chain variable region that specifically binds to an HIV protein; VL2 is a second immunoglobulin light chain variable region that specifically binds to an HIV protein; VL3 is a third immunoglobulin light chain variable region that specifically binds to an HIV protein; VH1 is a first immunoglobulin heavy chain variable region that specifically binds to an HIV protein; VH2 is a second immunoglobulin heavy chain variable region that specifically binds to an HIV protein; VH3 is a third immunoglobulin heavy chain variable region that specifically binds to an HIV protein; Fc is a region comprising an immunoglobulin heavy chain constant region 2 (CH2), an immunoglobulin heavy chain constant region 3 (CH3), and optionally, an immunoglobulin hinge; and L1, L2, L3, L4, L5 and L6 are amino acid linkers.

Provided herein is an antigen binding polypeptide complex comprising a first polypeptide, a second polypeptide, and a third polypeptide; wherein the first polypeptide has a structure represented by VL1-VL2-VH2-VH1-CH1; VH1-VH2-VL2-VL1-CH1; VL1-VL2-VH2-VH1-CL; VH1-VH2-VL2-VL1-CL; VL1-VL2-VH2-VH1-CH1-CL; VH1-VH2-VL2-VL1-CH1-CL; VL1-VL2-VH2-VH1-CL-CH1; VH1-VH2-VL2-VL1-CL-CH1; VL1-L1-VL2-L2-VH2-L3-VH1-L4-CH1; VH1-L1-VH2-L2-VL2-L3-VL1-L4-CH1; VL1-L1-VL2-L2-VH2-L3-VH1-L4-CL; VH1-L1-VH2-L2-VL2-L3-VL1-L4-CL; VL1-L1-VL2-L2-VH2-L3-VH1-L4-CH1-L5-CL; VH1-L1-VH2-L2-VL2-L3-VL1-L4-CH1-L5-CL; VL1-L1-VL2-L2-VH2-L3-VH1-L4-CL-L5-CH1; or VH1-L1-VH2-L2-VL2-L3-VL1-L4-CL-L5-CH1; wherein the second polypeptide has a structure represented by VL3-CH1; VL3-CL; VL3-L6-CH1; or VL3-L6-CL; wherein the third polypeptide has a structure represented by VH3-CH1; VH3-CL; VH3-L7-CH1; or VH3-L7-CL; wherein VL1 is a first immunoglobulin light chain variable region that specifically binds to an HIV protein; VL2 is a second immunoglobulin light chain variable region that specifically binds to an HIV protein; VL3 is a third immunoglobulin light chain variable region that specifically binds to an HIV protein; VH1 is a first immunoglobulin heavy chain variable region that specifically binds to an HIV protein; VH2 is a second immunoglobulin heavy chain variable region that specifically binds to an HIV protein; VH3 is a third immunoglobulin heavy chain variable region that specifically binds to an HIV protein; CH1 is an immunoglobulin heavy chain constant region 1; CL is an immunoglobulin light chain constant region; and L1, L2, L3, L4, L5, L6 and L7 are amino acid linkers.

Provided herein is an antigen binding polypeptide complex comprising a first polypeptide, a second polypeptide, and a third polypeptide; wherein the first polypeptide has a structure represented by VL1-VL2-VH2-VH1; VH1-VH2-VL2-VL1; VL1-L1-VL2-L2-VH2-L3-VH1; VH1-L1-VH2-L2-VL2-L3-VL1; VL1-VL2-VH2-VH1-Fc; VH1-VH2-VL2-VL1-Fc; VL1-L1-VL2-L2-VH2-L3-VH1-Fc; VH1-L1-VH2-L2-VL2-L3-VL1-Fc; VL1-L1-VL2-L2-VH2-L3-VH1-L4-Fc; VH1-L1-VH2-L2-VL2-L3-VL1-L4-Fc; VL1-VL2-VH2-VH1-CH1; VH1-VH2-VL2-VL1-CH1; VL1-VL2-VH2-VH1-CL; VH1-VH2-VL2-VL1-CL; VL1-VL2-VH2-VH1-CH1-CL; VH1-VH2-VL2-VL1-CH1-CL; VL1-VL2-VH2-VH1-CL-CH1; VH1-VH2-VL2-VL1-CL-CH1; VL1-L1-VL2-L2-VH2-L3-VH1-L4-CH1; VH1-L1-VH2-L2-VL2-L3-VL1-L4-CH1; VL1-L1-VL2-L2-VH2-L3-VH1-L4-CL; VH1-L1-VH2-L2-VL2-L3-VL1-L4-CL; VL1-L1-VL2-L2-VH2-L3-VH1-L4-CH1-L5-CL; VH1-L1-VH2-L2-VL2-L3-VL1-L4-CH1-L5-CL; VL1-L1-VL2-L2-VH2-L3-VH1-L4-CL-L5-CH1; VH1-L1-VH2-L2-VL2-L3-VL1-L4-CL-L5-CH1; VL1-VL2-VH2-VH1-CH1-Fc; VH1-VH2-VL2-VL1-CH1-Fc; VL1-VL2-VH2-VH1-CL-Fc; VH1-VH2-VL2-VL1-CL-Fc; VL1-VL2-VH2-VH1-CH1-CL-Fc; VH1-VH2-VL2-VL1-CH1-CL-Fc; VL1-VL2-VH2-VH1-CL-CH1-Fc; VH1-VH2-VL2-VL1-CL-CH1-Fc; VL1-L1-VL2-L2-VH2-L3-VH1-L4-CH1-Fc; VH1-L1-VH2-L2-VL2-L3-VL1-L4-CH1-Fc; VL1-L1-VL2-L2-VH2-L3-VH1-L4-CL-Fc; VH1-L1-VH2-L2-VL2-L3-VL1-L4-CL-Fc; VL1-L1-VL2-L2-VH2-L3-VH1-L4-CH1-L5-CL-Fc; VH1-L1-VH2-L2-VL2-L3-VL1-L4-CH1-L5-CL-Fc; VL1-L1-VL2-L2-VH2-L3-VH1-L4-CL-L5-CH1-Fc; VH1-L1-VH2-L2-VL2-L3-VL1-L4-CL-L5-CH1-Fc; VL1-L1-VL2-L2-VH2-L3-VH1-L4-CH1-L5-Fc; VH1-L1-VH2-L2-VL2-L3-VL1-L4-CH1-L5-Fc; VL1-L1-VL2-L2-VH2-L3-VH1-L4-CL-L5-Fc; VH1-L1-VH2-L2-VL2-L3-VL1-L4-CL-L5-Fc; VL1-L1-VL2-L2-VH2-L3-VH1-L4-CH1-L5-CL-L6-Fc; VH1-L1-VH2-L2-VL2-L3-VL1-L4-CH1-L5-CL-L6-Fc; VL1-L1-VL2-L2-VH2-L3-VH1-L4-CL-L5-CH1-L6-Fc; or VH1-L1-VH2-L2-VL2-L3-VL1-L4-CL-L5-CH1-L6-Fc; wherein the second polypeptide has a structure represented by VL3; VL3-Fc; VL3-CH1; VL3-CL; VL3-CH1-CL; VL3-CL-CH1; VL3-CH1-Fc; VL3-CL-Fc; VL3-CH1-CL-Fc; VL3-CL-CH1-Fc; VL3-L7-Fc; VL3-L7-CH1; VL3-L7-CL; VL3-L7-CH1-L8-CL; VL3-L7-CL-L8-CH1; VL3-L7-CH1-L8-Fc; VL3-L7-CL-L8-Fc; VL3-L7-CH1-L8-CL-Fc; VL3-L7-CL-L8-CH1-Fc; VL3-L7-CH1-L8-CL-L9-Fc; or VL3-L7-CL-L8-CH1-L9-Fc; wherein the third polypeptide has a structure represented by VH3; VH3-Fc; VH3-CH1; VH3-CL; VH3-CH1-CL; VH3-CL-CH1; VH3-CH1-Fc; VH3-CL-Fc; VH3-CH1-CL-Fc; VH3-CL-CH1-Fc; VH3-L10-Fc; VH3-L10-CH1; VH3-L10-CL; VH3-L10-CH1-L11-CL; VH3-L10-CL-L11-CH1; VH3-L10-CH1-L11-Fc; VH3-L10-CL-L11-Fc; VH3-L10-CH1-L11-CL-Fc; VH3-L10-CL-L11-CH1-Fc; VH3-L10-CH1-L11-CL-L12-Fc; or VH3-L10-CL-L11-CH1-L12-Fc; wherein VL1 is a first immunoglobulin light chain variable region that specifically binds to an HIV protein; VL2 is a second immunoglobulin light chain variable region that specifically binds to an HIV protein; VL3 is a third immunoglobulin light chain variable region that specifically binds to an HIV protein; VH1 is a first immunoglobulin heavy chain variable region that specifically binds to an HIV protein; VH2 is a second immunoglobulin heavy chain variable region that specifically binds to an HIV protein; VH3 is a third immunoglobulin heavy chain variable region that specifically binds to an HIV protein; Fc is a region comprising an immunoglobulin heavy chain constant region 2 (CH2), an immunoglobulin heavy chain constant region 3 (CH3), and optionally, an immunoglobulin hinge; CH1 is an immunoglobulin heavy chain constant region 1; CL is an immunoglobulin light chain constant region; and L1, L2, L3, L4, L5, L6, L7, L8, L9, L10, L11 and L12 are amino acid linkers.

Provided herein is an antigen binding polypeptide having a structure represented by VL1-VL2-VL3-VH3-VH2-VH1; VH1-VH2-VH3-VL3-VL2-VL1; VL1-VH2-VL3-VH3-VL2-VH1; VH1-VL2-VH3-VL3-VH2-VL1; VL1-VL2-VH3-VL3-VH2-VH1; VH1-VH2-VL3-VH3-VL2-VL1; VL1-VH2-VH3-VL3-VL2-VH1; VH1-VL2-VL3-VH3-VH2-VL1; VL1-L1-VL2-L2-VL3-L3-VH3-L4-VH2-L5-VH1; VH1-L1-VH2-L2-VH3-L3-VL3-L4-VL2-L5-VL1; VL1-L1-VH2-L2-VL3-L3-VH3-L4-VL2-L5-VH1; VH1-L1-VL2-L2-VH3-L3-VL3-L4-VH2-L5-VL1; VL1-L1-VL2-L2-VH3-L3-VL3-L4-VH2-L5-VH1; VH1-L1-VH2-L2-VL3-L3-VH3-L4-VL2-L5-VL1; VL1-L1-VH2-L2-VH3-L3-VL3-L4-VL2-L5-VH1; or VH1-L1-VL2-L2-VL3-L3-VH3-L4-VH2-L5-VL1; wherein VL1 is a first immunoglobulin light chain variable region that specifically binds to an HIV protein; VL2 is a second immunoglobulin light chain variable region that specifically binds to an HIV protein; VL3 is a third immunoglobulin light chain variable region that specifically binds to an HIV protein; VH1 is a first immunoglobulin heavy chain variable region that specifically binds to an HIV protein; VH2 is a second immunoglobulin heavy chain variable region that specifically binds to an HIV protein; VH3 is a third immunoglobulin heavy chain variable region that specifically binds to an HIV protein; and L1, L2, L3, L4 and L5 are amino acid linkers.

Provided herein is an antigen binding polypeptide complex comprising a first polypeptide and a second polypeptide; wherein the first polypeptide has a structure represented by VL1-VL2-VL3-VH3-VH2-VH1; VH1-VH2-VH3-VL3-VL2-VL1; VL1-VH2-VL3-VH3-VL2-VH1; VH1-VL2-VH3-VL3-VH2-VL1; VL1-VL2-VH3-VL3-VH2-VH1; VH1-VH2-VL3-VH3-VL2-VL1; VL1-VH2-VH3-VL3-VL2-VH1; VH1-VL2-VL3-VH3-VH2-VL1; VL1-L1-VL2-L2-VL3-L3-VH3-L4-VH2-L5-VH1; VH1-L1-VH2-L2-VH3-L3-VL3-L4-VL2-L5-VL1; VL1-L1-VH2-L2-VL3-L3-VH3-L4-VL2-L5-VH1; VH1-L1-VL2-L2-VH3-L3-VL3-L4-VH2-L5-VL1; VL1-L1-VL2-L2-VH3-L3-VL3-L4-VH2-L5-VH1; VH1-L1-VH2-L2-VL3-L3-VH3-L4-VL2-L5-VL1; VL1-L1-VH2-L2-VH3-L3-VL3-L4-VL2-L5-VH1; or VH1-L1-VL2-L2-VL3-L3-VH3-L4-VH2-L5-VL1; wherein the second polypeptide has a structure represented by VL4-VH4; VH4-VL4; VL4-L6-VH4; VH4-L6-VL4; VL4-VL5-VH5-VH4; VH4-VH5-VL5-VL4; VL4-L6-VL5-L7-VH5-L8-VH4; VH4-L6-VH5-L7-VL5-L8-VL4; VL4-VL5-VL6-VH6-VH5-VH4; VH4-VH5-VH6-VL6-VL5-VL4; VL4-VH5-VL6-VH6-VL5-VH4; VH4-VL5-VH6-VL6-VH5-VL4; VL4-VL5-VH6-VL6-VH5-VH4; VH4-VH5-VL6-VH6-VL5-VL4; VL4-VH5-VH6-VL6-VL5-VH4; VH4-VL5-VL6-VH6-VH5-VL4; VL4-L6-VL5-L7-VL6-L8-VH6-L9-VH5-L10-VH4; VH4-L6-VH5-L7-VH6-L8-VL6-L9-VL5-L10-VL4; VL4-L6-VH5-L7-VL6-L8-VH6-L9-VL5-L10-VH4; VH4-L6-VL5-L7-VH6-L8-VL6-L9-VH5-L10-VL4; VL4-L6-VL5-L7-VH6-L8-VL6-L9-VH5-L10-VH4; VH4-L6-VH5-L7-VL6-L8-VH6-L9-VL5-L10-VL4; VL4-L6-VH5-L7-VH6-L8-VL6-L9-VL5-L10-VH4; or VH4-L6-VL5-L7-VL6-L8-VH6-L9-VH5-L10-VL4; wherein VL1 is a first immunoglobulin light chain variable region that specifically binds to an HIV protein; VL2 is a second immunoglobulin light chain variable region that specifically binds to an HIV protein; VL3 is a third immunoglobulin light chain variable region that specifically binds to an HIV protein; VL4 is a fourth immunoglobulin light chain variable region that specifically binds to an HIV protein; VL5 is a fifth immunoglobulin light chain variable region that specifically binds to an HIV protein; VL6 is a sixth immunoglobulin light chain variable region that specifically binds to an HIV protein; VH1 is a first immunoglobulin heavy chain variable region that specifically binds to an HIV protein; VH2 is a second immunoglobulin heavy chain variable region that specifically binds to an HIV protein; VH3 is a third immunoglobulin heavy chain variable region that specifically binds to an HIV protein; VH4 is a fourth immunoglobulin heavy chain variable region that specifically binds to an HIV protein; VH5 is a fifth immunoglobulin heavy chain variable region that specifically binds to an HIV protein; VH6 is a sixth immunoglobulin heavy chain variable region that specifically binds to an HIV protein; and L1, L2, L3, L4, L5, L6, L7, L8, L9 and L10 are amino acid linkers.

Provided herein is an antigen binding polypeptide having a structure represented by VL1-VL2-VL3-VH3-VH2-VH1-Fc; VH1-VH2-VH3-VL3-VL2-VL1-Fc; VL1-VH2-VL3-VH3-VL2-VH1-Fc; VH1-VL2-VH3-VL3-VH2-VL1-Fc; VL1-VL2-VH3-VL3-VH2-VH1-Fc; VH1-VH2-VL3-VH3-VL2-VL1-Fc; VL1-VH2-VH3-VL3-VL2-VH1-Fc; VH1-VL2-VL3-VH3-VH2-VL1-Fc; VL1-L1-VL2-L2-VL3-L3-VH3-L4-VH2-L5-VH1-Fc; VL1-L1-VL2-L2-VL3-L3-VH3-L4-VH2-L5-VH1-L6-Fc; VH1-L1-VH2-L2-VH3-L3-VL3-L4-VL2-L5-VL1-Fc; VH1-L1-VH2-L2-VH3-L3-VL3-L4-VL2-L5-VL1-L6-Fc; VL1-L1-VH2-L2-VL3-L3-VH3-L4-VL2-L5-VH1-Fc; VL1-L1-VH2-L2-VL3-L3-VH3-L4-VL2-L5-VH1-L6-Fc; VH1-L1-VL2-L2-VH3-L3-VL3-L4-VH2-L5-VL1-Fc; VH1-L1-VL2-L2-VH3-L3-VL3-L4-VH2-L5-VL1-L6-Fc; VL1-L1-VL2-L2-VH3-L3-VL3-L4-VH2-L5-VH1-Fc; VL1-L1-VL2-L2-VH3-L3-VL3-L4-VH2-L5-VH1-L6-Fc; VH1-L1-VH2-L2-VL3-L3-VH3-L4-VL2-L5-VL1-Fc; VH1-L1-VH2-L2-VL3-L3-VH3-L4-VL2-L5-VL1-L6-Fc; VL1-L1-VH2-L2-VH3-L3-VL3-L4-VL2-L5-VH1-Fc; VL1-L1-VH2-L2-VH3-L3-VL3-L4-VL2-L5-VH1-L6-Fc; VH1-L1-VL2-L2-VL3-L3-VH3-L4-VH2-L5-VL1-Fc; or VH1-L1-VL2-L2-VL3-L3-VH3-L4-VH2-L5-VL1-L6-Fc; wherein VL1 is a first immunoglobulin light chain variable region that specifically binds to an HIV protein; VL2 is a second immunoglobulin light chain variable region that specifically binds to an HIV protein; VL3 is a third immunoglobulin light chain variable region that specifically binds to an HIV protein; VH1 is a first immunoglobulin heavy chain variable region that specifically binds to an HIV protein; VH2 is a second immunoglobulin heavy chain variable region that specifically binds to an HIV protein; VH3 is a third immunoglobulin heavy chain variable region that specifically binds to an HIV protein; Fc is a region comprising an immunoglobulin heavy chain constant region 2 (CH2), an immunoglobulin heavy chain constant region 3 (CH3), and optionally, an immunoglobulin hinge; and L1, L2, L3, L4, L5 and L6 are amino acid linkers.

Provided herein is an antigen binding polypeptide having a structure represented by VL1-VL2-VL3-VH3-VH2-VH1-Fc-Fc; VH1-VH2-VH3-VL3-VL2-VL1-Fc-Fc; VL1-VH2-VL3-VH3-VL2-VH1-Fc-Fc; VH1-VL2-VH3-VL3-VH2-VL1-Fc-Fc; VL1-VL2-VH3-VL3-VH2-VH1-Fc-Fc; VH1-VH2-VL3-VH3-VL2-VL1-Fc-Fc; VL1-VH2-VH3-VL3-VL2-VH1-Fc-Fc; VH1-VL2-VL3-VH3-VH2-VL1-Fc-Fc; VL1-L1-VL2-L2-VL3-L3-VH3-L4-VH2-L5-VH1-Fc-Fc; VL1-L1-VL2-L2-VL3-L3-VH3-L4-VH2-L5-VH1-L6-Fc-Fc; VL1-L1-VL2-L2-VL3-L3-VH3-L4-VH2-L5-VH1-L6-Fc-L7-Fc; VH1-L1-VH2-L2-VH3-L3-VL3-L4-VL2-L5-VL1-Fc-Fc; VH1-L1-VH2-L2-VH3-L3-VL3-L4-VL2-L5-VL1-L6-Fc-Fc; VH1-L1-VH2-L2-VH3-L3-VL3-L4-VL2-L5-VL1-L6-Fc-L7-Fc; VL1-L1-VH2-L2-VL3-L3-VH3-L4-VL2-L5-VH1-Fc-Fc; VL1-L1-VH2-L2-VL3-L3-VH3-L4-VL2-L5-VH1-L6-Fc-Fc; VL1-L1-VH2-L2-VL3-L3-VH3-L4-VL2-L5-VH1-L6-Fc-L7-Fc; VH1-L1-VL2-L2-VH3-L3-VL3-L4-VH2-L5-VL1-Fc-Fc; VH1-L1-VL2-L2-VH3-L3-VL3-L4-VH2-L5-VL1-L6-Fc-Fc; VH1-L1-VL2-L2-VH3-L3-VL3-L4-VH2-L5-VL1-L6-Fc-L7-Fc; VL1-L1-VL2-L2-VH3-L3-VL3-L4-VH2-L5-VH1-Fc-Fc; VL1-L1-VL2-L2-VH3-L3-VL3-L4-VH2-L5-VH1-L6-Fc-Fc; VL1-L1-VL2-L2-VH3-L3-VL3-L4-VH2-L5-VH1-L6-Fc-L7-Fc; VH1-L1-VH2-L2-VL3-L3-VH3-L4-VL2-L5-VL1-Fc-Fc; VH1-L1-VH2-L2-VL3-L3-VH3-L4-VL2-L5-VL1-L6-Fc-Fc; VH1-L1-VH2-L2-VL3-L3-VH3-L4-VL2-L5-VL1-L6-Fc-L7-Fc; L1-L1-VH2-L2-VH3-L3-VL3-L4-VL2-L5-VH1-Fc-Fc; VL1-L1-VH2-L2-VH3-L3-VL3-L4-VL2-L5-VH1-L6-Fc-Fc; VL1-L1-VH2-L2-VH3-L3-VL3-L4-VL2-L5-VH1-L6-Fc-L7-Fc; VH1-L1-VL2-L2-VL3-L3-VH3-L4-VH2-L5-VL1-Fc-Fc; VH1-L1-VL2-L2-VL3-L3-VH3-L4-VH2-L5-VL1-L6-Fc-Fc; or VH1-L1-VL2-L2-VL3-L3-VH3-L4-VH2-L5-VL1-L6-Fc-L7-Fc; wherein VL1 is a first immunoglobulin light chain variable region that specifically binds to an HIV protein; VL2 is a second immunoglobulin light chain variable region that specifically binds to an HIV protein; VL3 is a third immunoglobulin light chain variable region that specifically binds to an HIV protein; VH1 is a first immunoglobulin heavy chain variable region that specifically binds to an HIV protein; VH2 is a second immunoglobulin heavy chain variable region that specifically binds to an HIV protein; VH3 is a third immunoglobulin heavy chain variable region that specifically binds to an HIV protein; Fc is a region comprising an immunoglobulin heavy chain constant region 2 (CH2), an immunoglobulin heavy chain constant region 3 (CH3), and optionally, an immunoglobulin hinge; and L1, L2, L3, L4, L5, L6 and L7 are amino acid linkers.

Provided herein is an antigen binding polypeptide complex comprising a first polypeptide and a second polypeptide; wherein the first polypeptide has a structure represented by VL1-VL2-VL3-VH3-VH2-VH1-Fc; VH1-VH2-VH3-VL3-VL2-VL1-Fc; VL1-VH2-VL3-VH3-VL2-VH1-Fc; VH1-VL2-VH3-VL3-VH2-VL1-Fc; VL1-VL2-VH3-VL3-VH2-VH1-Fc; VH1-VH2-VL3-VH3-VL2-VL1-Fc; VL1-VH2-VH3-VL3-VL2-VH1-Fc; VH1-VL2-VL3-VH3-VH2-VL1-Fc; VL1-L1-VL2-L2-VL3-L3-VH3-L4-VH2-L5-VH1-Fc; VL1-L1-VL2-L2-VL3-L3-VH3-L4-VH2-L5-VH1-L6-Fc; VH1-L1-VH2-L2-VH3-L3-VL3-L4-VL2-L5-VL1-Fc; VH1-L1-VH2-L2-VH3-L3-VL3-L4-VL2-L5-VL1-L6-Fc; VL1-L1-VH2-L2-VL3-L3-VH3-L4-VL2-L5-VH1-Fc; VL1-L1-VH2-L2-VL3-L3-VH3-L4-VL2-L5-VH1-L6-Fc; VH1-L1-VL2-L2-VH3-L3-VL3-L4-VH2-L5-VL1-Fc; VH1-L1-VL2-L2-VH3-L3-VL3-L4-VH2-L5-VL1-L6-Fc; VL1-L1-VL2-L2-VH3-L3-VL3-L4-VH2-L5-VH1-Fc; VL1-L1-VL2-L2-VH3-L3-VL3-L4-VH2-L5-VH1-L6-Fc; VH1-L1-VH2-L2-VL3-L3-VH3-L4-VL2-L5-VL1-Fc; VH1-L1-VH2-L2-VL3-L3-VH3-L4-VL2-L5-VL1-L6-Fc; VL1-L1-VH2-L2-VH3-L3-VL3-L4-VL2-L5-VH1-Fc; VL1-L1-VH2-L2-VH3-L3-VL3-L4-VL2-L5-VH1-L6-Fc; VH1-L1-VL2-L2-VL3-L3-VH3-L4-VH2-L5-VL1-Fc; or VH1-L1-VL2-L2-VL3-L3-VH3-L4-VH2-L5-VL1-L6-Fc; wherein the second polypeptide has a structure represented by Fc; VL4-VH4-Fc; VH4-VL4-Fc; VL4-L7-VH4-Fc; VH4-L7-VL4-Fc; VL4-L7-VH4-L8-Fc; VH4-L7-VL4-L8-Fc; VL4-VL5-VH5-VH4-Fc; VH4-VH5-VL5-VL4-Fc; VL4-L7-VL5-L8-VH5-L9-VH4-Fc; VH4-L7-VH5-L8-VL5-L9-VL4-Fc; VL4-L7-VL5-L8-VH5-L9-VH4-L10-Fc; VH4-L7-VH5-L8-VL5-L9-VL4-L10-Fc; VL4-VL5-VL6-VH6-VH5-VH4-Fc; VH4-VH5-VH6-VL6-VL5-VL4-Fc; VL4-VH5-VL6-VH6-VL5-VH4-Fc; VH4-VL5-VH6-VL6-VH5-VL4-Fc; VL4-VL5-VH6-VL6-VH5-VH4-Fc; VH4-VH5-VL6-VH6-VL5-VL4-Fc; VL4-VH5-VH6-VL6-VL5-VH4-Fc; VH4-VL5-VL6-VH6-VH5-VL4-Fc; VL4-L7-VL5-L8-VL6-L9-VH6-L10-VH5-L11-VH4-Fc; VH4-L7-VH5-L8-VH6-L9-VL6-L10-VL5-L11-VL4-Fc; VL4-L7-VH5-L8-VL6-L9-VH6-L10-VL5-L11-VH4-Fc; VH4-L7-VL5-L8-VH6-L9-VL6-L10-VH5-L11-VL4-Fc; VL4-L7-VL5-L8-VH6-L9-VL6-L10-VH5-L11-VH4-Fc; VH4-L7-VH5-L8-VL6-L9-VH6-L10-VL5-L11-VL4-Fc; VL4-L7-VH5-L8-VH6-L9-VL6-L10-VL5-L11-VH4-Fc; VH4-L7-VL5-L8-VL6-L9-VH6-L10-VH5-L11-VL4-Fc; VL4-L7-VL5-L8-VL6-L9-VH6-L10-VH5-L11-VH4-L12-Fc; VH4-L7-VH5-L8-VH6-L9-VL6-L10-VL5-L11-VL4-L12-Fc; VL4-L7-VH5-L8-VL6-L9-VH6-L10-VL5-L11-VH4-L12-Fc; VH4-L7-VL5-L8-VH6-L9-VL6-L10-VH5-L11-VL4-L12-Fc; VL4-L7-VL5-L8-VH6-L9-VL6-L10-VH5-L11-VH4-L12-Fc; VH4-L7-VH5-L8-VL6-L9-VH6-L10-VL5-L11-VL4-L12-Fc; VL4-L7-VH5-L8-VH6-L9-VL6-L10-VL5-L11-VH4-L12-Fc; or VH4-L7-VL5-L8-VL6-L9-VH6-L10-VH5-L11-VL4-L12-Fc; wherein VL1 is a first immunoglobulin light chain variable region that specifically binds to an HIV protein; VL2 is a second immunoglobulin light chain variable region that specifically binds to an HIV protein; VL3 is a third immunoglobulin light chain variable region that specifically binds to an HIV protein; VL4 is a fourth immunoglobulin light chain variable region that specifically binds to an HIV protein; VL5 is a fifth immunoglobulin light chain variable region that specifically binds to an HIV protein; VL6 is a sixth immunoglobulin light chain variable region that specifically binds to an HIV protein; VH1 is a first immunoglobulin heavy chain variable region that specifically binds to an HIV protein; VH2 is a second immunoglobulin heavy chain variable region that specifically binds to an HIV protein; VH3 is a third immunoglobulin heavy chain variable region that specifically binds to an HIV protein; VH4 is a fourth immunoglobulin heavy chain variable region that specifically binds to an HIV protein; VH5 is a fifth immunoglobulin heavy chain variable region that specifically binds to an HIV protein; VH6 is a sixth immunoglobulin heavy chain variable region that specifically binds to an HIV protein; Fc is a region comprising an immunoglobulin heavy chain constant region 2 (CH2), an immunoglobulin heavy chain constant region 3 (CH3), and optionally, an immunoglobulin hinge; and L1, L2, L3, L4, L5, L6, L7, L8, L9, L10, L11 and L12 are amino acid linkers.

Provided herein is an antigen binding polypeptide having a structure represented by VL1-VL2-VL3-VH3-VH2-VH1-CH1-CL; VL1-VL2-VL3-VH3-VH2-VH1-CL-CH1; VH1-VH2-VH3-VL3-VL2-VL1-CH1-CL; VH1-VH2-VH3-VL3-VL2-VL1-CL-CH1; VL1-VH2-VL3-VH3-VL2-VH1-CH1-CL; VL1-VH2-VL3-VH3-VL2-VH1-CL-CH1; VH1-VL2-VH3-VL3-VH2-VL1-CH1-CL; VH1-VL2-VH3-VL3-VH2-VL1-CL-CH1; VL1-VL2-VH3-VL3-VH2-VH1-CH1-CL; VL1-VL2-VH3-VL3-VH2-VH1-CL-CH1; VH1-VH2-VL3-VH3-VL2-VL1-CH1-CL; VH1-VH2-VL3-VH3-VL2-VL1-CL-CH1; VL1-VH2-VH3-VL3-VL2-VH1-CH1-CL; VL1-VH2-VH3-VL3-VL2-VH1-CL-CH1; VH1-VL2-VL3-VH3-VH2-VL1-CH1-CL; VH1-VL2-VL3-VH3-VH2-VL1-CL-CH1; VL1-L1-VL2-L2-VL3-L3-VH3-L4-VH2-L5-VH1-CH1-CL; VL1-L1-VL2-L2-VL3-L3-VH3-L4-VH2-L5-VH1-L6-CH1-CL; VL1-L1-VL2-L2-VL3-L3-VH3-L4-VH2-L5-VH1-L6-CH1-L7-CL; VL1-L1-VL2-L2-VL3-L3-VH3-L4-VH2-L5-VH1-CL-CH1; VL1-L1-VL2-L2-VL3-L3-VH3-L4-VH2-L5-VH1-L6-CL-CH1; VL1-L1-VL2-L2-VL3-L3-VH3-L4-VH2-L5-VH1-L6-CL-L7-CH1; VH1-L1-VH2-L2-VH3-L3-VL3-L4-VL2-L5-VL1-CH1-CL; VH1-L1-VH2-L2-VH3-L3-VL3-L4-VL2-L5-VL1-L6-CH1-CL; VH1-L1-VH2-L2-VH3-L3-VL3-L4-VL2-L5-VL1-L6-CH1-L7-CL; VH1-L1-VH2-L2-VH3-L3-VL3-L4-VL2-L5-VL1-CL-CH1; VH1-L1-VH2-L2-VH3-L3-VL3-L4-VL2-L5-VL1-L6-CL-CH1; VH1-L1-VH2-L2-VH3-L3-VL3-L4-VL2-L5-VL1-L6-CL-L7-CH1; VL1-L1-VH2-L2-VL3-L3-VH3-L4-VL2-L5-VH1-CH1-CL; VL1-L1-VH2-L2-VL3-L3-VH3-L4-VL2-L5-VH1-L6-CH1-CL; VL1-L1-VH2-L2-VL3-L3-VH3-L4-VL2-L5-VH1-L6-CH1-L7-CL; VL1-L1-VH2-L2-VL3-L3-VH3-L4-VL2-L5-VH1-CL-CH1; VL1-L1-VH2-L2-VL3-L3-VH3-L4-VL2-L5-VH1-L6-CL-CH1; VL1-L1-VH2-L2-VL3-L3-VH3-L4-VL2-L5-VH1-L6-CL-L7-CH1; VH1-L1-VL2-L2-VH3-L3-VL3-L4-VH2-L5-VL1-CH1-CL; VH1-L1-VL2-L2-VH3-L3-VL3-L4-VH2-L5-VL1-L6-CH1-CL; VH1-L1-VL2-L2-VH3-L3-VL3-L4-VH2-L5-VL1-L6-CH1-L7-CL; VH1-L1-VL2-L2-VH3-L3-VL3-L4-VH2-L5-VL1-CL-CH1; VH1-L1-VL2-L2-VH3-L3-VL3-L4-VH2-L5-VL1-L6-CL-CH1; VH1-L1-VL2-L2-VH3-L3-VL3-L4-VH2-L5-VL1-L6-CL-L7-CH1; VL1-L1-VL2-L2-VH3-L3-VL3-L4-VH2-L5-VH1-CH1-CL; VL1-L1-VL2-L2-VH3-L3-VL3-L4-VH2-L5-VH1-L6-CH1-CL; VL1-L1-VL2-L2-VH3-L3-VL3-L4-VH2-L5-VH1-L6-CH1-L7-CL; VL1-L1-VL2-L2-VH3-L3-VL3-L4-VH2-L5-VH1-CL-CH1; VL1-L1-VL2-L2-VH3-L3-VL3-L4-VH2-L5-VH1-L6-CL-CH1; VL1-L1-VL2-L2-VH3-L3-VL3-L4-VH2-L5-VH1-L6-CL-L7-CH1; VH1-L1-VH2-L2-VL3-L3-VH3-L4-VL2-L5-VL1-CH1-CL; VH1-L1-VH2-L2-VL3-L3-VH3-L4-VL2-L5-VL1-L6-CH1-CL; VH1-L1-VH2-L2-VL3-L3-VH3-L4-VL2-L5-VL1-L6-CH1-L7-CL; VH1-L1-VH2-L2-VL3-L3-VH3-L4-VL2-L5-VL1-CL-CH1; VH1-L1-VH2-L2-VL3-L3-VH3-L4-VL2-L5-VL1-L6-CL-CH1; VH1-L1-VH2-L2-VL3-L3-VH3-L4-VL2-L5-VL1-L6-CL-L7-CH1; VL1-L1-VH2-L2-VH3-L3-VL3-L4-VL2-L5-VH1-CH1-CL; VL1-L1-VH2-L2-VH3-L3-VL3-L4-VL2-L5-VH1-L6-CH1-CL; VL1-L1-VH2-L2-VH3-L3-VL3-L4-VL2-L5-VH1-L6-CH1-L7-CL; VL1-L1-VH2-L2-VH3-L3-VL3-L4-VL2-L5-VH1-CL-CH1; VL1-L1-VH2-L2-VH3-L3-VL3-L4-VL2-L5-VH1-L6-CL-CH1; VL1-L1-VH2-L2-VH3-L3-VL3-L4-VL2-L5-VH1-L6-CL-L7-CH1; VH1-L1-VL2-L2-VL3-L3-VH3-L4-VH2-L5-VL1-CH1-CL; VH1-L1-VL2-L2-VL3-L3-VH3-L4-VH2-L5-VL1-L6-CH1-CL; VH1-L1-VL2-L2-VL3-L3-VH3-L4-VH2-L5-VL1-L6-CH1-L7-CL; VH1-L1-VL2-L2-VL3-L3-VH3-L4-VH2-L5-VL1-CL-CH1; VH1-L1-VL2-L2-VL3-L3-VH3-L4-VH2-L5-VL1-L6-CL-CH1; or VH1-L1-VL2-L2-VL3-L3-VH3-L4-VH2-L5-VL1-L6-CL-L7-CH1; wherein VL1 is a first immunoglobulin light chain variable region that specifically binds to an HIV protein; VL2 is a second immunoglobulin light chain variable region that specifically binds to an HIV protein; VL3 is a third immunoglobulin light chain variable region that specifically binds to an HIV protein; VH1 is a first immunoglobulin heavy chain variable region that specifically binds to an HIV protein; VH2 is a second immunoglobulin heavy chain variable region that specifically binds to an HIV protein; VH3 is a third immunoglobulin heavy chain variable region that specifically binds to an HIV protein; CH1 is a heavy chain constant region 1; CL is a light chain constant region; and L1, L2, L3, L4, L5, L6 and L7 are amino acid linkers.

Provided herein is an antigen binding polypeptide complex comprising a first polypeptide and a second polypeptide; wherein the first polypeptide has a structure represented by VL1-VL2-VL3-VH3-VH2-VH1-CH1; VL1-VL2-VL3-VH3-VH2-VH1-CL; VL1-VL2-VL3-VH3-VH2-VH1-CH1-CL; VL1-VL2-VL3-VH3-VH2-VH1-CL-CH1; VH1-VH2-VH3-VL3-VL2-VL1-CH1; VH1-VH2-VH3-VL3-VL2-VL1-CL; VH1-VH2-VH3-VL3-VL2-VL1-CH1-CL; VH1-VH2-VH3-VL3-VL2-VL1-CL-CH1; VL1-VH2-VL3-VH3-VL2-VH1-CH1; VL1-VH2-VL3-VH3-VL2-VH1-CL; VL1-VH2-VL3-VH3-VL2-VH1-CH1-CL; VL1-VH2-VL3-VH3-VL2-VH1-CL-CH1; VH1-VL2-VH3-VL3-VH2-VL1-CH1; VH1-VL2-VH3-VL3-VH2-VL1-CL; VH1-VL2-VH3-VL3-VH2-VL1-CH1-CL; VH1-VL2-VH3-VL3-VH2-VL1-CL-CH1; VL1-VL2-VH3-VL3-VH2-VH1-CH1; VL1-VL2-VH3-VL3-VH2-VH1-CL; VL1-VL2-VH3-VL3-VH2-VH1-CH1-CL; VL1-VL2-VH3-VL3-VH2-VH1-CL-CH1; VH1-VH2-VL3-VH3-VL2-VL1-CH1; VH1-VH2-VL3-VH3-VL2-VL1-CL; VH1-VH2-VL3-VH3-VL2-VL1-CH1-CL; VH1-VH2-VL3-VH3-VL2-VL1-CL-CH1; VL1-VH2-VH3-VL3-VL2-VH1-CH1; VL1-VH2-VH3-VL3-VL2-VH1-CL; VL1-VH2-VH3-VL3-VL2-VH1-CH1-CL; VL1-VH2-VH3-VL3-VL2-VH1-CL-CH1; VH1-VL2-VL3-VH3-VH2-VL1-CH1; VH1-VL2-VL3-VH3-VH2-VL1-CL; VH1-VL2-VL3-VH3-VH2-VL1-CH1-CL; VH1-VL2-VL3-VH3-VH2-VL1-CL-CH1; VL1-L1-VL2-L2-VL3-L3-VH3-L4-VH2-L5-VH1-CH1; VL1-L1-VL2-L2-VL3-L3-VH3-L4-VH2-L5-VH1-L6-CH1; VL1-L1-VL2-L2-VL3-L3-VH3-L4-VH2-L5-VH1-CL; VL1-L1-VL2-L2-VL3-L3-VH3-L4-VH2-L5-VH1-L6-CL; VL1-L1-VL2-L2-VL3-L3-VH3-L4-VH2-L5-VH1-CH1-CL; VL1-L1-VL2-L2-VL3-L3-VH3-L4-VH2-L5-VH1-L6-CH1-CL; VL1-L1-VL2-L2-VL3-L3-VH3-L4-VH2-L5-VH1-L6-CH1-L7-CL; VL1-L1-VL2-L2-VL3-L3-VH3-L4-VH2-L5-VH1-CL-CH1; VL1-L1-VL2-L2-VL3-L3-VH3-L4-VH2-L5-VH1-L6-CL-CH1; VL1-L1-VL2-L2-VL3-L3-VH3-L4-VH2-L5-VH1-L6-CL-L7-CH1; VH1-L1-VH2-L2-VH3-L3-VL3-L4-VL2-L5-VL1-CH1; VH1-L1-VH2-L2-VH3-L3-VL3-L4-VL2-L5-VL1-L6-CH1; VH1-L1-VH2-L2-VH3-L3-VL3-L4-VL2-L5-VL1-CL; VH1-L1-VH2-L2-VH3-L3-VL3-L4-VL2-L5-VL1-L6-CL; VH1-L1-VH2-L2-VH3-L3-VL3-L4-VL2-L5-VL1-CH1-CL; VH1-L1-VH2-L2-VH3-L3-VL3-L4-VL2-L5-VL1-L6-CH1-CL; VH1-L1-VH2-L2-VH3-L3-VL3-L4-VL2-L5-VL1-L6-CH1-L7-CL; VH1-L1-VH2-L2-VH3-L3-VL3-L4-VL2-L5-VL1-CL-CH1; VH1-L1-VH2-L2-VH3-L3-VL3-L4-VL2-L5-VL1-L6-CL-CH1; VH1-L1-VH2-L2-VH3-L3-VL3-L4-VL2-L5-VL1-L6-CL-L7-CH1; VL1-L1-VH2-L2-VL3-L3-VH3-L4-VL2-L5-VH1-CH1; VL1-L1-VH2-L2-VL3-L3-VH3-L4-VL2-L5-VH1-L6-CH1; VL1-L1-VH2-L2-VL3-L3-VH3-L4-VL2-L5-VH1-CL; VL1-L1-VH2-L2-VL3-L3-VH3-L4-VL2-L5-VH1-L6-CL; VL1-L1-VH2-L2-VL3-L3-VH3-L4-VL2-L5-VH1-CH1-CL; VL1-L1-VH2-L2-VL3-L3-VH3-L4-VL2-L5-VH1-L6-CH1-CL; VL1-L1-VH2-L2-VL3-L3-VH3-L4-VL2-L5-VH1-L6-CH1-L7-CL; VL1-L1-VH2-L2-VL3-L3-VH3-L4-VL2-L5-VH1-CL-CH1; VL1-L1-VH2-L2-VL3-L3-VH3-L4-VL2-L5-VH1-L6-CL-CH1; VL1-L1-VH2-L2-VL3-L3-VH3-L4-VL2-L5-VH1-L6-CL-L7-CH1; VH1-L1-VL2-L2-VH3-L3-VL3-L4-VH2-L5-VL1-CH1; VH1-L1-VL2-L2-VH3-L3-VL3-L4-VH2-L5-VL1-L6-CH1; VH1-L1-VL2-L2-VH3-L3-VL3-L4-VH2-L5-VL1-CL; VH1-L1-VL2-L2-VH3-L3-VL3-L4-VH2-L5-VL1-L6-CL; VH1-L1-VL2-L2-VH3-L3-VL3-L4-VH2-L5-VL1-CH1-CL; VH1-L1-VL2-L2-VH3-L3-VL3-L4-VH2-L5-VL1-L6-CH1-CL; VH1-L1-VL2-L2-VH3-L3-VL3-L4-VH2-L5-VL1-L6-CH1-L7-CL; VH1-L1-VL2-L2-VH3-L3-VL3-L4-VH2-L5-VL1-CL-CH1; VH1-L1-VL2-L2-VH3-L3-VL3-L4-VH2-L5-VL1-L6-CL-CH1; VH1-L1-VL2-L2-VH3-L3-VL3-L4-VH2-L5-VL1-L6-CL-L7-CH1; VL1-L1-VL2-L2-VH3-L3-VL3-L4-VH2-L5-VH1-CH1; VL1-L1-VL2-L2-VH3-L3-VL3-L4-VH2-L5-VH1-L6-CH1; VL1-L1-VL2-L2-VH3-L3-VL3-L4-VH2-L5-VH1-CL; VL1-L1-VL2-L2-VH3-L3-VL3-L4-VH2-L5-VH1-L6-CL; VL1-L1-VL2-L2-VH3-L3-VL3-L4-VH2-L5-VH1-CH1-CL; VL1-L1-VL2-L2-VH3-L3-VL3-L4-VH2-L5-VH1-L6-CH1-CL; VL1-L1-VL2-L2-VH3-L3-VL3-L4-VH2-L5-VH1-L6-CH1-L7-CL; VL1-L1-VL2-L2-VH3-L3-VL3-L4-VH2-L5-VH1-CL-CH1; VL1-L1-VL2-L2-VH3-L3-VL3-L4-VH2-L5-VH1-L6-CL-CH1; VL1-L1-VL2-L2-VH3-L3-VL3-L4-VH2-L5-VH1-L6-CL-L7-CH1; VH1-L1-VH2-L2-VL3-L3-VH3-L4-VL2-L5-VL1-CH1; VH1-L1-VH2-L2-VL3-L3-VH3-L4-VL2-L5-VL1-L6-CH1; VH1-L1-VH2-L2-VL3-L3-VH3-L4-VL2-L5-VL1-CL; VH1-L1-VH2-L2-VL3-L3-VH3-L4-VL2-L5-VL1-L6-CL; VH1-L1-VH2-L2-VL3-L3-VH3-L4-VL2-L5-VL1-CH1-CL; VH1-L1-VH2-L2-VL3-L3-VH3-L4-VL2-L5-VL1-L6-CH1-CL; VH1-L1-VH2-L2-VL3-L3-VH3-L4-VL2-L5-VL1-L6-CH1-L7-CL; VH1-L1-VH2-L2-VL3-L3-VH3-L4-VL2-L5-VL1-CL-CH1; VH1-L1-VH2-L2-VL3-L3-VH3-L4-VL2-L5-VL1-L6-CL-CH1; VH1-L1-VH2-L2-VL3-L3-VH3-L4-VL2-L5-VL1-L6-CL-L7-CH1; VL1-L1-VH2-L2-VH3-L3-VL3-L4-VL2-L5-VH1-CH1; VL1-L1-VH2-L2-VH3-L3-VL3-L4-VL2-L5-VH1-L6-CH1; VL1-L1-VH2-L2-VH3-L3-VL3-L4-VL2-L5-VH1-CL; VL1-L1-VH2-L2-VH3-L3-VL3-L4-VL2-L5-VH1-L6-CL; VL1-L1-VH2-L2-VH3-L3-VL3-L4-VL2-L5-VH1-CH1-CL; VL1-L1-VH2-L2-VH3-L3-VL3-L4-VL2-L5-VH1-L6-CH1-CL; VL1-L1-VH2-L2-VH3-L3-VL3-L4-VL2-L5-VH1-L6-CH1-L7-CL; VL1-L1-VH2-L2-VH3-L3-VL3-L4-VL2-L5-VH1-CL-CH1; VL1-L1-VH2-L2-VH3-L3-VL3-L4-VL2-L5-VH1-L6-CL-CH1; VL1-L1-VH2-L2-VH3-L3-VL3-L4-VL2-L5-VH1-L6-CL-L7-CH1; VH1-L1-VL2-L2-VL3-L3-VH3-L4-VH2-L5-VL1-CH1; VH1-L1-VL2-L2-VL3-L3-VH3-L4-VH2-L5-VL1-L6-CH1; VH1-L1-VL2-L2-VL3-L3-VH3-L4-VH2-L5-VL1-CL; VH1-L1-VL2-L2-VL3-L3-VH3-L4-VH2-L5-VL1-L6-CL; VH1-L1-VL2-L2-VL3-L3-VH3-L4-VH2-L5-VL1-CH1-CL; VH1-L1-VL2-L2-VL3-L3-VH3-L4-VH2-L5-VL1-L6-CH1-CL; VH1-L1-VL2-L2-VL3-L3-VH3-L4-VH2-L5-VL1-L6-CH1-L7-CL; VH1-L1-VL2-L2-VL3-L3-VH3-L4-VH2-L5-VL1-CL-CH1; VH1-L1-VL2-L2-VL3-L3-VH3-L4-VH2-L5-VL1-L6-CL-CH1; or VH1-L1-VL2-L2-VL3-L3-VH3-L4-VH2-L5-VL1-L6-CL-L7-CH1; wherein the second polypeptide has a structure represented by VL4-VH4-CH1; VL4-VH4-CL; VL4-VH4-CH1-CL; VL4-VH4-CL-CH1; VH4-VL4-CH1; VH4-VL4-CL; VH4-VL4-CH1-CL; VH4-VL4-CL-CH1; VL4-L8-VH4-CH1; VL4-L8-VH4-CL; VL4-L8-VH4-CH1-CL; VL4-L8-VH4-CL-CH1; VH4-L8-VL4-CH1; VH4-L8-VL4-CL; VH4-L8-VH4-CH1-CL; VH4-L8-VH4-CL-CH1; VL4-VL5-VH5-VH4-CH1; VL4-VL5-VH5-VH4-CL; VL4-VL5-VH5-VH4-CH1-CL; VL4-VL5-VH5-VH4-CL-CH1; VH4-VH5-VL5-VL4-CH1; VH4-VH5-VL5-VL4-CL; VH4-VH5-VL5-VL4-CH1-CL; VH4-VH5-VL5-VL4-CL-CH1; VL4-L8-VL5-L9-VH5-L10-VH4-CH1; VL4-L8-VL5-L9-VH5-L10-VH4-CL; VL4-L8-VL5-L9-VH5-L10-VH4-CH1-CL; VL4-L8-VL5-L9-VH5-L10-VH4-CL-CH1; VH4-L8-VH5-L9-VL5-L10-VL4-CH1; VH4-L8-VH5-L9-VL5-L10-VL4-CL; VH4-L8-VH5-L9-VL5-L10-VL4-CH1-CL; VH4-L8-VH5-L9-VL5-L10-VL4-CL-CH1; VL4-VL5-VL6-VH6-VH5-VH4-CH1; VL4-VL5-VL6-VH6-VH5-VH4-CL; VL4-VL5-VL6-VH6-VH5-VH4-CH1-CL; VL4-VL5-VL6-VH6-VH5-VH4-CL-CH1; VH4-VH5-VH6-VL6-VL5-VL4-CH1; VH4-VH5-VH6-VL6-VL5-VL4-CL; VH4-VH5-VH6-VL6-VL5-VL4-CH1-CL; VH4-VH5-VH6-VL6-VL5-VL4-CL-CH1; VL4-VH5-VL6-VH6-VL5-VH4-CH1; VL4-VH5-VL6-VH6-VL5-VH4-CL; VL4-VH5-VL6-VH6-VL5-VH4-CH1-CL; VL4-VH5-VL6-VH6-VL5-VH4-CL-CH1; VH4-VL5-VH6-VL6-VH5-VL4-CH1; VH4-VL5-VH6-VL6-VH5-VL4-CL; VH4-VL5-VH6-VL6-VH5-VL4-CH1-CL; VH4-VL5-VH6-VL6-VH5-VL4-CL-CH1; VL4-VL5-VH6-VL6-VH5-VH4-CH1; VL4-VL5-VH6-VL6-VH5-VH4-CL; VL4-VL5-VH6-VL6-VH5-VH4-CH1-CL; VL4-VL5-VH6-VL6-VH5-VH4-CL-CH1; VH4-VH5-VL6-VH6-VL5-VL4-CH1; VH4-VH5-VL6-VH6-VL5-VL4-CL; VH4-VH5-VL6-VH6-VL5-VL4-CH1-CL; VH4-VH5-VL6-VH6-VL5-VL4-CL-CH1; VL4-VH5-VH6-VL6-VL5-VH4-CH1; VL4-VH5-VH6-VL6-VL5-VH4-CL; VL4-VH5-VH6-VL6-VL5-VH4-CH1-CL; VL4-VH5-VH6-VL6-VL5-VH4-CL-CH1; VH4-VL5-VL6-VH6-VH5-VL4-CH1; VH4-VL5-VL6-VH6-VH5-VL4-CL; VH4-VL5-VL6-VH6-VH5-VL4-CH1-CL; VH4-VL5-VL6-VH6-VH5-VL4-CL-CH1; VL4-L8-VL5-L9-VL6-L10-VH6-L11-VH5-L12-VH4-CH1; VL4-L8-VL5-L9-VL6-L10-VH6-L11-VH5-L12-VH4-CL; VL4-L8-VL5-L9-VL6-L10-VH6-L11-VH5-L12-VH4-CH1-CL; VL4-L8-VL5-L9-VL6-L10-VH6-L11-VH5-L12-VH4-CL-CH1; VH4-L8-VH5-L9-VH6-L10-VL6-L11-VL5-L12-VL4-CH1; VH4-L8-VH5-L9-VH6-L10-VL6-L11-VL5-L12-VL4-CL; VH4-L8-VH5-L9-VH6-L10-VL6-L11-VL5-L12-VL4-CH1-CL; VH4-L8-VH5-L9-VH6-L10-VL6-L11-VL5-L12-VL4-CL-CH1; VL4-L8-VH5-L9-VL6-L10-VH6-L11-VL5-L12-VH4-CH1; VL4-L8-VH5-L9-VL6-L10-VH6-L11-VL5-L12-VH4-CL; VL4-L8-VH5-L9-VL6-L10-VH6-L11-VL5-L12-VH4-CH1-CL; VL4-L8-VH5-L9-VL6-L10-VH6-L11-VL5-L12-VH4-CL-CH1; VH4-L8-VL5-L9-VH6-L10-VL6-L11-VH5-L12-VL4-CH1; VH4-L8-VL5-L9-VH6-L10-VL6-L11-VH5-L12-VL4-CL; VH4-L8-VL5-L9-VH6-L10-VL6-L11-VH5-L12-VL4-CH1-CL; VH4-L8-VL5-L9-VH6-L10-VL6-L11-VH5-L12-VL4-CL-CH1; VL4-L8-VL5-L9-VH6-L10-VL6-L11-VH5-L12-VH4-CH1; VL4-L8-VL5-L9-VH6-L10-VL6-L11-VH5-L12-VH4-CL; VL4-L8-VL5-L9-VH6-L10-VL6-L11-VH5-L12-VH4-CH1-CL; VL4-L8-VL5-L9-VH6-L10-VL6-L11-VH5-L12-VH4-CL-CH1; VH4-L8-VH5-L9-VL6-L10-VH6-L11-VL5-L12-VL4-CH1; VH4-L8-VH5-L9-VL6-L10-VH6-L11-VL5-L12-VL4-CL; VH4-L8-VH5-L9-VL6-L10-VH6-L11-VL5-L12-VL4-CH1-CL; VH4-L8-VH5-L9-VL6-L10-VH6-L11-VL5-L12-VL4-CL-CH1; VL4-L8-VH5-L9-VH6-L10-VL6-L11-VL5-L12-VH4-CH1; VL4-L8-VH5-L9-VH6-L10-VL6-L11-VL5-L12-VH4-CL; VL4-L8-VH5-L9-VH6-L10-VL6-L11-VL5-L12-VH4-CH1-CL; VL4-L8-VH5-L9-VH6-L10-VL6-L11-VL5-L12-VH4-CL-CH1; VH4-L8-VL5-L9-VL6-L10-VH6-L11-VH5-L12-VL4-CH1; VH4-L8-VL5-L9-VL6-L10-VH6-L11-VH5-L12-VL4-CL; VH4-L8-VL5-L9-VL6-L10-VH6-L11-VH5-L12-VL4-CH1-CL; VH4-L8-VL5-L9-VL6-L10-VH6-L11-VH5-L12-VL4-CL-CH1; VL4-L8-VL5-L9-VL6-L10-VH6-L11-VH5-L12-VH4-L13-CH1; VL4-L8-VL5-L9-VL6-L10-VH6-L11-VH5-L12-VH4-L13-CL; VL4-L8-VL5-L9-VL6-L10-VH6-L11-VH5-L12-VH4-L13-CH1-CL; VL4-L8-VL5-L9-VL6-L10-VH6-L11-VH5-L12-VH4-L13-CL-CH1; VH4-L8-VH5-L9-VH6-L10-VL6-L11-VL5-L12-VL4-L13-CH1; VH4-L8-VH5-L9-VH6-L10-VL6-L11-VL5-L12-VL4-L13-CL; VH4-L8-VH5-L9-VH6-L10-VL6-L11-VL5-L12-VL4-L13-CH1-CL; VH4-L8-VH5-L9-VH6-L10-VL6-L11-VL5-L12-VL4-L13-CL-CH1; VL4-L8-VH5-L9-VL6-L10-VH6-L11-VL5-L12-VH4-L13-CH1; VL4-L8-VH5-L9-VL6-L10-VH6-L11-VL5-L12-VH4-L13-CL; VL4-L8-VH5-L9-VL6-L10-VH6-L11-VL5-L12-VH4-L13-CH1-CL; VL4-L8-VH5-L9-VL6-L10-VH6-L11-VL5-L12-VH4-L13-CL-CH1; VH4-L8-VL5-L9-VH6-L10-VL6-L11-VH5-L12-VL4-L13-CH1; VH4-L8-VL5-L9-VH6-L10-VL6-L11-VH5-L12-VL4-L13-CL; VH4-L8-VL5-L9-VH6-L10-VL6-L11-VH5-L12-VL4-L13-CH1-CL; VH4-L8-VL5-L9-VH6-L10-VL6-L11-VH5-L12-VL4-L13-CL-CH1; VL4-L8-VL5-L9-VH6-L10-VL6-L11-VH5-L12-VH4-L13-CH1; VL4-L8-VL5-L9-VH6-L10-VL6-L11-VH5-L12-VH4-L13-CL; VL4-L8-VL5-L9-VH6-L10-VL6-L11-VH5-L12-VH4-L13-CH1-CL; VL4-L8-VL5-L9-VH6-L10-VL6-L11-VH5-L12-VH4-L13-CL-CH1; VH4-L8-VH5-L9-VL6-L10-VH6-L11-VL5-L12-VL4-L13-CH1; VH4-L8-VH5-L9-VL6-L10-VH6-L11-VL5-L12-VL4-L13-CL; VH4-L8-VH5-L9-VL6-L10-VH6-L11-VL5-L12-VL4-L13-CH1-CL; VH4-L8-VH5-L9-VL6-L10-VH6-L11-VL5-L12-VL4-L13-CL-CH1; VL4-L8-VH5-L9-VH6-L10-VL6-L11-VL5-L12-VH4-L13-CH1; VL4-L8-VH5-L9-VH6-L10-VL6-L11-VL5-L12-VH4-L13-CL; VL4-L8-VH5-L9-VH6-L10-VL6-L11-VL5-L12-VH4-L13-CH1-CL; VL4-L8-VH5-L9-VH6-L10-VL6-L11-VL5-L12-VH4-L13-CH1; VH4-L8-VL5-L9-VL6-L10-VH6-L11-VH5-L12-VL4-L13-CH1; VH4-L8-VL5-L9-VL6-L10-VH6-L11-VH5-L12-VL4-L13-CL; VH4-L8-VL5-L9-VL6-L10-VH6-L11-VH5-L12-VL4-L13-CH1-CL; or VH4-L8-VL5-L9-VL6-L10-VH6-L11-VH5-L12-VL4-L13-CL-CH1; wherein VL1 is a first immunoglobulin light chain variable region that specifically binds to an HIV protein; VL2 is a second immunoglobulin light chain variable region that specifically binds to an HIV protein; VL3 is a third immunoglobulin light chain variable region that specifically binds to an HIV protein; VL4 is a fourth immunoglobulin light chain variable region that specifically binds to an HIV protein; VL5 is a fifth immunoglobulin light chain variable region that specifically binds to an HIV protein; VL6 is a sixth immunoglobulin light chain variable region that specifically binds to an HIV protein; VH1 is a first immunoglobulin heavy chain variable region that specifically binds to an HIV protein; VH2 is a second immunoglobulin heavy chain variable region that specifically binds to an HIV protein; VH3 is a third immunoglobulin heavy chain variable region that specifically binds to an HIV protein; VH4 is a fourth immunoglobulin heavy chain variable region that specifically binds to an HIV protein; VH5 is a fifth immunoglobulin heavy chain variable region that specifically binds to an HIV protein; VH6 is a sixth immunoglobulin heavy chain variable region that specifically binds to an HIV protein; CH1 is a heavy chain constant region 1; CL is a light chain constant region; and L1, L2, L3, L4, L5, L6, L7, L8, L9, L10, L11, L12 and L13 are amino acid linkers.

Provided herein is an antigen binding polypeptide complex comprising a first polypeptide and a second polypeptide; wherein the first polypeptide has a structure represented by VL1-VL2-VL3-VH3-VH2-VH1-CH1-Fc; VH1-VH2-VH3-VL3-VL2-VL1-CH1-Fc; VL1-VH2-VL3-VH3-VL2-VH1-CH1-Fc; VH1-VL2-VH3-VL3-VH2-VL1-CH1-Fc; VL1-VL2-VH3-VL3-VH2-VH1-CH1-Fc; VH1-VH2-VL3-VH3-VL2-VL1-CH1-Fc; VL1-VH2-VH3-VL3-VL2-VH1-CH1-Fc; VH1-VL2-VL3-VH3-VH2-VL1-CH1-Fc; VL1-VL2-VL3-VH3-VH2-VH1-CL-Fc; VH1-VH2-VH3-VL3-VL2-VL1-CL-Fc; VL1-VH2-VL3-VH3-VL2-VH1-CL-Fc; VH1-VL2-VH3-VL3-VH2-VL1-CL-Fc; VL1-VL2-VH3-VL3-VH2-VH1-CL-Fc; VH1-VH2-VL3-VH3-VL2-VL1-CL-Fc; VL1-VH2-VH3-VL3-VL2-VH1-CL-Fc; VH1-VL2-VL3-VH3-VH2-VL1-CL-Fc; VL1-VL2-VL3-VH3-VH2-VH1-CH1-CL-Fc; VH1-VH2-VH3-VL3-VL2-VL1-CH1-CL-Fc; VL1-VH2-VL3-VH3-VL2-VH1-CH1-CL-Fc; VH1-VL2-VH3-VL3-VH2-VL1-CH1-CL-Fc; VL1-VL2-VH3-VL3-VH2-VH1-CH1-CL-Fc; VH1-VH2-VL3-VH3-VL2-VL1-CH1-CL-Fc; VL1-VH2-VH3-VL3-VL2-VH1-CH1-CL-Fc; VH1-VL2-VL3-VH3-VH2-VL1-CH1-CL-Fc; VL1-VL2-VL3-VH3-VH2-VH1-CL-CH1-Fc; VH1-VH2-VH3-VL3-VL2-VL1-CL-CH1-Fc; VL1-VH2-VL3-VH3-VL2-VH1-CL-CH1-Fc; VH1-VL2-VH3-VL3-VH2-VL1-CL-CH1-Fc; VL1-VL2-VH3-VL3-VH2-VH1-CL-CH1-Fc; VH1-VH2-VL3-VH3-VL2-VL1-CL-CH1-Fc; VL1-VH2-VH3-VL3-VL2-VH1-CL-CH1-Fc; VH1-VL2-VL3-VH3-VH2-VL1-CL-CH1-Fc; VL1-L1-VL2-L2-VL3-L3-VH3-L4-VH2-L5-VH1-CH1-Fc; VH1-L1-VH2-L2-VH3-L3-VL3-L4-VL2-L5-VL1-CH1-Fc; VL1-L1-VH2-L2-VL3-L3-VH3-L4-VL2-L5-VH1-CH1-Fc; VH1-L1-VL2-L2-VH3-L3-VL3-L4-VH2-L5-VL1-CH1-Fc; VL1-L1-VL2-L2-VH3-L3-VL3-L4-VH2-L5-VH1-CH1-Fc; VH1-L1-VH2-L2-VL3-L3-VH3-L4-VL2-L5-VL1-CH1-Fc; VL1-L1-VH2-L2-VH3-L3-VL3-L4-VL2-L5-VH1-CH1-Fc; VH1-L1-VL2-L2-VL3-L3-VH3-L4-VH2-L5-VL1-CH1-Fc; VL1-L1-VL2-L2-VL3-L3-VH3-L4-VH2-L5-VH1-CL-Fc; VH1-L1-VH2-L2-VH3-L3-VL3-L4-VL2-L5-VL1-CL-Fc; VL1-L1-VH2-L2-VL3-L3-VH3-L4-VL2-L5-VH1-CL-Fc; VH1-L1-VL2-L2-VH3-L3-VL3-L4-VH2-L5-VL1-CL-Fc; VL1-L1-VL2-L2-VH3-L3-VL3-L4-VH2-L5-VH1-CL-Fc; VH1-L1-VH2-L2-VL3-L3-VH3-L4-VL2-L5-VL1-CL-Fc; VL1-L1-VH2-L2-VH3-L3-VL3-L4-VL2-L5-VH1-CL-Fc; VH1-L1-VL2-L2-VL3-L3-VH3-L4-VH2-L5-VL1-CL-Fc; VL1-L1-VL2-L2-VL3-L3-VH3-L4-VH2-L5-VH1-CH1-CL-Fc; VH1-L1-VH2-L2-VH3-L3-VL3-L4-VL2-L5-VL1-CH1-CL-Fc; VL1-L1-VH2-L2-VL3-L3-VH3-L4-VL2-L5-VH1-CH1-CL-Fc; VH1-L1-VL2-L2-VH3-L3-VL3-L4-VH2-L5-VL1-CH1-CL-Fc; VL1-L1-VL2-L2-VH3-L3-VL3-L4-VH2-L5-VH1-CH1-CL-Fc; VH1-L1-VH2-L2-VL3-L3-VH3-L4-VL2-L5-VL1-CH1-CL-Fc; VL1-L1-VH2-L2-VH3-L3-VL3-L4-VL2-L5-VH1-CH1-CL-Fc; VH1-L1-VL2-L2-VL3-L3-VH3-L4-VH2-L5-VL1-CH1-CL-Fc; VL1-L1-VL2-L2-VL3-L3-VH3-L4-VH2-L5-VH1-CL-CH1-Fc; VH1-L1-VH2-L2-VH3-L3-VL3-L4-VL2-L5-VL1-CL-CH1-Fc; VL1-L1-VH2-L2-VL3-L3-VH3-L4-VL2-L5-VH1-CL-CH1-Fc; VH1-L1-VL2-L2-VH3-L3-VL3-L4-VH2-L5-VL1-CL-CH1-Fc; VL1-L1-VL2-L2-VH3-L3-VL3-L4-VH2-L5-VH1-CL-CH1-Fc; VH1-L1-VH2-L2-VL3-L3-VH3-L4-VL2-L5-VL1-CL-CH1-Fc; VL1-L1-VH2-L2-VH3-L3-VL3-L4-VL2-L5-VH1-CL-CH1-Fc; or VH1-L1-VL2-L2-VL3-L3-VH3-L4-VH2-L5-VL1-CL-CH1-Fc; wherein the second polypeptide has a structure represented by Fc; VL4-VH4-CH1-Fc; VL4-VH4-CL-Fc; VL4-VH4-CH1-CL-Fc; VL4-VH4-CL-CH1-Fc; VH4-VL4-CH1-Fc; VH4-VL4-CL-Fc; VH4-VL4-CH1-CL-Fc; VH4-VL4-CL-CH1-Fc; VL4-L6-VH4-CH1-Fc; VL4-L6-VH4-CL-Fc; VL4-L6-VH4-CH1-CL-Fc; VL4-L6-VH4-CL-CH1-Fc; VH4-L6-VL4-CH1-Fc; VH4-L6-VL4-CL-Fc; VH4-L6-VL4-CH1-CL-Fc; VH4-L6-VL4-CL-CH1-Fc; VL4-CL-VH4-CH1-Fc; VH4-CL-VL4-CH1-Fc; VL4-CH1-VH4-CL-Fc; VH4-CH1-VL4-CL-Fc; VL4-L6-CL-L7-VH4-L8-CH1-Fc; VL4-L6-CL-L7-VH4-L8-CH1-L9-Fc; VH4-L6-CL-L7-VL4-L8-CH1-Fc; VH4-L6-CL-L7-VL4-L8-CH1-L9-Fc; VL4-L6-CH1-L7-VH4-L8-CL-Fc; VL4-L6-CH1-L7-VH4-L8-CL-L9-Fc; VH4-L6-CH1-L7-VL4-L8-CL-Fc; VH4-L6-CH1-L7-VL4-L8-CL-L9-Fc; VL4-VL5-VH5-VH4-CH1-Fc; VL4-VL5-VH5-VH4-CL-Fc; VL4-VL5-VH5-VH4-CH1-CL-Fc; VL4-VL5-VH5-VH4-CL-CH1-Fc; VH4-VH5-VL5-VL4-CH1-Fc; VH4-VH5-VL5-VL4-CL-Fc; VH4-VH5-VL5-VL4-CH1-CL-Fc; VH4-VH5-VL5-VL4-CL-CH1-Fc; VL4-L6-VL5-L7-VH5-L8-VH4-CH1-Fc; VL4-L6-VL5-L7-VH5-L8-VH4-CL-Fc; VL4-L6-VL5-L7-VH5-L8-VH4-CH1-CL-Fc; VL4-L6-VL5-L7-VH5-L8-VH4-CL-CH1-Fc; VH4-L6-VH5-L7-VL5-L8-VL4-CH1-Fc; VH4-L6-VH5-L7-VL5-L8-VL4-CL-Fc; VH4-L6-VH5-L7-VL5-L8-VL4-CH1-CL-Fc; VH4-L6-VH5-L7-VL5-L8-VL4-CL-CH1-Fc; VL4-VL5-VL6-VH6-VH5-VH4-CH1-Fc; VL4-VL5-VL6-VH6-VH5-VH4-CL-Fc; VL4-VL5-VL6-VH6-VH5-VH4-CH1-CL-Fc; VL4-VL5-VL6-VH6-VH5-VH4-CL-CH1-Fc; VH4-VH5-VH6-VL6-VL5-VL4-CH1-Fc; VH4-VH5-VH6-VL6-VL5-VL4-CL-Fc; VH4-VH5-VH6-VL6-VL5-VL4-CH1-CL-Fc; VH4-VH5-VH6-VL6-VL5-VL4-CL-CH1-Fc; VL4-VH5-VL6-VH6-VL5-VH4-CH1-Fc; VL4-VH5-VL6-VH6-VL5-VH4-CL-Fc; VL4-VH5-VL6-VH6-VL5-VH4-CH1-CL-Fc; VL4-VH5-VL6-VH6-VL5-VH4-CL-CH1-Fc; VH4-VL5-VH6-VL6-VH5-VL4-CH1-Fc; VH4-VL5-VH6-VL6-VH5-VL4-CL-Fc; VH4-VL5-VH6-VL6-VH5-VL4-CH1-CL-Fc; VH4-VL5-VH6-VL6-VH5-VL4-CL-CH1-Fc; VL4-VL5-VH6-VL6-VH5-VH4-CH1-Fc; VL4-VL5-VH6-VL6-VH5-VH4-CL-Fc; VL4-VL5-VH6-VL6-VH5-VH4-CH1-CL-Fc; VL4-VL5-VH6-VL6-VH5-VH4-CL-CH1-Fc; VH4-VH5-VL6-VH6-VL5-VL4-CH1-Fc; VH4-VH5-VL6-VH6-VL5-VL4-CL-Fc; VH4-VH5-VL6-VH6-VL5-VL4-CH1-CL-Fc; VH4-VH5-VL6-VH6-VL5-VL4-CL-CH1-Fc; VL4-VH5-VH6-VL6-VL5-VH4-CH1-Fc; VL4-VH5-VH6-VL6-VL5-VH4-CL-Fc; VL4-VH5-VH6-VL6-VL5-VH4-CH1-CL-Fc; VL4-VH5-VH6-VL6-VL5-VH4-CL-CH1-Fc; VH4-VL5-VL6-VH6-VH5-VL4-CH1-Fc; VH4-VL5-VL6-VH6-VH5-VL4-CL-Fc; VH4-VL5-VL6-VH6-VH5-VL4-CH1-CL-Fc; VH4-VL5-VL6-VH6-VH5-VL4-CL-CH1-Fc; VL4-L6-VL5-L7-VL6-L8-VH6-L9-VH5-L10-VH4-CH1-Fc; VL4-L6-VL5-L7-VL6-L8-VH6-L9-VH5-L10-VH4-CL-Fc; VL4-L6-VL5-L7-VL6-L8-VH6-L9-VH5-L10-VH4-CH1-CL-Fc; VL4-L6-VL5-L7-VL6-L8-VH6-L9-VH5-L10-VH4-CL-CH1-Fc; VH4-L6-VH5-L7-VH6-L8-VL6-L9-VL5-L10-VL4-CH1-Fc; VH4-L6-VH5-L7-VH6-L8-VL6-L9-VL5-L10-VL4-CL-Fc; VH4-L6-VH5-L7-VH6-L8-VL6-L9-VL5-L10-VL4-CH1-CL-Fc; VH4-L6-VH5-L7-VH6-L8-VL6-L9-VL5-L10-VL4-CL-CH1-Fc; VL4-L6-VH5-L7-VL6-L8-VH6-L9-VL5-L10-VH4-CH1-Fc; VL4-L6-VH5-L7-VL6-L8-VH6-L9-VL5-L10-VH4-CL-Fc; VL4-L6-VH5-L7-VL6-L8-VH6-L9-VL5-L10-VH4-CH1-CL-Fc; VL4-L6-VH5-L7-VL6-L8-VH6-L9-VL5-L10-VH4-CL-CH1-Fc; VH4-L6-VL5-L7-VH6-L8-VL6-L9-VH5-L10-VL4-CH1-Fc; VH4-L6-VL5-L7-VH6-L8-VL6-L9-VH5-L10-VL4-CL-Fc; VH4-L6-VL5-L7-VH6-L8-VL6-L9-VH5-L10-VL4-CH1-CL-Fc; VH4-L6-VL5-L7-VH6-L8-VL6-L9-VH5-L10-VL4-CL-CH1-Fc; VL4-L6-VL5-L7-VH6-L8-VL6-L9-VH5-L10-VH4-CH1-Fc; VL4-L6-VL5-L7-VH6-L8-VL6-L9-VH5-L10-VH4-CL-Fc; VL4-L6-VL5-L7-VH6-L8-VL6-L9-VH5-L10-VH4-CH1-CL-Fc; VL4-L6-VL5-L7-VH6-L8-VL6-L9-VH5-L10-VH4-CL-CH1-Fc; VH4-L6-VH5-L7-VL6-L8-VH6-L9-VL5-L10-VL4-CH1-Fc; VH4-L6-VH5-L7-VL6-L8-VH6-L9-VL5-L10-VL4-CL-Fc; VH4-L6-VH5-L7-VL6-L8-VH6-L9-VL5-L10-VL4-CH1-CL-Fc; VH4-L6-VH5-L7-VL6-L8-VH6-L9-VL5-L10-VL4-CL-CH1-Fc; VL4-L6-VH5-L7-VH6-L8-VL6-L9-VL5-L10-VH4-CH1-Fc; VL4-L6-VH5-L7-VH6-L8-VL6-L9-VL5-L10-VH4-CL-Fc; VL4-L6-VH5-L7-VH6-L8-VL6-L9-VL5-L10-VH4-CH1-CL-Fc; VL4-L6-VH5-L7-VH6-L8-VL6-L9-VL5-L10-VH4-CL-CH1-Fc; VH4-L6-VL5-L7-VL6-L8-VH6-L9-VH5-L10-VL4-CH1-Fc; VH4-L6-VL5-L7-VL6-L8-VH6-L9-VH5-L10-VL4-CL-Fc; VH4-L6-VL5-L7-VL6-L8-VH6-L9-VH5-L10-VL4-CH1-CL-Fc; VH4-L6-VL5-L7-VL6-L8-VH6-L9-VH5-L10-VL4-CL-CH1-Fc; VL4-L6-VL5-L7-VL6-L8-VH6-L9-VH5-L10-VH4-L11-CH1-Fc; VL4-L6-VL5-L7-VL6-L8-VH6-L9-VH5-L10-VH4-L11-CL-Fc; VL4-L6-VL5-L7-VL6-L8-VH6-L9-VH5-L10-VH4-L11-CH1-CL-Fc; VL4-L6-VL5-L7-VL6-L8-VH6-L9-VH5-L10-VH4-L11-CL-CH1-Fc; VH4-L6-VH5-L7-VH6-L8-VL6-L9-VL5-L10-VL4-L11-CH1-Fc; VH4-L6-VH5-L7-VH6-L8-VL6-L9-VL5-L10-VL4-L11-CL-Fc; VH4-L6-VH5-L7-VH6-L8-VL6-L9-VL5-L10-VL4-L11-CH1-CL-Fc; VH4-L6-VH5-L7-VH6-L8-VL6-L9-VL5-L10-VL4-L11-CL-CH1-Fc; VL4-L6-VH5-L7-VL6-L8-VH6-L9-VL5-L10-VH4-L11-CH1-Fc; VL4-L6-VH5-L7-VL6-L8-VH6-L9-VL5-L10-VH4-L11-CL-Fc; VL4-L6-VH5-L7-VL6-L8-VH6-L9-VL5-L10-VH4-L11-CH1-CL-Fc; VL4-L6-VH5-L7-VL6-L8-VH6-L9-VL5-L10-VH4-L11-CL-CH1-Fc; VH4-L6-VL5-L7-VH6-L8-VL6-L9-VH5-L10-VL4-L11-CH1-Fc; VH4-L6-VL5-L7-VH6-L8-VL6-L9-VH5-L10-VL4-L11-CL-Fc; VH4-L6-VL5-L7-VH6-L8-VL6-L9-VH5-L10-VL4-L11-CH1-CL-Fc; VH4-L6-VL5-L7-VH6-L8-VL6-L9-VH5-L10-VL4-L11-CL-CH1-Fc; VL4-L6-VL5-L7-VH6-L8-VL6-L9-VH5-L10-VH4-L11-CH1-Fc; VL4-L6-VL5-L7-VH6-L8-VL6-L9-VH5-L10-VH4-L11-CL-Fc; VL4-L6-VL5-L7-VH6-L8-VL6-L9-VH5-L10-VH4-L11-CH1-CL-Fc; VL4-L6-VL5-L7-VH6-L8-VL6-L9-VH5-L10-VH4-L11-CL-CH1-Fc; VH4-L6-VH5-L7-VL6-L8-VH6-L9-VL5-L10-VL4-L11-CH1-Fc; VH4-L6-VH5-L7-VL6-L8-VH6-L9-VL5-L10-VL4-L11-CL-Fc; VH4-L6-VH5-L7-VL6-L8-VH6-L9-VL5-L10-VL4-L11-CH1-CL-Fc; VH4-L6-VH5-L7-VL6-L8-VH6-L9-VL5-L10-VL4-L11-CL-CH1-Fc; VL4-L6-VH5-L7-VH6-L8-VL6-L9-VL5-L10-VH4-L11-CH1-Fc; VL4-L6-VH5-L7-VH6-L8-VL6-L9-VL5-L10-VH4-L11-CL-Fc; VL4-L6-VH5-L7-VH6-L8-VL6-L9-VL5-L10-VH4-L11-CH1-CL-Fc; VL4-L6-VH5-L7-VH6-L8-VL6-L9-VL5-L10-VH4-L11-CL-CH1-Fc; VH4-L6-VL5-L7-VL6-L8-VH6-L9-VH5-L10-VL4-L11-CH1-Fc; VH4-L6-VL5-L7-VL6-L8-VH6-L9-VH5-L10-VL4-L11-CL-Fc; VH4-L6-VL5-L7-VL6-L8-VH6-L9-VH5-L10-VL4-L11-CH1-CL-Fc; or VH4-L6-VL5-L7-VL6-L8-VH6-L9-VH5-L10-VL4-L11-CL-CH1-Fc; wherein VL1 is a first immunoglobulin light chain variable region that specifically binds to an HIV protein; VL2 is a second immunoglobulin light chain variable region that specifically binds to an HIV protein; VL3 is a third immunoglobulin light chain variable region that specifically binds to an HIV protein; VL4 is a fourth immunoglobulin light chain variable region that specifically binds to an HIV protein; VL5 is a fifth immunoglobulin light chain variable region that specifically binds to an HIV protein; VL6 is a sixth immunoglobulin light chain variable region that specifically binds to an HIV protein; VH1 is a first immunoglobulin heavy chain variable region that specifically binds to an HIV protein; VH2 is a second immunoglobulin heavy chain variable region that specifically binds to an HIV protein; VH3 is a third immunoglobulin heavy chain variable region that specifically binds to an HIV protein; VH4 is a fourth immunoglobulin heavy chain variable region that specifically binds to an HIV protein; VH5 is a fifth immunoglobulin heavy chain variable region that specifically binds to an HIV protein; VH6 is a sixth immunoglobulin heavy chain variable region that specifically binds to an HIV protein; Fc is a region comprising an immunoglobulin heavy chain constant region 2 (CH2), an immunoglobulin heavy chain constant region 3 (CH3), and optionally, an immunoglobulin hinge; CH1 is a heavy chain constant region 1; CL is a light chain constant region; and L1, L2, L3, L4, L5, L6, L7, L8, L9, L10 and L11 are amino acid linkers.

Provided herein is an antigen binding polypeptide having a structure represented by VL1-VL2-VL3-VH3-VH2-VH1-CH3-CH3; VH1-VH2-VH3-VL3-VL2-VL1-CH3-CH3; VL1-VH2-VL3-VH3-VL2-VH1-CH3-CH3; VH1-VL2-VH3-VL3-VH2-VL1-CH3-CH3; VL1-VL2-VH3-VL3-VH2-VH1-CH3-CH3; VH1-VH2-VL3-VH3-VL2-VL1-CH3-CH3; VL1-VH2-VH3-VL3-VL2-VH1-CH3-CH3; VH1-VL2-VL3-VH3-VH2-VL1-CH3-CH3; VL1-L1-VL2-L2-VL3-L3-VH3-L4-VH2-L5-VH1-CH3-CH3; VH1-L1-VH2-L2-VH3-L3-VL3-L4-VL2-L5-VL1-CH3-CH3; VL1-L1-VH2-L2-VL3-L3-VH3-L4-VL2-L5-VH1-CH3-CH3; VH1-L1-VL2-L2-VH3-L3-VL3-L4-VH2-L5-VL1-CH3-CH3; VL1-L1-VL2-L2-VH3-L3-VL3-L4-VH2-L5-VH1-CH3-CH3; VH1-L1-VH2-L2-VL3-L3-VH3-L4-VL2-L5-VL1-CH3-CH3; VL1-L1-VH2-L2-VH3-L3-VL3-L4-VL2-L5-VH1-CH3-CH3; VH1-L1-VL2-L2-VL3-L3-VH3-L4-VH2-L5-VL1-CH3-CH3; VL1-L1-VL2-L2-VL3-L3-VH3-L4-VH2-L5-VH1-L6-CH3-CH3; VH1-L1-VH2-L2-VH3-L3-VL3-L4-VL2-L5-VL1-L6-CH3-CH3; VL1-L1-VH2-L2-VL3-L3-VH3-L4-VL2-L5-VH1-L6-CH3-CH3; VH1-L1-VL2-L2-VH3-L3-VL3-L4-VH2-L5-VL1-L6-CH3-CH3; VL1-L1-VL2-L2-VH3-L3-VL3-L4-VH2-L5-VH1-L6-CH3-CH3; VH1-L1-VH2-L2-VL3-L3-VH3-L4-VL2-L5-VL1-L6-CH3-CH3; VL1-L1-VH2-L2-VH3-L3-VL3-L4-VL2-L5-VH1-L6-CH3-CH3; VH1-L1-VL2-L2-VL3-L3-VH3-L4-VH2-L5-VL1-L6-CH3-CH3; VL1-L1-VL2-L2-VL3-L3-VH3-L4-VH2-L5-VH1-L6-CH3-L7-CH3; VH1-L1-VH2-L2-VH3-L3-VL3-L4-VL2-L5-VL1-L6-CH3-L7-CH3; VL1-L1-VH2-L2-VL3-L3-VH3-L4-VL2-L5-VH1-L6-CH3-L7-CH3; VH1-L1-VL2-L2-VH3-L3-VL3-L4-VH2-L5-VL1-L6-CH3-L7-CH3; VL1-L1-VL2-L2-VH3-L3-VL3-L4-VH2-L5-VH1-L6-CH3-L7-CH3; VH1-L1-VH2-L2-VL3-L3-VH3-L4-VL2-L5-VL1-L6-CH3-L7-CH3; VL1-L1-VH2-L2-VH3-L3-VL3-L4-VL2-L5-VH1-L6-CH3-L7-CH3; or VH1-L1-VL2-L2-VL3-L3-VH3-L4-VH2-L5-VL1-L6-CH3-L7-CH3; wherein VL1 is a first immunoglobulin light chain variable region that specifically binds to an HIV protein; VL2 is a second immunoglobulin light chain variable region that specifically binds to an HIV protein; VL3 is a third immunoglobulin light chain variable region that specifically binds to an HIV protein; VH1 is a first immunoglobulin heavy chain variable region that specifically binds to an HIV protein; VH2 is a second immunoglobulin heavy chain variable region that specifically binds to an HIV protein; VH3 is a third immunoglobulin heavy chain variable region that specifically binds to an HIV protein; CH3 is an immunoglobulin heavy chain constant region 3; and L1, L2, L3, L4, L5, L6 and L7 are amino acid linkers.

Provided herein is an antigen binding polypeptide complex comprising a first polypeptide, a second polypeptide, and a third polypeptide; wherein the first polypeptide has a structure represented by VL1-VL2-VL3-VH3-VH2-VH1; VH1-VH2-VH3-VL3-VL2-VL1; VL1-VH2-VL3-VH3-VL2-VH1; VH1-VL2-VH3-VL3-VH2-VL1; VL1-VL2-VH3-VL3-VH2-VH1; VH1-VH2-VL3-VH3-VL2-VL1; VL1-VH2-VH3-VL3-VL2-VH1; VH1-VL2-VL3-VH3-VH2-VL1; VL1-L1-VL2-L2-VL3-L3-VH3-L4-VH2-L5-VH1; VH1-L1-VH2-L2-VH3-L3-VL3-L4-VL2-L5-VL1; VL1-L1-VH2-L2-VL3-L3-VH3-L4-VL2-L5-VH1; VH1-L1-VL2-L2-VH3-L3-VL3-L4-VH2-L5-VL1; VL1-L1-VL2-L2-VH3-L3-VL3-L4-VH2-L5-VH1; VH1-L1-VH2-L2-VL3-L3-VH3-L4-VL2-L5-VL1; VL1-L1-VH2-L2-VH3-L3-VL3-L4-VL2-L5-VH1; or VH1-L1-VL2-L2-VL3-L3-VH3-L4-VH2-L5-VL1; wherein the second polypeptide has a structure represented by VL4-VL5; VL4-L6-VL5; VL4-VL5-VL6; VL4-L6-VL5-L7-VL6; VL4-CL; VL4-L6-CL; VL4-VL5-CL; VL4-L6-VL5-CL; VL4-L6-VL5-L7-CL; VL4-VL5-VL6-CL; VL4-L6-VL5-L7-VL6-CL; VL4-L6-VL5-L7-VL6-L8-CL; VL4-CH1; VL4-L6-CH1; VL4-VL5-CH1; VL4-L6-VL5-CH1; VL4-L6-VL5-L7-CH1; VL4-VL5-VL6-CH1; VL4-L6-VL5-L7-VL6-CH1; or VL4-L6-VL5-L7-VL6-L8-CH1; wherein the third polypeptide has a structure represented by VH4-VH5; VH4-L9-VH5; VH4-VH5-VH6; VH4-L9-VH5-L10-VH6; VH4-CH1; VH4-L9-CH1; VH4-VH5-CH1; VH4-L9-VH5-CH1; VH4-L9-VH5-L10-CH1; VH4-VH5-VH6-CH1; VH4-L9-VH5-L10-VH6-CH1; VH4-L9-VH5-L10-VH6-L11-CH1; VH4-CL; VH4-L9-CL; VH4-VH5-CL; VH4-L9-VH5-CL; VH4-L9-VH5-L10-CL; VH4-VH5-VH6-CL; VH4-L9-VH5-L10-VH6-CL; or VH4-L9-VH5-L10-VH6-L11-CL; wherein VL1 is a first immunoglobulin light chain variable region that specifically binds to an HIV protein; VL2 is a second immunoglobulin light chain variable region that specifically binds to an HIV protein; VL3 is a third immunoglobulin light chain variable region that specifically binds to an HIV protein; VL4 is a fourth immunoglobulin light chain variable region that specifically binds to an HIV protein; VL5 is a fifth immunoglobulin light chain variable region that specifically binds to an HIV protein; VL6 is a sixth immunoglobulin light chain variable region that specifically binds to an HIV protein; VH1 is a first immunoglobulin heavy chain variable region that specifically binds to an HIV protein; VH2 is a second immunoglobulin heavy chain variable region that specifically binds to an HIV protein; VH3 is a third immunoglobulin heavy chain variable region that specifically binds to an HIV protein; VH4 is a fourth immunoglobulin heavy chain variable region that specifically binds to an HIV protein; VH5 is a fifth immunoglobulin heavy chain variable region that specifically binds to an HIV protein; VH6 is a sixth immunoglobulin heavy chain variable region that specifically binds to an HIV protein; CH1 is a heavy chain constant region 1; CL is a light chain constant region; and L1, L2, L3, L4, L5, L6, L7, L8, L9, L10 and L11 are amino acid linkers.

Provided herein is an antigen binding polypeptide complex comprising a first polypeptide, a second polypeptide, and a third polypeptide; wherein the first polypeptide has a structure represented by VL1-VL2-VL3-VH3-VH2-VH1-Fc; VH1-VH2-VH3-VL3-VL2-VL1-Fc; VL1-VH2-VL3-VH3-VL2-VH1-Fc; VH1-VL2-VH3-VL3-VH2-VL1-Fc; VL1-VL2-VH3-VL3-VH2-VH1-Fc; VH1-VH2-VL3-VH3-VL2-VL1-Fc; VL1-VH2-VH3-VL3-VL2-VH1-Fc; VH1-VL2-VL3-VH3-VH2-VL1-Fc; VL1-L1-VL2-L2-VL3-L3-VH3-L4-VH2-L5-VH1-Fc; VL1-L1-VL2-L2-VL3-L3-VH3-L4-VH2-L5-VH1-L6-Fc; VH1-L1-VH2-L2-VH3-L3-VL3-L4-VL2-L5-VL1-Fc; VH1-L1-VH2-L2-VH3-L3-VL3-L4-VL2-L5-VL1-L6-Fc; VL1-L1-VH2-L2-VL3-L3-VH3-L4-VL2-L5-VH1-Fc; VL1-L1-VH2-L2-VL3-L3-VH3-L4-VL2-L5-VH1-L6-Fc; VH1-L1-VL2-L2-VH3-L3-VL3-L4-VH2-L5-VL1-Fc; VH1-L1-VL2-L2-VH3-L3-VL3-L4-VH2-L5-VL1-L6-Fc; VL1-L1-VL2-L2-VH3-L3-VL3-L4-VH2-L5-VH1-Fc; VL1-L1-VL2-L2-VH3-L3-VL3-L4-VH2-L5-VH1-L6-Fc; VH1-L1-VH2-L2-VL3-L3-VH3-L4-VL2-L5-VL1-Fc; VH1-L1-VH2-L2-VL3-L3-VH3-L4-VL2-L5-VL1-L6-Fc; VL1-L1-VH2-L2-VH3-L3-VL3-L4-VL2-L5-VH1-Fc; VL1-L1-VH2-L2-VH3-L3-VL3-L4-VL2-L5-VH1-L6-Fc; VH1-L1-VL2-L2-VL3-L3-VH3-L4-VH2-L5-VL1-Fc; or VH1-L1-VL2-L2-VL3-L3-VH3-L4-VH2-L5-VL1-L6-Fc; wherein the second polypeptide has a structure represented by VL4-VL5; VL4-L7-VL5; VL4-CL; VL4-L7-CL; VL4-CH1; VL4-L7-CH1; VH4-VH5; VH4-L7-VH5; VH4-CL; VH4-L7-CL; VH4-CH1; VH4-L7-CH1; VL4-VL5-VL6; VL4-L7-VL5-L8-VL6; VL4-VL5-VL6-CL; VL4-L7-VL5-L8-VL6-CL; VL4-L7-VL5-L8-VL6-L9-CL; VL4-VL5-VL6-CH1; VL4-L7-VL5-L8-VL6-CH1; VL4-L7-VL5-L8-VL6-L9-CH1; VH4-VH5-VH6; VH4-L7-VH5-L8-VH6; VH4-VH5-VH6-CL; VH4-L7-VH5-L8-VH6-CL; VH4-L7-VH5-L8-VH6-L9-CL; VH4-VH5-VH6-CH1; VH4-L7-VH5-L8-VH6-CH1; or VH4-L7-VH5-L8-VH6-L9-CH1; wherein the third polypeptide has a structure represented by VH4-VH5-Fc; VH4-L10-VH5-Fc; VH4-L10-VH5-L11-Fc; VH4-CH1-Fc; VH4-L10-CH1-Fc; VH4-L10-CH1-L11-Fc; VH4-CL-Fc; VH4-L10-CL-Fc; VH4-L10-CL-L11-Fc; VH4-VH5-Fc; VH4-L10-VH5-Fc; VH4-L10-VH5-L11-Fc; VH4-VH5-VH6-Fc; VH4-L10-VH5-L11-VH6-Fc; VH4-L10-VH5-L11-VH6-L12-Fc; VH4-VH5-VH6-CH1-Fc; VH4-L10-VH5-L11-VH6-CH1-Fc; VH4-L10-VH5-L11-VH6-L12-CH1-Fc; VH4-L10-VH5-L11-VH6-L12-CH1-L13-Fc; VH4-VH5-VH6-CL-Fc; VH4-L10-VH5-L11-VH6-CL-Fc; VH4-L10-VH5-L11-VH6-L12-CL-Fc; VH4-L10-VH5-L11-VH6-L12-CL-L13-Fc; VL4-VL5-VL6-Fc; VL4-L10-VL5-L11-VL6-Fc; VL4-L10-VL5-L11-VL6-L12-Fc; VL4-VL5-VL6-CH1-Fc; VL4-L10-VL5-L11-VL6-CH1-Fc; VL4-L10-VL5-L11-VL6-L12-CH1-Fc; VL4-L10-VL5-L11-VL6-L12-CH1-L13-Fc; VL4-VL5-VL6-CL-Fc; VL4-L10-VL5-L11-VL6-CL-Fc; VL4-L10-VL5-L11-VL6-L12-CL-Fc; or VL4-L10-VL5-L11-VL6-L12-CL-L13-Fc; wherein VL1 is a first immunoglobulin light chain variable region that specifically binds to an HIV protein; VL2 is a second immunoglobulin light chain variable region that specifically binds to an HIV protein; VL3 is a third immunoglobulin light chain variable region that specifically binds to an HIV protein; VL4 is a fourth immunoglobulin light chain variable region that specifically binds to an HIV protein; VL5 is a fifth immunoglobulin light chain variable region that specifically binds to an HIV protein; VL6 is a sixth immunoglobulin light chain variable region that specifically binds to an HIV protein; VH1 is a first immunoglobulin heavy chain variable region that specifically binds to an HIV protein; VH2 is a second immunoglobulin heavy chain variable region that specifically binds to an HIV protein; VH3 is a third immunoglobulin heavy chain variable region that specifically binds to an HIV protein; VH4 is a fourth immunoglobulin heavy chain variable region that specifically binds to an HIV protein; VH5 is a fifth immunoglobulin heavy chain variable region that specifically binds to an HIV protein; VH6 is a sixth immunoglobulin heavy chain variable region that specifically binds to an HIV protein; Fc is a region comprising an immunoglobulin heavy chain constant region 2 (CH2), an immunoglobulin heavy chain constant region 3 (CH3), and optionally, an immunoglobulin hinge; CH1 is a heavy chain constant region 1; CL is a light chain constant region; and L1, L2, L3, L4, L5, L6, L7, L8, L9, L10, L11, L12 and L13 are amino acid linkers.

Provided herein is an antigen binding polypeptide complex comprising a first polypeptide, a second polypeptide, and a third polypeptide; wherein the first polypeptide has a structure represented by VL1-VL2-VL3-VH3-VH2-VH1-Fc; VH1-VH2-VH3-VL3-VL2-VL1-Fc; VL1-VH2-VL3-VH3-VL2-VH1-Fc; VH1-VL2-VH3-VL3-VH2-VL1-Fc; VL1-VL2-VH3-VL3-VH2-VH1-Fc; VH1-VH2-VL3-VH3-VL2-VL1-Fc; VL1-VH2-VH3-VL3-VL2-VH1-Fc; VH1-VL2-VL3-VH3-VH2-VL1-Fc; VL1-L1-VL2-L2-VL3-L3-VH3-L4-VH2-L5-VH1-Fc; VL1-L1-VL2-L2-VL3-L3-VH3-L4-VH2-L5-VH1-L6-Fc; VH1-L1-VH2-L2-VH3-L3-VL3-L4-VL2-L5-VL1-Fc; VH1-L1-VH2-L2-VH3-L3-VL3-L4-VL2-L5-VL1-L6-Fc; VL1-L1-VH2-L2-VL3-L3-VH3-L4-VL2-L5-VH1-Fc; VL1-L1-VH2-L2-VL3-L3-VH3-L4-VL2-L5-VH1-L6-Fc; VH1-L1-VL2-L2-VH3-L3-VL3-L4-VH2-L5-VL1-Fc; VH1-L1-VL2-L2-VH3-L3-VL3-L4-VH2-L5-VL1-L6-Fc; VL1-L1-VL2-L2-VH3-L3-VL3-L4-VH2-L5-VH1-Fc; VL1-L1-VL2-L2-VH3-L3-VL3-L4-VH2-L5-VH1-L6-Fc; VH1-L1-VH2-L2-VL3-L3-VH3-L4-VL2-L5-VL1-Fc; VH1-L1-VH2-L2-VL3-L3-VH3-L4-VL2-L5-VL1-L6-Fc; VL1-L1-VH2-L2-VH3-L3-VL3-L4-VL2-L5-VH1-Fc; VL1-L1-VH2-L2-VH3-L3-VL3-L4-VL2-L5-VH1-L6-Fc; VH1-L1-VL2-L2-VL3-L3-VH3-L4-VH2-L5-VL1-Fc; or VH1-L1-VL2-L2-VL3-L3-VH3-L4-VH2-L5-VL1-L6-Fc; wherein the second polypeptide has a structure represented by VL4-VL5-Fc; VL4-L7-VL5-Fc; VL4-L7-VL5-L8-Fc; VL4-CL-Fc; VL4-L7-CL-Fc; VL4-L7-CL-L8-Fc; VL4-CH1-Fc; VL4-L7-CH1-Fc; VL4-L7-CH1-L8-Fc; VH4-VH5-Fc; VH4-L7-VH5-Fc; VH4-L7-VH5-L8-Fc; VH4-CL-Fc; VH4-L7-CL-Fc; VH4-L7-CL-L8-Fc; VH4-CH1-Fc; VH4-L7-CH1-Fc; VH4-L7-CH1-L8-Fc; VL4-VL5-VL6-Fc; VL4-L7-VL5-L8-VL6-Fc; VL4-L7-VL5-L8-VL6-L9-Fc; VL4-VL5-VL6-CL-Fc; VL4-L7-VL5-L8-VL6-CL-Fc; VL4-L7-VL5-L8-VL6-L9-CL-Fc; VL4-L7-VL5-L8-VL6-L9-CL-L10-Fc; VL4-VL5-VL6-CH1-Fc; VL4-L7-VL5-L8-VL6-CH1-Fc; VL4-L7-VL5-L8-VL6-L9-CH1-Fc; VL4-L7-VL5-L8-VL6-L9-CH1-L10-Fc; VH4-VH5-VH6-Fc; VH4-L7-VH5-L8-VH6-Fc; VH4-L7-VH5-L8-VH6-L9-Fc; VH4-VH5-VH6-CL-Fc; VH4-L7-VH5-L8-VH6-CL-Fc; VH4-L7-VH5-L8-VH6-L9-CL-Fc; VH4-L7-VH5-L8-VH6-L9-CL-L10-Fc; VH4-VH5-VH6-CH1-Fc; VH4-L7-VH5-L8-VH6-CH1-Fc; VH4-L7-VH5-L8-VH6-L9-CH1-Fc; or VH4-L7-VH5-L8-VH6-L9-CH1-L10-Fc; wherein the third polypeptide has a structure represented by VH4-VH5; VH4-L11-VH5; VH4-CH1; VH4-L11-CH1; VH4-CL; VH4-L11-CL; VH4-VH5; VH4-L11-VH5; VH4-VH5-VH6; VH4-L11-VH5-L12-VH6; VH4-VH5-VH6-CH1; VH4-L11-VH5-L12-VH6-CH1; VH4-L11-VH5-L12-VH6-L13-CH1; VH4-VH5-VH6-CL; VH4-L11-VH5-L12-VH6-CL; VH4-L11-VH5-L12-VH6-L13-CL; VL4-VL5-VL6; VL4-L11-VL5-L12-VL6; VL4-VL5-VL6-CH1; VL4-L11-VL5-L12-VL6-CH1; VL4-L11-VL5-L12-VL6-L13-CH1; VL4-VL5-VL6-CL; VL4-L11-VL5-L12-VL6-CL; or VL4-L11-VL5-L12-VL6-L13-CL; wherein VL1 is a first immunoglobulin light chain variable region that specifically binds to an HIV protein; VL2 is a second immunoglobulin light chain variable region that specifically binds to an HIV protein; VL3 is a third immunoglobulin light chain variable region that specifically binds to an HIV protein; VL4 is a fourth immunoglobulin light chain variable region that specifically binds to an HIV protein; VL5 is a fifth immunoglobulin light chain variable region that specifically binds to an HIV protein; VL6 is a sixth immunoglobulin light chain variable region that specifically binds to an HIV protein; VH1 is a first immunoglobulin heavy chain variable region that specifically binds to an HIV protein; VH2 is a second immunoglobulin heavy chain variable region that specifically binds to an HIV protein; VH3 is a third immunoglobulin heavy chain variable region that specifically binds to an HIV protein; VH4 is a fourth immunoglobulin heavy chain variable region that specifically binds to an HIV protein; VH5 is a fifth immunoglobulin heavy chain variable region that specifically binds to an HIV protein; VH6 is a sixth immunoglobulin heavy chain variable region that specifically binds to an HIV protein; Fc is a region comprising an immunoglobulin heavy chain constant region 2 (CH2), an immunoglobulin heavy chain constant region 3 (CH3), and optionally, an immunoglobulin hinge; CH1 is a heavy chain constant region 1; CL is a light chain constant region; and L1, L2, L3, L4, L5, L6, L7, L8, L9, L10, L11, L12 and L13 are amino acid linkers.

In some aspects, an HIV protein to which the heavy and light chain variable regions specifically bind is an HIV envelope protein, an HIV structural protein, an HIV functional protein, or an HIV accessory protein. In other aspects, the HIV envelope protein is HIV envelope glycoprotein (Env), HIV envelope glycoprotein gp160, HIV envelope surface glycoprotein gp120, or HIV transmembrane envelope protein gp41. In other aspects, the HIV structural protein is p17, p24, p7 or p55. In other aspects, the HIV functional protein is p66, HIV-1 protease (PR) or p31. In other aspects, the HIV accessory protein is Nef, Tat, Rev, Vif, Vpr or Vpu.

Provided herein is an antibody or antigen binding fragment thereof comprising an antigen binding polypeptide or antigen binding polypeptide complex described herein.

Provided herein is a polypeptide having at least 90% identity, at least 95% identity, or 100% identity to any one of SEQ ID NOs:32-47, 84, 86, 88, 90, 92, 94, 96 and 98. Also provided herein is a polypeptide encoded by a polynucleotide having at least 90% identity, at least 95% identity, or 100% identity to any one of SEQ ID NOs:48-59, 85, 87, 89, 91, 93, 95, 97 and 99.

Provided herein is a polynucleotide encoding an antigen binding polypeptide or antigen binding polypeptide complex described herein. Also provided herein is a polynucleotide having at least 90% identity, at least 95% identity, or 100% identity to any one of SEQ ID NOs:48-59, 85, 87, 89, 91, 93, 95, 97 and 99. Also provided herein is a polynucleotide encoding a polypeptide having at least 90% identity, at least 95% identity, or 100% identity to any one of SEQ ID NOs:32-47, 84, 86, 88, 90, 92, 94, 96 and 98.

Provided herein is a vector comprising a polynucleotide described herein.

Provided herein is a host cell comprising a polynucleotide or vector described herein.

Provided herein is a chimeric antigen receptor (CAR) comprising an antigen binding polypeptide or antigen binding polypeptide complex described herein.

Provided herein is an immune cell comprising a CAR described herein.

Provided herein is a pharmaceutical composition comprising (i) an antigen binding polypeptide or antigen binding polypeptide complex, antibody or antigen binding fragment thereof, polypeptide, polynucleotide, vector, host cell, CAR, or immune cell described herein, or a combination thereof, and (ii) a pharmaceutically acceptable carrier.

Provided herein is a kit comprising an antigen binding polypeptide or antigen binding polypeptide complex, antibody or antigen binding fragment thereof, polypeptide, polynucleotide, vector, host cell, CAR, immune cell, or pharmaceutical composition described herein, or a combination thereof.

Provided herein is a method of treating or preventing human immunodeficiency virus (HIV) infection, comprising administering to a subject in need thereof a therapeutically effective amount of an antigen binding polypeptide or antigen binding polypeptide complex, antibody or antigen binding fragment thereof, polypeptide, polynucleotide, vector, host cell, CAR, immune cell, or pharmaceutical composition described herein, or a combination thereof.

Provided herein is a method of treating or preventing acquired immune deficiency syndrome (AIDS), comprising administering to a subject in need thereof a therapeutically effective amount of an antigen binding polypeptide or antigen binding polypeptide complex, antibody or antigen binding fragment thereof, polypeptide, polynucleotide, vector, host cell, CAR, immune cell, or pharmaceutical composition described herein, or a combination thereof.

Provided herein is a method of treating or preventing AIDS-related complex (ARC), comprising administering to a subject in need thereof a therapeutically effective amount of an antigen binding polypeptide or antigen binding polypeptide complex, antibody or antigen binding fragment thereof, polypeptide, polynucleotide, vector, host cell, CAR, immune cell, or pharmaceutical composition described herein, or a combination thereof.

Provided herein is a method of treating or preventing an HIV-related opportunistic infection, comprising administering to a subject in need thereof a therapeutically effective amount of an antigen binding polypeptide or antigen binding polypeptide complex, antibody or antigen binding fragment thereof, polypeptide, polynucleotide, vector, host cell, CAR, immune cell, or pharmaceutical composition described herein, or a combination thereof.

### DETAILED DESCRIPTION OF THE INVENTION

The invention is directed to antigen binding polypeptides and antigen binding polypeptide complexes (e.g., antibodies or antigen binding fragments thereof) having improved features. In some aspects, the invention enables the generation of multispecific and multifunctional antigen binding polypeptides and antigen binding polypeptide complexes through the expression of complementary self-assembling heavy and light chains expressed with a single polypeptide per arm and, optionally, with the addition of specific amino acid linkers. Because of this multifunctionality, antigen binding polypeptides and antigen binding polypeptide complexes of the invention can bind to specific combinations of target molecules for selectivity or breadth/neutralization, bring together two or more cell types, bring together targets and deliver activation signals, modify the disease microenvironment, and enhance avidity of binding for improved potency.

Various terms relating to aspects of disclosure are used throughout the specification and claims. Such terms are to be given their ordinary meaning in the art, unless otherwise indicated. Other specifically defined terms are to be construed in a manner consistent with the definition provided herein.

### Definitions

As used herein, the term “antigen binding polypeptide” refers to a polypeptide having the ability to specifically bind to one or more substances that induce an immune response (i.e., one or more antigens or epitopes).

As used herein, the term “antigen binding polypeptide complex” refers to a group of two, three, four, or more associated polypeptides, wherein at least one polypeptide has the ability to specifically bind to one or more antigens. An antigen binding polypeptide complex, includes, but is not limited to, an antibody or antigen binding fragment thereof.

The term “antibody” includes, without limitation, a glycoprotein immunoglobulin which binds specifically to an antigen and comprises at least two heavy (H) chains and two light (L) chains interconnected by disulfide bonds. Each H chain comprises a heavy chain variable region (abbreviated herein as VH) and a heavy chain constant region. The heavy chain constant region comprises three constant domains, CH1, CH2 and CH3. Each light chain comprises a light chain variable region (abbreviated herein as VL) and a light chain constant region. The light chain constant region comprises one constant domain, CL. The VH and VL regions can be further subdivided into regions of hypervariability, termed complementarity determining regions (CDRs), interspersed with regions that are more conserved, termed framework regions (FR). Each VH and VL comprises three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system. A heavy chain may have the C-terminal lysine or not. Unless specified otherwise herein, the amino acids in the variable regions are numbered using the Kabat numbering system and those in the constant regions are numbered using the EU system.

The term “monoclonal antibody,” as used herein, refers to an antibody that is produced by a single clone of B-cells and binds to the same epitope. In contrast, the term “polyclonal antibody” refers to a population of antibodies that are produced by different B-cells and bind to different epitopes of the same antigen. The term “antibody” includes, by way of example, monoclonal and polyclonal antibodies; chimeric and humanized antibodies; human or non-human antibodies; wholly synthetic antibodies; and single chain antibodies. A non-human antibody can be humanized by recombinant methods to reduce its immunogenicity in man.

The antibody can be an antibody that has been altered (e.g., by mutation, deletion, substitution, conjugation to a non-antibody moiety). For example, an antibody can include one or more variant amino acids (compared to a naturally occurring antibody) which change a property (e.g., a functional property) of the antibody. For example, several such alterations are known in the art which affect, e.g., half-life, effector function, and/or immune responses to the antibody in a patient. The term antibody also includes artificial polypeptide constructs which comprise at least one antibody-derived antigen binding site.

An “antigen binding fragment” of an antibody refers to one or more fragments or portions of an antibody that retain the ability to bind specifically to the antigen bound by the whole antibody. It has been shown that the antigen-binding function of an antibody can be performed by fragments or portions of a full-length antibody. An antigen binding fragment can contain the antigenic determining regions of an intact antibody (e.g., the complementarity determining regions (CDRs)). Examples of antigen binding fragments of antibodies include, but are not limited to, Fab, Fab′, F(ab′)2, and Fv fragments, linear antibodies, and single chain antibodies. An antigen binding fragment of an antibody can be derived from any animal species, such as rodents (e.g., mouse, rat, or hamster) and humans or can be artificially produced.

Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see, e.g., Bird et al. (1988) Science 242:423-426; and Huston et al. (1988) Proc. Natl. Acad. Sci. USA 85:5879-5883). Such single chain antibodies are also intended to be encompassed within the term “antigen binding fragment” of an antibody.

Antigen binding fragments are obtained using conventional techniques known to those with skill in the art, and the fragments are screened for utility in the same manner as are intact antibodies. Antigen binding fragments can be produced by recombinant DNA techniques, or by enzymatic or chemical cleavage of intact immunoglobulins.

As used herein, the term “variable region” typically refers to a portion of an antibody, generally, a portion of a light or heavy chain, typically about the amino-terminal 110 to 120 amino acids, or 110 to 125 amino acids in the mature heavy chain and about 90 to 115 amino acids in the mature light chain, which differ extensively in sequence among antibodies and are used in the binding and specificity of a particular antibody for its particular antigen. The variability in sequence is concentrated in those regions called complementarity determining regions (CDRs) while the more highly conserved regions in the variable domain are called framework regions (FR). Without wishing to be bound by any particular mechanism or theory, it is believed that the CDRs of the light and heavy chains are primarily responsible for the interaction and specificity of an antibody with antigen. In some aspects, the variable region is a mammalian variable region, e.g., a human, mouse or rabbit variable region. In some aspects, the variable region comprises rodent or murine CDRs and human framework regions (FRs). In some aspects, the variable region is a primate (e.g., non-human primate) variable region. In some aspects, the variable region comprises rodent or murine CDRs and primate (e.g., non-human primate) FRs.

The terms “complementarity determining region” or “CDR”, as used herein, refer to each of the regions of an antibody variable domain which are hypervariable in sequence and/or form structurally defined loops (hypervariable loops) and/or contain the antigen-contacting residues. Antibodies can comprise six CDRs, e.g., three in the VH and three in the VL.

The terms “VL”, “VL region,” and “VL domain” are used herein interchangeably to refer to the light chain variable region of an antigen binding polypeptide, antigen binding polypeptide complex, antibody or antigen binding fragment thereof. In some aspects, a VL region is referred to herein as VL1 to denote a first light chain variable region, VL2 to denote a second light chain variable region, VL3 to denote a third light chain variable region, and so on. An enumerated VL region (e.g., VL1) can have the same or different antigen binding properties and/or the same or different sequence as another enumerated VL region (e.g., VL2).

The terms “VH”, “VH region,” and “VH domain” are used herein interchangeably to refer to the heavy chain variable region of an antigen binding polypeptide, antigen binding polypeptide complex, antibody or antigen binding fragment thereof. In some aspects, a VH region is referred to herein as VH1 to denote a first heavy chain variable region, VH2 to denote a second heavy chain variable region, VH3 to denote a third heavy chain variable region, and so on. An enumerated VH region (e.g., VH1) can have the same or different antigen binding properties and/or the same or different sequence as another enumerated VH region (e.g., VH2).

As used herein, “Kabat numbering” and like terms are recognized in the art and refer to a system of numbering amino acid residues in the heavy and light chain variable regions of an antibody or antigen binding fragment thereof. In some aspects, CDRs can be determined according to the Kabat numbering system (see, e.g., Kabat E A & Wu T T (1971) Ann. NY Acad. Sci. 190: 382-391 and Kabat E A et al., (1991) Sequences of Proteins of Immunological Interest, Fifth Edition, U.S. Department of Health and Human Services, NIH Publication No. 91-3242). Using the Kabat numbering system, CDRs within an antibody heavy chain molecule are typically present at amino acid positions 31 to 35, which optionally can include one or two additional amino acids, following 35 (referred to in the Kabat numbering scheme as 35A and 35B) (CDR1), amino acid positions 50 to 65 (CDR2), and amino acid positions 95 to 102 (CDR3). Using the Kabat numbering system, CDRs within an antibody light chain molecule are typically present at amino acid positions 24 to 34 (CDR1), amino acid positions 50 to 56 (CDR2), and amino acid positions 89 to 97 (CDR3).

As used herein, the terms “constant region” or “constant domain” are used interchangeably to refer to a portion of an antigen binding polypeptide, antigen binding polypeptide complex, antibody or antigen binding fragment thereof, e.g., a carboxyl terminal portion of a light and/or heavy chain which is not directly involved in binding of an antibody to antigen but which can exhibit various effector functions, such as interaction with the Fc region. The constant region generally has a more conserved amino acid sequence relative to a variable region. In some aspects, an antigen binding polypeptide, antigen binding polypeptide complex, antibody or antigen binding fragment thereof comprises a constant region or portion thereof that is sufficient for antibody-dependent cell-mediated cytotoxicity (ADCC), antibody-dependent cellular phagocytosis (ADCP), and complement-dependent cytotoxicity (CDC).

As used herein, the terms “fragment crystallizable region,” “Fc region,” or “Fc domain” are used interchangeably herein to refer to the tail region of an antibody that interacts with cell surface receptors called Fc receptors and some proteins of the complement system. Fc regions typically comprise CH2 and CH3 regions, and, optionally, an immunoglobulin hinge.

As used herein, the terms “immunoglobulin hinge,” “hinge,” “hinge domain” or “hinge region” are used interchangeably to refer to a stretch of heavy chains between the Fab and Fc portions of an antigen binding polypeptide, antigen binding polypeptide complex, antibody or antigen binding fragment thereof. A hinge provides structure, position and flexibility, which assist with normal functioning of antibodies (e.g., for crosslinking two antigens or binding two antigenic determinants on the same antigen molecule). An immunoglobulin hinge is divided into upper, middle and lower hinge regions that can be separated based on structural and/or genetic components. An immunoglobulin hinge of the invention can contain one, two or all three of these regions. Structurally, the upper hinge region stretches from the C terminal end of CH1 to the first hinge disulfide bond. The middle hinge region stretches from the first cysteine to the last cysteine in the hinge. The lower hinge region extends from the last cysteine to the glycine of CH2. The cysteines present in the hinge form interchain disulfide bonds that link the immunoglobulin monomers.

As used herein, the term “Fab” refers to a region of an antibody that binds to an antigen. It is typically composed of one constant and one variable domain of each of the heavy and the light chain.

As used herein, the term “heavy chain” refers to a portion of an antigen binding polypeptide, antigen binding polypeptide complex, antibody or antigen binding fragment thereof typically composed of a heavy chain variable region (VH), a heavy chain constant region 1 (CH1), a heavy chain constant region 2 (CH2), and a heavy chain constant region 3 (CH3). A typical antibody is composed of two heavy chains and two light chains. When used in reference to an antibody, a heavy chain can refer to any distinct type, e.g., alpha (a), delta (6), epsilon (F), gamma (γ), and mu (p), based on the amino acid sequence of the constant region, which gives rise to IgA, IgD, IgE, IgG, and IgM classes of antibodies, respectively, including subclasses of IgG, e.g., IgG1, IgG2, IgG3, and IgG4. Heavy chain amino acid sequences are known in the art. In some aspects, the heavy chain is a human heavy chain.

As used herein, the term “light chain” refers to a portion of an antigen binding polypeptide, antigen binding polypeptide complex, antibody or antigen binding fragment thereof typically composed of a light chain variable region (VL) and a light chain constant region (CL). A typical antibody is composed of two light chains and two heavy chains. When used in reference to an antibody, a light chain can refer to any distinct type, e.g., kappa (κ) or lambda (λ), based on the amino acid sequence of the constant region. Light chain amino acid sequences are known in the art. In some aspects, the light chain is a human light chain.

The term “chimeric” antibody or antigen binding fragment thereof refers to an antibody or antigen binding fragments thereof wherein the amino acid sequence is derived from two or more species. Typically, the variable region of both light and heavy chains corresponds to the variable region of antibodies or antigen binding fragments thereof derived from one species of mammals (e.g., mouse, rat, rabbit, etc.) with the desired specificity, affinity and capability, while the constant regions are homologous to the sequences in antibodies or antigen binding fragments thereof derived from another (usually human) to avoid eliciting an immune response in that species.

The term “humanized” antibody or antigen binding fragment thereof refers to forms of non-human (e.g., murine) antibodies or antigen binding fragments that are specific immunoglobulin chains, chimeric immunoglobulins, or fragments thereof that contain minimal non-human (e.g., murine) sequences. Typically, humanized antibodies or antigen binding fragments thereof are human immunoglobulins in which residues from a complementary determining region (CDR) are replaced by residues from a CDR of a non-human species (e.g., mouse, rat, rabbit, hamster) that have the desired specificity, affinity, and capability (Jones et al., Nature 321:522-525 (1986); Riechmann et al., Nature 332:323-327 (1988); Verhoeyen et al., Science 239:1534-1536 (1988)). In some aspects, the Fv framework region (FR) residues of a human immunoglobulin are replaced with the corresponding residues in an antibody or fragment from a non-human species that has the desired specificity, affinity, and capability. The humanized antibody or antigen binding fragment thereof can be further modified by the substitution of additional residues either in the Fv framework region and/or within the replaced non-human residues to refine and optimize antibody or antigen-binding fragment thereof specificity, affinity, and/or capability. In general, a humanized antibody or antigen binding fragment thereof will comprise substantially all of at least one, and typically two or three, variable domains containing all or substantially all of the CDR regions that correspond to the non-human immunoglobulin whereas all or substantially all of the FR regions are those of a human immunoglobulin consensus sequence. A humanized antibody or antigen binding fragment thereof can also comprise at least a portion of a constant region, typically that of a human immunoglobulin. Examples of methods used to generate humanized antibodies are known and described, for example, in U.S. Pat. No. 5,225,539; Roguska et al., Proc. Natl. Acad. Sci., USA, 91(3):969-973 (1994), and Roguska et al., Protein Eng. 9(10):895-904 (1996).

The term “human” antibody or antigen binding fragment thereof, as used herein, means an antibody or antigen binding fragment thereof having an amino acid sequence derived from a human immunoglobulin gene locus, where such antibody or antigen binding fragment is made using recombinant techniques known in the art. This definition of a human antibody or antigen binding fragment thereof includes intact or full-length antibodies and fragments thereof.

A polypeptide, polypeptide complex, antibody, antigen binding fragment thereof, polynucleotide, vector or host cell which is “isolated” is a polypeptide, polypeptide complex, antibody, antigen binding fragment thereof, polynucleotide, vector or host cell which is in a form not found in nature. Isolated polypeptides, polypeptide complexes, antibodies, antigen binding fragments thereof, polynucleotides, vectors or host cells include those which have been purified to a degree that they are no longer in a form in which they are found in nature. In some aspects, a polypeptide, polypeptide complex, antibody, antigen binding fragment thereof, polynucleotide, vector or host cell which is isolated is substantially pure. As used herein, “substantially pure” refers to material which is at least 50% pure (i.e., free from contaminants), at least 90% pure, at least 95% pure, at least 98% pure, or at least 99% pure.

The terms “polypeptide,” “peptide,” and “protein” are used interchangeably herein to refer to polymers of amino acids of any length. The polymer can be linear or branched, it can comprise modified amino acids, and it can be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. It is understood that, because the polypeptides of this invention are based upon antibodies, in some aspects, the polypeptides can occur as single chains or associated chains.

The use of the alternative (e.g., “or”) should be understood to mean either one, both, or any combination thereof of the alternatives. As used herein, the indefinite articles “a” or “an” should be understood to refer to “one or more” of any recited or enumerated component.

As used herein, the term “and/or” is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” “having,” or the like, otherwise analogous aspects described in terms of “consisting of” and/or “consisting essentially of” are also provided.

As used herein, the term “about” refers to a value or composition that is within an acceptable error range for the particular value or composition as determined by one of ordinary skill in the art, which will depend in part on how the value or composition is measured or determined, i.e., the limitations of the measurement system. For example, “about” can mean within 1 or more than 1 standard deviation per the practice in the art. Alternatively, “about” can mean a range of up to 10% or 20% (i.e., ±10% or ±20%). For example, about 3 mg can include any number between 2.7 mg and 3.3 mg (for 10%) or between 2.4 mg and 3.6 mg (for 20%). Furthermore, particularly with respect to biological systems or processes, the terms can mean up to an order of magnitude or up to 5-fold of a value. When particular values or compositions are provided in the application and claims, unless otherwise stated, the meaning of “about” should be assumed to be within an acceptable error range for that particular value or composition.

As described herein, any numerical range, concentration range, percentage range, ratio range or integer range is to be understood to include the value of any integer within the recited range and, when appropriate, fractions thereof (such as one-tenth and one-hundredth of an integer), unless otherwise indicated.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 5th ed., 2013, Academic Press; and the Oxford Dictionary Of Biochemistry And Molecular Biology, 2006, Oxford University Press, provide one of skill with a general dictionary of many of the terms used in this disclosure.

Units, prefixes, and symbols are denoted in their Système International de Unites (SI) accepted form. Numeric ranges are inclusive of the numbers defining the range. The headings provided herein are not limitations of the various aspects of the disclosure, which can be had by reference to the specification as a whole. Accordingly, the terms defined herein are more fully defined by reference to the specification in its entirety.

Various aspects are described in further detail in the following sections.

### Antigen Binding Polypeptides and Antigen Binding Polypeptide Complexes

In some aspects, the invention is directed to antigen binding polypeptides and antigen binding polypeptide complexes having certain structural features.

### Bispecific Constructs

In some aspects, the invention is directed to antigen binding polypeptides and antigen binding polypeptide complexes having a structure represented by VL1-VL2-VH2-VH1 or VH1-VH2-VL2-VL1. In some aspects, the antigen binding polypeptide or antigen binding polypeptide complex contains an amino acid linker between any two regions denoted in a structure described herein. In some aspects, the antigen binding polypeptide or antigen binding polypeptide complex can contain one or more Fc region, CH1 region, or CL region, or any combination thereof. In some aspects, the antigen binding polypeptide complex is an antibody or antigen binding fragment thereof.

The antigen binding polypeptides and antigen binding polypeptide complexes described herein specifically bind to an HIV protein. This includes specific binding to one or more HIV proteins and specific binding to one or more epitopes on the same HIV protein. In some aspects, the HIV protein is selected from the group consisting of an HIV envelope protein, an HIV structural protein, an HIV functional protein, or an HIV accessory protein. In some aspects, the HIV envelope protein is HIV envelope glycoprotein (Env), HIV envelope glycoprotein gp160, HIV envelope surface glycoprotein gp120, or HIV transmembrane envelope protein gp41. In some aspects, the HIV structural protein is p17, p24, p7 or p55. In some aspects, the HIV functional protein is p66, HIV-1 protease (PR) or p31. In some aspects, the HIV accessory protein is Nef, Tat, Rev, Vif, Vpr or Vpu.

In some aspects, an antigen binding polypeptide of the invention has a structure represented by VL1-VL2-VH2-VH1; VH1-VH2-VL2-VL1; VL1-L1-VL2-L2-VH2-L3-VH1; or VH1-L1-VH2-L2-VL2-L3-VL1; wherein VL1 is a first immunoglobulin light chain variable region that specifically binds to an HIV protein; VL2 is a second immunoglobulin light chain variable region that specifically binds to an HIV protein; VH1 is a first immunoglobulin heavy chain variable region that specifically binds to an HIV protein; VH2 is a second immunoglobulin heavy chain variable region that specifically binds to an HIV protein; and L1, L2 and L3 are amino acid linkers.

In some aspects, an antigen binding polypeptide complex of the invention comprises a first polypeptide and a second polypeptide; wherein the first polypeptide has a structure represented by VL1-VL2-VH2-VH1; VH1-VH2-VL2-VL1; VL1-L1-VL2-L2-VH2-L3-VH1; or VH1-L1-VH2-L2-VL2-L3-VL1; wherein the second polypeptide has a structure represented by VL1-VL2-VH2-VH1; VH1-VH2-VL2-VL1; VL1-L1-VL2-L2-VH2-L3-VH1; or VH1-L1-VH2-L2-VL2-L3-VL1; wherein VL1 is a first immunoglobulin light chain variable region that specifically binds to an HIV protein; VL2 is a second immunoglobulin light chain variable region that specifically binds to an HIV protein; VH1 is a first immunoglobulin heavy chain variable region that specifically binds to an HIV protein; VH2 is a second immunoglobulin heavy chain variable region that specifically binds to an HIV protein; and L1, L2 and L3 are amino acid linkers.

In some aspects, an antigen binding polypeptide of the invention has a structure represented by VL1-VL2-VH2-VH1-Fc; VH1-VH2-VL2-VL1-Fc; VL1-L1-VL2-L2-VH2-L3-VH1-Fc; VH1-L1-VH2-L2-VL2-L3-VL1-Fc; VL1-L1-VL2-L2-VH2-L3-VH1-L4-Fc; or VH1-L1-VH2-L2-VL2-L3-VL1-L4-Fc; wherein VL1 is a first immunoglobulin light chain variable region that specifically binds to an HIV protein; VL2 is a second immunoglobulin light chain variable region that specifically binds to an HIV protein; VH1 is a first immunoglobulin heavy chain variable region that specifically binds to an HIV protein; VH2 is a second immunoglobulin heavy chain variable region that specifically binds to an HIV protein; Fc is a region comprising an immunoglobulin heavy chain constant region 2 (CH2), an immunoglobulin heavy chain constant region 3 (CH3), and optionally, an immunoglobulin hinge; and L1, L2, L3 and L4 are amino acid linkers.

In some aspects, an antigen binding polypeptide complex of the invention comprises a first polypeptide and a second polypeptide; wherein the first polypeptide has a structure represented by VL1-VL2-VH2-VH1-Fc; VH1-VH2-VL2-VL1-Fc; VL1-L1-VL2-L2-VH2-L3-VH1-Fc; VH1-L1-VH2-L2-VL2-L3-VL1-Fc; VL1-L1-VL2-L2-VH2-L3-VH1-L4-Fc; or VH1-L1-VH2-L2-VL2-L3-VL1-L4-Fc; wherein the second polypeptide has a structure represented by Fc; VL1-VL2-VH2-VH1-Fc; VH1-VH2-VL2-VL1-Fc; VL1-L1-VL2-L2-VH2-L3-VH1-Fc; VH1-L1-VH2-L2-VL2-L3-VL1-Fc; VL1-L1-VL2-L2-VH2-L3-VH1-L4-Fc; or VH1-L1-VH2-L2-VL2-L3-VL1-L4-Fc; wherein VL1 is a first immunoglobulin light chain variable region that specifically binds to an HIV protein; VL2 is a second immunoglobulin light chain variable region that specifically binds to an HIV protein; VH1 is a first immunoglobulin heavy chain variable region that specifically binds to an HIV protein; VH2 is a second immunoglobulin heavy chain variable region that specifically binds to an HIV protein; Fc is a region comprising an immunoglobulin heavy chain constant region 2 (CH2), an immunoglobulin heavy chain constant region 3 (CH3), and optionally, an immunoglobulin hinge; and L1, L2, L3 and L4 are amino acid linkers.

In some aspects, an antigen binding polypeptide of the invention has a structure represented by VL1-VL2-VH2-VH1-CH1-CL; VH1-VH2-VL2-VL1-CH1-CL; VL1-VL2-VH2-VH1-CL-CH1; VH1-VH2-VL2-VL1-CL-CH1; VL1-L1-VL2-L2-VH2-L3-VH1-L4-CH1-L5-CL; VH1-L1-VH2-L2-VL2-L3-VL1-L4-CH1-L5-CL; VL1-L1-VL2-L2-VH2-L3-VH1-L4-CL-L5-CH1; or VH1-L1-VH2-L2-VL2-L3-VL1-L4-CL-L5-CH1; wherein VL1 is a first immunoglobulin light chain variable region that specifically binds to an HIV protein; VL2 is a second immunoglobulin light chain variable region that specifically binds to an HIV protein; VH1 is a first immunoglobulin heavy chain variable region that specifically binds to an HIV protein; VH2 is a second immunoglobulin heavy chain variable region that specifically binds to an HIV protein; CH1 is an immunoglobulin heavy chain constant region 1; CL is an immunoglobulin light chain constant region; and L1, L2, L3, L4 and L5 are amino acid linkers.

In some aspects, an antigen binding polypeptide complex of the invention comprises a first polypeptide and a second polypeptide; wherein the first polypeptide has a structure represented by VL1-VL2-VH2-VH1-CH1; VH1-VH2-VL2-VL1-CH1; VL1-VL2-VH2-VH1-CL; VH1-VH2-VL2-VL1-CL; VL1-VL2-VH2-VH1-CH1-CL; VH1-VH2-VL2-VL1-CH1-CL; VL1-VL2-VH2-VH1-CL-CH1; VH1-VH2-VL2-VL1-CL-CH1; VL1-L1-VL2-L2-VH2-L3-VH1-L4-CH1; VH1-L1-VH2-L2-VL2-L3-VL1-L4-CH1; VL1-L1-VL2-L2-VH2-L3-VH1-L4-CL; VH1-L1-VH2-L2-VL2-L3-VL1-L4-CL; VL1-L1-VL2-L2-VH2-L3-VH1-L4-CH1-L5-CL; VH1-L1-VH2-L2-VL2-L3-VL1-L4-CH1-L5-CL; VL1-L1-VL2-L2-VH2-L3-VH1-L4-CL-L5-CH1; or VH1-L1-VH2-L2-VL2-L3-VL1-L4-CL-L5-CH1; wherein the second polypeptide has a structure represented by VL1-VL2-VH2-VH1-CH1; VL1-VL2-VH2-VH1-CH1; VH1-VH2-VL2-VL1-CH1; VL1-VL2-VH2-VH1-CL; VH1-VH2-VL2-VL1-CL; VL1-VL2-VH2-VH1-CH1-CL; VH1-VH2-VL2-VL1-CH1-CL; VL1-VL2-VH2-VH1-CL-CH1; VH1-VH2-VL2-VL1-CL-CH1; VL1-L1-VL2-L2-VH2-L3-VH1-L4-CH1; VH1-L1-VH2-L2-VL2-L3-VL1-L4-CH1; VL1-L1-VL2-L2-VH2-L3-VH1-L4-CL; VH1-L1-VH2-L2-VL2-L3-VL1-L4-CL; VL1-L1-VL2-L2-VH2-L3-VH1-L4-CH1-L5-CL; VH1-L1-VH2-L2-VL2-L3-VL1-L4-CH1-L5-CL; VL1-L1-VL2-L2-VH2-L3-VH1-L4-CL-L5-CH1; or VH1-L1-VH2-L2-VL2-L3-VL1-L4-CL-L5-CH1; wherein VL1 is a first immunoglobulin light chain variable region that specifically binds to an HIV protein; VL2 is a second immunoglobulin light chain variable region that specifically binds to an HIV protein; VH1 is a first immunoglobulin heavy chain variable region that specifically binds to an HIV protein; VH2 is a second immunoglobulin heavy chain variable region that specifically binds to an HIV protein; CH1 is an immunoglobulin heavy chain constant region 1; CL is an immunoglobulin light chain constant region; and L1, L2, L3, L4 and L5 are amino acid linkers.

In some aspects, an antigen binding polypeptide of the invention has a structure represented by VL1-VL2-VH2-VH1-CH1-CL-Fc; VH1-VH2-VL2-VL1-CH1-CL-Fc; VL1-VL2-VH2-VH1-CL-CH1-Fc; VH1-VH2-VL2-VL1-CL-CH1-Fc; VL1-L1-VL2-L2-VH2-L3-VH1-L4-CH1-L5-CL-Fc; VH1-L1-VH2-L2-VL2-L3-VL1-L4-CH1-L5-CL-Fc; VL1-L1-VL2-L2-VH2-L3-VH1-L4-CL-L5-CH1-Fc; VH1-L1-VH2-L2-VL2-L3-VL1-L4-CL-L5-CH1-Fc; VL1-L1-VL2-L2-VH2-L3-VH1-L4-CH1-L5-CL-L6-Fc; VH1-L1-VH2-L2-VL2-L3-VL1-L4-CH1-L5-CL-L6-Fc; VL1-L1-VL2-L2-VH2-L3-VH1-L4-CL-L5-CH1-L6-Fc; or VH1-L1-VH2-L2-VL2-L3-VL1-L4-CL-L5-CH1-L6-Fc; wherein VL1 is a first immunoglobulin light chain variable region that specifically binds to an HIV protein; VL2 is a second immunoglobulin light chain variable region that specifically binds to an HIV protein; VH1 is a first immunoglobulin heavy chain variable region that specifically binds to an HIV protein; VH2 is a second immunoglobulin heavy chain variable region that specifically binds to an HIV protein; CH1 is an immunoglobulin heavy chain constant region 1; CL is an immunoglobulin light chain constant region; Fc is a region comprising an immunoglobulin heavy chain constant region 2 (CH2), an immunoglobulin heavy chain constant region 3 (CH3), and optionally, an immunoglobulin hinge; and L1, L2, L3, L4, L5 and L6 are amino acid linkers.

In some aspects, an antigen binding polypeptide complex of the invention comprises a first polypeptide and a second polypeptide; wherein the first polypeptide has a structure represented by VL1-VL2-VH2-VH1-CH1-Fc; VH1-VH2-VL2-VL1-CH1-Fc; VL1-VL2-VH2-VH1-CL-Fc; VH1-VH2-VL2-VL1-CL-Fc; VL1-VL2-VH2-VH1-CH1-CL-Fc; VH1-VH2-VL2-VL1-CH1-CL-Fc; VL1-VL2-VH2-VH1-CL-CH1-Fc; VH1-VH2-VL2-VL1-CL-CH1-Fc; VL1-L1-VL2-L2-VH2-L3-VH1-L4-CH1-Fc; VH1-L1-VH2-L2-VL2-L3-VL1-L4-CH1-Fc; VL1-L1-VL2-L2-VH2-L3-VH1-L4-CL-Fc; VH1-L1-VH2-L2-VL2-L3-VL1-L4-CL-Fc; VL1-L1-VL2-L2-VH2-L3-VH1-L4-CH1-L5-CL-Fc; VH1-L1-VH2-L2-VL2-L3-VL1-L4-CH1-L5-CL-Fc; VL1-L1-VL2-L2-VH2-L3-VH1-L4-CL-L5-CH1-Fc; VH1-L1-VH2-L2-VL2-L3-VL1-L4-CL-L5-CH1-Fc; VL1-L1-VL2-L2-VH2-L3-VH1-L4-CH1-L5-Fc; VH1-L1-VH2-L2-VL2-L3-VL1-L4-CH1-L5-Fc; VL1-L1-VL2-L2-VH2-L3-VH1-L4-CL-L5-Fc; VH1-L1-VH2-L2-VL2-L3-VL1-L4-CL-L5-Fc; VL1-L1-VL2-L2-VH2-L3-VH1-L4-CH1-L5-CL-L6-Fc; VH1-L1-VH2-L2-VL2-L3-VL1-L4-CH1-L5-CL-L6-Fc; VL1-L1-VL2-L2-VH2-L3-VH1-L4-CL-L5-CH1-L6-Fc; or VH1-L1-VH2-L2-VL2-L3-VL1-L4-CL-L5-CH1-L6-Fc; wherein the second polypeptide has a structure represented by Fc; VL1-VL2-VH2-VH1-CH1-Fc; VH1-VH2-VL2-VL1-CH1-Fc; VL1-VL2-VH2-VH1-CL-Fc; VH1-VH2-VL2-VL1-CL-Fc; VL1-VL2-VH2-VH1-CH1-CL-Fc; VH1-VH2-VL2-VL1-CH1-CL-Fc; VL1-VL2-VH2-VH1-CL-CH1-Fc; VH1-VH2-VL2-VL1-CL-CH1-Fc; VL1-L1-VL2-L2-VH2-L3-VH1-L4-CH1-Fc; VH1-L1-VH2-L2-VL2-L3-VL1-L4-CH1-Fc; VL1-L1-VL2-L2-VH2-L3-VH1-L4-CL-Fc; VH1-L1-VH2-L2-VL2-L3-VL1-L4-CL-Fc; VL1-L1-VL2-L2-VH2-L3-VH1-L4-CH1-L5-CL-Fc; VH1-L1-VH2-L2-VL2-L3-VL1-L4-CH1-L5-CL-Fc; VL1-L1-VL2-L2-VH2-L3-VH1-L4-CL-L5-CH1-Fc; VH1-L1-VH2-L2-VL2-L3-VL1-L4-CL-L5-CH1-Fc; VL1-L1-VL2-L2-VH2-L3-VH1-L4-CH1-L5-Fc; VH1-L1-VH2-L2-VL2-L3-VL1-L4-CH1-L5-Fc; VL1-L1-VL2-L2-VH2-L3-VH1-L4-CL-L5-Fc; VH1-L1-VH2-L2-VL2-L3-VL1-L4-CL-L5-Fc; VL1-L1-VL2-L2-VH2-L3-VH1-L4-CH1-L5-CL-L6-Fc; VH1-L1-VH2-L2-VL2-L3-VL1-L4-CH1-L5-CL-L6-Fc; VL1-L1-VL2-L2-VH2-L3-VH1-L4-CL-L5-CH1-L6-Fc; or VH1-L1-VH2-L2-VL2-L3-VL1-L4-CL-L5-CH1-L6-Fc; wherein VL1 is a first immunoglobulin light chain variable region that specifically binds to an HIV protein; VL2 is a second immunoglobulin light chain variable region that specifically binds to an HIV protein; VH1 is a first immunoglobulin heavy chain variable region that specifically binds to an HIV protein; VH2 is a second immunoglobulin heavy chain variable region that specifically binds to an HIV protein; CH1 is an immunoglobulin heavy chain constant region 1; CL is an immunoglobulin light chain constant region; Fc is a region comprising an immunoglobulin heavy chain constant region 2 (CH2), an immunoglobulin heavy chain constant region 3 (CH3), and optionally, an immunoglobulin hinge; and L1, L2, L3, L4, L5 and L6 are amino acid linkers.

In some aspects, an antigen binding polypeptide complex of the invention comprises a first polypeptide and a second polypeptide; wherein the first polypeptide has a structure represented by: VL1-VL2-VH2-VH1; VH1-VH2-VL2-VL1; VL1-L1-VL2-L2-VH2-L3-VH1; VH1-L1-VH2-L2-VL2-L3-VL1; VL1-VL2-VH2-VH1-Fc; VH1-VH2-VL2-VL1-Fc; VL1-L1-VL2-L2-VH2-L3-VH1-Fc; VH1-L1-VH2-L2-VL2-L3-VL1-Fc; VL1-L1-VL2-L2-VH2-L3-VH1-L4-Fc; VH1-L1-VH2-L2-VL2-L3-VL1-L4-Fc; VL1-VL2-VH2-VH1-CH1; VH1-VH2-VL2-VL1-CH1; VL1-VL2-VH2-VH1-CL; VH1-VH2-VL2-VL1-CL; VL1-VL2-VH2-VH1-CH1-CL; VH1-VH2-VL2-VL1-CH1-CL; VL1-VL2-VH2-VH1-CL-CH1; VH1-VH2-VL2-VL1-CL-CH1; VL1-L1-VL2-L2-VH2-L3-VH1-L4-CH1; VH1-L1-VH2-L2-VL2-L3-VL1-L4-CH1; VL1-L1-VL2-L2-VH2-L3-VH1-L4-CL; VH1-L1-VH2-L2-VL2-L3-VL1-L4-CL; VL1-L1-VL2-L2-VH2-L3-VH1-L4-CH1-L5-CL; VH1-L1-VH2-L2-VL2-L3-VL1-L4-CH1-L5-CL; VL1-L1-VL2-L2-VH2-L3-VH1-L4-CL-L5-CH1; VH1-L1-VH2-L2-VL2-L3-VL1-L4-CL-L5-CH1; VL1-VL2-VH2-VH1-CH1-Fc; VH1-VH2-VL2-VL1-CH1-Fc; VL1-VL2-VH2-VH1-CL-Fc; VH1-VH2-VL2-VL1-CL-Fc; VL1-VL2-VH2-VH1-CH1-CL-Fc; VH1-VH2-VL2-VL1-CH1-CL-Fc; VL1-VL2-VH2-VH1-CL-CH1-Fc; VH1-VH2-VL2-VL1-CL-CH1-Fc; VL1-L1-VL2-L2-VH2-L3-VH1-L4-CH1-Fc; VH1-L1-VH2-L2-VL2-L3-VL1-L4-CH1-Fc; VL1-L1-VL2-L2-VH2-L3-VH1-L4-CL-Fc; VH1-L1-VH2-L2-VL2-L3-VL1-L4-CL-Fc; VL1-L1-VL2-L2-VH2-L3-VH1-L4-CH1-L5-CL-Fc; VH1-L1-VH2-L2-VL2-L3-VL1-L4-CH1-L5-CL-Fc; VL1-L1-VL2-L2-VH2-L3-VH1-L4-CL-L5-CH1-Fc; VH1-L1-VH2-L2-VL2-L3-VL1-L4-CL-L5-CH1-Fc; VL1-L1-VL2-L2-VH2-L3-VH1-L4-CH1-L5-Fc; VH1-L1-VH2-L2-VL2-L3-VL1-L4-CH1-L5-Fc; VL1-L1-VL2-L2-VH2-L3-VH1-L4-CL-L5-Fc; VH1-L1-VH2-L2-VL2-L3-VL1-L4-CL-L5-Fc; VL1-L1-VL2-L2-VH2-L3-VH1-L4-CH1-L5-CL-L6-Fc; VH1-L1-VH2-L2-VL2-L3-VL1-L4-CH1-L5-CL-L6-Fc; VL1-L1-VL2-L2-VH2-L3-VH1-L4-CL-L5-CH1-L6-Fc; or VH1-L1-VH2-L2-VL2-L3-VL1-L4-CL-L5-CH1-L6-Fc; wherein the second polypeptide has a structure represented by VL1-VL2-VH2-VH1; VH1-VH2-VL2-VL1; VL1-L1-VL2-L2-VH2-L3-VH1; VH1-L1-VH2-L2-VL2-L3-VL1; VL1-VL2-VH2-VH1-Fc; VH1-VH2-VL2-VL1-Fc; VL1-L1-VL2-L2-VH2-L3-VH1-Fc; VH1-L1-VH2-L2-VL2-L3-VL1-Fc; VL1-L1-VL2-L2-VH2-L3-VH1-L4-Fc; VH1-L1-VH2-L2-VL2-L3-VL1-L4-Fc; VL1-VL2-VH2-VH1-CH1; VH1-VH2-VL2-VL1-CH1; VL1-VL2-VH2-VH1-CL; VH1-VH2-VL2-VL1-CL; VL1-VL2-VH2-VH1-CH1-CL; VH1-VH2-VL2-VL1-CH1-CL; VL1-VL2-VH2-VH1-CL-CH1; VH1-VH2-VL2-VL1-CL-CH1; VL1-L1-VL2-L2-VH2-L3-VH1-L4-CH1; VH1-L1-VH2-L2-VL2-L3-VL1-L4-CH1; VL1-L1-VL2-L2-VH2-L3-VH1-L4-CL; VH1-L1-VH2-L2-VL2-L3-VL1-L4-CL; VL1-L1-VL2-L2-VH2-L3-VH1-L4-CH1-L5-CL; VH1-L1-VH2-L2-VL2-L3-VL1-L4-CH1-L5-CL; VL1-L1-VL2-L2-VH2-L3-VH1-L4-CL-L5-CH1; VH1-L1-VH2-L2-VL2-L3-VL1-L4-CL-L5-CH1; VL1-VL2-VH2-VH1-CH1-Fc; VH1-VH2-VL2-VL1-CH1-Fc; VL1-VL2-VH2-VH1-CL-Fc; VH1-VH2-VL2-VL1-CL-Fc; VL1-VL2-VH2-VH1-CH1-CL-Fc; VH1-VH2-VL2-VL1-CH1-CL-Fc; VL1-VL2-VH2-VH1-CL-CH1-Fc; VH1-VH2-VL2-VL1-CL-CH1-Fc; VL1-L1-VL2-L2-VH2-L3-VH1-L4-CH1-Fc; VH1-L1-VH2-L2-VL2-L3-VL1-L4-CH1-Fc; VL1-L1-VL2-L2-VH2-L3-VH1-L4-CL-Fc; VH1-L1-VH2-L2-VL2-L3-VL1-L4-CL-Fc; VL1-L1-VL2-L2-VH2-L3-VH1-L4-CH1-L5-CL-Fc; VH1-L1-VH2-L2-VL2-L3-VL1-L4-CH1-L5-CL-Fc; VL1-L1-VL2-L2-VH2-L3-VH1-L4-CL-L5-CH1-Fc; VH1-L1-VH2-L2-VL2-L3-VL1-L4-CL-L5-CH1-Fc; VL1-L1-VL2-L2-VH2-L3-VH1-L4-CH1-L5-Fc; VH1-L1-VH2-L2-VL2-L3-VL1-L4-CH1-L5-Fc; VL1-L1-VL2-L2-VH2-L3-VH1-L4-CL-L5-Fc; VH1-L1-VH2-L2-VL2-L3-VL1-L4-CL-L5-Fc; VL1-L1-VL2-L2-VH2-L3-VH1-L4-CH1-L5-CL-L6-Fc; VH1-L1-VH2-L2-VL2-L3-VL1-L4-CH1-L5-CL-L6-Fc; VL1-L1-VL2-L2-VH2-L3-VH1-L4-CL-L5-CH1-L6-Fc; or VH1-L1-VH2-L2-VL2-L3-VL1-L4-CL-L5-CH1-L6-Fc; wherein VL1 is a first immunoglobulin light chain variable region that specifically binds to an HIV protein; VL2 is a second immunoglobulin light chain variable region that specifically binds to an HIV protein; VH1 is a first immunoglobulin heavy chain variable region that specifically binds to an HIV protein; VH2 is a second immunoglobulin heavy chain variable region that specifically binds to an HIV protein; CH1 is an immunoglobulin heavy chain constant region 1; CL is an immunoglobulin light chain constant region; Fc is a region comprising an immunoglobulin heavy chain constant region 2 (CH2), an immunoglobulin heavy chain constant region 3 (CH3), and optionally, an immunoglobulin hinge; and L1, L2, L3, L4, L5 and L6 are amino acid linkers.

In other aspects, the invention is directed to an antigen binding polypeptide or antigen binding polypeptide complex comprising a polypeptide having a structure represented by VL1-VL2-VH2-VH1 or VH1-VH2-VL2-VL1 which has two Fc regions. In some aspects, an antigen binding polypeptide or antigen binding polypeptide complex comprises a polypeptide having a structure represented by VL1-VL2-VH2-VH1-Fc-Fc; VH1-VH2-VL2-VL1-Fc-Fc; VL1-L1-VL2-L2-VH2-L3-VH1-Fc-Fc; VH1-L1-VH2-L2-VL2-L3-VL1-Fc-Fc; VL1-L1-VL2-L2-VH2-L3-VH1-L4-Fc-Fc; VH1-L1-VH2-L2-VL2-L3-VL1-L4-Fc-Fc; VL1-L1-VL2-L2-VH2-L3-VH1-L4-Fc-L5-Fc; or VH1-L1-VH2-L2-VL2-L3-VL1-L4-Fc-L5-Fc; wherein VL1 is a first immunoglobulin light chain variable region that specifically binds to an HIV protein; VL2 is a second immunoglobulin light chain variable region that specifically binds to an HIV protein; VH1 is a first immunoglobulin heavy chain variable region that specifically binds to an HIV protein; VH2 is a second immunoglobulin heavy chain variable region that specifically binds to an HIV protein; Fc is a region comprising an immunoglobulin heavy chain constant region 2 (CH2), an immunoglobulin heavy chain constant region 3 (CH3), and optionally, an immunoglobulin hinge; and L1, L2, L3, L4 and L5 are amino acid linkers.

In other aspects, the invention is directed to an antigen binding polypeptide or antigen binding polypeptide complex comprising a polypeptide having a structure represented by VL1-VL2-VH2-VH1 or VH1-VH2-VL2-VL1 which has one or two CH3 regions. In some aspects, the antigen binding polypeptide or antigen binding polypeptide complex comprises a polypeptide having a structure represented by VL1-VL2-VH2-VH1-CH3; VH1-VH2-VL2-VL1-CH3; VL1-L1-VL2-L2-VH2-L3-VH1-CH3; VH1-L1-VH2-L2-VL2-L3-VL1-CH3; VL1-L1-VL2-L2-VH2-L3-VH1-L4-CH3; VH1-L1-VH2-L2-VL2-L3-VL1-L4-CH3; VL1-VL2-VH2-VH1-CH3-CH3; VH1-VH2-VL2-VL1-CH3-CH3; VL1-L1-VL2-L2-VH2-L3-VH1-CH3-CH3; VH1-L1-VH2-L2-VL2-L3-VL1-CH3-CH3; VL1-L1-VL2-L2-VH2-L3-VH1-L4-CH3-CH3; VH1-L1-VH2-L2-VL2-L3-VL1-L4-CH3-CH3; VL1-L1-VL2-L2-VH2-L3-VH1-L4-CH3-L5-CH3; or VH1-L1-VH2-L2-VL2-L3-VL1-L4-CH3-L5-CH3; wherein VL1 is a first immunoglobulin light chain variable region that specifically binds to an HIV protein; VL2 is a second immunoglobulin light chain variable region that specifically binds to an HIV protein; VH1 is a first immunoglobulin heavy chain variable region that specifically binds to an HIV protein; VH2 is a second immunoglobulin heavy chain variable region that specifically binds to an HIV protein; CH3 is an immunoglobulin heavy chain constant region 3; and L1, L2, L3, L4 and L5 are amino acid linkers.

In some aspects of an antigen binding polypeptide or antigen binding polypeptide complex of the invention, VH1 and VH2 each comprise a heavy chain variable region from the PGT121, VRC01, 10E8v4 or PG16 antibody or a variant thereof. In some aspects, VL1 and VL2 each comprise a light chain variable region from the PGT121, VRC01, 10E8v4 or PG16 antibody or a variant thereof. In some aspects, VH1 and VH2 each comprise a heavy chain variable region from the PGT121, VRC01, 10E8v4 or PG16 antibody or a variant thereof, and VL1 and VL2 each comprise a light chain variable region from the PGT121, VRC01, 10E8v4 or PG16 antibody or a variant thereof.

In some aspects, antigen binding polypeptides or antigen binding polypeptide complexes comprise VH and VL sequences from broadly neutralizing antibodies that target CD4bs inclusive of VRC01, VRC03, 3BNC117, N6, N49P7, 3BNC60, VRC-PG04, VRC-PG20, NIH45-46, VRC-CH31, 12A12, CH103, 8ANC131, VRC13 and VRC16.

In some aspects, VH1 and VH2 of an antigen binding polypeptide or antigen binding polypeptide complex of the invention each comprise an amino acid sequence having at least 90% identity, at least 95% identity or 100% identity to any one of SEQ ID NOs:20-23. In some aspects, VL1 and VL2 each comprise an amino acid sequence having at least 90% identity, at least 95% identity or 100% identity to any one of SEQ ID NOs:24-27. In some aspects, VH1 and VH2 of an antigen binding polypeptide or antigen binding polypeptide complex of the invention each comprise an amino acid sequence having at least 90% identity, at least 95% identity or 100% identity to any one of SEQ ID NOs:20-23, and VL1 and VL2 each comprise an amino acid sequence having at least 90% identity, at least 95% identity or 100% identity to any one of SEQ ID NOs:24-27.

In some aspects, VH1 and VH2 of an antigen binding polypeptide or antigen binding polypeptide complex of the invention each comprise a CDR1 having an amino acid sequence with at least 90% identity, at least 95% identity or 100% identity to any one of SEQ ID NOs:60, 63, 66 and 69; a CDR2 having an amino acid sequence with at least 90% identity, at least 95% identity or 100% identity to any one of SEQ ID NOs:61, 64, 67 and 70; and a CDR3 having an amino acid sequence with at least 90% identity, at least 95% identity or 100% identity to any one of SEQ ID NOs:62, 65, 68 and 71; and/or VL1 and VL2 each comprise a CDR1 having an amino acid sequence with at least 90% identity, at least 95% identity or 100% identity to any one of SEQ ID NOs:72, 75, 78 and 81; a CDR2 having an amino acid sequence with at least 90% identity, at least 95% identity or 100% identity to any one of SEQ ID NOs:73, 76, 79 and 82; and a CDR3 having an amino acid sequence with at least 90% identity, at least 95% identity or 100% identity to any one of SEQ ID NOs:74, 77, 80 and 83.

### Trispecific Constructs

In some aspects, the invention is directed to an antigen binding polypeptide complex (e.g., an antibody or antigen binding fragment thereof) comprising a first polypeptide having a structure represented by VL1-VL2-VH2-VH1 or VH1-VH2-VL2-VL1 and a second polypeptide having a structure of VL3-VH3 or VH3-VL3. In some aspects, the invention is directed to an antigen binding polypeptide complex comprising a first polypeptide having a structure represented by VL1-VL2-VH2-VH1 or VH1-VH2-VL2-VL1; a second polypeptide having a structure represented by VL3; and a third polypeptide having a structure represented by VH3. In some aspects, the antigen binding polypeptide complex contains an amino acid linker between any two regions denoted in a structure described herein. In some aspects, the antigen binding polypeptide complex contains an Fc region, CH1 region, CL region, or any combination thereof. In some aspects, the antigen binding polypeptide complex is an antibody or antigen binding fragment thereof.

The antigen binding polypeptides and antigen binding polypeptide complexes described herein specifically bind to an HIV protein. This includes specific binding to one or more HIV proteins and specific binding to one or more epitopes on the same HIV protein. In some aspects, the HIV protein is selected from the group consisting of an HIV envelope protein, an HIV structural protein, an HIV functional protein, or an HIV accessory protein. In some aspects, the HIV envelope protein is HIV envelope glycoprotein (Env), HIV envelope glycoprotein gp160, HIV envelope surface glycoprotein gp120, or HIV transmembrane envelope protein gp41. In some aspects, the HIV structural protein is p17, p24, p7 or p55. In some aspects, the HIV functional protein is p66, HIV-1 protease (PR) or p31. In some aspects, the HIV accessory protein is Nef, Tat, Rev, Vif, Vpr or Vpu.

In some aspects, the invention is directed to an antigen binding polypeptide complex comprising a first polypeptide and a second polypeptide; wherein the first polypeptide has a structure represented by VL1-VL2-VH2-VH1; VH1-VH2-VL2-VL1; VL1-L1-VL2-L2-VH2-L3-VH1; or VH1-L1-VH2-L2-VL2-L3-VL1; wherein the second polypeptide has a structure represented by VL3-VH3; VH3-VL3; VL3-L4-VH3; or VH3-L4-VL3; wherein VL1 is a first immunoglobulin light chain variable region that specifically binds to an HIV protein; VL2 is a second immunoglobulin light chain variable region that specifically binds to an HIV protein; VL3 is a third immunoglobulin light chain variable region that specifically binds to an HIV protein; VH1 is a first immunoglobulin heavy chain variable region that specifically binds to an HIV protein; VH2 is a second immunoglobulin heavy chain variable region that specifically binds to an HIV protein; VH3 is a third immunoglobulin heavy chain variable region that specifically binds to an HIV protein; and L1, L2, L3 and L4 are amino acid linkers.

In other aspects, the invention is directed to an antigen binding polypeptide complex comprising a first polypeptide and a second polypeptide; wherein the first polypeptide has a structure represented by VL1-VL2-VH2-VH1-Fc; VH1-VH2-VL2-VL1-Fc; VL1-L1-VL2-L2-VH2-L3-VH1-Fc; VH1-L1-VH2-L2-VL2-L3-VL1-Fc; VL1-L1-VL2-L2-VH2-L3-VH1-L4-Fc; or VH1-L1-VH2-L2-VL2-L3-VL1-L4-Fc; wherein the second polypeptide has a structure represented by VL3-VH3-Fc; VH3-VL3-Fc; VL3-L5-VH3-Fc; VH3-L5-VL3-Fc; VL3-L5-VH3-L6-Fc; or VH3-L5-VL3-L6-Fc; wherein VL1 is a first immunoglobulin light chain variable region that specifically binds to an HIV protein; VL2 is a second immunoglobulin light chain variable region that specifically binds to an HIV protein; VL3 is a third immunoglobulin light chain variable region that specifically binds to an HIV protein; VH1 is a first immunoglobulin heavy chain variable region that specifically binds to an HIV protein; VH2 is a second immunoglobulin heavy chain variable region that specifically binds to an HIV protein; VH3 is a third immunoglobulin heavy chain variable region that specifically binds to an HIV protein; Fc is a region comprising an immunoglobulin heavy chain constant region 2 (CH2), an immunoglobulin heavy chain constant region 3 (CH3), and optionally, an immunoglobulin hinge; and L1, L2, L3, L4, L5 and L6 are amino acid linkers.

In other aspects, the invention is directed to an antigen binding polypeptide complex comprising a first polypeptide and a second polypeptide; wherein the first polypeptide has a structure represented by VL1-VL2-VH2-VH1-CH1; VH1-VH2-VL2-VL1-CH1; VL1-VL2-VH2-VH1-CL; VH1-VH2-VL2-VL1-CL; VL1-VL2-VH2-VH1-CH1-CL; VH1-VH2-VL2-VL1-CH1-CL; VL1-VL2-VH2-VH1-CL-CH1; VH1-VH2-VL2-VL1-CL-CH1; VL1-L1-VL2-L2-VH2-L3-VH1-L4-CH1; VH1-L1-VH2-L2-VL2-L3-VL1-L4-CH1; VL1-L1-VL2-L2-VH2-L3-VH1-L4-CL; VH1-L1-VH2-L2-VL2-L3-VL1-L4-CL; VL1-L1-VL2-L2-VH2-L3-VH1-L4-CH1-L5-CL; VH1-L1-VH2-L2-VL2-L3-VL1-L4-CH1-L5-CL; VL1-L1-VL2-L2-VH2-L3-VH1-L4-CL-L5-CH1; or VH1-L1-VH2-L2-VL2-L3-VL1-L4-CL-L5-CH1; wherein the second polypeptide has a structure represented by VL3-VH3-CH1; VH3-VL3-CH1; VL3-VH3-CL; VH3-VL3-CL; VL3-VH3-CH1-CL; VH3-VL3-CH1-CL; VL3-VH3-CL-CH1; VH3-VL3-CL-CH1; VL3-CL-VH3-CH1; VL3-CH1-VH3-CL; VH3-CH1-VL3-CL; VH3-CL-VL3-CH1; VL3-L6-VH3-L7-CH1; VH3-L6-VL3-L7-CH1; VL3-L6-VH3-L7-CL; VH3-L6-VL3-L7-CL; VL3-L6-VH3-L7-CH1-L8-CL; VH3-L6-VL3-L7-CH1-L8-CL; VL3-L6-VH3-L7-CL-L8-CH1; VH3-L6-VL3-L7-CL-L8-CH1; VL3-L6-CL-L7-VH3-L8-CH1; VL3-L6-CH1-L7-VH3-L8-CL; VH3-L6-CH1-L7-VL3-L8-CL; VH3-L6-CL-L7-VL3-L8-CH1; VL3-VH3-L6-CH1-CL; VH3-VL3-L6-CH1-CL; VL3-VH3-L6-CL-CH1; VH3-VL3-L6-CL-CH1; VL3-CL-L6-VH3-CH1; VL3-CH1-L6-VH3-CL; VH3-CH1-L6-VL3-CL; or VH3-CL-L6-VL3-CH1; wherein VL1 is a first immunoglobulin light chain variable region that specifically binds to an HIV protein; VL2 is a second immunoglobulin light chain variable region that specifically binds to an HIV protein; VL3 is a third immunoglobulin light chain variable region that specifically binds to an HIV protein; VH1 is a first immunoglobulin heavy chain variable region that specifically binds to an HIV protein; VH2 is a second immunoglobulin heavy chain variable region that specifically binds to an HIV protein; VH3 is a third immunoglobulin heavy chain variable region that specifically binds to an HIV protein; CH1 is an immunoglobulin heavy chain constant region 1; CL is an immunoglobulin light chain constant region; and L1, L2, L3, L4, L5, L6, L7 and L8 are amino acid linkers.

In other aspects, the invention is directed to an antigen binding polypeptide complex comprising a first polypeptide and a second polypeptide; wherein the first polypeptide has a structure represented by VL1-VL2-VH2-VH1-CH1-Fc; VH1-VH2-VL2-VL1-CH1-Fc; VL1-VL2-VH2-VH1-CL-Fc; VH1-VH2-VL2-VL1-CL-Fc; VL1-VL2-VH2-VH1-CH1-CL-Fc; VH1-VH2-VL2-VL1-CH1-CL-Fc; VL1-VL2-VH2-VH1-CL-CH1-Fc; VH1-VH2-VL2-VL1-CL-CH1-Fc; VL1-L1-VL2-L2-VH2-L3-VH1-L4-CH1-Fc; VH1-L1-VH2-L2-VL2-L3-VL1-L4-CH1-Fc; VL1-L1-VL2-L2-VH2-L3-VH1-L4-CL-Fc; VH1-L1-VH2-L2-VL2-L3-VL1-L4-CL-Fc; VL1-L1-VL2-L2-VH2-L3-VH1-L4-CH1-L5-CL-Fc; VH1-L1-VH2-L2-VL2-L3-VL1-L4-CH1-L5-CL-Fc; VL1-L1-VL2-L2-VH2-L3-VH1-L4-CL-L5-CH1-Fc; VH1-L1-VH2-L2-VL2-L3-VL1-L4-CL-L5-CH1-Fc; VL1-L1-VL2-L2-VH2-L3-VH1-L4-CH1-L5-Fc; VH1-L1-VH2-L2-VL2-L3-VL1-L4-CH1-L5-Fc; VL1-L1-VL2-L2-VH2-L3-VH1-L4-CL-L5-Fc; VH1-L1-VH2-L2-VL2-L3-VL1-L4-CL-L5-Fc; VL1-L1-VL2-L2-VH2-L3-VH1-L4-CH1-L5-CL-L6-Fc; VH1-L1-VH2-L2-VL2-L3-VL1-L4-CH1-L5-CL-L6-Fc; VL1-L1-VL2-L2-VH2-L3-VH1-L4-CL-L5-CH1-L6-Fc; or VH1-L1-VH2-L2-VL2-L3-VL1-L4-CL-L5-CH1-L6-Fc; wherein the second polypeptide has a structure represented by VL3-VH3-CH1-Fc; VH3-VL3-CH1-Fc; VL3-VH3-CL-Fc; VH3-VL3-CL-Fc; VL3-VH3-CH1-CL-Fc; VH3-VL3-CH1-CL-Fc; VL3-VH3-CL-CH1-Fc; VH3-VL3-CL-CH1-Fc; VL3-CL-VH3-CH1-Fc; VL3-CH1-VH3-CL-Fc; VH3-CH1-VL3-CL-Fc; VH3-CL-VL3-CH1-Fc; VL3-L7-VH3-L8-CH1-Fc; VH3-L7-VL3-L8-CH1-Fc; VL3-L7-VH3-L8-CL-Fc; VH3-L7-VL3-L8-CL-Fc; VL3-L7-VH3-L8-CH1-L9-CL-Fc; VH3-L7-VL3-L8-CH1-L9-CL-Fc; VL3-L7-VH3-L8-CL-L9-CH1-Fc; VH3-L7-VL3-L8-CL-L9-CH1-Fc; VL3-L7-CL-L8-VH3-L9-CH1-Fc; VL3-L7-CH1-L8-VH3-L9-CL-Fc; VH3-L7-CH1-L8-VL3-L9-CL-Fc; VH3-L7-CL-L8-VL3-L9-CH1-Fc; VL3-L7-VH3-L8-CH1-L9-CL-L10-Fc; VH3-L7-VL3-L8-CH1-L9-CL-L10-Fc; VL3-L7-VH3-L8-CL-L9-CH1-L10-Fc; VH3-L7-VL3-L8-CL-L9-CH1-L10-Fc; VL3-L7-CL-L8-VH3-L9-CH1-L10-Fc; VL3-L7-CH1-L8-VH3-L9-CL-L10-Fc; VH3-L7-CH1-L8-VL3-L9-CL-L10-Fc; VH3-L7-CL-L8-VL3-L9-CH1-L10-Fc; VL3-VH3-L7-CH1-CL-Fc; VH3-VL3-L7-CH1-CL-Fc; VL3-VH3-L7-CL-CH1-Fc; VH3-VL3-L7-CL-CH1-Fc; VL3-CL-L7-VH3-CH1-Fc; VL3-CH1-L7-VH3-CL-Fc; VH3-CH1-L7-VL3-CL-Fc; or VH3-CL-L7-VL3-CH1-Fc; wherein VL1 is a first immunoglobulin light chain variable region that specifically binds to an HIV protein; VL2 is a second immunoglobulin light chain variable region that specifically binds to an HIV protein; VL3 is a third immunoglobulin light chain variable region that specifically binds to an HIV protein; VH1 is a first immunoglobulin heavy chain variable region that specifically binds to an HIV protein; VH2 is a second immunoglobulin heavy chain variable region that specifically binds to an HIV protein; VH3 is a third immunoglobulin heavy chain variable region that specifically binds to an HIV protein; Fc is a region comprising an immunoglobulin heavy chain constant region 2 (CH2), an immunoglobulin heavy chain constant region 3 (CH3), and optionally, an immunoglobulin hinge; CH1 is an immunoglobulin heavy chain constant region 1; CL is an immunoglobulin light chain constant region; and L1, L2, L3, L4, L5, L6, L7, L8, L9 and L10 are amino acid linkers.

In other aspects, the invention is directed to an antigen binding polypeptide complex comprising a first polypeptide and a second polypeptide; wherein the first polypeptide has a structure represented by VL1-VL2-VH2-VH1; VH1-VH2-VL2-VL1; VL1-L1-VL2-L2-VH2-L3-VH1; VH1-L1-VH2-L2-VL2-L3-VL1; VL1-VL2-VH2-VH1-Fc; VH1-VH2-VL2-VL1-Fc; VL1-L1-VL2-L2-VH2-L3-VH1-Fc; VH1-L1-VH2-L2-VL2-L3-VL1-Fc; VL1-L1-VL2-L2-VH2-L3-VH1-L4-Fc; VH1-L1-VH2-L2-VL2-L3-VL1-L4-Fc; VL1-VL2-VH2-VH1-CH1; VH1-VH2-VL2-VL1-CH1; VL1-VL2-VH2-VH1-CL; VH1-VH2-VL2-VL1-CL; VL1-VL2-VH2-VH1-CH1-CL; VH1-VH2-VL2-VL1-CH1-CL; VL1-VL2-VH2-VH1-CL-CH1; VH1-VH2-VL2-VL1-CL-CH1; VL1-L1-VL2-L2-VH2-L3-VH1-L4-CH1; VH1-L1-VH2-L2-VL2-L3-VL1-L4-CH1; VL1-L1-VL2-L2-VH2-L3-VH1-L4-CL; VH1-L1-VH2-L2-VL2-L3-VL1-L4-CL; VL1-L1-VL2-L2-VH2-L3-VH1-L4-CH1-L5-CL; VH1-L1-VH2-L2-VL2-L3-VL1-L4-CH1-L5-CL; VL1-L1-VL2-L2-VH2-L3-VH1-L4-CL-L5-CH1; VH1-L1-VH2-L2-VL2-L3-VL1-L4-CL-L5-CH1; VL1-VL2-VH2-VH1-CH1-Fc; VH1-VH2-VL2-VL1-CH1-Fc; VL1-VL2-VH2-VH1-CL-Fc; VH1-VH2-VL2-VL1-CL-Fc; VL1-VL2-VH2-VH1-CH1-CL-Fc; VH1-VH2-VL2-VL1-CH1-CL-Fc; VL1-VL2-VH2-VH1-CL-CH1-Fc; VH1-VH2-VL2-VL1-CL-CH1-Fc; VL1-L1-VL2-L2-VH2-L3-VH1-L4-CH1-Fc; VH1-L1-VH2-L2-VL2-L3-VL1-L4-CH1-Fc; VL1-L1-VL2-L2-VH2-L3-VH1-L4-CL-Fc; VH1-L1-VH2-L2-VL2-L3-VL1-L4-CL-Fc; VL1-L1-VL2-L2-VH2-L3-VH1-L4-CH1-L5-CL-Fc; VH1-L1-VH2-L2-VL2-L3-VL1-L4-CH1-L5-CL-Fc; VL1-L1-VL2-L2-VH2-L3-VH1-L4-CL-L5-CH1-Fc; VH1-L1-VH2-L2-VL2-L3-VL1-L4-CL-L5-CH1-Fc; VL1-L1-VL2-L2-VH2-L3-VH1-L4-CH1-L5-Fc; VH1-L1-VH2-L2-VL2-L3-VL1-L4-CH1-L5-Fc; VL1-L1-VL2-L2-VH2-L3-VH1-L4-CL-L5-Fc; VH1-L1-VH2-L2-VL2-L3-VL1-L4-CL-L5-Fc; VL1-L1-VL2-L2-VH2-L3-VH1-L4-CH1-L5-CL-L6-Fc; VH1-L1-VH2-L2-VL2-L3-VL1-L4-CH1-L5-CL-L6-Fc; VL1-L1-VL2-L2-VH2-L3-VH1-L4-CL-L5-CH1-L6-Fc; or VH1-L1-VH2-L2-VL2-L3-VL1-L4-CL-L5-CH1-L6-Fc; wherein the second polypeptide has a structure represented by VL3-VH3; VH3-VL3; VL3-L4-VH3; VH3-L4-VL3; VL3-VH3-Fc; VH3-VL3-Fc; VL3-L4-VH3-Fc; VH3-L4-VL3-Fc; VL3-VH3-CH1; VH3-VL3-CH1; VL3-VH3-CL; VH3-VL3-CL; VL3-VH3-CH1-CL; VH3-VL3-CH1-CL; VL3-VH3-CL-CH1; VH3-VL3-CL-CH1; VL3-CL-VH3-CH1; VL3-CH1-VH3-CL; VH3-CH1-VL3-CL; VH3-CL-VL3-CH1; VL3-L7-VH3-L8-CH1; VH3-L7-VL3-L8-CH1; VL3-L7-VH3-L8-CL; VH3-L7-VL3-L8-CL; VL3-L7-VH3-L8-CH1-L9-CL; VH3-L7-VL3-L8-CH1-L9-CL; VL3-L7-VH3-L8-CL-L9-CH1; VH3-L7-VL3-L8-CL-L9-CH1; VL3-L7-CL-L8-VH3-L9-CH1; VL3-L7-CH1-L8-VH3-L9-CL; VH3-L7-CH1-L8-VL3-L9-CL; VH3-L7-CL-L8-VL3-L9-CH1; VL3-VH3-L7-CH1-CL; VH3-VL3-L7-CH1-CL; VL3-VH3-L7-CL-CH1; VH3-VL3-L7-CL-CH1; VL3-CL-L7-VH3-CH1; VL3-CH1-L7-VH3-CL; VH3-CH1-L7-VL3-CL; VH3-CL-L7-VL3-CH1; VL3-VH3-CH1-Fc; VH3-VL3-CH1-Fc; VL3-VH3-CL-Fc; VH3-VL3-CL-Fc; VL3-VH3-CH1-CL-Fc; VH3-VL3-CH1-CL-Fc; VL3-VH3-CL-CH1-Fc; VH3-VL3-CL-CH1-Fc; VL3-CL-VH3-CH1-Fc; VL3-CH1-VH3-CL-Fc; VH3-CH1-VL3-CL-Fc; VH3-CL-VL3-CH1-Fc; VL3-L7-VH3-L8-CH1-Fc; VH3-L7-VL3-L8-CH1-Fc; VL3-L7-VH3-L8-CL-Fc; VH3-L7-VL3-L8-CL-Fc; VL3-L7-VH3-L8-CH1-L9-CL-Fc; VH3-L7-VL3-L8-CH1-L9-CL-Fc; VL3-L7-VH3-L8-CL-L9-CH1-Fc; VH3-L7-VL3-L8-CL-L9-CH1-Fc; VL3-L7-CL-L8-VH3-L9-CH1-Fc; VL3-L7-CH1-L8-VH3-L9-CL-Fc; VH3-L7-CH1-L8-VL3-L9-CL-Fc; VH3-L7-CL-L8-VL3-L9-CH1-Fc; VL3-L7-VH3-L8-CH1-L9-Fc; VH3-L7-VL3-L8-CH1-L9-Fc; VL3-L7-VH3-L8-CL-L9-Fc; VH3-L7-VL3-L8-CL-L9-Fc; VL3-L7-VH3-L8-CH1-L9-CL-L10-Fc; VH3-L7-VL3-L8-CH1-L9-CL-L10-Fc; VL3-L7-VH3-L8-CL-L9-CH1-L10-Fc; VH3-L7-VL3-L8-CL-L9-CH1-L10-Fc; VL3-L7-CL-L8-VH3-L9-CH1-L10-Fc; VL3-L7-CH1-L8-VH3-L9-CL-L10-Fc; VH3-L7-CH1-L8-VL3-L9-CL-L10-Fc; VH3-L7-CL-L8-VL3-L9-CH1-L10-Fc; VL3-VH3-L7-CH1-CL-Fc; VH3-VL3-L7-CH1-CL-Fc; VL3-VH3-L7-CL-CH1-Fc; VH3-VL3-L7-CL-CH1-Fc; VL3-CL-L7-VH3-CH1-Fc; VL3-CH1-L7-VH3-CL-Fc; VH3-CH1-L7-VL3-CL-Fc; or VH3-CL-L7-VL3-CH1-Fc; wherein VL1 is a first immunoglobulin light chain variable region that specifically binds to an HIV protein; VL2 is a second immunoglobulin light chain variable region that specifically binds to an HIV protein; VL3 is a third immunoglobulin light chain variable region that specifically binds to an HIV protein; VH1 is a first immunoglobulin heavy chain variable region that specifically binds to an HIV protein; VH2 is a second immunoglobulin heavy chain variable region that specifically binds to an HIV protein; VH3 is a third immunoglobulin heavy chain variable region that specifically binds to an HIV protein; Fc is a region comprising an immunoglobulin heavy chain constant region 2 (CH2), an immunoglobulin heavy chain constant region 3 (CH3), and optionally, an immunoglobulin hinge; CH1 is an immunoglobulin heavy chain constant region 1; CL is an immunoglobulin light chain constant region; and L1, L2, L3, L4, L5, L6, L7, L8, L9 and L10 are amino acid linkers.

In other aspects, the invention is directed to an antigen binding polypeptide complex comprising a first polypeptide, a second polypeptide, and a third polypeptide; wherein the first polypeptide has a structure represented by VL1-VL2-VH2-VH1; VH1-VH2-VL2-VL1; VL1-L1-VL2-L2-VH2-L3-VH1; or VH1-L1-VH2-L2-VL2-L3-VL1; wherein the second polypeptide has a structure represented by VL3; wherein the third polypeptide has a structure represented by VH3; wherein VL1 is a first immunoglobulin light chain variable region that specifically binds to an HIV protein; VL2 is a second immunoglobulin light chain variable region that specifically binds to an HIV protein; VL3 is a third immunoglobulin light chain variable region that specifically binds to an HIV protein; VH1 is a first immunoglobulin heavy chain variable region that specifically binds to an HIV protein; VH2 is a second immunoglobulin heavy chain variable region that specifically binds to an HIV protein; VH3 is a third immunoglobulin heavy chain variable region that specifically binds to an HIV protein; and L1, L2 and L3 are amino acid linkers.

In other aspects, the invention is directed to an antigen binding polypeptide complex comprising a first polypeptide, a second polypeptide, and a third polypeptide; wherein the first polypeptide has a structure represented by VL1-VL2-VH2-VH1-Fc; VH1-VH2-VL2-VL1-Fc; VL1-L1-VL2-L2-VH2-L3-VH1-Fc; VH1-L1-VH2-L2-VL2-L3-VL1-Fc; VL1-L1-VL2-L2-VH2-L3-VH1-L4-Fc; or VH1-L1-VH2-L2-VL2-L3-VL1-L4-Fc; wherein the second polypeptide has a structure represented by VL3; or VL3-L5; wherein the third polypeptide has a structure represented by VH3-Fc; or VH3-L6-Fc; wherein VL1 is a first immunoglobulin light chain variable region that specifically binds to an HIV protein; VL2 is a second immunoglobulin light chain variable region that specifically binds to an HIV protein; VL3 is a third immunoglobulin light chain variable region that specifically binds to an HIV protein; VH1 is a first immunoglobulin heavy chain variable region that specifically binds to an HIV protein; VH2 is a second immunoglobulin heavy chain variable region that specifically binds to an HIV protein; VH3 is a third immunoglobulin heavy chain variable region that specifically binds to an HIV protein; Fc is a region comprising an immunoglobulin heavy chain constant region 2 (CH2), an immunoglobulin heavy chain constant region 3 (CH3), and optionally, an immunoglobulin hinge; and L1, L2, L3, L4, L5 and L6 are amino acid linkers.

In other aspects, the invention is directed to an antigen binding polypeptide complex comprising a first polypeptide, a second polypeptide, and a third polypeptide; wherein the first polypeptide has a structure represented by VL1-VL2-VH2-VH1-Fc; VH1-VH2-VL2-VL1-Fc; VL1-L1-VL2-L2-VH2-L3-VH1-Fc; VH1-L1-VH2-L2-VL2-L3-VL1-Fc; VL1-L1-VL2-L2-VH2-L3-VH1-L4-Fc; or VH1-L1-VH2-L2-VL2-L3-VL1-L4-Fc; wherein the second polypeptide has a structure represented by VL3-Fc; or VL3-L5-Fc; wherein the third polypeptide has a structure represented by VH3; or VH3-L6; wherein VL1 is a first immunoglobulin light chain variable region that specifically binds to an HIV protein; VL2 is a second immunoglobulin light chain variable region that specifically binds to an HIV protein; VL3 is a third immunoglobulin light chain variable region that specifically binds to an HIV protein; VH1 is a first immunoglobulin heavy chain variable region that specifically binds to an HIV protein; VH2 is a second immunoglobulin heavy chain variable region that specifically binds to an HIV protein; VH3 is a third immunoglobulin heavy chain variable region that specifically binds to an HIV protein; Fc is a region comprising an immunoglobulin heavy chain constant region 2 (CH2), an immunoglobulin heavy chain constant region 3 (CH3), and optionally, an immunoglobulin hinge; and L1, L2, L3, L4, L5 and L6 are amino acid linkers.

In other aspects, the invention is directed to an antigen binding polypeptide complex comprising a first polypeptide, a second polypeptide, and a third polypeptide; wherein the first polypeptide has a structure represented by VL1-VL2-VH2-VH1-CH1; VH1-VH2-VL2-VL1-CH1; VL1-VL2-VH2-VH1-CL; VH1-VH2-VL2-VL1-CL; VL1-VL2-VH2-VH1-CH1-CL; VH1-VH2-VL2-VL1-CH1-CL; VL1-VL2-VH2-VH1-CL-CH1; VH1-VH2-VL2-VL1-CL-CH1; VL1-L1-VL2-L2-VH2-L3-VH1-L4-CH1; VH1-L1-VH2-L2-VL2-L3-VL1-L4-CH1; VL1-L1-VL2-L2-VH2-L3-VH1-L4-CL; VH1-L1-VH2-L2-VL2-L3-VL1-L4-CL; VL1-L1-VL2-L2-VH2-L3-VH1-L4-CH1-L5-CL; VH1-L1-VH2-L2-VL2-L3-VL1-L4-CH1-L5-CL; VL1-L1-VL2-L2-VH2-L3-VH1-L4-CL-L5-CH1; or VH1-L1-VH2-L2-VL2-L3-VL1-L4-CL-L5-CH1; wherein the second polypeptide has a structure represented by VL3-CH1; VL3-CL; VL3-L6-CH1; or VL3-L6-CL; wherein the third polypeptide has a structure represented by VH3-CH1; VH3-CL; VH3-L7-CH1; or VH3-L7-CL; wherein VL1 is a first immunoglobulin light chain variable region that specifically binds to an HIV protein; VL2 is a second immunoglobulin light chain variable region that specifically binds to an HIV protein; VL3 is a third immunoglobulin light chain variable region that specifically binds to an HIV protein; VH1 is a first immunoglobulin heavy chain variable region that specifically binds to an HIV protein; VH2 is a second immunoglobulin heavy chain variable region that specifically binds to an HIV protein; VH3 is a third immunoglobulin heavy chain variable region that specifically binds to an HIV protein; CH1 is an immunoglobulin heavy chain constant region 1; CL is an immunoglobulin light chain constant region; and L1, L2, L3, L4, L5, L6 and L7 are amino acid linkers.

In other aspects, the invention is directed to an antigen binding polypeptide complex comprising a first polypeptide, a second polypeptide, and a third polypeptide; wherein the first polypeptide has a structure represented by VL1-VL2-VH2-VH1; VH1-VH2-VL2-VL1; VL1-L1-VL2-L2-VH2-L3-VH1; VH1-L1-VH2-L2-VL2-L3-VL1; VL1-VL2-VH2-VH1-Fc; VH1-VH2-VL2-VL1-Fc; VL1-L1-VL2-L2-VH2-L3-VH1-Fc; VH1-L1-VH2-L2-VL2-L3-VL1-Fc; VL1-L1-VL2-L2-VH2-L3-VH1-L4-Fc; VH1-L1-VH2-L2-VL2-L3-VL1-L4-Fc; VL1-VL2-VH2-VH1-CH1; VH1-VH2-VL2-VL1-CH1; VL1-VL2-VH2-VH1-CL; VH1-VH2-VL2-VL1-CL; VL1-VL2-VH2-VH1-CH1-CL; VH1-VH2-VL2-VL1-CH1-CL; VL1-VL2-VH2-VH1-CL-CH1; VH1-VH2-VL2-VL1-CL-CH1; VL1-L1-VL2-L2-VH2-L3-VH1-L4-CH1; VH1-L1-VH2-L2-VL2-L3-VL1-L4-CH1; VL1-L1-VL2-L2-VH2-L3-VH1-L4-CL; VH1-L1-VH2-L2-VL2-L3-VL1-L4-CL; VL1-L1-VL2-L2-VH2-L3-VH1-L4-CH1-L5-CL; VH1-L1-VH2-L2-VL2-L3-VL1-L4-CH1-L5-CL; VL1-L1-VL2-L2-VH2-L3-VH1-L4-CL-L5-CH1; VH1-L1-VH2-L2-VL2-L3-VL1-L4-CL-L5-CH1; VL1-VL2-VH2-VH1-CH1-Fc; VH1-VH2-VL2-VL1-CH1-Fc; VL1-VL2-VH2-VH1-CL-Fc; VH1-VH2-VL2-VL1-CL-Fc; VL1-VL2-VH2-VH1-CH1-CL-Fc; VH1-VH2-VL2-VL1-CH1-CL-Fc; VL1-VL2-VH2-VH1-CL-CH1-Fc; VH1-VH2-VL2-VL1-CL-CH1-Fc; VL1-L1-VL2-L2-VH2-L3-VH1-L4-CH1-Fc; VH1-L1-VH2-L2-VL2-L3-VL1-L4-CH1-Fc; VL1-L1-VL2-L2-VH2-L3-VH1-L4-CL-Fc; VH1-L1-VH2-L2-VL2-L3-VL1-L4-CL-Fc; VL1-L1-VL2-L2-VH2-L3-VH1-L4-CH1-L5-CL-Fc; VH1-L1-VH2-L2-VL2-L3-VL1-L4-CH1-L5-CL-Fc; VL1-L1-VL2-L2-VH2-L3-VH1-L4-CL-L5-CH1-Fc; VH1-L1-VH2-L2-VL2-L3-VL1-L4-CL-L5-CH1-Fc; VL1-L1-VL2-L2-VH2-L3-VH1-L4-CH1-L5-Fc; VH1-L1-VH2-L2-VL2-L3-VL1-L4-CH1-L5-Fc; VL1-L1-VL2-L2-VH2-L3-VH1-L4-CL-L5-Fc; VH1-L1-VH2-L2-VL2-L3-VL1-L4-CL-L5-Fc; VL1-L1-VL2-L2-VH2-L3-VH1-L4-CH1-L5-CL-L6-Fc; VH1-L1-VH2-L2-VL2-L3-VL1-L4-CH1-L5-CL-L6-Fc; VL1-L1-VL2-L2-VH2-L3-VH1-L4-CL-L5-CH1-L6-Fc; or VH1-L1-VH2-L2-VL2-L3-VL1-L4-CL-L5-CH1-L6-Fc; wherein the second polypeptide has a structure represented by VL3; VL3-Fc; VL3-CH1; VL3-CL; VL3-CH1-CL; VL3-CL-CH1; VL3-CH1-Fc; VL3-CL-Fc; VL3-CH1-CL-Fc; VL3-CL-CH1-Fc; VL3-L7-Fc; VL3-L7-CH1; VL3-L7-CL; VL3-L7-CH1-L8-CL; VL3-L7-CL-L8-CH1; VL3-L7-CH1-L8-Fc; VL3-L7-CL-L8-Fc; VL3-L7-CH1-L8-CL-Fc; VL3-L7-CL-L8-CH1-Fc; VL3-L7-CH1-L8-CL-L9-Fc; or VL3-L7-CL-L8-CH1-L9-Fc; wherein the third polypeptide has a structure represented by VH3; VH3-Fc; VH3-CH1; VH3-CL; VH3-CH1-CL; VH3-CL-CH1; VH3-CH1-Fc; VH3-CL-Fc; VH3-CH1-CL-Fc; VH3-CL-CH1-Fc; VH3-L10-Fc; VH3-L10-CH1; VH3-L10-CL; VH3-L10-CH1-L11-CL; VH3-L10-CL-L11-CH1; VH3-L10-CH1-L11-Fc; VH3-L10-CL-L11-Fc; VH3-L10-CH1-L11-CL-Fc; VH3-L10-CL-L11-CH1-Fc; VH3-L10-CH1-L11-CL-L12-Fc; or VH3-L10-CL-L11-CH1-L12-Fc; wherein VL1 is a first immunoglobulin light chain variable region that specifically binds to an HIV protein; VL2 is a second immunoglobulin light chain variable region that specifically binds to an HIV protein; VL3 is a third immunoglobulin light chain variable region that specifically binds to an HIV protein; VH1 is a first immunoglobulin heavy chain variable region that specifically binds to an HIV protein; VH2 is a second immunoglobulin heavy chain variable region that specifically binds to an HIV protein; VH3 is a third immunoglobulin heavy chain variable region that specifically binds to an HIV protein; Fc is a region comprising an immunoglobulin heavy chain constant region 2 (CH2), an immunoglobulin heavy chain constant region 3 (CH3), and optionally, an immunoglobulin hinge; CH1 is an immunoglobulin heavy chain constant region 1; CL is an immunoglobulin light chain constant region; and L1, L2, L3, L4, L5, L6, L7, L8, L9, L10, L11 and L12 are amino acid linkers.

In some aspects, the invention is directed to an antigen binding polypeptide complex (e.g., an antibody or antigen binding fragment thereof) comprising a first polypeptide having a structure represented by VL1-VL2-VH2-VH1-Fc, VH1-VH2-VL2-VL1-Fc, VL1-L1-VL2-L2-VH2-L3-VH1-Fc, VH1-L1-VH2-L2-VL2-L3-VH1-Fc, VL1-L1-VL2-L2-VH2-L3-VH1-L4-Fc, or VH1-L1-VH2-L2-VL2-L3-VL1-L4-Fc; and a second polypeptide having a structure represented by VL3-VH3-Fc, VL3-L5-VH3-Fc, VH3-VL3-Fc, VH3-L5-VL3-Fc, VL3-L5-VH3-L6-Fc, or VH3-L5-VL3-L6-Fc; wherein VL1 is a first immunoglobulin light chain variable region that specifically binds to an HIV protein; VL2 is a second immunoglobulin light chain variable region that specifically binds to an HIV protein; VL3 is a third immunoglobulin light chain variable region that specifically binds to an HIV protein; VH1 is a first immunoglobulin heavy chain variable region that specifically binds to an HIV protein; VH2 is a second immunoglobulin heavy chain variable region that specifically binds to an HIV protein; VH3 is a third immunoglobulin heavy chain variable region that specifically binds to an HIV protein; Fc is a region comprising an immunoglobulin heavy chain constant region 2 (CH2), an immunoglobulin heavy chain constant region 3 (CH3), and optionally, an immunoglobulin hinge; and L1, L2, L3, L4, L5 and L6 are amino acid linkers.

In some aspects, the invention is directed to an antigen binding polypeptide complex (e.g., an antibody or antigen binding fragment thereof) comprising a first polypeptide having a structure represented by VL1-VL2-VH2-VH1-Fc, VH1-VH2-VL2-VL1-Fc, VL1-L1-VL2-L2-VH2-L3-VH1-Fc, VH1-L1-VH2-L2-VL2-L3-VH1-Fc, VL1-L1-VL2-L2-VH2-L3-VH1-L4-Fc, or VH1-L1-VH2-L2-VL2-L3-VL1-L4-Fc; a second polypeptide having a structure represented by VH3-CH1-Fc, VH3-L5-CH1-Fc, VH3-L5-CH1-L6-Fc, VL3-CH1-Fc, VL3-L5-CH1-Fc, or VL3-L5-CH1-L6-Fc; and a third polypeptide having a structure represented by VL3-CL, VL3-L7-CL, VH3-CL, or VH3-L7-CL; wherein VL1 is a first immunoglobulin light chain variable region that specifically binds to an HIV protein; VL2 is a second immunoglobulin light chain variable region that specifically binds to an HIV protein; VL3 is a third immunoglobulin light chain variable region that specifically binds to an HIV protein; VH1 is a first immunoglobulin heavy chain variable region that specifically binds to an HIV protein; VH2 is a second immunoglobulin heavy chain variable region that specifically binds to an HIV protein; VH3 is a third immunoglobulin heavy chain variable region that specifically binds to an HIV protein; Fc is a region comprising an immunoglobulin heavy chain constant region 2 (CH2), an immunoglobulin heavy chain constant region 3 (CH3), and optionally, an immunoglobulin hinge; CH1 is an immunoglobulin heavy chain constant region 1; CL is an immunoglobulin light chain constant region; and L1, L2, L3, L4, L5, L6 and L7 are amino acid linkers.

In some aspects, the invention is directed to an antigen binding polypeptide complex (e.g., an antibody or antigen binding fragment thereof) comprising a first polypeptide having a structure represented by VL1-VL2-VH2-VH1-Fc, VH1-VH2-VL2-VL1-Fc, VL1-L1-VL2-L2-VH2-L3-VH1-Fc, VH1-L1-VH2-L2-VL2-L3-VH1-Fc; VL1-L1-VL2-L2-VH2-L3-VH1-L4-Fc; or VH1-L1-VH2-L2-VL2-L3-VL1-L4-Fc; and a second polypeptide having a structure represented by CL-VL3-VH3-CH1-Fc, CL-L5-VL3-L6-VH3-L7-CH1-Fc, CL-L5-VL3-L6-VH3-L7-CH1-L8-Fc, CL-VH3-VL3-CH1-Fc; CL-L5-VH3-L6-VL3-L7-CH1-Fc, CL-L5-VH3-L6-VL3-L7-CH1-L8-Fc, CH1-VL3-VH3-CL-Fc, CH1-L5-VL3-L6-VH3-L7-CL-Fc, CH1-L5-VL3-L6-VH3-L7-CL-L8-Fc, CH1-VH3-VL3-CL-Fc; CH1-L5-VH3-L6-VL3-L7-CL-Fc, or CH1-L5-VH3-L6-VL3-L7-CL-L8-Fc; wherein VL1 is a first immunoglobulin light chain variable region that specifically binds to an HIV protein; VL2 is a second immunoglobulin light chain variable region that specifically binds to an HIV protein; VL3 is a third immunoglobulin light chain variable region that specifically binds to an HIV protein; VH1 is a first immunoglobulin heavy chain variable region that specifically binds to an HIV protein; VH2 is a second immunoglobulin heavy chain variable region that specifically binds to an HIV protein; VH3 is a third immunoglobulin heavy chain variable region that specifically binds to an HIV protein; Fc is a region comprising an immunoglobulin heavy chain constant region 2 (CH2), an immunoglobulin heavy chain constant region 3 (CH3), and optionally, an immunoglobulin hinge; CH1 is an immunoglobulin heavy chain constant region 1; CL is an immunoglobulin light chain constant region; and L1, L2, L3, L4, L5, L6, L7 and L8 are amino acid linkers.

In some aspects, the invention is directed to an antigen binding polypeptide complex (e.g., an antibody or antigen binding fragment thereof) comprising a first polypeptide having a structure represented by VL1-VL2-VH2-VH1-Fc, VH1-VH2-VL2-VL1-Fc, VL1-L1-VL2-L2-VH2-L3-VH1-Fc, VH1-L1-VH2-L2-VL2-L3-VH1-Fc, VL1-L1-VL2-L2-VH2-L3-VH1-L4-Fc, or VH1-L1-VH2-L2-VL2-L3-VL1-L4-Fc, and a second polypeptide having a structure represented by VL3-CL-VH3-CH1-Fc, VL3-L5-CL-L6-VH3-L7-CH1-Fc, VL3-L5-CL-L6-VH3-L7-CH1-L8-Fc, VH3-CL-VL3-CH1-Fc, VH3-L5-CL-L6-VL3-L7-CH1-Fc, VH3-L5-CL-L6-VL3-L7-CH1-L8-Fc, VL3-CH1-VH3-CL-Fc, VL3-L5-CH1-L6-VH3-L7-CL-Fc, VL3-L5-CH1-L6-VH3-L7-CL-L8-Fc, VH3-CH1-VL3-CL-Fc, VH3-L5-CH1-L6-VL3-L7-CL-Fc, or VH3-L5-CH1-L6-VL3-L7-CL-L8-Fc; wherein VL1 is a first immunoglobulin light chain variable region that specifically binds to an HIV protein; VL2 is a second immunoglobulin light chain variable region that specifically binds to an HIV protein; VL3 is a third immunoglobulin light chain variable region that specifically binds to an HIV protein; VH1 is a first immunoglobulin heavy chain variable region that specifically binds to an HIV protein; VH2 is a second immunoglobulin heavy chain variable region that specifically binds to an HIV protein; VH3 is a third immunoglobulin heavy chain variable region that specifically binds to an HIV protein; Fc is a region comprising an immunoglobulin heavy chain constant region 2 (CH2), an immunoglobulin heavy chain constant region 3 (CH3), and optionally, an immunoglobulin hinge; CH1 is an immunoglobulin heavy chain constant region 1; CL is an immunoglobulin light chain constant region; and L1, L2, L3, L4, L5, L6, L7 and L8 are amino acid linkers.

In some aspects, the invention is directed to an antigen binding polypeptide complex (e.g., an antibody or antigen binding fragment thereof) comprising a first polypeptide having a structure represented by VL1-VL2-VH2-VH1-Fc, VH1-VH2-VL2-VL1-Fc, VL1-L1-VL2-L2-VH2-L3-VH1-Fc, VH1-L1-VH2-L2-VL2-L3-VH1-Fc, VL1-L1-VL2-L2-VH2-L3-VH1-L4-Fc, or VH1-L1-VH2-L2-VL2-L3-VL1-L4-Fc; and a second polypeptide having a structure represented by VL3-VH3-CL-CH1-Fc, VL3-L5-VH3-L6-CL-CH1-Fc, VL3-L5-VH3-L6-CL-L7-CH1-Fc, VL3-L5-VH3-L6-CL-L7-CH1-L8-Fc, VH3-VL3-CL-CH1-Fc, VH3-L5-VL3-L6-CL-CH1-Fc, VH3-L5-VL3-L6-CL-L7-CH1-Fc, VH3-L5-VL3-L6-CL-L7-CH1-L8-Fc, VL3-VH3-CH1-CL-Fc, VL3-L5-VH3-L6-CH1-CL-Fc, VL3-L5-VH3-L6-CH1-L7-CL-Fc, VL3-L5-VH3-L6-CH1-L7-CL-L8-Fc, VH3-VL3-CH1-CL-Fc, VH3-L5-VL3-L6-CH1-CL-Fc; VH3-L5-VL3-L6-CH1-L7-CL-Fc; or VH3-L5-VL3-L6-CH1-L7-CL-L8-Fc; wherein VL1 is a first immunoglobulin light chain variable region that specifically binds to an HIV protein; VL2 is a second immunoglobulin light chain variable region that specifically binds to an HIV protein; VL3 is a third immunoglobulin light chain variable region that specifically binds to an HIV protein; VH1 is a first immunoglobulin heavy chain variable region that specifically binds to an HIV protein; VH2 is a second immunoglobulin heavy chain variable region that specifically binds to an HIV protein; VH3 is a third immunoglobulin heavy chain variable region that specifically binds to an HIV protein; Fc is a region comprising an immunoglobulin heavy chain constant region 2 (CH2), an immunoglobulin heavy chain constant region 3 (CH3), and optionally, an immunoglobulin hinge; CH1 is an immunoglobulin heavy chain constant region 1; CL is an immunoglobulin light chain constant region; and L1, L2, L3, L4, L5, L6, L7 and L8 are amino acid linkers.

In some aspects, the invention is directed to an antigen binding polypeptide complex (e.g., an antibody or antigen binding fragment thereof) comprising a first polypeptide having a structure represented by VL1-VL2-VH2-VH1-CH1-Fc, VH1-VH2-VL2-VL1-CH1-Fc, VL1-L1-VL2-L2-VH2-L3-VH1-CH1-Fc, VH1-L1-VH2-L2-VL2-L3-VH1-CH1-Fc, VL1-L1-VL2-L2-VH2-L3-VH1-L4-CH1-Fc, VH1-L1-VH2-L2-VL2-L3-VH1-L4-CH1-Fc, VL1-L1-VL2-L2-VH2-L3-VH1-L4-CH1-L5-Fc, VH1-L1-VH2-L2-VL2-L3-VH1-L4-CH1-L5-Fc, VL1-VL2-VH2-VH1-CL-Fc, VH1-VH2-VL2-VL1-CL-Fc, VL1-L1-VL2-L2-VH2-L3-VH1-CL-Fc, VH1-L1-VH2-L2-VL2-L3-VH1-CL-Fc, VL1-L1-VL2-L2-VH2-L3-VH1-L4-CL-Fc, VH1-L1-VH2-L2-VL2-L3-VH1-L4-CL-Fc, VL1-L1-VL2-L2-VH2-L3-VH1-L4-CL-L5-Fc, or VH1-L1-VH2-L2-VL2-L3-VH1-L4-CL-L5-Fc; and a second polypeptide having a structure represented by VL3-VH3-CL-Fc, VL3-L6-VH3-L7-CL-Fc, VL3-L6-VH3-L7-CL-L8-Fc, VH3-VL3-CL-Fc, VH3-L6-VL3-L7-CL-Fc, VH3-L6-VL3-L7-CL-L8-Fc, VL3-VH3-CH1-Fc, VL3-L6-VH3-L7-CH1-Fc, VL3-L6-VH3-L7-CH1-L8-Fc, VH3-VL3-CH1-Fc, VH3-L6-VL3-L7-CH1-Fc, or VH3-L6-VL3-L7-CH1-L8-Fc; wherein VL1 is a first immunoglobulin light chain variable region that specifically binds to an HIV protein; VL2 is a second immunoglobulin light chain variable region that specifically binds to an HIV protein; VL3 is a third immunoglobulin light chain variable region that specifically binds to an HIV protein; VH1 is a first immunoglobulin heavy chain variable region that specifically binds to an HIV protein; VH2 is a second immunoglobulin heavy chain variable region that specifically binds to an HIV protein; VH3 is a third immunoglobulin heavy chain variable region that specifically binds to an HIV protein; Fc is a region comprising an immunoglobulin heavy chain constant region 2 (CH2), an immunoglobulin heavy chain constant region 3 (CH3), and optionally, an immunoglobulin hinge; CH1 is an immunoglobulin heavy chain constant region 1; CL is an immunoglobulin light chain constant region; and L1, L2, L3, L4, L5, L6, L7 and L8 are amino acid linkers.

In some aspects, the invention is directed to an antigen binding polypeptide complex (e.g., an antibody or antigen binding fragment thereof) comprising a first polypeptide having a structure represented by VL1-VL2-VH2-VH1-CL-CH1-Fc, VL1-L1-VL2-L2-VH2-L3-VH1-CL-CH1-Fc, VL1-L1-VL2-L2-VH2-L3-VH1-L4-CL-CH1-Fc, VL1-L1-VL2-L2-VH2-L3-VH1-L4-CL-L5-CH1-Fc, VL1-L1-VL2-L2-VH2-L3-VH1-L4-CL-L5-CH1-L6-Fc, VH1-VH2-VL2-VL1-CL-CH1-Fc, VH1-L1-VH2-L2-VL2-L3-VL1-CL-CH1-Fc, VH1-L1-VH2-L2-VL2-L3-VL1-L4-CL-CH1-Fc, VH1-L1-VH2-L2-VL2-L3-VL1-L4-CL-L5-CH1-Fc, VH1-L1-VH2-L2-VL2-L3-VL1-L4-CL-L5-CH1-L6-Fc, VL1-VL2-VH2-VH1-CH1-CL-Fc, VL1-L1-VL2-L2-VH2-L3-VH1-CH1-CL-Fc, VL1-L1-VL2-L2-VH2-L3-VH1-L4-CH1-CL-Fc, VL1-L1-VL2-L2-VH2-L3-VH1-L4-CH1-L5-CL-Fc, VL1-L1-VL2-L2-VH2-L3-VH1-L4-CH1-L5-CL-L6-Fc, VH1-VH2-VL2-VL1-CH1-CL-Fc, VH1-L1-VH2-L2-VL2-L3-VL1-CH1-CL-Fc, VH1-L1-VH2-L2-VL2-L3-VL1-L4-CH1-CL-Fc, VH1-L1-VH2-L2-VL2-L3-VL1-L4-CH1-L5-CL-Fc, or VH1-L1-VH2-L2-VL2-L3-VL1-L4-CH1-L5-CL-L6-Fc; and a second polypeptide having a structure represented by VL3-VH3-Fc, VL3-L7-VH3-Fc, VL3-L7-VH3-L8-Fc, VH3-VL3-Fc, VH3-L7-VL3-Fc, or VH4-L7-VL3-L8-Fc; wherein VL1 is a first immunoglobulin light chain variable region that specifically binds to an HIV protein; VL2 is a second immunoglobulin light chain variable region that specifically binds to an HIV protein; VL3 is a third immunoglobulin light chain variable region that specifically binds to an HIV protein; VH1 is a first immunoglobulin heavy chain variable region that specifically binds to an HIV protein; VH2 is a second immunoglobulin heavy chain variable region that specifically binds to an HIV protein; VH3 is a third immunoglobulin heavy chain variable region that specifically binds to an HIV protein; Fc is a region comprising an immunoglobulin heavy chain constant region 2 (CH2), an immunoglobulin heavy chain constant region 3 (CH3), and optionally, an immunoglobulin hinge; CH1 is an immunoglobulin heavy chain constant region 1; CL is an immunoglobulin light chain constant region; and L1, L2, L3, L4, L5, L6, L7 and L8 are amino acid linkers.

In other aspects, the invention is directed to antigen binding polypeptides and antigen binding polypeptide complexes comprising a polypeptide having a structure represented by VL1-VL2-VL3-VH3-VH2-VH1; VH1-VH2-VH3-VL3-VL2-VL1; VL1-VH2-VL3-VH3-VL2-VH1; VH1-VL2-VH3-VL3-VH2-VL1; VL1-VL2-VH3-VL3-VH2-VH1; VH1-VH2-VL3-VH3-VL2-VL1; VL1-VH2-VH3-VL3-VL2-VH1; or VH1-VL2-VL3-VH3-VH2-VL1. In some aspects, the antigen binding polypeptide or antigen binding polypeptide complex contains an amino acid linker between any two regions denoted in a structure described herein. In some aspects, the antigen binding polypeptide or antigen binding polypeptide complex can contain an Fc region, CH1 region, CL region or any combination thereof. In some aspects, the antigen binding polypeptide complex is an antibody or antigen binding fragment thereof.

In some aspects, an antigen binding polypeptide of antigen binding polypeptide complex of the invention comprises a polypeptide having a structure represented by VL1-VL2-VL3-VH3-VH2-VH1; VH1-VH2-VH3-VL3-VL2-VL1; VL1-VH2-VL3-VH3-VL2-VH1; VH1-VL2-VH3-VL3-VH2-VL1; VL1-VL2-VH3-VL3-VH2-VH1; VH1-VH2-VL3-VH3-VL2-VL1; VL1-VH2-VH3-VL3-VL2-VH1; VH1-VL2-VL3-VH3-VH2-VL1; VL1-L1-VL2-L2-VL3-L3-VH3-L4-VH2-L5-VH1; VH1-L1-VH2-L2-VH3-L3-VL3-L4-VL2-L5-VL1; VL1-L1-VH2-L2-VL3-L3-VH3-L4-VL2-L5-VH1; VH1-L1-VL2-L2-VH3-L3-VL3-L4-VH2-L5-VL1; VL1-L1-VL2-L2-VH3-L3-VL3-L4-VH2-L5-VH1; VH1-L1-VH2-L2-VL3-L3-VH3-L4-VL2-L5-VL1; VL1-L1-VH2-L2-VH3-L3-VL3-L4-VL2-L5-VH1; or VH1-L1-VL2-L2-VL3-L3-VH3-L4-VH2-L5-VL1; wherein VL1 is a first immunoglobulin light chain variable region that specifically binds to an HIV protein; VL2 is a second immunoglobulin light chain variable region that specifically binds to an HIV protein; VL3 is a third immunoglobulin light chain variable region that specifically binds to an HIV protein; VH1 is a first immunoglobulin heavy chain variable region that specifically binds to an HIV protein; VH2 is a second immunoglobulin heavy chain variable region that specifically binds to an HIV protein; VH3 is a third immunoglobulin heavy chain variable region that specifically binds to an HIV protein; and L1, L2, L3, L4 and L5 are amino acid linkers.

In some aspects, the antigen binding polypeptide complex comprises a first polypeptide and a second polypeptide; wherein the first polypeptide has a structure represented by VL1-VL2-VL3-VH3-VH2-VH1; VH1-VH2-VH3-VL3-VL2-VL1; VL1-VH2-VL3-VH3-VL2-VH1; VH1-VL2-VH3-VL3-VH2-VL1; VL1-VL2-VH3-VL3-VH2-VH1; VH1-VH2-VL3-VH3-VL2-VL1; VL1-VH2-VH3-VL3-VL2-VH1; VH1-VL2-VL3-VH3-VH2-VL1; VL1-L1-VL2-L2-VL3-L3-VH3-L4-VH2-L5-VH1; VH1-L1-VH2-L2-VH3-L3-VL3-L4-VL2-L5-VL1; VL1-L1-VH2-L2-VL3-L3-VH3-L4-VL2-L5-VH1; VH1-L1-VL2-L2-VH3-L3-VL3-L4-VH2-L5-VL1; VL1-L1-VL2-L2-VH3-L3-VL3-L4-VH2-L5-VH1; VH1-L1-VH2-L2-VL3-L3-VH3-L4-VL2-L5-VL1; VL1-L1-VH2-L2-VH3-L3-VL3-L4-VL2-L5-VH1; or VH1-L1-VL2-L2-VL3-L3-VH3-L4-VH2-L5-VL1; wherein the second polypeptide has a structure represented by VL4-VH4; VH4-VL4; VL4-L6-VH4; VH4-L6-VL4; VL4-L6-VH4-L7; VH4-L6-VL4-L7; VL4-VL5-VH5-VH4; VH4-VH5-VL5-VL4; VL4-L6-VL5-L7-VH5-L8-VH4; VH4-L6-VH5-L7-VL5-L8-VL4; VL4-VL5-VL6-VH6-VH5-VH4; VH4-VH5-VH6-VL6-VL5-VL4; VL4-VH5-VL6-VH6-VL5-VH4; VH4-VL5-VH6-VL6-VH5-VL4; VL4-VL5-VH6-VL6-VH5-VH4; VH4-VH5-VL6-VH6-VL5-VL4; VL4-VH5-VH6-VL6-VL5-VH4; VH4-VL5-VL6-VH6-VH5-VL4; VL4-L6-VL5-L7-VL6-L8-VH6-L9-VH5-L10-VH4; VH4-L6-VH5-L7-VH6-L8-VL6-L9-VL5-L10-VL4; VL4-L6-VH5-L7-VL6-L8-VH6-L9-VL5-L10-VH4; VH4-L6-VL5-L7-VH6-L8-VL6-L9-VH5-L10-VL4; VL4-L6-VL5-L7-VH6-L8-VL6-L9-VH5-L10-VH4; VH4-L6-VH5-L7-VL6-L8-VH6-L9-VL5-L10-VL4; VL4-L6-VH5-L7-VH6-L8-VL6-L9-VL5-L10-VH4; or VH4-L6-VL5-L7-VL6-L8-VH6-L9-VH5-L10-VL4; wherein VL1 is a first immunoglobulin light chain variable region that specifically binds to an HIV protein; VL2 is a second immunoglobulin light chain variable region that specifically binds to an HIV protein; VL3 is a third immunoglobulin light chain variable region that specifically binds to an HIV protein; VL4 is a fourth immunoglobulin light chain variable region that specifically binds to an HIV protein; VL5 is a fifth immunoglobulin light chain variable region that specifically binds to an HIV protein; VL6 is a sixth immunoglobulin light chain variable region that specifically binds to an HIV protein; VH1 is a first immunoglobulin heavy chain variable region that specifically binds to an HIV protein; VH2 is a second immunoglobulin heavy chain variable region that specifically binds to an HIV protein; VH3 is a third immunoglobulin heavy chain variable region that specifically binds to an HIV protein; VH4 is a fourth immunoglobulin heavy chain variable region that specifically binds to an HIV protein; VH5 is a fifth immunoglobulin heavy chain variable region that specifically binds to an HIV protein; VH6 is a sixth immunoglobulin heavy chain variable region that specifically binds to an HIV protein; and L1, L2, L3, L4, L5, L6, L7, L8, L9 and L10 are amino acid linkers.

In some aspects, the antigen binding polypeptide or antigen binding polypeptide complex comprises a polypeptide having a structure represented by VL1-VL2-VL3-VH3-VH2-VH1-Fc; VH1-VH2-VH3-VL3-VL2-VL1-Fc; VL1-VH2-VL3-VH3-VL2-VH1-Fc; VH1-VL2-VH3-VL3-VH2-VL1-Fc; VL1-VL2-VH3-VL3-VH2-VH1-Fc; VH1-VH2-VL3-VH3-VL2-VL1-Fc; VL1-VH2-VH3-VL3-VL2-VH1-Fc; VH1-VL2-VL3-VH3-VH2-VL1-Fc; VL1-L1-VL2-L2-VL3-L3-VH3-L4-VH2-L5-VH1-Fc; VL1-L1-VL2-L2-VL3-L3-VH3-L4-VH2-L5-VH1-L6-Fc; VH1-L1-VH2-L2-VH3-L3-VL3-L4-VL2-L5-VL1-Fc; VH1-L1-VH2-L2-VH3-L3-VL3-L4-VL2-L5-VL1-L6-Fc; VL1-L1-VH2-L2-VL3-L3-VH3-L4-VL2-L5-VH1-Fc; VL1-L1-VH2-L2-VL3-L3-VH3-L4-VL2-L5-VH1-L6-Fc; VH1-L1-VL2-L2-VH3-L3-VL3-L4-VH2-L5-VL1-Fc; VH1-L1-VL2-L2-VH3-L3-VL3-L4-VH2-L5-VL1-L6-Fc; VL1-L1-VL2-L2-VH3-L3-VL3-L4-VH2-L5-VH1-Fc; VL1-L1-VL2-L2-VH3-L3-VL3-L4-VH2-L5-VH1-L6-Fc; VH1-L1-VH2-L2-VL3-L3-VH3-L4-VL2-L5-VL1-Fc; VH1-L1-VH2-L2-VL3-L3-VH3-L4-VL2-L5-VL1-L6-Fc; VL1-L1-VH2-L2-VH3-L3-VL3-L4-VL2-L5-VH1-Fc; VL1-L1-VH2-L2-VH3-L3-VL3-L4-VL2-L5-VH1-L6-Fc; VH1-L1-VL2-L2-VL3-L3-VH3-L4-VH2-L5-VL1-Fc; or VH1-L1-VL2-L2-VL3-L3-VH3-L4-VH2-L5-VL1-L6-Fc; wherein VL1 is a first immunoglobulin light chain variable region that specifically binds to an HIV protein; VL2 is a second immunoglobulin light chain variable region that specifically binds to an HIV protein; VL3 is a third immunoglobulin light chain variable region that specifically binds to an HIV protein; VH1 is a first immunoglobulin heavy chain variable region that specifically binds to an HIV protein; VH2 is a second immunoglobulin heavy chain variable region that specifically binds to an HIV protein; VH3 is a third immunoglobulin heavy chain variable region that specifically binds to an HIV protein; Fc is a region comprising an immunoglobulin heavy chain constant region 2 (CH2), an immunoglobulin heavy chain constant region 3 (CH3), and optionally, an immunoglobulin hinge; and L1, L2, L3, L4, L5 and L6 are amino acid linkers.

In some aspects, the antigen binding polypeptide or antigen binding polypeptide complex comprises a polypeptide having a structure represented by VL1-VL2-VL3-VH3-VH2-VH1-Fc-Fc; VH1-VH2-VH3-VL3-VL2-VL1-Fc-Fc; VL1-VH2-VL3-VH3-VL2-VH1-Fc-Fc; VH1-VL2-VH3-VL3-VH2-VL1-Fc-Fc; VL1-VL2-VH3-VL3-VH2-VH1-Fc-Fc; VH1-VH2-VL3-VH3-VL2-VL1-Fc-Fc; VL1-VH2-VH3-VL3-VL2-VH1-Fc-Fc; VH1-VL2-VL3-VH3-VH2-VL1-Fc-Fc; VL1-L1-VL2-L2-VL3-L3-VH3-L4-VH2-L5-VH1-Fc-Fc; VL1-L1-VL2-L2-VL3-L3-VH3-L4-VH2-L5-VH1-L6-Fc-Fc; VL1-L1-VL2-L2-VL3-L3-VH3-L4-VH2-L5-VH1-L6-Fc-L7-Fc; VH1-L1-VH2-L2-VH3-L3-VL3-L4-VL2-L5-VL1-Fc-Fc; VH1-L1-VH2-L2-VH3-L3-VL3-L4-VL2-L5-VL1-L6-Fc-Fc; VH1-L1-VH2-L2-VH3-L3-VL3-L4-VL2-L5-VL1-L6-Fc-L7-Fc; VL1-L1-VH2-L2-VL3-L3-VH3-L4-VL2-L5-VH1-Fc-Fc; VL1-L1-VH2-L2-VL3-L3-VH3-L4-VL2-L5-VH1-L6-Fc-Fc; VL1-L1-VH2-L2-VL3-L3-VH3-L4-VL2-L5-VH1-L6-Fc-L7-Fc; VH1-L1-VL2-L2-VH3-L3-VL3-L4-VH2-L5-VL1-Fc-Fc; VH1-L1-VL2-L2-VH3-L3-VL3-L4-VH2-L5-VL1-L6-Fc-Fc; VH1-L1-VL2-L2-VH3-L3-VL3-L4-VH2-L5-VL1-L6-Fc-L7-Fc; VL1-L1-VL2-L2-VH3-L3-VL3-L4-VH2-L5-VH1-Fc-Fc; VL1-L1-VL2-L2-VH3-L3-VL3-L4-VH2-L5-VH1-L6-Fc-Fc; VL1-L1-VL2-L2-VH3-L3-VL3-L4-VH2-L5-VH1-L6-Fc-L7-Fc; VH1-L1-VH2-L2-VL3-L3-VH3-L4-VL2-L5-VL1-Fc-Fc; VH1-L1-VH2-L2-VL3-L3-VH3-L4-VL2-L5-VL1-L6-Fc-Fc; VH1-L1-VH2-L2-VL3-L3-VH3-L4-VL2-L5-VL1-L6-Fc-L7-Fc; L1-L1-VH2-L2-VH3-L3-VL3-L4-VL2-L5-VH1-Fc-Fc; VL1-L1-VH2-L2-VH3-L3-VL3-L4-VL2-L5-VH1-L6-Fc-Fc; VL1-L1-VH2-L2-VH3-L3-VL3-L4-VL2-L5-VH1-L6-Fc-L7-Fc; VH1-L1-VL2-L2-VL3-L3-VH3-L4-VH2-L5-VL1-Fc-Fc; VH1-L1-VL2-L2-VL3-L3-VH3-L4-VH2-L5-VL1-L6-Fc-Fc; or VH1-L1-VL2-L2-VL3-L3-VH3-L4-VH2-L5-VL1-L6-Fc-L7-Fc; wherein VL1 is a first immunoglobulin light chain variable region that specifically binds to an HIV protein; VL2 is a second immunoglobulin light chain variable region that specifically binds to an HIV protein; VL3 is a third immunoglobulin light chain variable region that specifically binds to an HIV protein; VH1 is a first immunoglobulin heavy chain variable region that specifically binds to an HIV protein; VH2 is a second immunoglobulin heavy chain variable region that specifically binds to an HIV protein; VH3 is a third immunoglobulin heavy chain variable region that specifically binds to an HIV protein; Fc is a region comprising an immunoglobulin heavy chain constant region 2 (CH2), an immunoglobulin heavy chain constant region 3 (CH3), and optionally, an immunoglobulin hinge; and L1, L2, L3, L4, L5, L6 and L7 are amino acid linkers.

In some aspects, the antigen binding polypeptide complex comprises a first polypeptide and a second polypeptide; wherein the first polypeptide has a structure represented by VL1-VL2-VL3-VH3-VH2-VH1-Fc; VH1-VH2-VH3-VL3-VL2-VL1-Fc; VL1-VH2-VL3-VH3-VL2-VH1-Fc; VH1-VL2-VH3-VL3-VH2-VL1-Fc; VL1-VL2-VH3-VL3-VH2-VH1-Fc; VH1-VH2-VL3-VH3-VL2-VL1-Fc; VL1-VH2-VH3-VL3-VL2-VH1-Fc; VH1-VL2-VL3-VH3-VH2-VL1-Fc; VL1-L1-VL2-L2-VL3-L3-VH3-L4-VH2-L5-VH1-Fc; VL1-L1-VL2-L2-VL3-L3-VH3-L4-VH2-L5-VH1-L6-Fc; VH1-L1-VH2-L2-VH3-L3-VL3-L4-VL2-L5-VL1-Fc; VH1-L1-VH2-L2-VH3-L3-VL3-L4-VL2-L5-VL1-L6-Fc; VL1-L1-VH2-L2-VL3-L3-VH3-L4-VL2-L5-VH1-Fc; VL1-L1-VH2-L2-VL3-L3-VH3-L4-VL2-L5-VH1-L6-Fc; VH1-L1-VL2-L2-VH3-L3-VL3-L4-VH2-L5-VL1-Fc; VH1-L1-VL2-L2-VH3-L3-VL3-L4-VH2-L5-VL1-L6-Fc; VL1-L1-VL2-L2-VH3-L3-VL3-L4-VH2-L5-VH1-Fc; VL1-L1-VL2-L2-VH3-L3-VL3-L4-VH2-L5-VH1-L6-Fc; VH1-L1-VH2-L2-VL3-L3-VH3-L4-VL2-L5-VL1-Fc; VH1-L1-VH2-L2-VL3-L3-VH3-L4-VL2-L5-VL1-L6-Fc; VL1-L1-VH2-L2-VH3-L3-VL3-L4-VL2-L5-VH1-Fc; VL1-L1-VH2-L2-VH3-L3-VL3-L4-VL2-L5-VH1-L6-Fc; VH1-L1-VL2-L2-VL3-L3-VH3-L4-VH2-L5-VL1-Fc; or VH1-L1-VL2-L2-VL3-L3-VH3-L4-VH2-L5-VL1-L6-Fc; wherein the second polypeptide has a structure represented by VL4-VH4-Fc; VH4-VL4-Fc; VL4-L7-VH4-Fc; VH4-L7-VL4-Fc; VL4-L7-VH4-L8-Fc; VH4-L7-VL4-L8-Fc; VL4-CL-VH4-CH1-Fc; VH4-CL-VL4-CH1-Fc; VL4-CH1-VH4-CL-Fc; VH4-CH1-VL4-CL-Fc; VL4-L7-CL-L8-VH4-L9-CH1-Fc; VL4-L7-CL-L8-VH4-L9-CH1-L10-Fc; VH4-L7-CL-L8-VL4-L9-CH1-Fc; VH4-L7-CL-L8-VL4-L9-CH1-L10-Fc; VL4-L7-CH1-L8-VH4-L9-CL-Fc; VL4-L7-CH1-L8-VH4-L9-CL-L10-Fc; VH4-L7-CH1-L8-VL4-L9-CL-Fc; VH4-L7-CH1-L8-VL4-L9-CL-L10-Fc; VL4-VL5-VH5-VH4-Fc; VH4-VH5-VL5-VL4-Fc; VL4-L7-VL5-L8-VH5-L9-VH4-Fc; VH4-L7-VH5-L8-VL5-L9-VL4-Fc; VL4-L7-VL5-L8-VH5-L9-VH4-L10-Fc; VH4-L7-VH5-L8-VL5-L9-VL4-L10-Fc; VL4-VL5-VL6-VH6-VH5-VH4-Fc; VH4-VH5-VH6-VL6-VL5-VL4-Fc; VL4-VH5-VL6-VH6-VL5-VH4-Fc; VH4-VL5-VH6-VL6-VH5-VL4-Fc; VL4-VL5-VH6-VL6-VH5-VH4-Fc; VH4-VH5-VL6-VH6-VL5-VL4-Fc; VL4-VH5-VH6-VL6-VL5-VH4-Fc; VH4-VL5-VL6-VH6-VH5-VL4-Fc; VL4-L7-VL5-L8-VL6-L9-VH6-L10-VH5-L11-VH4-Fc; VH4-L7-VH5-L8-VH6-L9-VL6-L10-VL5-L11-VL4-Fc; VL4-L7-VH5-L8-VL6-L9-VH6-L10-VL5-L11-VH4-Fc; VH4-L7-VL5-L8-VH6-L9-VL6-L10-VH5-L11-VL4-Fc; VL4-L7-VL5-L8-VH6-L9-VL6-L10-VH5-L11-VH4-Fc; VH4-L7-VH5-L8-VL6-L9-VH6-L10-VL5-L11-VL4-Fc; VL4-L7-VH5-L8-VH6-L9-VL6-L10-VL5-L11-VH4-Fc; VH4-L7-VL5-L8-VL6-L9-VH6-L10-VH5-L11-VL4-Fc; VL4-L7-VL5-L8-VL6-L9-VH6-L10-VH5-L11-VH4-L12-Fc; VH4-L7-VH5-L8-VH6-L9-VL6-L10-VL5-L11-VL4-L12-Fc; VL4-L7-VH5-L8-VL6-L9-VH6-L10-VL5-L11-VH4-L12-Fc; VH4-L7-VL5-L8-VH6-L9-VL6-L10-VH5-L11-VL4-L12-Fc; VL4-L7-VL5-L8-VH6-L9-VL6-L10-VH5-L11-VH4-L12-Fc; VH4-L7-VH5-L8-VL6-L9-VH6-L10-VL5-L11-VL4-L12-Fc; VL4-L7-VH5-L8-VH6-L9-VL6-L10-VL5-L11-VH4-L12-Fc; or VH4-L7-VL5-L8-VL6-L9-VH6-L10-VH5-L11-VL4-L12-Fc; wherein VL1 is a first immunoglobulin light chain variable region that specifically binds to an HIV protein; VL2 is a second immunoglobulin light chain variable region that specifically binds to an HIV protein; VL3 is a third immunoglobulin light chain variable region that specifically binds to an HIV protein; VL4 is a fourth immunoglobulin light chain variable region that specifically binds to an HIV protein; VL5 is a fifth immunoglobulin light chain variable region that specifically binds to an HIV protein; VL6 is a sixth immunoglobulin light chain variable region that specifically binds to an HIV protein; VH1 is a first immunoglobulin heavy chain variable region that specifically binds to an HIV protein; VH2 is a second immunoglobulin heavy chain variable region that specifically binds to an HIV protein; VH3 is a third immunoglobulin heavy chain variable region that specifically binds to an HIV protein; VH4 is a fourth immunoglobulin heavy chain variable region that specifically binds to an HIV protein; VH5 is a fifth immunoglobulin heavy chain variable region that specifically binds to an HIV protein; VH6 is a sixth immunoglobulin heavy chain variable region that specifically binds to an HIV protein; Fc is a region comprising an immunoglobulin heavy chain constant region 2 (CH2), an immunoglobulin heavy chain constant region 3 (CH3), and optionally, an immunoglobulin hinge; and L1, L2, L3, L4, L5, L6, L7, L8, L9, L10, L11 and L12 are amino acid linkers.

In some aspects, the antigen binding polypeptide has a structure represented by VL1-VL2-VL3-VH3-VH2-VH1-CH1-CL; VL1-VL2-VL3-VH3-VH2-VH1-CL-CH1; VH1-VH2-VH3-VL3-VL2-VL1-CH1-CL; VH1-VH2-VH3-VL3-VL2-VL1-CL-CH1; VL1-VH2-VL3-VH3-VL2-VH1-CH1-CL; VL1-VH2-VL3-VH3-VL2-VH1-CL-CH1; VH1-VL2-VH3-VL3-VH2-VL1-CH1-CL; VH1-VL2-VH3-VL3-VH2-VL1-CL-CH1; VL1-VL2-VH3-VL3-VH2-VH1-CH1-CL; VL1-VL2-VH3-VL3-VH2-VH1-CL-CH1; VH1-VH2-VL3-VH3-VL2-VL1-CH1-CL; VH1-VH2-VL3-VH3-VL2-VL1-CL-CH1; VL1-VH2-VH3-VL3-VL2-VH1-CH1-CL; VL1-VH2-VH3-VL3-VL2-VH1-CL-CH1; VH1-VL2-VL3-VH3-VH2-VL1-CH1-CL; VH1-VL2-VL3-VH3-VH2-VL1-CL-CH1; VL1-L1-VL2-L2-VL3-L3-VH3-L4-VH2-L5-VH1-CH1-CL; VL1-L1-VL2-L2-VL3-L3-VH3-L4-VH2-L5-VH1-L6-CH1-CL; VL1-L1-VL2-L2-VL3-L3-VH3-L4-VH2-L5-VH1-CL-CH1; VL1-L1-VL2-L2-VL3-L3-VH3-L4-VH2-L5-VH1-L6-CL-CH1; VL1-L1-VL2-L2-VL3-L3-VH3-L4-VH2-L5-VH1-L6-CL-L7-CH1; VH1-L1-VH2-L2-VH3-L3-VL3-L4-VL2-L5-VL1-CH1-CL; VH1-L1-VH2-L2-VH3-L3-VL3-L4-VL2-L5-VL1-L6-CH1-CL; VH1-L1-VH2-L2-VH3-L3-VL3-L4-VL2-L5-VL1-L6-CH1-L7-CL; VH1-L1-VH2-L2-VH3-L3-VL3-L4-VL2-L5-VL1-CL-CH1; VH1-L1-VH2-L2-VH3-L3-VL3-L4-VL2-L5-VL1-L6-CL-CH1; VL1-L1-VH2-L2-VL3-L3-VH3-L4-VL2-L5-VH1-CH1-CL; VL1-L1-VH2-L2-VL3-L3-VH3-L4-VL2-L5-VH1-L6-CH1-CL; VL1-L1-VH2-L2-VL3-L3-VH3-L4-VL2-L5-VH1-L6-CH1-L7-CL; VL1-L1-VH2-L2-VL3-L3-VH3-L4-VL2-L5-VH1-CL-CH1; VL1-L1-VH2-L2-VL3-L3-VH3-L4-VL2-L5-VH1-L6-CL-CH1; VH1-L1-VL2-L2-VH3-L3-VL3-L4-VH2-L5-VL1-CH1-CL; VH1-L1-VL2-L2-VH3-L3-VL3-L4-VH2-L5-VL1-L6-CH1-CL; VH1-L1-VL2-L2-VH3-L3-VL3-L4-VH2-L5-VL1-L6-CH1-L7-CL; VH1-L1-VL2-L2-VH3-L3-VL3-L4-VH2-L5-VL1-CL-CH1; VH1-L1-VL2-L2-VH3-L3-VL3-L4-VH2-L5-VL1-L6-CL-CH1; VH1-L1-VL2-L2-VH3-L3-VL3-L4-VH2-L5-VL1-L6-CL-L7-CH1; VL1-L1-VL2-L2-VH3-L3-VL3-L4-VH2-L5-VH1-CH1-CL; VL1-L1-VL2-L2-VH3-L3-VL3-L4-VH2-L5-VH1-L6-CH1-CL; VL1-L1-VL2-L2-VH3-L3-VL3-L4-VH2-L5-VH1-L6-CH1-L7-CL; VL1-L1-VL2-L2-VH3-L3-VL3-L4-VH2-L5-VH1-CL-CH1; VL1-L1-VL2-L2-VH3-L3-VL3-L4-VH2-L5-VH1-L6-CL-CH1; VL1-L1-VL2-L2-VH3-L3-VL3-L4-VH2-L5-VH1-L6-CL-L7-CH1; VH1-L1-VH2-L2-VL3-L3-VH3-L4-VL2-L5-VL1-CH1-CL; VH1-L1-VH2-L2-VL3-L3-VH3-L4-VL2-L5-VL1-L6-CH1-CL; VH1-L1-VH2-L2-VL3-L3-VH3-L4-VL2-L5-VL1-L6-CH1-L7-CL; VH1-L1-VH2-L2-VL3-L3-VH3-L4-VL2-L5-VL1-CL-CH1; VH1-L1-VH2-L2-VL3-L3-VH3-L4-VL2-L5-VL1-L6-CL-CH1; VH1-L1-VH2-L2-VL3-L3-VH3-L4-VL2-L5-VL1-L6-CL-L7-CH1; VL1-L1-VH2-L2-VH3-L3-VL3-L4-VL2-L5-VH1-CH1-CL; VL1-L1-VH2-L2-VH3-L3-VL3-L4-VL2-L5-VH1-L6-CH1-CL; VL1-L1-VH2-L2-VH3-L3-VL3-L4-VL2-L5-VH1-L6-CH1-L7-CL; VL1-L1-VH2-L2-VH3-L3-VL3-L4-VL2-L5-VH1-CL-CH1; VL1-L1-VH2-L2-VH3-L3-VL3-L4-VL2-L5-VH1-L6-CL-CH1; VL1-L1-VH2-L2-VH3-L3-VL3-L4-VL2-L5-VH1-L6-CL-L7-CH1; VH1-L1-VL2-L2-VL3-L3-VH3-L4-VH2-L5-VL1-CH1-CL; VH1-L1-VL2-L2-VL3-L3-VH3-L4-VH2-L5-VL1-L6-CH1-CL; VH1-L1-VL2-L2-VL3-L3-VH3-L4-VH2-L5-VL1-L6-CH1-L7-CL; VH1-L1-VL2-L2-VL3-L3-VH3-L4-VH2-L5-VL1-CL-CH1; VH1-L1-VL2-L2-VL3-L3-VH3-L4-VH2-L5-VL1-L6-CL-CH1; or VH1-L1-VL2-L2-VL3-L3-VH3-L4-VH2-L5-VL1-L6-CL-L7-CH1; wherein VL1 is a first immunoglobulin light chain variable region that specifically binds to an HIV protein; VL2 is a second immunoglobulin light chain variable region that specifically binds to an HIV protein; VL3 is a third immunoglobulin light chain variable region that specifically binds to an HIV protein; VH1 is a first immunoglobulin heavy chain variable region that specifically binds to an HIV protein; VH2 is a second immunoglobulin heavy chain variable region that specifically binds to an HIV protein; VH3 is a third immunoglobulin heavy chain variable region that specifically binds to an HIV protein; CH1 is a heavy chain constant region 1; CL is a light chain constant region; and L1, L2, L3, L4, L5, L6 and L7 are amino acid linkers.

In some aspects, the antigen binding polypeptide complex comprises a first polypeptide and a second polypeptide; wherein the first polypeptide has a structure represented by VL1-VL2-VL3-VH3-VH2-VH1-CH1; VL1-VL2-VL3-VH3-VH2-VH1-CL; VL1-VL2-VL3-VH3-VH2-VH1-CH1-CL; VL1-VL2-VL3-VH3-VH2-VH1-CL-CH1; VH1-VH2-VH3-VL3-VL2-VL1-CH1; VH1-VH2-VH3-VL3-VL2-VL1-CL; VH1-VH2-VH3-VL3-VL2-VL1-CH1-CL; VH1-VH2-VH3-VL3-VL2-VL1-CL-CH1; VL1-VH2-VL3-VH3-VL2-VH1-CH1; VL1-VH2-VL3-VH3-VL2-VH1-CL; VL1-VH2-VL3-VH3-VL2-VH1-CH1-CL; VL1-VH2-VL3-VH3-VL2-VH1-CL-CH1; VH1-VL2-VH3-VL3-VH2-VL1-CH1; VH1-VL2-VH3-VL3-VH2-VL1-CL; VH1-VL2-VH3-VL3-VH2-VL1-CH1-CL; VH1-VL2-VH3-VL3-VH2-VL1-CL-CH1; VL1-VL2-VH3-VL3-VH2-VH1-CH1; VL1-VL2-VH3-VL3-VH2-VH1-CL; VL1-VL2-VH3-VL3-VH2-VH1-CH1-CL; VL1-VL2-VH3-VL3-VH2-VH1-CL-CH1; VH1-VH2-VL3-VH3-VL2-VL1-CH1; VH1-VH2-VL3-VH3-VL2-VL1-CL; VH1-VH2-VL3-VH3-VL2-VL1-CH1-CL; VH1-VH2-VL3-VH3-VL2-VL1-CL-CH1; VL1-VH2-VH3-VL3-VL2-VH1-CH1; VL1-VH2-VH3-VL3-VL2-VH1-CL; VL1-VH2-VH3-VL3-VL2-VH1-CH1-CL; VL1-VH2-VH3-VL3-VL2-VH1-CL-CH1; VH1-VL2-VL3-VH3-VH2-VL1-CH1; VH1-VL2-VL3-VH3-VH2-VL1-CL; VH1-VL2-VL3-VH3-VH2-VL1-CH1-CL; VH1-VL2-VL3-VH3-VH2-VL1-CL-CH1; VL1-L1-VL2-L2-VL3-L3-VH3-L4-VH2-L5-VH1-CH1; VL1-L1-VL2-L2-VL3-L3-VH3-L4-VH2-L5-VH1-L6-CH1; VL1-L1-VL2-L2-VL3-L3-VH3-L4-VH2-L5-VH1-CL; VL1-L1-VL2-L2-VL3-L3-VH3-L4-VH2-L5-VH1-L6-CL; VL1-L1-VL2-L2-VL3-L3-VH3-L4-VH2-L5-VH1-CH1-CL; VL1-L1-VL2-L2-VL3-L3-VH3-L4-VH2-L5-VH1-L6-CH1-CL; VL1-L1-VL2-L2-VL3-L3-VH3-L4-VH2-L5-VH1-L6-CH1-L7-CL; VL1-L1-VL2-L2-VL3-L3-VH3-L4-VH2-L5-VH1-CL-CH1; VL1-L1-VL2-L2-VL3-L3-VH3-L4-VH2-L5-VH1-L6-CL-CH1; VL1-L1-VL2-L2-VL3-L3-VH3-L4-VH2-L5-VH1-L6-CL-L7-CH1; VH1-L1-VH2-L2-VH3-L3-VL3-L4-VL2-L5-VL1-CH1; VH1-L1-VH2-L2-VH3-L3-VL3-L4-VL2-L5-VL1-L6-CH1; VH1-L1-VH2-L2-VH3-L3-VL3-L4-VL2-L5-VL1-CL; VH1-L1-VH2-L2-VH3-L3-VL3-L4-VL2-L5-VL1-L6-CL; VH1-L1-VH2-L2-VH3-L3-VL3-L4-VL2-L5-VL1-CH1-CL; VH1-L1-VH2-L2-VH3-L3-VL3-L4-VL2-L5-VL1-L6-CH1-CL; VH1-L1-VH2-L2-VH3-L3-VL3-L4-VL2-L5-VL1-L6-CH1-L7-CL; VH1-L1-VH2-L2-VH3-L3-VL3-L4-VL2-L5-VL1-CL-CH1; VH1-L1-VH2-L2-VH3-L3-VL3-L4-VL2-L5-VL1-L6-CL-CH1; VH1-L1-VH2-L2-VH3-L3-VL3-L4-VL2-L5-VL1-L6-CL-L7-CH1; VL1-L1-VH2-L2-VL3-L3-VH3-L4-VL2-L5-VH1-CH1; VL1-L1-VH2-L2-VL3-L3-VH3-L4-VL2-L5-VH1-L6-CH1; VL1-L1-VH2-L2-VL3-L3-VH3-L4-VL2-L5-VH1-CL; VL1-L1-VH2-L2-VL3-L3-VH3-L4-VL2-L5-VH1-L6-CL; VL1-L1-VH2-L2-VL3-L3-VH3-L4-VL2-L5-VH1-CH1-CL; VL1-L1-VH2-L2-VL3-L3-VH3-L4-VL2-L5-VH1-L6-CH1-CL; VL1-L1-VH2-L2-VL3-L3-VH3-L4-VL2-L5-VH1-L6-CH1-L7-CL; VL1-L1-VH2-L2-VL3-L3-VH3-L4-VL2-L5-VH1-CL-CH1; VL1-L1-VH2-L2-VL3-L3-VH3-L4-VL2-L5-VH1-L6-CL-CH1; VL1-L1-VH2-L2-VL3-L3-VH3-L4-VL2-L5-VH1-L6-CL-L7-CH1; VH1-L1-VL2-L2-VH3-L3-VL3-L4-VH2-L5-VL1-CH1; VH1-L1-VL2-L2-VH3-L3-VL3-L4-VH2-L5-VL1-L6-CH1; VH1-L1-VL2-L2-VH3-L3-VL3-L4-VH2-L5-VL1-CL; VH1-L1-VL2-L2-VH3-L3-VL3-L4-VH2-L5-VL1-L6-CL; VH1-L1-VL2-L2-VH3-L3-VL3-L4-VH2-L5-VL1-CH1-CL; VH1-L1-VL2-L2-VH3-L3-VL3-L4-VH2-L5-VL1-L6-CH1-CL; VH1-L1-VL2-L2-VH3-L3-VL3-L4-VH2-L5-VL1-L6-CH1-L7-CL; VH1-L1-VL2-L2-VH3-L3-VL3-L4-VH2-L5-VL1-CL-CH1; VH1-L1-VL2-L2-VH3-L3-VL3-L4-VH2-L5-VL1-L6-CL-CH1; VH1-L1-VL2-L2-VH3-L3-VL3-L4-VH2-L5-VL1-L6-CL-L7-CH1; VL1-L1-VL2-L2-VH3-L3-VL3-L4-VH2-L5-VH1-CH1; VL1-L1-VL2-L2-VH3-L3-VL3-L4-VH2-L5-VH1-L6-CH1; VL1-L1-VL2-L2-VH3-L3-VL3-L4-VH2-L5-VH1-CL; VL1-L1-VL2-L2-VH3-L3-VL3-L4-VH2-L5-VH1-L6-CL; VL1-L1-VL2-L2-VH3-L3-VL3-L4-VH2-L5-VH1-CH1-CL; VL1-L1-VL2-L2-VH3-L3-VL3-L4-VH2-L5-VH1-L6-CH1-CL; VL1-L1-VL2-L2-VH3-L3-VL3-L4-VH2-L5-VH1-L6-CH1-L7-CL; VL1-L1-VL2-L2-VH3-L3-VL3-L4-VH2-L5-VH1-CL-CH1; VL1-L1-VL2-L2-VH3-L3-VL3-L4-VH2-L5-VH1-L6-CL-CH1; VL1-L1-VL2-L2-VH3-L3-VL3-L4-VH2-L5-VH1-L6-CL-L7-CH1; VH1-L1-VH2-L2-VL3-L3-VH3-L4-VL2-L5-VL1-CH1; VH1-L1-VH2-L2-VL3-L3-VH3-L4-VL2-L5-VL1-L6-CH1; VH1-L1-VH2-L2-VL3-L3-VH3-L4-VL2-L5-VL1-CL; VH1-L1-VH2-L2-VL3-L3-VH3-L4-VL2-L5-VL1-L6-CL; VH1-L1-VH2-L2-VL3-L3-VH3-L4-VL2-L5-VL1-CH1-CL; VH1-L1-VH2-L2-VL3-L3-VH3-L4-VL2-L5-VL1-L6-CH1-CL; VH1-L1-VH2-L2-VL3-L3-VH3-L4-VL2-L5-VL1-L6-CH1-L7-CL; VH1-L1-VH2-L2-VL3-L3-VH3-L4-VL2-L5-VL1-CL-CH1; VH1-L1-VH2-L2-VL3-L3-VH3-L4-VL2-L5-VL1-L6-CL-CH1; VH1-L1-VH2-L2-VL3-L3-VH3-L4-VL2-L5-VL1-L6-CL-L7-CH1; VL1-L1-VH2-L2-VH3-L3-VL3-L4-VL2-L5-VH1-CH1; VL1-L1-VH2-L2-VH3-L3-VL3-L4-VL2-L5-VH1-L6-CH1; VL1-L1-VH2-L2-VH3-L3-VL3-L4-VL2-L5-VH1-CL; VL1-L1-VH2-L2-VH3-L3-VL3-L4-VL2-L5-VH1-L6-CL; VL1-L1-VH2-L2-VH3-L3-VL3-L4-VL2-L5-VH1-CH1-CL; VL1-L1-VH2-L2-VH3-L3-VL3-L4-VL2-L5-VH1-L6-CH1-CL; VL1-L1-VH2-L2-VH3-L3-VL3-L4-VL2-L5-VH1-L6-CH1-L7-CL; VL1-L1-VH2-L2-VH3-L3-VL3-L4-VL2-L5-VH1-CL-CH1; VL1-L1-VH2-L2-VH3-L3-VL3-L4-VL2-L5-VH1-L6-CL-CH1; VL1-L1-VH2-L2-VH3-L3-VL3-L4-VL2-L5-VH1-L6-CL-L7-CH1; VH1-L1-VL2-L2-VL3-L3-VH3-L4-VH2-L5-VL1-CH1; VH1-L1-VL2-L2-VL3-L3-VH3-L4-VH2-L5-VL1-L6-CH1; VH1-L1-VL2-L2-VL3-L3-VH3-L4-VH2-L5-VL1-CL; VH1-L1-VL2-L2-VL3-L3-VH3-L4-VH2-L5-VL1-L6-CL; VH1-L1-VL2-L2-VL3-L3-VH3-L4-VH2-L5-VL1-CH1-CL; VH1-L1-VL2-L2-VL3-L3-VH3-L4-VH2-L5-VL1-L6-CH1-CL; VH1-L1-VL2-L2-VL3-L3-VH3-L4-VH2-L5-VL1-L6-CH1-L7-CL; VH1-L1-VL2-L2-VL3-L3-VH3-L4-VH2-L5-VL1-CL-CH1; VH1-L1-VL2-L2-VL3-L3-VH3-L4-VH2-L5-VL1-L6-CL-CH1; or VH1-L1-VL2-L2-VL3-L3-VH3-L4-VH2-L5-VL1-L6-CL-L7-CH1; wherein the second polypeptide has a structure represented by VL4-VH4-CH1; VL4-VH4-CL; VL4-VH4-CH1-CL; VL4-VH4-CL-CH1; VH4-VL4-CH1; VH4-VL4-CL; VH4-VL4-CH1-CL; VH4-VL4-CL-CH1; VL4-L8-VH4-CH1; VL4-L8-VH4-CL; VL4-L8-VH4-CH1-CL; VL4-L8-VH4-CL-CH1; VH4-L8-VL4-CH1; VH4-L8-VL4-CL; VH4-L8-VH4-CH1-CL; VH4-L8-VH4-CL-CH1; VL4-VL5-VH5-VH4-CH1; VL4-VL5-VH5-VH4-CL; VL4-VL5-VH5-VH4-CH1-CL; VL4-VL5-VH5-VH4-CL-CH1; VH4-VH5-VL5-VL4-CH1; VH4-VH5-VL5-VL4-CL; VH4-VH5-VL5-VL4-CH1-CL; VH4-VH5-VL5-VL4-CL-CH1; VL4-L8-VL5-L9-VH5-L10-VH4-CH1; VL4-L8-VL5-L9-VH5-L10-VH4-CL; VL4-L8-VL5-L9-VH5-L10-VH4-CH1-CL; VL4-L8-VL5-L9-VH5-L10-VH4-CL-CH1; VH4-L8-VH5-L9-VL5-L10-VL4-CH1; VH4-L8-VH5-L9-VL5-L10-VL4-CL; VH4-L8-VH5-L9-VL5-L10-VL4-CH1-CL; VH4-L8-VH5-L9-VL5-L10-VL4-CL-CH1; VL4-VL5-VL6-VH6-VH5-VH4-CH1; VL4-VL5-VL6-VH6-VH5-VH4-CL; VL4-VL5-VL6-VH6-VH5-VH4-CH1-CL; VL4-VL5-VL6-VH6-VH5-VH4-CL-CH1; VH4-VH5-VH6-VL6-VL5-VL4-CH1; VH4-VH5-VH6-VL6-VL5-VL4-CL; VH4-VH5-VH6-VL6-VL5-VL4-CH1-CL; VH4-VH5-VH6-VL6-VL5-VL4-CL-CH1; VL4-VH5-VL6-VH6-VL5-VH4-CH1; VL4-VH5-VL6-VH6-VL5-VH4-CL; VL4-VH5-VL6-VH6-VL5-VH4-CH1-CL; VL4-VH5-VL6-VH6-VL5-VH4-CL-CH1; VH4-VL5-VH6-VL6-VH5-VL4-CH1; VH4-VL5-VH6-VL6-VH5-VL4-CL; VH4-VL5-VH6-VL6-VH5-VL4-CH1-CL; VH4-VL5-VH6-VL6-VH5-VL4-CL-CH1; VL4-VL5-VH6-VL6-VH5-VH4-CH1; VL4-VL5-VH6-VL6-VH5-VH4-CL; VL4-VL5-VH6-VL6-VH5-VH4-CH1-CL; VL4-VL5-VH6-VL6-VH5-VH4-CL-CH1; VH4-VH5-VL6-VH6-VL5-VL4-CH1; VH4-VH5-VL6-VH6-VL5-VL4-CL; VH4-VH5-VL6-VH6-VL5-VL4-CH1-CL; VH4-VH5-VL6-VH6-VL5-VL4-CL-CH1; VL4-VH5-VH6-VL6-VL5-VH4-CH1; VL4-VH5-VH6-VL6-VL5-VH4-CL; VL4-VH5-VH6-VL6-VL5-VH4-CH1-CL; VL4-VH5-VH6-VL6-VL5-VH4-CL-CH1; VH4-VL5-VL6-VH6-VH5-VL4-CH1; VH4-VL5-VL6-VH6-VH5-VL4-CL; VH4-VL5-VL6-VH6-VH5-VL4-CH1-CL; VH4-VL5-VL6-VH6-VH5-VL4-CL-CH1; VL4-L8-VL5-L9-VL6-L10-VH6-L11-VH5-L12-VH4-CH1; VL4-L8-VL5-L9-VL6-L10-VH6-L11-VH5-L12-VH4-CL; VL4-L8-VL5-L9-VL6-L10-VH6-L11-VH5-L12-VH4-CH1-CL; VL4-L8-VL5-L9-VL6-L10-VH6-L11-VH5-L12-VH4-CL-CH1; VH4-L8-VH5-L9-VH6-L10-VL6-L11-VL5-L12-VL4-CH1; VH4-L8-VH5-L9-VH6-L10-VL6-L11-VL5-L12-VL4-CL; VH4-L8-VH5-L9-VH6-L10-VL6-L11-VL5-L12-VL4-CH1-CL; VH4-L8-VH5-L9-VH6-L10-VL6-L11-VL5-L12-VL4-CL-CH1; VL4-L8-VH5-L9-VL6-L10-VH6-L11-VL5-L12-VH4-CH1; VL4-L8-VH5-L9-VL6-L10-VH6-L11-VL5-L12-VH4-CL; VL4-L8-VH5-L9-VL6-L10-VH6-L11-VL5-L12-VH4-CH1-CL; VL4-L8-VH5-L9-VL6-L10-VH6-L11-VL5-L12-VH4-CL-CH1; VH4-L8-VL5-L9-VH6-L10-VL6-L11-VH5-L12-VL4-CH1; VH4-L8-VL5-L9-VH6-L10-VL6-L11-VH5-L11-VL4-CL; VH4-L8-VL5-L9-VH6-L10-VL6-L11-VH5-L12-VL4-CH1-CL; VH4-L8-VL5-L9-VH6-L10-VL6-L11-VH5-L12-VL4-CL-CH1; VL4-L8-VL5-L9-VL6-L10-VH6-L11-VH5-L12-VH4-L13-CH1; VL4-L8-VL5-L9-VL6-L10-VH6-L11-VH5-L12-VH4-L13-CL; VL4-L8-VL5-L9-VL6-L10-VH6-L11-VH5-L12-VH4-L13-CH1-CL; VL4-L8-VL5-L9-VL6-L10-VH6-L11-VH5-L12-VH4-L13-CL-CH1; VH4-L8-VH5-L9-VH6-L10-VL6-L11-VL5-L12-VL4-L13-CH1; VH4-L8-VH5-L9-VH6-L10-VL6-L11-VL5-L12-VL4-L13-CL; VH4-L8-VH5-L9-VH6-L10-VL6-L11-VL5-L12-VL4-L13-CH1-CL; VH4-L8-VH5-L9-VH6-L10-VL6-L11-VL5-L12-VL4-L13-CL-CH1; VL4-L8-VH5-L9-VL6-L10-VH6-L11-VL5-L12-VH4-L13-CH1; VL4-L8-VH5-L9-VL6-L10-VH6-L11-VL5-L12-VH4-L13-CL; VL4-L8-VH5-L9-VL6-L10-VH6-L11-VL5-L12-VH4-L13-CH1-CL; VL4-L8-VH5-L9-VL6-L10-VH6-L11-VL5-L12-VH4-L13-CL-CH1; VH4-L8-VL5-L9-VH6-L10-VL6-L11-VH5-L12-VL4-L13-CH1; VH4-L8-VL5-L9-VH6-L10-VL6-L11-VH5-L12-VL4-L13-CL; VH4-L8-VL5-L9-VH6-L10-VL6-L11-VH5-L12-VL4-L13-CH1-CL; VH4-L8-VL5-L9-VH6-L10-VL6-L11-VH5-L12-VL4-L13-CL-CH1; VL4-L8-VL5-L9-VH6-L10-VL6-L11-VH5-L12-VH4-CH1; VL4-L8-VL5-L9-VH6-L10-VL6-L11-VH5-L12-VH4-CL; VL4-L8-VL5-L9-VH6-L10-VL6-L11-VH5-L12-VH4-CH1-CL; VL4-L8-VL5-L9-VH6-L10-VL6-L11-VH5-L12-VH4-CL-CH1; VH4-L8-VH5-L9-VL6-L10-VH6-L11-VL5-L12-VL4-CH1; VH4-L8-VH5-L9-VL6-L10-VH6-L11-VL5-L12-VL4-CL; VH4-L8-VH5-L9-VL6-L10-VH6-L11-VL5-L12-VL4-CH1-CL; VH4-L8-VH5-L9-VL6-L10-VH6-L11-VL5-L12-VL4-CL-CH1; VL4-L8-VL5-L9-VH6-L10-VL6-L11-VH5-L12-VH4-L13-CH1; VL4-L8-VL5-L9-VH6-L10-VL6-L11-VH5-L12-VH4-L13-CL; VL4-L8-VL5-L9-VH6-L10-VL6-L11-VH5-L12-VH4-L13-CH1-CL; VL4-L8-VL5-L9-VH6-L10-VL6-L11-VH5-L12-VH4-L13-CL-CH1; VH4-L8-VH5-L9-VL6-L10-VH6-L11-VL5-L12-VL4-L13-CH1; VH4-L8-VH5-L9-VL6-L10-VH6-L11-VL5-L12-VL4-L13-CL; VH4-L8-VH5-L9-VL6-L10-VH6-L11-VL5-L12-VL4-L13-CH1-CL; or VH4-L8-VH5-L9-VL6-L10-VH6-L11-VL5-L12-VL4-L13-CL-CH1; wherein VL1 is a first immunoglobulin light chain variable region that specifically binds to an HIV protein; VL2 is a second immunoglobulin light chain variable region that specifically binds to an HIV protein; VL3 is a third immunoglobulin light chain variable region that specifically binds to an HIV protein; VL4 is a fourth immunoglobulin light chain variable region that specifically binds to an HIV protein; VL5 is a fifth immunoglobulin light chain variable region that specifically binds to an HIV protein; VL6 is a sixth immunoglobulin light chain variable region that specifically binds to an HIV protein; VH1 is a first immunoglobulin heavy chain variable region that specifically binds to an HIV protein; VH2 is a second immunoglobulin heavy chain variable region that specifically binds to an HIV protein; VH3 is a third immunoglobulin heavy chain variable region that specifically binds to an HIV protein; VH4 is a fourth immunoglobulin heavy chain variable region that specifically binds to an HIV protein; VH5 is a fifth immunoglobulin heavy chain variable region that specifically binds to an HIV protein; VH6 is a sixth immunoglobulin heavy chain variable region that specifically binds to an HIV protein; CH1 is a heavy chain constant region 1; CL is a light chain constant region; and L1, L2, L3, L4, L5, L6, L7, L8, L9, L10, L11, L12 and L13 are amino acid linkers.

In some aspects, the antigen binding polypeptide complex comprises a first polypeptide and a second polypeptide; wherein the first polypeptide has a structure represented by VL1-VL2-VL3-VH3-VH2-VH1-CH1-Fc; VH1-VH2-VH3-VL3-VL2-VL1-CH1-Fc; VL1-VH2-VL3-VH3-VL2-VH1-CH1-Fc; VH1-VL2-VH3-VL3-VH2-VL1-CH1-Fc; VL1-VL2-VH3-VL3-VH2-VH1-CH1-Fc; VH1-VH2-VL3-VH3-VL2-VL1-CH1-Fc; VL1-VH2-VH3-VL3-VL2-VH1-CH1-Fc; VH1-VL2-VL3-VH3-VH2-VL1-CH1-Fc; VL1-VL2-VL3-VH3-VH2-VH1-CL-Fc; VH1-VH2-VH3-VL3-VL2-VL1-CL-Fc; VL1-VH2-VL3-VH3-VL2-VH1-CL-Fc; VH1-VL2-VH3-VL3-VH2-VL1-CL-Fc; VL1-VL2-VH3-VL3-VH2-VH1-CL-Fc; VH1-VH2-VL3-VH3-VL2-VL1-CL-Fc; VL1-VH2-VH3-VL3-VL2-VH1-CL-Fc; VH1-VL2-VL3-VH3-VH2-VL1-CL-Fc; VL1-VL2-VL3-VH3-VH2-VH1-CH1-CL-Fc; VH1-VH2-VH3-VL3-VL2-VL1-CH1-CL-Fc; VL1-VH2-VL3-VH3-VL2-VH1-CH1-CL-Fc; VH1-VL2-VH3-VL3-VH2-VL1-CH1-CL-Fc; VL1-VL2-VH3-VL3-VH2-VH1-CH1-CL-Fc; VH1-VH2-VL3-VH3-VL2-VL1-CH1-CL-Fc; VL1-VH2-VH3-VL3-VL2-VH1-CH1-CL-Fc; VH1-VL2-VL3-VH3-VH2-VL1-CH1-CL-Fc; VL1-VL2-VL3-VH3-VH2-VH1-CL-CH1-Fc; VH1-VH2-VH3-VL3-VL2-VL1-CL-CH1-Fc; VL1-VH2-VL3-VH3-VL2-VH1-CL-CH1-Fc; VH1-VL2-VH3-VL3-VH2-VL1-CL-CH1-Fc; VL1-VL2-VH3-VL3-VH2-VH1-CL-CH1-Fc; VH1-VH2-VL3-VH3-VL2-VL1-CL-CH1-Fc; VL1-VH2-VH3-VL3-VL2-VH1-CL-CH1-Fc; VH1-VL2-VL3-VH3-VH2-VL1-CL-CH1-Fc; VL1-L1-VL2-L2-VL3-L3-VH3-L4-VH2-L5-VH1-CH1-Fc; VH1-L1-VH2-L2-VH3-L3-VL3-L4-VL2-L5-VL1-CH1-Fc; VL1-L1-VH2-L2-VL3-L3-VH3-L4-VL2-L5-VH1-CH1-Fc; VH1-L1-VL2-L2-VH3-L3-VL3-L4-VH2-L5-VL1-CH1-Fc; VL1-L1-VL2-L2-VH3-L3-VL3-L4-VH2-L5-VH1-CH1-Fc; VH1-L1-VH2-L2-VL3-L3-VH3-L4-VL2-L5-VL1-CH1-Fc; VL1-L1-VH2-L2-VH3-L3-VL3-L4-VL2-L5-VH1-CH1-Fc; VH1-L1-VL2-L2-VL3-L3-VH3-L4-VH2-L5-VL1-CH1-Fc; VL1-L1-VL2-L2-VL3-L3-VH3-L4-VH2-L5-VH1-CL-Fc; VH1-L1-VH2-L2-VH3-L3-VL3-L4-VL2-L5-VL1-CL-Fc; VL1-L1-VH2-L2-VL3-L3-VH3-L4-VL2-L5-VH1-CL-Fc; VH1-L1-VL2-L2-VH3-L3-VL3-L4-VH2-L5-VL1-CL-Fc; VL1-L1-VL2-L2-VH3-L3-VL3-L4-VH2-L5-VH1-CL-Fc; VH1-L1-VH2-L2-VL3-L3-VH3-L4-VL2-L5-VL1-CL-Fc; VL1-L1-VH2-L2-VH3-L3-VL3-L4-VL2-L5-VH1-CL-Fc; VH1-L1-VL2-L2-VL3-L3-VH3-L4-VH2-L5-VL1-CL-Fc; VL1-L1-VL2-L2-VL3-L3-VH3-L4-VH2-L5-VH1-CH1-CL-Fc; VH1-L1-VH2-L2-VH3-L3-VL3-L4-VL2-L5-VL1-CH1-CL-Fc; VL1-L1-VH2-L2-VL3-L3-VH3-L4-VL2-L5-VH1-CH1-CL-Fc; VH1-L1-VL2-L2-VH3-L3-VL3-L4-VH2-L5-VL1-CH1-CL-Fc; VL1-L1-VL2-L2-VH3-L3-VL3-L4-VH2-L5-VH1-CH1-CL-Fc; VH1-L1-VH2-L2-VL3-L3-VH3-L4-VL2-L5-VL1-CH1-CL-Fc; VL1-L1-VH2-L2-VH3-L3-VL3-L4-VL2-L5-VH1-CH1-CL-Fc; VH1-L1-VL2-L2-VL3-L3-VH3-L4-VH2-L5-VL1-CH1-CL-Fc; VL1-L1-VL2-L2-VL3-L3-VH3-L4-VH2-L5-VH1-CL-CH1-Fc; VH1-L1-VH2-L2-VH3-L3-VL3-L4-VL2-L5-VL1-CL-CH1-Fc; VL1-L1-VH2-L2-VL3-L3-VH3-L4-VL2-L5-VH1-CL-CH1-Fc; VH1-L1-VL2-L2-VH3-L3-VL3-L4-VH2-L5-VL1-CL-CH1-Fc; VL1-L1-VL2-L2-VH3-L3-VL3-L4-VH2-L5-VH1-CL-CH1-Fc; VH1-L1-VH2-L2-VL3-L3-VH3-L4-VL2-L5-VL1-CL-CH1-Fc; VL1-L1-VH2-L2-VH3-L3-VL3-L4-VL2-L5-VH1-CL-CH1-Fc; or VH1-L1-VL2-L2-VL3-L3-VH3-L4-VH2-L5-VL1-CL-CH1-Fc; wherein the second polypeptide has a structure represented by Fc; VL4-VH4-CH1-Fc; VL4-VH4-CL-Fc; VL4-VH4-CH1-CL-Fc; VL4-VH4-CL-CH1-Fc; VH4-VL4-CH1-Fc; VH4-VL4-CL-Fc; VH4-VL4-CH1-CL-Fc; VH4-VL4-CL-CH1-Fc; VL4-L6-VH4-CH1-Fc; VL4-L6-VH4-CL-Fc; VL4-L6-VH4-CH1-CL-Fc; VL4-L6-VH4-CL-CH1-Fc; VH4-L6-VL4-CH1-Fc; VH4-L6-VL4-CL-Fc; VH4-L6-VL4-CH1-CL-Fc; VH4-L6-VL4-CL-CH1-Fc; VL4-CL-VH4-CH1-Fc; VH4-CL-VL4-CH1-Fc; VL4-CH1-VH4-CL-Fc; VH4-CH1-VL4-CL-Fc; VL4-L6-CL-L7-VH4-L8-CH1-Fc; VL4-L6-CL-L7-VH4-L8-CH1-L9-Fc; VH4-L6-CL-L7-VL4-L8-CH1-Fc; VH4-L6-CL-L7-VL4-L8-CH1-L9-Fc; VL4-L6-CH1-L7-VH4-L8-CL-Fc; VL4-L6-CH1-L7-VH4-L8-CL-L9-Fc; VH4-L6-CH1-L7-VL4-L8-CL-Fc; VH4-L6-CH1-L7-VL4-L8-CL-L9-Fc; VL4-VL5-VH5-VH4-CH1-Fc; VL4-VL5-VH5-VH4-CL-Fc; VL4-VL5-VH5-VH4-CH1-CL-Fc; VL4-VL5-VH5-VH4-CL-CH1-Fc; VH4-VH5-VL5-VL4-CH1-Fc; VH4-VH5-VL5-VL4-CL-Fc; VH4-VH5-VL5-VL4-CH1-CL-Fc; VH4-VH5-VL5-VL4-CL-CH1-Fc; VL4-L6-VL5-L7-VH5-L8-VH4-CH1-Fc; VL4-L6-VL5-L7-VH5-L8-VH4-CL-Fc; VL4-L6-VL5-L7-VH5-L8-VH4-CH1-CL-Fc; VL4-L6-VL5-L7-VH5-L8-VH4-CL-CH1-Fc; VH4-L6-VH5-L7-VL5-L8-VL4-CH1-Fc; VH4-L6-VH5-L7-VL5-L8-VL4-CL-Fc; VH4-L6-VH5-L7-VL5-L8-VL4-CH1-CL-Fc; VH4-L6-VH5-L7-VL5-L8-VL4-CL-CH1-Fc; VL4-VL5-VL6-VH6-VH5-VH4-CH1-Fc; VL4-VL5-VL6-VH6-VH5-VH4-CL-Fc; VL4-VL5-VL6-VH6-VH5-VH4-CH1-CL-Fc; VL4-VL5-VL6-VH6-VH5-VH4-CL-CH1-Fc; VH4-VH5-VH6-VL6-VL5-VL4-CH1-Fc; VH4-VH5-VH6-VL6-VL5-VL4-CL-Fc; VH4-VH5-VH6-VL6-VL5-VL4-CH1-CL-Fc; VH4-VH5-VH6-VL6-VL5-VL4-CL-CH1-Fc; VL4-VH5-VL6-VH6-VL5-VH4-CH1-Fc; VL4-VH5-VL6-VH6-VL5-VH4-CL-Fc; VL4-VH5-VL6-VH6-VL5-VH4-CH1-CL-Fc; VL4-VH5-VL6-VH6-VL5-VH4-CL-CH1-Fc; VH4-VL5-VH6-VL6-VH5-VL4-CH1-Fc; VH4-VL5-VH6-VL6-VH5-VL4-CL-Fc; VH4-VL5-VH6-VL6-VH5-VL4-CH1-CL-Fc; VH4-VL5-VH6-VL6-VH5-VL4-CL-CH1-Fc; VL4-VL5-VH6-VL6-VH5-VH4-CH1-Fc; VL4-VL5-VH6-VL6-VH5-VH4-CL-Fc; VL4-VL5-VH6-VL6-VH5-VH4-CH1-CL-Fc; VL4-VL5-VH6-VL6-VH5-VH4-CL-CH1-Fc; VH4-VH5-VL6-VH6-VL5-VL4-CH1-Fc; VH4-VH5-VL6-VH6-VL5-VL4-CL-Fc; VH4-VH5-VL6-VH6-VL5-VL4-CH1-CL-Fc; VH4-VH5-VL6-VH6-VL5-VL4-CL-CH1-Fc; VL4-VH5-VH6-VL6-VL5-VH4-CH1-Fc; VL4-VH5-VH6-VL6-VL5-VH4-CL-Fc; VL4-VH5-VH6-VL6-VL5-VH4-CH1-CL-Fc; VL4-VH5-VH6-VL6-VL5-VH4-CL-CH1-Fc; VH4-VL5-VL6-VH6-VH5-VL4-CH1-Fc; VH4-VL5-VL6-VH6-VH5-VL4-CL-Fc; VH4-VL5-VL6-VH6-VH5-VL4-CH1-CL-Fc; VH4-VL5-VL6-VH6-VH5-VL4-CL-CH1-Fc; VL4-L6-VL5-L7-VL6-L8-VH6-L9-VH5-L10-VH4-CH1-Fc; VL4-L6-VL5-L7-VL6-L8-VH6-L9-VH5-L10-VH4-CL-Fc; VL4-L6-VL5-L7-VL6-L8-VH6-L9-VH5-L10-VH4-CH1-CL-Fc; VL4-L6-VL5-L7-VL6-L8-VH6-L9-VH5-L10-VH4-CL-CH1-Fc; VH4-L6-VH5-L7-VH6-L8-VL6-L9-VL5-L10-VL4-CH1-Fc; VH4-L6-VH5-L7-VH6-L8-VL6-L9-VL5-L10-VL4-CL-Fc; VH4-L6-VH5-L7-VH6-L8-VL6-L9-VL5-L10-VL4-CH1-CL-Fc; VH4-L6-VH5-L7-VH6-L8-VL6-L9-VL5-L10-VL4-CL-CH1-Fc; VL4-L6-VH5-L7-VL6-L8-VH6-L9-VL5-L10-VH4-CH1-Fc; VL4-L6-VH5-L7-VL6-L8-VH6-L9-VL5-L10-VH4-CL-Fc; VL4-L6-VH5-L7-VL6-L8-VH6-L9-VL5-L10-VH4-CH1-CL-Fc; VL4-L6-VH5-L7-VL6-L8-VH6-L9-VL5-L10-VH4-CL-CH1-Fc; VH4-L6-VL5-L7-VH6-L8-VL6-L9-VH5-L10-VL4-CH1-Fc; VH4-L6-VL5-L7-VH6-L8-VL6-L9-VH5-L10-VL4-CL-Fc; VH4-L6-VL5-L7-VH6-L8-VL6-L9-VH5-L10-VL4-CH1-CL-Fc; VH4-L6-VL5-L7-VH6-L8-VL6-L9-VH5-L10-VL4-CL-CH1-Fc; VL4-L6-VL5-L7-VH6-L8-VL6-L9-VH5-L10-VH4-CH1-Fc; VL4-L6-VL5-L7-VH6-L8-VL6-L9-VH5-L10-VH4-CL-Fc; VL4-L6-VL5-L7-VH6-L8-VL6-L9-VH5-L10-VH4-CH1-CL-Fc; VL4-L6-VL5-L7-VH6-L8-VL6-L9-VH5-L10-VH4-CL-CH1-Fc; VH4-L6-VH5-L7-VL6-L8-VH6-L9-VL5-L10-VL4-CH1-Fc; VH4-L6-VH5-L7-VL6-L8-VH6-L9-VL5-L10-VL4-CL-Fc; VH4-L6-VH5-L7-VL6-L8-VH6-L9-VL5-L10-VL4-CH1-CL-Fc; VH4-L6-VH5-L7-VL6-L8-VH6-L9-VL5-L10-VL4-CL-CH1-Fc; VL4-L6-VH5-L7-VH6-L8-VL6-L9-VL5-L10-VH4-CH1-Fc; VL4-L6-VH5-L7-VH6-L8-VL6-L9-VL5-L10-VH4-CL-Fc; VL4-L6-VH5-L7-VH6-L8-VL6-L9-VL5-L10-VH4-CH1-CL-Fc; VL4-L6-VH5-L7-VH6-L8-VL6-L9-VL5-L10-VH4-CL-CH1-Fc; VH4-L6-VL5-L7-VL6-L8-VH6-L9-VH5-L10-VL4-CH1-Fc; VH4-L6-VL5-L7-VL6-L8-VH6-L9-VH5-L10-VL4-CL-Fc; VH4-L6-VL5-L7-VL6-L8-VH6-L9-VH5-L10-VL4-CH1-CL-Fc; VH4-L6-VL5-L7-VL6-L8-VH6-L9-VH5-L10-VL4-CL-CH1-Fc; VL4-L6-VL5-L7-VL6-L8-VH6-L9-VH5-L10-VH4-L11-CH1-Fc; VL4-L6-VL5-L7-VL6-L8-VH6-L9-VH5-L10-VH4-L11-CL-Fc; VL4-L6-VL5-L7-VL6-L8-VH6-L9-VH5-L10-VH4-L11-CH1-CL-Fc; VL4-L6-VL5-L7-VL6-L8-VH6-L9-VH5-L10-VH4-L11-CL-CH1-Fc; VH4-L6-VH5-L7-VH6-L8-VL6-L9-VL5-L10-VL4-L11-CH1-Fc; VH4-L6-VH5-L7-VH6-L8-VL6-L9-VL5-L10-VL4-L11-CL-Fc; VH4-L6-VH5-L7-VH6-L8-VL6-L9-VL5-L10-VL4-L11-CH1-CL-Fc; VH4-L6-VH5-L7-VH6-L8-VL6-L9-VL5-L10-VL4-L11-CL-CH1-Fc; VL4-L6-VH5-L7-VL6-L8-VH6-L9-VL5-L10-VH4-L11-CH1-Fc; VL4-L6-VH5-L7-VL6-L8-VH6-L9-VL5-L10-VH4-L11-CL-Fc; VL4-L6-VH5-L7-VL6-L8-VH6-L9-VL5-L10-VH4-L11-CH1-CL-Fc; VL4-L6-VH5-L7-VL6-L8-VH6-L9-VL5-L10-VH4-L11-CL-CH1-Fc; VH4-L6-VL5-L7-VH6-L8-VL6-L9-VH5-L10-VL4-L11-CH1-Fc; VH4-L6-VL5-L7-VH6-L8-VL6-L9-VH5-L10-VL4-L11-CL-Fc; VH4-L6-VL5-L7-VH6-L8-VL6-L9-VH5-L10-VL4-L11-CH1-CL-Fc; VH4-L6-VL5-L7-VH6-L8-VL6-L9-VH5-L10-VL4-L11-CL-CH1-Fc; VL4-L6-VL5-L7-VH6-L8-VL6-L9-VH5-L10-VH4-L11-CH1-Fc; VL4-L6-VL5-L7-VH6-L8-VL6-L9-VH5-L10-VH4-L11-CL-Fc; VL4-L6-VL5-L7-VH6-L8-VL6-L9-VH5-L10-VH4-L11-CH1-CL-Fc; VL4-L6-VL5-L7-VH6-L8-VL6-L9-VH5-L10-VH4-L11-CL-CH1-Fc; VH4-L6-VH5-L7-VL6-L8-VH6-L9-VL5-L10-VL4-L11-CH1-Fc; VH4-L6-VH5-L7-VL6-L8-VH6-L9-VL5-L10-VL4-L11-CL-Fc; VH4-L6-VH5-L7-VL6-L8-VH6-L9-VL5-L10-VL4-L11-CH1-CL-Fc; VH4-L6-VH5-L7-VL6-L8-VH6-L9-VL5-L10-VL4-L11-CL-CH1-Fc; VL4-L6-VH5-L7-VH6-L8-VL6-L9-VL5-L10-VH4-L11-CH1-Fc; VL4-L6-VH5-L7-VH6-L8-VL6-L9-VL5-L10-VH4-L11-CL-Fc; VL4-L6-VH5-L7-VH6-L8-VL6-L9-VL5-L10-VH4-L11-CH1-CL-Fc; VL4-L6-VH5-L7-VH6-L8-VL6-L9-VL5-L10-VH4-L11-CL-CH1-Fc; VH4-L6-VL5-L7-VL6-L8-VH6-L9-VH5-L10-VL4-L11-CH1-Fc; VH4-L6-VL5-L7-VL6-L8-VH6-L9-VH5-L10-VL4-L11-CL-Fc; VH4-L6-VL5-L7-VL6-L8-VH6-L9-VH5-L10-VL4-L11-CH1-CL-Fc; or VH4-L6-VL5-L7-VL6-L8-VH6-L9-VH5-L10-VL4-L11-CL-CH1-Fc; wherein VL1 is a first immunoglobulin light chain variable region that specifically binds to an HIV protein; VL2 is a second immunoglobulin light chain variable region that specifically binds to an HIV protein; VL3 is a third immunoglobulin light chain variable region that specifically binds to an HIV protein; VL4 is a fourth immunoglobulin light chain variable region that specifically binds to an HIV protein; VL5 is a fifth immunoglobulin light chain variable region that specifically binds to an HIV protein; VL6 is a sixth immunoglobulin light chain variable region that specifically binds to an HIV protein; VH1 is a first immunoglobulin heavy chain variable region that specifically binds to an HIV protein; VH2 is a second immunoglobulin heavy chain variable region that specifically binds to an HIV protein; VH3 is a third immunoglobulin heavy chain variable region that specifically binds to an HIV protein; VH4 is a fourth immunoglobulin heavy chain variable region that specifically binds to an HIV protein; VH5 is a fifth immunoglobulin heavy chain variable region that specifically binds to an HIV protein; VH6 is a sixth immunoglobulin heavy chain variable region that specifically binds to an HIV protein; Fc is a region comprising an immunoglobulin heavy chain constant region 2 (CH2), an immunoglobulin heavy chain constant region 3 (CH3), and optionally, an immunoglobulin hinge; CH1 is a heavy chain constant region 1; CL is a light chain constant region; and L1, L2, L3, L4, L5, L6, L7, L8, L9, L10 and L11 are amino acid linkers.

In some aspects, the invention is directed to an antigen binding polypeptide or antigen binding polypeptide complex comprising a polypeptide having a structure represented by VL1-VL2-VL3-VH3-VH2-VH1-CH3-CH3; VH1-VH2-VH3-VL3-VL2-VL1-CH3-CH3; VL1-VH2-VL3-VH3-VL2-VH1-CH3-CH3; VH1-VL2-VH3-VL3-VH2-VL1-CH3-CH3; VL1-VL2-VH3-VL3-VH2-VH1-CH3-CH3; VH1-VH2-VL3-VH3-VL2-VL1-CH3-CH3; VL1-VH2-VH3-VL3-VL2-VH1-CH3-CH3; VH1-VL2-VL3-VH3-VH2-VL1-CH3-CH3; VL1-L1-VL2-L2-VL3-L3-VH3-L4-VH2-L5-VH1-CH3-CH3; VH1-L1-VH2-L2-VH3-L3-VL3-L4-VL2-L5-VL1-CH3-CH3; VL1-L1-VH2-L2-VL3-L3-VH3-L4-VL2-L5-VH1-CH3-CH3; VH1-L1-VL2-L2-VH3-L3-VL3-L4-VH2-L5-VL1-CH3-CH3; VL1-L1-VL2-L2-VH3-L3-VL3-L4-VH2-L5-VH1-CH3-CH3; VH1-L1-VH2-L2-VL3-L3-VH3-L4-VL2-L5-VL1-CH3-CH3; VL1-L1-VH2-L2-VH3-L3-VL3-L4-VL2-L5-VH1-CH3-CH3; VH1-L1-VL2-L2-VL3-L3-VH3-L4-VH2-L5-VL1-CH3-CH3; VL1-L1-VL2-L2-VL3-L3-VH3-L4-VH2-L5-VH1-L6-CH3-CH3; VH1-L1-VH2-L2-VH3-L3-VL3-L4-VL2-L5-VL1-L6-CH3-CH3; VL1-L1-VH2-L2-VL3-L3-VH3-L4-VL2-L5-VH1-L6-CH3-CH3; VH1-L1-VL2-L2-VH3-L3-VL3-L4-VH2-L5-VL1-L6-CH3-CH3; VL1-L1-VL2-L2-VH3-L3-VL3-L4-VH2-L5-VH1-L6-CH3-CH3; VH1-L1-VH2-L2-VL3-L3-VH3-L4-VL2-L5-VL1-L6-CH3-CH3; VL1-L1-VH2-L2-VH3-L3-VL3-L4-VL2-L5-VH1-L6-CH3-CH3; VH1-L1-VL2-L2-VL3-L3-VH3-L4-VH2-L5-VL1-L6-CH3-CH3; VL1-L1-VL2-L2-VL3-L3-VH3-L4-VH2-L5-VH1-L6-CH3-L7-CH3; VH1-L1-VH2-L2-VH3-L3-VL3-L4-VL2-L5-VL1-L6-CH3-L7-CH3; VL1-L1-VH2-L2-VL3-L3-VH3-L4-VL2-L5-VH1-L6-CH3-L7-CH3; VH1-L1-VL2-L2-VH3-L3-VL3-L4-VH2-L5-VL1-L6-CH3-L7-CH3; VL1-L1-VL2-L2-VH3-L3-VL3-L4-VH2-L5-VH1-L6-CH3-L7-CH3; VH1-L1-VH2-L2-VL3-L3-VH3-L4-VL2-L5-VL1-L6-CH3-L7-CH3; VL1-L1-VH2-L2-VH3-L3-VL3-L4-VL2-L5-VH1-L6-CH3-L7-CH3; or VH1-L1-VL2-L2-VL3-L3-VH3-L4-VH2-L5-VL1-L6-CH3-L7-CH3; wherein VL1 is a first immunoglobulin light chain variable region that specifically binds to an HIV protein; VL2 is a second immunoglobulin light chain variable region that specifically binds to an HIV protein; VL3 is a third immunoglobulin light chain variable region that specifically binds to an HIV protein; VH1 is a first immunoglobulin heavy chain variable region that specifically binds to an HIV protein; VH2 is a second immunoglobulin heavy chain variable region that specifically binds to an HIV protein; VH3 is a third immunoglobulin heavy chain variable region that specifically binds to an HIV protein; CH3 is an immunoglobulin heavy chain constant region 3; and L1, L2, L3, L4, L5, L6 and L7 are amino acid linkers.

In some aspects, an antigen binding polypeptide complex of the invention comprises first polypeptide, a second polypeptide, and a third polypeptide; wherein the first polypeptide has a structure represented by VL1-VL2-VL3-VH3-VH2-VH1; VH1-VH2-VH3-VL3-VL2-VL1; VL1-VH2-VL3-VH3-VL2-VH1; VH1-VL2-VH3-VL3-VH2-VL1; VL1-VL2-VH3-VL3-VH2-VH1; VH1-VH2-VL3-VH3-VL2-VL1; VL1-VH2-VH3-VL3-VL2-VH1; VH1-VL2-VL3-VH3-VH2-VL1; VL1-L1-VL2-L2-VL3-L3-VH3-L4-VH2-L5-VH1; VH1-L1-VH2-L2-VH3-L3-VL3-L4-VL2-L5-VL1; VL1-L1-VH2-L2-VL3-L3-VH3-L4-VL2-L5-VH1; VH1-L1-VL2-L2-VH3-L3-VL3-L4-VH2-L5-VL1; VL1-L1-VL2-L2-VH3-L3-VL3-L4-VH2-L5-VH1; VH1-L1-VH2-L2-VL3-L3-VH3-L4-VL2-L5-VL1; VL1-L1-VH2-L2-VH3-L3-VL3-L4-VL2-L5-VH1; or VH1-L1-VL2-L2-VL3-L3-VH3-L4-VH2-L5-VL1; wherein the second polypeptide has a structure represented by VL4-VL5; VL4-L6-VL5; VL4-VL5-VL6; or VL4-L6-VL5-L7-VL6; wherein the third polypeptide has a structure represented by VH4-VH5; VH4-L6-VH5; VH4-VH5-VH6; or VH4-L6-VH5-L7-VH6; wherein VL1 is a first immunoglobulin light chain variable region that specifically binds to an HIV protein; VL2 is a second immunoglobulin light chain variable region that specifically binds to an HIV protein; VL3 is a third immunoglobulin light chain variable region that specifically binds to an HIV protein; VL4 is a fourth immunoglobulin light chain variable region that specifically binds to an HIV protein; VL5 is a fifth immunoglobulin light chain variable region that specifically binds to an HIV protein; VL6 is a sixth immunoglobulin light chain variable region that specifically binds to an HIV protein; VH1 is a first immunoglobulin heavy chain variable region that specifically binds to an HIV protein; VH2 is a second immunoglobulin heavy chain variable region that specifically binds to an HIV protein; VH3 is a third immunoglobulin heavy chain variable region that specifically binds to an HIV protein; VH4 is a fourth immunoglobulin heavy chain variable region that specifically binds to an HIV protein; VH5 is a fifth immunoglobulin heavy chain variable region that specifically binds to an HIV protein; VH6 is a sixth immunoglobulin heavy chain variable region that specifically binds to an HIV protein; and L1, L2, L3, L4, L5, L6 and L7 are amino acid linkers.

In some aspects, an antigen binding polypeptide complex of the invention comprises a first polypeptide, a second polypeptide, and a third polypeptide; wherein the first polypeptide has a structure represented by VL1-VL2-VL3-VH3-VH2-VH1-Fc; VH1-VH2-VH3-VL3-VL2-VL1-Fc; VL1-VH2-VL3-VH3-VL2-VH1-Fc; VH1-VL2-VH3-VL3-VH2-VL1-Fc; VL1-VL2-VH3-VL3-VH2-VH1-Fc; VH1-VH2-VL3-VH3-VL2-VL1-Fc; VL1-VH2-VH3-VL3-VL2-VH1-Fc; VH1-VL2-VL3-VH3-VH2-VL1-Fc; VL1-L1-VL2-L2-VL3-L3-VH3-L4-VH2-L5-VH1-Fc; VL1-L1-VL2-L2-VL3-L3-VH3-L4-VH2-L5-VH1-L6-Fc; VH1-L1-VH2-L2-VH3-L3-VL3-L4-VL2-L5-VL1-Fc; VH1-L1-VH2-L2-VH3-L3-VL3-L4-VL2-L5-VL1-L6-Fc; VL1-L1-VH2-L2-VL3-L3-VH3-L4-VL2-L5-VH1-Fc; VL1-L1-VH2-L2-VL3-L3-VH3-L4-VL2-L5-VH1-L6-Fc; VH1-L1-VL2-L2-VH3-L3-VL3-L4-VH2-L5-VL1-Fc; VH1-L1-VL2-L2-VH3-L3-VL3-L4-VH2-L5-VL1-L6-Fc; VL1-L1-VL2-L2-VH3-L3-VL3-L4-VH2-L5-VH1-Fc; VL1-L1-VL2-L2-VH3-L3-VL3-L4-VH2-L5-VH1-L6-Fc; VH1-L1-VH2-L2-VL3-L3-VH3-L4-VL2-L5-VL1-Fc; VH1-L1-VH2-L2-VL3-L3-VH3-L4-VL2-L5-VL1-L6-Fc; VL1-L1-VH2-L2-VH3-L3-VL3-L4-VL2-L5-VH1-Fc; VL1-L1-VH2-L2-VH3-L3-VL3-L4-VL2-L5-VH1-L6-Fc; VH1-L1-VL2-L2-VL3-L3-VH3-L4-VH2-L5-VL1-Fc; or VH1-L1-VL2-L2-VL3-L3-VH3-L4-VH2-L5-VL1-L6-Fc; wherein the second polypeptide has a structure represented by VL4-VL5; VL4-L7-VL5; VL4-CL; VL4-L7-CL; VL4-CH1; VL4-L7-CH1; VH4-VH5; VH4-L7-VH5; VH4-CL; VH4-L7-CL; VH4-CH1; VH4-L7-CH1; VL4-VL5-VL6; VL4-L7-VL5-L8-VL6; VL4-VL5-VL6-CL; VL4-L7-VL5-L8-VL6-CL; VL4-L7-VL5-L8-VL6-L9-CL; VL4-VL5-VL6-CH1; VL4-L7-VL5-L8-VL6-CH1; VL4-L7-VL5-L8-VL6-L9-CH1; VH4-VH5-VH6; VH4-L7-VH5-L8-VH6; VH4-VH5-VH6-CL; VH4-L7-VH5-L8-VH6-CL; VH4-L7-VH5-L8-VH6-L9-CL; VH4-VH5-VH6-CH1; VH4-L7-VH5-L8-VH6-CH1; or VH4-L7-VH5-L8-VH6-L9-CH1; wherein the third polypeptide has a structure represented by VH4-VH5-Fc; VH4-L10-VH5-Fc; VH4-L10-VH5-L11-Fc; VH4-CH1-Fc; VH4-L10-CH1-Fc; VH4-L10-CH1-L11-Fc; VH4-CL-Fc; VH4-L10-CL-Fc; VH4-L10-CL-L11-Fc; VH4-VH5-Fc; VH4-L10-VH5-Fc; VH4-L10-VH5-L11-Fc; VH4-VH5-VH6-Fc; VH4-L10-VH5-L11-VH6-Fc; VH4-L10-VH5-L11-VH6-L12-Fc; VH4-VH5-VH6-CH1-Fc; VH4-L10-VH5-L11-VH6-CH1-Fc; VH4-L10-VH5-L11-VH6-L12-CH1-Fc; VH4-L10-VH5-L11-VH6-L12-CH1-L13-Fc; VH4-VH5-VH6-CL-Fc; VH4-L10-VH5-L11-VH6-CL-Fc; VH4-L10-VH5-L11-VH6-L12-CL-Fc; VH4-L10-VH5-L11-VH6-L12-CL-L13-Fc; VL4-VL5-VL6-Fc; VL4-L10-VL5-L11-VL6-Fc; VL4-L10-VL5-L11-VL6-L12-Fc; VL4-VL5-VL6-CH1-Fc; VL4-L10-VL5-L11-VL6-CH1-Fc VL4-L10-VL5-L11-VL6-L12-CH1-Fc; VL4-L10-VL5-L11-VL6-L12-CH1-L13-Fc; VL4-VL5-VL6-CL-Fc; VL4-L10-VL5-L11-VL6-CL-Fc; VL4-L10-VL5-L11-VL6-L12-CL-Fc; or VL4-L10-VL5-L11-VL6-L12-CL-L13-Fc; wherein VL1 is a first immunoglobulin light chain variable region that specifically binds to an HIV protein; VL2 is a second immunoglobulin light chain variable region that specifically binds to an HIV protein; VL3 is a third immunoglobulin light chain variable region that specifically binds to an HIV protein; VL4 is a fourth immunoglobulin light chain variable region that specifically binds to an HIV protein; VL5 is a fifth immunoglobulin light chain variable region that specifically binds to an HIV protein; VL6 is a sixth immunoglobulin light chain variable region that specifically binds to an HIV protein; VH1 is a first immunoglobulin heavy chain variable region that specifically binds to an HIV protein; VH2 is a second immunoglobulin heavy chain variable region that specifically binds to an HIV protein; VH3 is a third immunoglobulin heavy chain variable region that specifically binds to an HIV protein; VH4 is a fourth immunoglobulin heavy chain variable region that specifically binds to an HIV protein; VH5 is a fifth immunoglobulin heavy chain variable region that specifically binds to an HIV protein; VH6 is a sixth immunoglobulin heavy chain variable region that specifically binds to an HIV protein; CH1 is a heavy chain constant region 1; CL is a light chain constant region; Fc is a region comprising an immunoglobulin heavy chain constant region 2 (CH2), an immunoglobulin heavy chain constant region 3 (CH3), and optionally, an immunoglobulin hinge; and L1, L2, L3, L4, L5, L6, L7, L8, L9, L10, L11, L12 and L13 are amino acid linkers.

In some aspects, one or more of VH1, VH2, VH3, VH4, VH5 and VH6 of an antigen binding polypeptide or antigen binding polypeptide complex described herein can specifically bind to the same antigen or different antigens. In some aspects, one or more of VL1, VL2, VL3, VL4, VL5 and VL6 of an antigen binding polypeptide or antigen binding polypeptide complex described herein can specifically bind to the same antigen or different antigens.

In some aspects, VH1, VL1, VH4 and VL4 of an antigen binding polypeptide or antigen binding polypeptide complex described herein specifically bind to the same antigen. In some aspects, VH2, VL2, VH5 and VL5 of an antigen binding polypeptide or antigen binding polypeptide complex described herein specifically bind to the same antigen. In some aspects, VH3, VL3, VH6 and VL6 of an antigen binding polypeptide or antigen binding polypeptide complex described herein specifically bind to the same antigen. In some aspects, VH1, VL1, VH4 and VL4 of an antigen binding polypeptide or antigen binding polypeptide complex described herein specifically bind to the same antigen; VH2, VL2, VH5 and VL5 of an antigen binding polypeptide or antigen binding polypeptide complex described herein specifically bind to the same antigen; and VH3, VL3, VH6 and VL6 of an antigen binding polypeptide or antigen binding polypeptide complex described herein specifically bind to the same antigen.

In some aspects of an antigen binding polypeptide or antigen binding polypeptide complex of the invention, VH1, VH2 and VH3 each comprise a heavy chain variable region from the PGT121, VRC01, 10E8v4 or PG16 antibody or a variant thereof, and/or VL1, VL2 and VL3 each comprise a light chain variable region from the PGT121, VRC01, 10E8v4 or PG16 antibody or a variant thereof.

In some aspects of an antigen binding polypeptide or antigen binding polypeptide complex of the invention, VH1, VH2, VH3 and VH4 each comprise a heavy chain variable region from the PGT121, VRC01, 10E8v4 or PG16 antibody or a variant thereof, and/or VL1, VL2, VL3 and VL4 each comprise a light chain variable region from the PGT121, VRC01, 10E8v4 or PG16 antibody or a variant thereof.

In some aspects of an antigen binding polypeptide or antigen binding polypeptide complex of the invention, VH1, VH2, VH3, VH4 and VH5 each comprise a heavy chain variable region from the PGT121, VRC01, 10E8v4 or PG16 antibody or a variant thereof, and/or VL1, VL2, VL3, VL4 and VL5 each comprise a light chain variable region from the PGT121, VRC01, 10E8v4 or PG16 antibody or a variant thereof.

In some aspects of an antigen binding polypeptide or antigen binding polypeptide complex of the invention, VH1, VH2, VH3, VH4, VH5 and VH6 each comprise a heavy chain variable region from the PGT121, VRC01, 10E8v4 or PG16 antibody or a variant thereof, and/or VL1, VL2, VL3, VL4, VL5 and VL6 each comprise a light chain variable region from the PGT121, VRC01, 10E8v4 or PG16 antibody or a variant thereof.

In some aspects, VH1, VH2 and VH3 of an antigen binding polypeptide or antigen binding polypeptide complex of the invention each comprise an amino acid sequence having at least 90% identity, at least 95% identity or 100% identity to any one of SEQ ID NOs:20-23; and/or VL1, VL2 and VL3 each comprise an amino acid sequence having at least 90% identity, at least 95% identity or 100% identity to any one of SEQ ID NOs:24-27.

In some aspects, VH1, VH2, VH3 and VH4 of an antigen binding polypeptide or antigen binding polypeptide complex of the invention each comprise an amino acid sequence having at least 90% identity, at least 95% identity or 100% identity to any one of SEQ ID NOs:20-23; and/or VL1, VL2, VL3 and VL4 each comprise an amino acid sequence having at least 90% identity, at least 95% identity or 100% identity to any one of SEQ ID NOs:24-27.

In some aspects, VH1, VH2, VH3, VH4 and VH5 of an antigen binding polypeptide or antigen binding polypeptide complex of the invention each comprise an amino acid sequence having at least 90% identity, at least 95% identity or 100% identity to any one of SEQ ID NOs:20-24; and/or VL1, VL2, VL3, VL4 and VL5 each comprise an amino acid sequence having at least 90% identity, at least 95% identity or 100% identity to any one of SEQ ID NOs:24-27.

In some aspects, VH1, VH2, VH3, VH4, VH5 and VH6 of an antigen binding polypeptide or antigen binding polypeptide complex of the invention each comprise an amino acid sequence having at least 90% identity, at least 95% identity or 100% identity to any one of SEQ ID NOs:20-23; and/or VL1, VL2, VL3, VL4, VL5 and VL6 each comprise an amino acid sequence having at least 90% identity, at least 95% identity or 100% identity to any one of SEQ ID NOs:24-27.

In some aspects, VH1, VH2 and VH3 of an antigen binding polypeptide or antigen binding polypeptide complex of the invention each comprise a CDR1 having an amino acid sequence with at least 90% identity, at least 95% identity or 100% identity to any one of SEQ ID NOs:60, 63, 66 and 69; a CDR2 having an amino acid sequence with at least 90% identity, at least 95% identity or 100% identity to any one of SEQ ID NOs:61, 64, 67 and 70; and a CDR3 having an amino acid sequence with at least 90% identity, at least 95% identity or 100% identity to any one of SEQ ID NOs:62, 65, 68 and 71; and VL1, VL2 and VL3 each comprise a CDR1 having an amino acid sequence with at least 90% identity, at least 95% identity or 100% identity to any one of SEQ ID NOs:72, 75, 78 and 81; a CDR2 having an amino acid sequence with at least 90% identity, at least 95% identity or 100% identity to any one of SEQ ID NOs:73, 76, 79 and 82; and a CDR3 having an amino acid sequence with at least 90% identity, at least 95% identity or 100% identity to any one of SEQ ID NOs:74, 77, 80 and 83.

In some aspects, VH1, VH2, VH3 and VH4 of an antigen binding polypeptide or antigen binding polypeptide complex of the invention each comprise a CDR1 having an amino acid sequence with at least 90% identity, at least 95% identity or 100% identity to any one of SEQ ID NOs:60, 63, 66 and 69; a CDR2 having an amino acid sequence with at least 90% identity, at least 95% identity or 100% identity to any one of SEQ ID NOs:61, 64, 67 and 70; and a CDR3 having an amino acid sequence with at least 90% identity, at least 95% identity or 100% identity to any one of SEQ ID NOs:62, 65, 68 and 71; and VL1, VL2, VL3 and VL4 each comprise a CDR1 having an amino acid sequence with at least 90% identity, at least 95% identity or 100% identity to any one of SEQ ID NOs:72, 75, 78 and 81; a CDR2 having an amino acid sequence with at least 90% identity, at least 95% identity or 100% identity to any one of SEQ ID NOs:73, 76, 79 and 82; and a CDR3 having an amino acid sequence with at least 90% identity, at least 95% identity or 100% identity to any one of SEQ ID NOs:74, 77, 80 and 83.

In some aspects, VH1, VH2, VH3, VH4 and VH5 of an antigen binding polypeptide or antigen binding polypeptide complex of the invention each comprise a CDR1 having an amino acid sequence with at least 90% identity, at least 95% identity or 100% identity to any one of SEQ ID NOs:60, 63, 66 and 69; a CDR2 having an amino acid sequence with at least 90% identity, at least 95% identity or 100% identity to any one of SEQ ID NOs:61, 64, 67 and 70; and a CDR3 having an amino acid sequence with at least 90% identity, at least 95% identity or 100% identity to any one of SEQ ID NOs:62, 65, 68 and 71; and VL1, VL2, VL3, VL4 and VL5 each comprise a CDR1 having an amino acid sequence with at least 90% identity, at least 95% identity or 100% identity to any one of SEQ ID NOs:72, 75, 78 and 81; a CDR2 having an amino acid sequence with at least 90% identity, at least 95% identity or 100% identity to any one of SEQ ID NOs:73, 76, 79 and 82; and a CDR3 having an amino acid sequence with at least 90% identity, at least 95% identity or 100% identity to any one of SEQ ID NOs:74, 77, 80 and 83.

In some aspects, VH1, VH2, VH3, VH4, VH5 and VH6 of an antigen binding polypeptide or antigen binding polypeptide complex of the invention each comprise a CDR1 having an amino acid sequence with at least 90% identity, at least 95% identity or 100% identity to any one of SEQ ID NOs:60, 63, 66 and 69; a CDR2 having an amino acid sequence with at least 90% identity, at least 95% identity or 100% identity to any one of SEQ ID NOs:61, 64, 67 and 70; and a CDR3 having an amino acid sequence with at least 90% identity, at least 95% identity or 100% identity to any one of SEQ ID NOs:62, 65, 68 and 71; and VL1, VL2, VL3, VL4, VL5 and VL6 each comprise a CDR1 having an amino acid sequence with at least 90% identity, at least 95% identity or 100% identity to any one of SEQ ID NOs:72, 75, 78 and 81; a CDR2 having an amino acid sequence with at least 90% identity, at least 95% identity or 100% identity to any one of SEQ ID NOs:73, 76, 79 and 82; and a CDR3 having an amino acid sequence with at least 90% identity, at least 95% identity or 100% identity to any one of SEQ ID NOs:74, 77, 80 and 83.

### Tetraspecific Constructs

In yet other aspects, the invention is directed to antigen binding polypeptide complexes having a first polypeptide and a second polypeptide, wherein the first polypeptide has a structure represented by VL1-VL2-VH2-VH1 or VH1-VH2-VL2-VL1, and the second polypeptide has a structure represented by VL3-VL4-VH4-VH3 or VH3-VH4-VL4-VL3. In some aspects, the antigen binding polypeptide complex contains an amino acid linker between any two regions denoted in a structure described herein. In some aspects, the antigen binding polypeptide complex can contain an Fc region, CH1 region, CL region, or any combination thereof. In some aspects, the antigen binding polypeptide complex is an antibody or antigen binding fragment thereof.

The antigen binding polypeptides and antigen binding polypeptide complexes described herein specifically bind to an HIV protein. This includes specific binding to one or more HIV proteins and specific binding to one or more epitopes on the same HIV protein. In some aspects, the HIV protein is selected from the group consisting of an HIV envelope protein, an HIV structural protein, an HIV functional protein, or an HIV accessory protein. In some aspects, the HIV envelope protein is HIV envelope glycoprotein (Env), HIV envelope glycoprotein gp160, HIV envelope surface glycoprotein gp120, or HIV transmembrane envelope protein gp41. In some aspects, the HIV structural protein is p17, p24, p7 or p55. In some aspects, the HIV functional protein is p66, HIV-1 protease (PR) or p31. In some aspects, the HIV accessory protein is Nef, Tat, Rev, Vif, Vpr or Vpu.

In some aspects, the invention is directed to an antigen binding polypeptide complex comprising a first polypeptide and a second polypeptide; wherein the first polypeptide has a structure represented by VL1-VL2-VH2-VH1; VH1-VH2-VL2-VL1; VL1-L1-VL2-L2-VH2-L3-VH1; or VH1-L1-VH2-L2-VL2-L3-VL1; wherein the second polypeptide has a structure represented by VL3-VL4-VH4-VH3; VH3-VH4-VL4-VL3; VL3-L4-VL4-L5-VH4-L6-VH3; or VH3-L4-VH4-L5-VL4-L6-VL3; wherein VL1 is a first immunoglobulin light chain variable region that specifically binds to an HIV protein; VL2 is a second immunoglobulin light chain variable region that specifically binds to an HIV protein; VL3 is a third immunoglobulin light chain variable region that specifically binds to an HIV protein; VL4 is a fourth immunoglobulin light chain variable region that specifically binds to an HIV protein; VH1 is a first immunoglobulin heavy chain variable region that specifically binds to an HIV protein; VH2 is a second immunoglobulin heavy chain variable region that specifically binds to an HIV protein; VH3 is a third immunoglobulin heavy chain variable region that specifically binds to an HIV protein; VH4 is a fourth immunoglobulin heavy chain variable region that specifically binds to an HIV protein; and L1, L2, L3, L4, L5 and L6 are amino acid linkers.

In other aspects, the invention is directed to an antigen binding polypeptide complex comprising a first polypeptide and a second polypeptide; wherein the first polypeptide has a structure represented by VL1-VL2-VH2-VH1-Fc; VH1-VH2-VL2-VL1-Fc; VL1-L1-VL2-L2-VH2-L3-VH1-Fc; VH1-L1-VH2-L2-VL2-L3-VL1-Fc; VL1-L1-VL2-L2-VH2-L3-VH1-L4-Fc; or VH1-L1-VH2-L2-VL2-L3-VL1-L4-Fc; wherein the second polypeptide has a structure represented by VL3-VL4-VH4-VH3-Fc; VH3-VH4-VL4-VL3-Fc; VL3-L5-VL4-L6-VH4-L7-VH3-Fc; VH3-L5-VH4-L6-VL4-L7-VL3-Fc; VL3-L5-VL4-L6-VH4-L7-VH3-L8-Fc; or VH3-L5-VH4-L6-VL4-L7-VL3-L8-Fc; wherein VL1 is a first immunoglobulin light chain variable region that specifically binds to an HIV protein; VL2 is a second immunoglobulin light chain variable region that specifically binds to an HIV protein; VL3 is a third immunoglobulin light chain variable region that specifically binds to an HIV protein; VL4 is a fourth immunoglobulin light chain variable region that specifically binds to an HIV protein; VH1 is a first immunoglobulin heavy chain variable region that specifically binds to an HIV protein; VH2 is a second immunoglobulin heavy chain variable region that specifically binds to an HIV protein; VH3 is a third immunoglobulin heavy chain variable region that specifically binds to an HIV protein; VH4 is a fourth immunoglobulin heavy chain variable region that specifically binds to an HIV protein; Fc is a region comprising an immunoglobulin heavy chain constant region 2 (CH2), an immunoglobulin heavy chain constant region 3 (CH3), and optionally, an immunoglobulin hinge; and L1, L2, L3, L4, L5, L6, L7 and L8 are amino acid linkers.

In other aspects, the invention is directed to an antigen binding polypeptide complex comprising a first polypeptide and a second polypeptide; wherein the first polypeptide has a structure represented by VL1-VL2-VH2-VH1-CH1; VH1-VH2-VL2-VL1-CH1; VL1-VL2-VH2-VH1-CL; VH1-VH2-VL2-VL1-CL; VL1-VL2-VH2-VH1-CH1-CL; VH1-VH2-VL2-VL1-CH1-CL; VL1-VL2-VH2-VH1-CL-CH1; VH1-VH2-VL2-VL1-CL-CH1; VL1-L1-VL2-L2-VH2-L3-VH1-L4-CH1; VH1-L1-VH2-L2-VL2-L3-VL1-L4-CH1; VL1-L1-VL2-L2-VH2-L3-VH1-L4-CL; VH1-L1-VH2-L2-VL2-L3-VL1-L4-CL; VL1-L1-VL2-L2-VH2-L3-VH1-L4-CH1-L5-CL; VH1-L1-VH2-L2-VL2-L3-VL1-L4-CH1-L5-CL; VL1-L1-VL2-L2-VH2-L3-VH1-L4-CL-L5-CH1; or VH1-L1-VH2-L2-VL2-L3-VL1-L4-CL-L5-CH1; wherein the second polypeptide has a structure represented by VL3-VL4-VH4-VH3-CH1; VH3-VH4-VL4-VL3-CH1; VL3-VL4-VH4-VH3-CL; VH3-VH4-VL4-VL3-CL; VL3-VL4-VH4-VH3-CH1-CL; VH3-VH4-VL4-VL3-CH1-CL; VL3-VL4-VH4-VH3-CL-CH1; VH3-VH4-VL4-VL3-CL-CH1; VL3-L6-VL4-L7-VH4-L8-VH3-L9-CH1; VH3-L6-VH4-L7-VL4-L8-VL3-L9-CH1; VL3-L6-VL4-L7-VH4-L8-VH3-L9-CL; VH3-L6-VH4-L7-VL4-L8-VL3-L9-CL; VL3-L6-VL4-L7-VH4-L8-VH3-L9-CH1-L10-CL; VH3-L6-VH4-L7-VL4-L8-VL3-L9-CH1-L10-CL; VL3-L6-VL4-L7-VH4-L8-VH3-L9-CL-L10-CH1; or VH3-L6-VH4-L7-VL4-L8-VL3-L9-CL-L10-CH1; wherein VL1 is a first immunoglobulin light chain variable region that specifically binds to an HIV protein; VL2 is a second immunoglobulin light chain variable region that specifically binds to an HIV protein; VL3 is a third immunoglobulin light chain variable region that specifically binds to an HIV protein; VL4 is a fourth immunoglobulin light chain variable region that specifically binds to an HIV protein; VH1 is a first immunoglobulin heavy chain variable region that specifically binds to an HIV protein; VH2 is a second immunoglobulin heavy chain variable region that specifically binds to an HIV protein; VH3 is a third immunoglobulin heavy chain variable region that specifically binds to an HIV protein; VH4 is a fourth immunoglobulin heavy chain variable region that specifically binds to an HIV protein; CH1 is an immunoglobulin heavy chain constant region 1; CL is an immunoglobulin light chain constant region; and L1, L2, L3, L4, L5, L6, L7, L8, L9 and L10 are amino acid linkers.

In other aspects, the invention is directed to an antigen binding polypeptide complex comprising a first polypeptide and a second polypeptide; wherein the first polypeptide has a structure represented by VL1-VL2-VH2-VH1-CH1-Fc; VH1-VH2-VL2-VL1-CH1-Fc; VL1-VL2-VH2-VH1-CL-Fc; VH1-VH2-VL2-VL1-CL-Fc; VL1-VL2-VH2-VH1-CH1-CL-Fc; VH1-VH2-VL2-VL1-CH1-CL-Fc; VL1-VL2-VH2-VH1-CL-CH1-Fc; VH1-VH2-VL2-VL1-CL-CH1-Fc; VL1-L1-VL2-L2-VH2-L3-VH1-L4-CH1-Fc; VH1-L1-VH2-L2-VL2-L3-VL1-L4-CH1-Fc; VL1-L1-VL2-L2-VH2-L3-VH1-L4-CL-Fc; VH1-L1-VH2-L2-VL2-L3-VL1-L4-CL-Fc; VL1-L1-VL2-L2-VH2-L3-VH1-L4-CH1-L5-CL-Fc; VH1-L1-VH2-L2-VL2-L3-VL1-L4-CH1-L5-CL-Fc; VL1-L1-VL2-L2-VH2-L3-VH1-L4-CL-L5-CH1-Fc; or VH1-L1-VH2-L2-VL2-L3-VL1-L4-CL-L5-CH1-Fc; wherein the second polypeptide has a structure represented by VL3-VL4-VH4-VH3-CH1-Fc; VH3-VH4-VL4-VL3-CH1-Fc; VL3-VL4-VH4-VH3-CL-Fc; VH3-VH4-VL4-VL3-CL-Fc; VL3-VL4-VH4-VH3-CH1-CL-Fc; VH3-VH4-VL4-VL3-CH1-CL-Fc; VL3-VL4-VH4-VH3-CL-CH1-Fc; VH3-VH4-VL4-VL3-CL-CH1-Fc; VL3-L6-VL4-L7-VH4-L8-VH3-L9-CH1-Fc; VH3-L6-VH4-L7-VL4-L8-VL3-L9-CH1-Fc; VL3-L6-VL4-L7-VH4-L8-VH3-L9-CL-Fc; VH3-L6-VH4-L7-VL4-L8-VL3-L9-CL-Fc; VL3-L6-VL4-L7-VH4-L8-VH3-L9-CH1-L10-CL-Fc; VH3-L6-VH4-L7-VL4-L8-VL3-L9-CH1-L10-CL-Fc; VL3-L6-VL4-L7-VH4-L8-VH3-L9-CL-L10-CH1-Fc; or VH3-L6-VH4-L7-VL4-L8-VL3-L9-CL-L10-CH1-Fc; wherein VL1 is a first immunoglobulin light chain variable region that specifically binds to an HIV protein; VL2 is a second immunoglobulin light chain variable region that specifically binds to an HIV protein; VL3 is a third immunoglobulin light chain variable region that specifically binds to an HIV protein; VL4 is a fourth immunoglobulin light chain variable region that specifically binds to an HIV protein; VH1 is a first immunoglobulin heavy chain variable region that specifically binds to an HIV protein; VH2 is a second immunoglobulin heavy chain variable region that specifically binds to an HIV protein; VH3 is a third immunoglobulin heavy chain variable region that specifically binds to an HIV protein; VH4 is a fourth immunoglobulin heavy chain variable region that specifically binds to an HIV protein; Fc is a region comprising an immunoglobulin heavy chain constant region 2 (CH2), an immunoglobulin heavy chain constant region 3 (CH3), and optionally, an immunoglobulin hinge; CH1 is an immunoglobulin heavy chain constant region 1; CL is an immunoglobulin light chain constant region; and L1, L2, L3, L4, L5, L6, L7, L8, L9, L10, L11 and L12 are amino acid linkers.

In other aspects, the invention is directed to an antigen binding polypeptide complex comprising a first polypeptide and a second polypeptide; wherein the first polypeptide has a structure represented by VL1-VL2-VH2-VH1; VH1-VH2-VL2-VL1; VL1-VL2-VH2-VH1-Fc; VH1-VH2-VL2-VL1-Fc; VL1-VL2-VH2-VH1-CH1; VH1-VH2-VL2-VL1-CH1; VL1-VL2-VH2-VH1-CL; VH1-VH2-VL2-VL1-CL; VL1-VL2-VH2-VH1-CH1-CL; VH1-VH2-VL2-VL1-CH1-CL; VL1-VL2-VH2-VH1-CL-CH1; VH1-VH2-VL2-VL1-CL-CH1; VL1-VL2-VH2-VH1-CH1-Fc; VH1-VH2-VL2-VL1-CH1-Fc; VL1-VL2-VH2-VH1-CL-Fc; VH1-VH2-VL2-VL1-CL-Fc; VL1-VL2-VH2-VH1-CH1-CL-Fc; VH1-VH2-VL2-VL1-CH1-CL-Fc; VL1-VL2-VH2-VH1-CL-CH1-Fc; VH1-VH2-VL2-VL1-CL-CH1-Fc; VL1-L1-VL2-L2-VH2-L3-VH1; VH1-L1-VH2-L2-VL2-L3-VL1; VL1-L1-VL2-L2-VH2-L3-VH1-Fc; VH1-L1-VH2-L2-VL2-L3-VL1-Fc; VL1-L1-VL2-L2-VH2-L3-VH1-L4-Fc; VH1-L1-VH2-L2-VL2-L3-VL1-L4-Fc; VL1-L1-VL2-L2-VH2-L3-VH1-L4-CH1; VH1-L1-VH2-L2-VL2-L3-VL1-L4-CH1; VL1-L1-VL2-L2-VH2-L3-VH1-L4-CL; VH1-L1-VH2-L2-VL2-L3-VL1-L4-CL; VL1-L1-VL2-L2-VH2-L3-VH1-L4-CH1-L5-CL; VH1-L1-VH2-L2-VL2-L3-VL1-L4-CH1-L5-CL; VL1-L1-VL2-L2-VH2-L3-VH1-L4-CL-L5-CH1; VH1-L1-VH2-L2-VL2-L3-VL1-L4-CL-L5-CH1; VL1-L1-VL2-L2-VH2-L3-VH1-L4-CH1-Fc; VH1-L1-VH2-L2-VL2-L3-VL1-L4-CH1-Fc; VL1-L1-VL2-L2-VH2-L3-VH1-L4-CL-Fc; VH1-L1-VH2-L2-VL2-L3-VL1-L4-CL-Fc; VL1-L1-VL2-L2-VH2-L3-VH1-L4-CH1-L5-CL-Fc; VH1-L1-VH2-L2-VL2-L3-VL1-L4-CH1-L5-CL-Fc; VL1-L1-VL2-L2-VH2-L3-VH1-L4-CL-L5-CH1-Fc; or VH1-L1-VH2-L2-VL2-L3-VL1-L4-CL-L5-CH1-Fc; wherein the second polypeptide has a structure represented by VL3-VL4-VH4-VH3; VH3-VH4-VL4-VL3; VL3-VL4-VH4-VH3-Fc; VH3-VH4-VL4-VL3-Fc; VL3-VL4-VH4-VH3-CH1; VH3-VH4-VL4-VL3-CH1; VL3-VL4-VH4-VH3-CL; VH3-VH4-VL4-VL3-CL; VL3-VL4-VH4-VH3-CH1-CL; VH3-VH4-VL4-VL3-CH1-CL; VL3-VL4-VH4-VH3-CL-CH1; VH3-VH4-VL4-VL3-CL-CH1; VL3-VL4-VH4-VH3-CH1-Fc; VH3-VH4-VL4-VL3-CH1-Fc; VL3-VL4-VH4-VH3-CL-Fc; VH3-VH4-VL4-VL3-CL-Fc; VL3-VL4-VH4-VH3-CH1-CL-Fc; VH3-VH4-VL4-VL3-CH1-CL-Fc; VL3-VL4-VH4-VH3-CL-CH1-Fc; VH3-VH4-VL4-VL3-CL-CH1-Fc; VL3-L6-VL4-L7-VH4-L8-VH3; VH3-L6-VH4-L7-VL4-L8-VL3; VL3-L6-VL4-L7-VH4-L8-VH3-Fc; VH3-L6-VH4-L7-VL4-L8-VL3-Fc; VL3-L6-VL4-L7-VH4-L8-VH3-L9-Fc; VH3-L6-VH4-L7-VL4-L8-VL3-L9-Fc; VL3-L6-VL4-L7-VH4-L8-VH3-L9-CH1; VH3-L6-VH4-L7-VL4-L8-VL3-L9-CH1; VL3-L6-VL4-L7-VH4-L8-VH3-L9-CL; VH3-L6-VH4-L7-VL4-L8-VL3-L9-CL; VL3-L6-VL4-L7-VH4-L8-VH3-L9-CH1-L10-CL; VH3-L6-VH4-L7-VL4-L8-VL3-L9-CH1-L10-CL; VL3-L6-VL4-L7-VH4-L8-VH3-L9-CL-L10-CH1; VH3-L6-VH4-L7-VL4-L8-VL3-L9-CL-L10-CH1; VL3-L6-VL4-L7-VH4-L8-VH3-L9-CH1-Fc; VH3-L6-VH4-L7-VL4-L8-VL3-L9-CH1-Fc; VL3-L6-VL4-L7-VH4-L8-VH3-L9-CL-Fc; VH3-L6-VH4-L7-VL4-L8-VL3-L9-CL-Fc; VL3-L6-VL4-L7-VH4-L8-VH3-L9-CH1-L10-CL-Fc; VH3-L6-VH4-L7-VL4-L8-VL3-L9-CH1-L10-CL-Fc; VL3-L6-VL4-L7-VH4-L8-VH3-L9-CL-L10-CH1-Fc; VH3-L6-VH4-L7-VL4-L8-VL3-L9-CL-L10-CH1-Fc; VL3-L6-VL4-L7-VH4-L8-VH3-L9-CH1-L10-Fc; VH3-L6-VH4-L7-VL4-L8-VL3-L9-CH1-L10-Fc; VL3-L6-VL4-L7-VH4-L8-VH3-L9-CL-L10-Fc; VH3-L6-VH4-L7-VL4-L8-VL3-L9-CL-L10-Fc; VL3-L6-VL4-L7-VH4-L8-VH3-L9-CH1-L10-CL-L11-Fc; VH3-L6-VH4-L7-VL4-L8-VL3-L9-CH1-L10-CL-L11-Fc; VL3-L6-VL4-L7-VH4-L8-VH3-L9-CL-L10-CH1-L11-Fc; or VH3-L6-VH4-L7-VL4-L8-VL3-L9-CL-L10-CH1-L11-Fc; wherein VL1 is a first immunoglobulin light chain variable region that specifically binds to an HIV protein; VL2 is a second immunoglobulin light chain variable region that specifically binds to an HIV protein; VL3 is a third immunoglobulin light chain variable region that specifically binds to an HIV protein; VL4 is a fourth immunoglobulin light chain variable region that specifically binds to an HIV protein; VH1 is a first immunoglobulin heavy chain variable region that specifically binds to an HIV protein; VH2 is a second immunoglobulin heavy chain variable region that specifically binds to an HIV protein; VH3 is a third immunoglobulin heavy chain variable region that specifically binds to an HIV protein; VH4 is a fourth immunoglobulin heavy chain variable region that specifically binds to an HIV protein; Fc is a region comprising an immunoglobulin heavy chain constant region 2 (CH2), an immunoglobulin heavy chain constant region 3 (CH3), and optionally, an immunoglobulin hinge; CH1 is an immunoglobulin heavy chain constant region 1; CL is an immunoglobulin light chain constant region; and L1, L2, L3, L4, L5, L6, L7, L8, L9, L10 or L11 are amino acid linkers.

In some aspects of an antigen binding polypeptide or antigen binding polypeptide complex of the invention, VH1, VH2, VH3 and VH4 each comprise a heavy chain variable region from the PGT121, VRC01, 10E8v4 or PG16 antibody or a variant thereof, and/or VL1, VL2, VL3 and VL4 each comprise a light chain variable region from the PGT121, VRC01, 10E8v4 or PG16 antibody or a variant thereof.

In some aspects, VH1, VH2, VH3 and VH4 of an antigen binding polypeptide or antigen binding polypeptide complex of the invention each comprise an amino acid sequence having at least 90% identity, at least 95% identity or 100% identity to any one of SEQ ID NOs:20-23; and/or VL1, VL2, VL3 and VL4 each comprise an amino acid sequence having at least 90% identity, at least 95% identity or 100% identity to any one of SEQ ID NOs:24-27.

In some aspects, VH1, VH2, VH3 and VH4 of an antigen binding polypeptide or antigen binding polypeptide complex of the invention each comprise a CDR1 having an amino acid sequence with at least 90% identity, at least 95% identity or 100% identity to any one of SEQ ID NOs:60, 63, 66 and 69; a CDR2 having an amino acid sequence with at least 90% identity, at least 95% identity or 100% identity to any one of SEQ ID NOs:61, 64, 67 and 70; and a CDR3 having an amino acid sequence with at least 90% identity, at least 95% identity or 100% identity to any one of SEQ ID NOs:62, 65, 68 and 71; and VL1, VL2, VL3 and VL4 each comprise a CDR1 having an amino acid sequence with at least 90% identity, at least 95% identity or 100% identity to any one of SEQ ID NOs:72, 75, 78 and 81; a CDR2 having an amino acid sequence with at least 90% identity, at least 95% identity or 100% identity to any one of SEQ ID NOs:73, 76, 79 and 82; and a CDR3 having an amino acid sequence with at least 90% identity, at least 95% identity or 100% identity to any one of SEQ ID NOs:74, 77, 80 and 83.

### Other General Aspects

The antigen binding polypeptides and antigen binding polypeptide complexes described herein specifically bind to an HIV protein. This includes specific binding to one or more HIV proteins and specific binding to one or more epitopes on the same HIV protein. In some aspects, the HIV protein is selected from the group consisting of an HIV envelope protein, an HIV structural protein, an HIV functional protein, or an HIV accessory protein. In some aspects, the HIV envelope protein is HIV envelope glycoprotein (Env), HIV envelope glycoprotein gp160, HIV envelope surface glycoprotein gp120, or HIV transmembrane envelope protein gp41. In some aspects, the HIV structural protein is p17, p24, p7 or p55. In some aspects, the HIV functional protein is p66, HIV-1 protease (PR) or p31. In some aspects, the HIV accessory protein is Nef, Tat, Rev, Vif, Vpr or Vpu.

Antigen binding sequences (e.g., CDR, VH, VL, heavy chain and light chain sequences from antibodies) for HIV proteins are well known. Such antibodies include, but are not limited to PGT145, PG9, PG16, PGT128, PGT121, 10-1074, 3BNC117, VRC01, PGT151, 4E10, 10E8, or a variant thereof (e.g., 10E8v4). In addition, molecular biology and recombinant DNA methods for making, screening and engineering antigen binding complexes and antibodies containing such sequences are well known and described, for example, in Adair et al. Human Antibodies, 5(1-2):41-47, 1994; Kostelny et al., J. Immunol., 148(5):1547-1553 (1992), Shiraiwa et al., Methods, 154:10-20, 2019; and Zola, “Monoclonal Antibodies: A Manual of Techniques,” 1987, 1st Ed., CRC Press; and Steinitz, Human Antibodies, 18(1-2):1-10, 2009.

In some aspects, an antigen binding polypeptide or antigen binding polypeptide complex of the invention does not specifically bind to an antigen associated with severe acute respiratory syndrome (SARS).

In some aspects, one or more of VH1, VH2, VH3, VH4, VH5 and VH6 of an antigen binding polypeptide or antigen binding polypeptide complex of the invention comprises an amino acid sequence encoded by a polynucleotide having at least 90% identity, at least 95% identity or 100% identity to SEQ ID NO:28 or 29; and/or one or more of VL1, VL2, VL3, VL4, VL5 and VL6 of an antigen binding polypeptide or antigen binding polypeptide complex of the invention comprises an amino acid sequence encoded by a polynucleotide having at least 90% identity, at least 95% identity or 100% identity to SEQ ID NO:30 or 31.

In some aspects, the invention is directed to an antigen binding polypeptide or antigen binding polypeptide complex (e.g., antibody or antigen binding fragment thereof) comprising one or more amino acid sequences having at least 90% identity, at least 95% identity, or 100% identity to any one of SEQ ID NOs:32-47, 84, 86, 88, 90, 92, 94, 96 and 98.

In other aspects, the invention is directed to an antigen binding polypeptide or antigen binding polypeptide complex (e.g., antibody or antigen binding fragment thereof) comprising one or more amino acid sequences encoded by a polynucleotide having at least 90% identity, at least 95% identity, or 100% identity to any one of SEQ ID NOs:48-59, 85, 87, 89, 91, 93, 95, 97 and 99.

In some aspects, an antigen binding polypeptide or antigen binding polypeptide complex (e.g., an antibody or antigen binding fragment thereof) comprises an immunoglobulin hinge. In some aspects, the immunoglobulin hinge comprises an upper hinge region, a middle hinge region, a lower hinge region, or a combination thereof.

As used herein, an antigen binding polypeptide, antigen binding polypeptide complex (e.g., an antibody or antigen binding fragment thereof), or region or domain thereof that “specifically binds” refers to its association with an epitope by its antigen binding domain, and that the binding entails some complementarity between the antigen binding domain and the epitope. Specific binding to an epitope occurs where there is binding to that epitope via its antigen binding domain more readily than there would be binding to a random, unrelated epitope.

As used herein, an “epitope” refers to a localized region of an antigen to which an antigen binding polypeptide or antigen binding polypeptide complex (e.g., antibody or antigen binding fragment thereof) can specifically bind. An epitope can be, for example, contiguous amino acids of a polypeptide (linear or contiguous epitope) or an epitope can, for example, come together from two or more non-contiguous regions of a polypeptide or polypeptides (conformational, non-linear, discontinuous, or non-contiguous epitope). In some aspects, the epitope to which an antibody or antigen-binding fragment thereof binds can be determined by, e.g., NMR spectroscopy, X-ray diffraction crystallography studies, ELISA assays, hydrogen/deuterium exchange coupled with mass spectrometry (e.g., liquid chromatography electrospray mass spectrometry), array-based oligo-peptide scanning assays, and/or mutagenesis mapping (e.g., site-directed mutagenesis mapping). See, e.g., Giege R et al., (1994) Acta Crystallogr. D Biol. Crystallogr. 50(Pt 4): 339-350; McPherson A (1990) Eur. J. Biochem. 189: 1-23; Chayen N E (1997) Structure 5: 1269-1274; McPherson A (1976) J. Biol. Chem. 251: 6300-6303; Meth. Enzymol. (1985) volumes 114 & 115, eds Wyckoff H W et al., U.S. Pub. No. 2004/0014194), Bricogne G (1993) Acta Crystallogr. D Biol. Crystallogr. 49(Pt 1): 37-60, Bricogne G (1997) Meth. Enzymol. 276A: 361-423, ed Carter C W, and Roversi et al., (2000) Acta Crystallogr. D Biol. Crystallogr. 56(Pt 10): 1316-1323 (X-ray diffraction crystallography studies); and Champe et al., (1995) J. Biol. Chem. 270: 1388-1394 and Cunningham B C & Wells J A (1989) Science 244: 1081-1085 (mutagenesis mapping).

Specific binding can be represented by a “binding affinity.” Binding affinity refers to an intrinsic binding affinity which reflects a 1:1 interaction between members of a binding pair (e.g., an antigen binding polypeptide or antigen binding polypeptide complex and an antigen). Binding affinity can be measured and/or expressed in several ways known in the art, including, but not limited to, equilibrium dissociation constant (K_{D}). K_{D} is calculated from the quotient of k_{off}/kₒₙ, where kₒₙ refers to the association rate constant of, e.g., an antigen binding polypeptide or antigen binding polypeptide complex to an antigen, and k_{off} refers to the dissociation of, e.g., an antigen binding polypeptide or antigen binding polypeptide complex from an antigen. The kₒₙ and k_{off} can be determined by techniques known to one of ordinary skill in the art, such as Octet BLI, BIAcore® or KinExA.

Accordingly, in some aspects, an antigen binding polypeptide complex of the invention is an antibody or antigen binding fragment thereof. In some aspects, the antibody or antigen binding fragment thereof comprises one, two or three antigen binding polypeptides described herein. In some aspects, the antibody or antigen binding fragment thereof is bispecific, trispecific, tetraspecific, pentaspecific or hexaspecific. In other aspects, the antibody or antigen binding fragment thereof is bivalent, trivalent, tetravalent, pentavalent or hexavalent.

In some aspects, the antibody or antigen binding fragment thereof specifically binds to an antigen with an equilibrium dissociation constant (K_{D}) of from about 10 μM to about 1 μM. In some aspects, the antibody is IgG, IgM, IgE, IgA or IgD. In some aspects, the IgG is IgG1, IgG2, IgG3 or IgG4. In some aspects, the antigen binding fragment is a Fab, scFab, Fab′, F(ab′)2, Fv or scFv. In yet another aspect, the antibody is human or humanized.

### Amino Acid Linkers

In some aspects, an antigen binding polypeptide or antigen binding polypeptide complex (e.g., an antibody or antigen binding fragment thereof) of the invention comprises one or more amino acid linkers between one or more regions of the antigen binding polypeptide or antigen binding polypeptide complex.

As used herein, an “amino acid linker” refers to a single amino acid or short amino acid sequence that is capable of joining two polypeptide regions of the invention described herein in a stable manner that maintains or promotes a function associated with the polypeptide regions. In some aspects, an amino acid linker is represented herein in a structure of an antigen binding polypeptide or antigen binding polypeptide complex by the abbreviation “l” or “L” and a number (e.g., L1 to denote a first linker, L2 to denote a second linker, L3 to denote a third linker, L4 to denote a fourth linker, L5 to denote a fifth linker, L6 to denote a sixth linker, L7 to denote a seventh linker, L8 to denote an eighth linker, L9 to denote a ninth linker, L10 to denote a tenth linker, L11 to denote an eleventh linker, L12 to denote a twelfth linker, L13 to denote a thirteenth linker, and so on). In some aspects, such enumerated amino acid linkers (e.g., L1) can have the same or different sequence as any other enumerated amino acid linker (e.g., L2, etc.). Furthermore, in other aspects, an enumerated amino acid linker present in one polypeptide (e.g., L1 on a first polypeptide of an antigen binding polypeptide and/or antigen binding polypeptide complex structure described herein) can have the same or different sequence as the same enumerated amino acid linker present in another polypeptide (e.g., L1 on a second polypeptide, third polypeptide, etc. of an antigen binding polypeptide and/or antigen binding polypeptide complex structure described herein).

In some aspects, an amino acid linker has a length of from about 1 amino acid to about 50 amino acids (e.g., one or more of L1, L2, L3, L4, L5, L6, L7, L8, L9, L10, L11, L12, L13 etc. of an antigen binding polypeptide or a first, second, third, etc. polypeptide of an antigen binding polypeptide complex structure described herein). In some aspects, the amino acid linker has a length of from about 1 amino acid to about 45 amino acids, about 1 amino acid to about 40 amino acids, about 1 amino acid to about 35 amino acids, about 1 amino acid to about 30 amino acids, about 1 amino acid to about 25 amino acids, about 1 amino acid to about 20 amino acids, 1 amino acid to about 15 amino acids, about 1 amino acid to about 10 amino acids, about 1 amino acid to about 5 amino acids, about 5 amino acids to about 50 amino acids, about 5 amino acids to about 45 amino acids, about 5 amino acids to about 40 amino acids, about 5 amino acids to about 35 amino acids, about 5 amino acids to about 30 amino acids, about 5 amino acids to about 25 amino acids, about 5 amino acids to about 20 amino acids, about 5 amino acids to about 15 amino acids, about 5 amino acids to about 10 amino acids, about 10 amino acids to about 50 amino acids, about 10 amino acids to about 45 amino acids, about 10 amino acids to about 40 amino acids, about 10 amino acids to about 35 amino acids, about 10 amino acids to about 30 amino acids, about 10 amino acids to about 25 amino acids, about 10 amino acids to about 20 amino acids, about 10 amino acids to about 15 amino acids, about 15 amino acids to about 50 amino acids, about 15 amino acids to about 45 amino acids, about 15 amino acids to about 40 amino acids, about 15 amino acids to about 35 amino acids, about 15 amino acids to about 30 amino acids, about 15 amino acids to about 25 amino acids, about 15 amino acids to about 20 amino acids, about 20 amino acids to about 50 amino acids, about 20 amino acids to about 45 amino acids, about 20 amino acids to about 40 amino acids, about 20 amino acids to about 35 amino acids, about 20 amino acids to about 30 amino acids, about 20 amino acids to about 25 amino acids, about 25 amino acids to about 50 amino acids, about 25 amino acids to about 45 amino acids, about 25 amino acids to about 40 amino acids, about 25 amino acids to about 35 amino acids, about 25 amino acids to about 30 amino acids, about 30 amino acids to about 50 amino acids, about 30 amino acids to about 45 amino acids, about 30 amino acids to about 40 amino acids, about 30 amino acids to about 35 amino acids, about 40 amino acids to about 50 amino acids, about 40 amino acids to about 45 amino acids, or about 45 amino acids to about 50 amino acids.

In some aspects, the amino acid linker has about 1, about 2, about 3, about 4, about 5, about 6, about 7, about 8, about 9, about 10, about 11, about 12, about 13, about 14, about 15, about 16, about 17, about 18, about 19, about 20, about 25, about 30, about 35, about 40, about 45, or about 50 amino acids (e.g., one or more of L1, L2, L3, L4, L5, L6, L7, L8, L9, L10, L11, L12, L13, etc. of an antigen binding polypeptide structure described herein or a first, second, third, etc. polypeptide of an antigen binding polypeptide complex structure described herein).

In some aspects, the amino acid linker consists of one or more amino acid residues (e.g., one or more of L1, L2, L3, L4, L5, L6, L7, L8, L9, L10, L11, L12, L13, etc. of an antigen binding polypeptide structure described herein or a first, second, third, etc. polypeptide of an antigen binding polypeptide complex structure described herein). In some aspects, the amino acid residues are selected from the group consisting of glycine, alanine, serine, threonine, cysteine, asparagine, glutamine, leucine, isoleucine, valine, proline, histidine, aspartic acid, glutamic acid, lysine, arginine, methionine, phenylalanine, tryptophan, and tyrosine.

In some aspects, an amino acid linker of the invention is non-immunogenic. In some aspects, the non-immunogenic linker consists of serine, glycine and/or alanine residues, or consists of serine and/or glycine residues. In some aspects, an amino acid linker of the invention does not contain a T cell epitope or consensus T cell epitope.

In some aspects, the amino acid linker consists of one or more residues of alanine, cysteine, glycine, isoleucine, leucine, methionine, phenylalanine, proline, tryptophan, tyrosine, valine (e.g., one or more of L1, L2, L3, L4, L5, L6, L7, L8, L9, L10, L11, L12, L13, etc. of an antigen binding polypeptide structure described herein or a first, second, third, etc. polypeptide of an antigen binding polypeptide complex structure described herein).

Amino acid linker sequences that can be used with the antigen binding polypeptides and antigen binding polypeptide complexes (e.g., an antibody or antigen binding fragment thereof) of the invention are well known and can be incorporated into antigen binding polypeptides and antigen binding polypeptide complexes of the invention using routine molecular biology and recombinant DNA techniques. See, e.g., Chen et al., Adv Drug Deliv Rev., 65(10):1357-1369, 2013; and Chichili et al., Protein Sci., 22(2):153-167, 2013.

In some aspects, the amino acid linker (e.g., one or more of L1, L2, L3, L4, L5, L6, L7, L8, L9, L10, L11, L12, L13, etc. of a first, second, third, etc. polypeptide of an antigen binding polypeptide or antigen binding polypeptide complex structure described herein) has the sequence of g, a, gss, asg, ggssg, gssgs, gtvaa, asggs, astgg, asggsg, ggsggssgss, sggsgssggs, ggsggsgsgggsasgsg, ggsggsgsggggsasgsg, gggssggggsggsgsggsgs, ggggsggsgsggggsasgsg, gggssggsgsggsgsggsgs, sggssggsgsggsgsggsgssg, gsgssggggsggsgsggsgssg, ggggsgsggsgggssggggsggggsggggsggggsggggs, ggggsggggsggggsggggsggggsggggsggggsggggs, ggggsgsggsgggssggggsggggsggggsggggsggggssss, ggggsgsggsgggssggggsggggsggggsggggsggggssssgs ggsgg, gsggsagsgsggggsasgsg, ggggs, or gsggsggsgsggggsasgsg (SEQ ID NOs:1-19 and 100-107), or a sequence having at least 50%, at least 60%, at least 70%, at least 80%, at least 90% or at least 95% identity to any one of SEQ ID NOs:1-19 and 100-107.

In some aspects of an antigen binding polypeptide or antigen binding polypeptide complex of the invention, L1 comprises the amino acid sequence of ggssg (SEQ ID NO:1) or an amino acid sequence having at least 90% identity or at least 95% identity to SEQ ID NO:1; L2 comprises the amino acid sequence of ggggsggsgsggggsasgsg (SEQ ID NO:12) or an amino acid sequence having at least 90% identity or at least 95% identity to SEQ ID NO:12; L3 comprises the amino acid sequence of SEQ ID NO:1 or an amino acid sequence having at least 90% identity or at least 95% identity to SEQ ID NO:1; and L4 comprises the amino acid sequence of asggsg (SEQ ID NO:6) or an amino acid sequence having at least 90% identity or at least 95% identity to SEQ ID NO:6.

In some aspects, the invention is directed to an antigen binding polypeptide complex comprising a first polypeptide and a second polypeptide; wherein the first polypeptide has a structure represented by VL1-VL2-VH2-VH1; VH1-VH2-VL2-VL1; VL1-L1-VL2-L2-VH2-L3-VH1; or VH1-L1-VH2-L2-VL2-L3-VL1; wherein the second polypeptide has a structure represented by VL3-VL4-VH4-VH3; VH3-VH4-VL4-VL3; VL3-L4-VL4-L5-VH4-L6-VH3; or VH3-L4-VH4-L5-VL4-L6-VL3; wherein VL1 is a first immunoglobulin light chain variable region that specifically binds to an HIV protein; VL2 is a second immunoglobulin light chain variable region that specifically binds to an HIV protein; VL3 is a third immunoglobulin light chain variable region that specifically binds to an HIV protein; VL4 is a fourth immunoglobulin light chain variable region that specifically binds to an HIV protein; VH1 is a first immunoglobulin heavy chain variable region that specifically binds to an HIV protein; VH2 is a second immunoglobulin heavy chain variable region that specifically binds to an HIV protein; VH3 is a third immunoglobulin heavy chain variable region that specifically binds to an HIV protein; VH4 is a fourth immunoglobulin heavy chain variable region that specifically binds to an HIV protein; L1, L2, L3, L4, L5 and L6 are amino acid linkers; L1 comprises the amino acid sequence of ggssg (SEQ ID NO:1) or an amino acid sequence having at least 90% identity or at least 95% identity to SEQ ID NO:1; L2 comprises the amino acid sequence of ggggsggsgsggggsasgsg (SEQ ID NO: 12) or an amino acid sequence having at least 90% identity or at least 95% identity to SEQ ID NO:12; L3 comprises the amino acid sequence of SEQ ID NO:1 or an amino acid sequence having at least 90% identity or at least 95% identity to SEQ ID NO:1; and L4 comprises the amino acid sequence of asggsg (SEQ ID NO:6) or an amino acid sequence having at least 90% identity or at least 95% identity to SEQ ID NO:6.

### Detectable Labels and Drug Conjugates

In some aspects, an antigen binding polypeptide or antigen binding polypeptide complex (e.g., an antibody or antigen binding fragment thereof) of the invention comprises one or more detectable labels. An antigen binding polypeptide or antigen binding polypeptide complex (e.g., an antibody or antigen binding fragment thereof) containing a detectable label is useful in therapeutic, diagnostic, imaging (e.g., radioimaging), or basic research applications.

In some aspects, the detectable label is a radioactive label. Examples of a radioactive label include, but are not limited to, the isotopes ³H, ¹⁴C, ³²P, ³⁵S, ³⁶Cl, ⁵¹Cr, ⁵⁷Co, ⁵⁸Co, ⁵⁹Fe, ⁹⁰Y, ¹²¹I, ¹²⁴I, ¹²⁵I, ¹³¹I, ¹¹¹In, ¹¹⁷Lu, ²¹¹At, ¹⁹⁸Au, ⁶⁷Cu, ²²⁵Ac, ²¹³Bi, ⁹⁹Tc, ¹⁸⁶Re and ⁸⁹Zr.

In some aspects, the detectable label is a chemiluminescent label, fluorescent label, enzyme, biotin, or a combination thereof.

In some aspects, the detectable label is a peptide tag. In some aspects, the peptide tag is located at the N-terminus of the polypeptide or polypeptide complex. In some aspects, the peptide tag is located at the C-terminus of the polypeptide or polypeptide complex. In some aspects, the peptide tag is an affinity tag or fusion tag.

In some aspects, the detectable label is a polyhistidine tag, polyarginine tag, glutathione-S-transferase (GST), maltose binding protein (MBP), chitin binding protein (CBP), Strep-tag, thioredoxin (TRX), poly(NANP), FLAG tag, ALFA-tag, V5-tag, Myc-tag, hemagglutinin (HA) tag, Spot tag, T7 tag, NE tag, or green fluorescence protein (GFP), or a combination thereof. In some aspects, the polyhistidine tag consists of from about 4 to about 10 histidine residues. In some aspects, the polyhistidine tag consists of about 4, about 5, about 6, about 7, about 8, about 9, or about 10 histidine residues.

Additional examples of detectable labels and methods for introducing detectable labels into a polypeptide are known and include routine chemical, molecular biology and recombinant DNA techniques. See, e.g., Hnatowich et al., Science, 220(4597):613-615, 1983; Yao et al., Int. J. Mol. Sci., 17(2):194, 2016; Kimple et al., Curr. Protoc. Protein Sci., 73:Unit 9.9, 2013; Sambrook J, Fritsch E F. Molecular Cloning: A Laboratory Manual. Cold Spring Harbor Laboratory Press; Cold Spring Harbor, N.Y.: 1989; Molecular Cell Biology, 4^{th} edition, Section 3.5, Purifying, Detecting and Characterizing Proteins; and Mahmoodi et al., Cogent Biology, 5(1):DOI: 10/1080/23312025.2019.1665406.

In other aspects, an antigen binding polypeptide or antigen binding polypeptide complex (e.g., an antibody or antigen binding fragment thereof) of the invention is conjugated to an agent as an antibody-drug conjugate (ADC). An ADC of the invention is useful in therapeutic, diagnostic, imaging (e.g., radioimaging), or basic research applications.

In some aspects, an antigen binding polypeptide or antigen binding polypeptide complex (e.g., an antibody or antigen binding fragment thereof) of the invention is conjugated to a cytotoxic agent, immunomodulating agent, imaging agent, or therapeutic protein, typically via a linker. The linker can comprise a cleavable unit or can be non-cleavable. Cleavable units include, for example, disulfide containing linkers that are cleavable through disulfide exchange, acid-labile linkers that are cleavable at acidic pH, and linkers that are cleavable by hydrolases, esterases, peptidases, and glucoronidases (e.g., peptide linkers and glucoronide linkers). Non-cleavable linkers are believed to release drug via a proteolytic antibody degradation mechanism.

Methods for making an ADC are known and include, but are not limited to, conjugation via thiols, amides, aldehydes, or azides, as well as other routine chemical, molecular biology and recombinant DNA techniques. See, e.g., Yao et al., Int. J. Mol. Sci., 17(2):194, 2016; Sambrook J, Fritsch E F. Molecular Cloning: A Laboratory Manual. Cold Spring Harbor Laboratory Press; Cold Spring Harbor, N.Y.: 1989; Molecular Cell Biology, 4^{th} edition, Section 3.5, Purifying, Detecting and Characterizing Proteins; and Mahmoodi et al., Cogent Biology, 5(1):DOI: 10/1080/23312025.2019.1665406.

### Modifications

In some aspects, the invention is directed to an antigen binding polypeptide or antigen binding polypeptide complex (e.g., an antibody or antigen binding fragment thereof) comprising an effector function mutation or half-life extension mutation.

Effector functions are an important part of the humoral immune response and form an link between innate and adaptive immunity. Most effector functions are induced via the Fc region of an antibody, which can interact with complement proteins and specialized Fc receptors. As used herein, an “effector function mutation,” refers to a change in the amino acid sequence, typically in the Fc region, which can increase or decrease effector function, for example, increase binding affinity of Fc for specific Fc receptors, or increase antibody-dependent cellular cytotoxicity (ADCC) activity.

“Half-life” of a pharmaceutically active substance is the time it takes for the amount of the substance, once administered to the body, to reduce by half A “half-life extension mutation” of an antigen binding polypeptide or antigen binding polypeptide complex of the invention refers to a change in the amino acid sequence, typically in the Fc region, which increases the half-life of the antigen binding polypeptide or antigen binding polypeptide complex (e.g., by increasing Fc receptor binding affinity, slowing off-rate for Fc and Fc receptors, and/or increased sialylation).

Examples of effector function mutations that increase function include, but are not limited to, the following substitutions in the Fc region, based on the EU numbering scheme: S298A/E333A/K334A, S239D/I332E, S239D/A330L/I332E, and G236A/S239D/I332E. Examples of effector function mutations that decrease function include, but are not limited to, the following substitutions in the Fc region, based on the EU numbering scheme: N297A and L234A/L235A. Additional examples of effector function mutations, half-life extension mutations and methods for incorporating the same into an amino acid sequence are known and described, for example, in Saunders, “Conceptual Approaches to Modulating Antibody Effector Functions and Circulation Half-Life,” Front. Immunol. Jun. 7, 2019.

In some aspects, the invention is directed to an antigen binding polypeptide or antigen binding polypeptide complex (e.g., an antibody or antigen binding fragment thereof) comprising one or more knob-into-hole modifications.

The term “knob-into-hole modification” as used herein, refers to a genetic modification that directs the pairing of two polypeptides to promote heterodimerization. In some aspects, the modification introduces a protuberance (knob) into one polypeptide and a cavity (hole) into the other polypeptide at an interface in which the two polypeptides interact. In some aspects, a knob-into-hole modification can be created by introducing only a hole modification, for example, by replacing an amino acid residue with a smaller side chain than the original amino acid residue (e.g., a substitution of one or more serine, threonine, valine or alanine residues, or a combination thereof). In yet another aspect, a knob-into-hole modification can be created by introducing only a knob modification, for example, by replacing an amino acid residue with a larger side chain than the original amino acid residue (e.g., a substitution of one or more tryptophan or tyrosine residues, or a combination thereof).

In some aspects, the knob-into-hole modification is in the binding interface of two Fc regions, the binding interface of two CH2 regions, the binding interface of two CH3 regions, the binding interface of a CL region and a CH1 region, or the binding interface of a VH region and a VL region. See, e.g., U.S. Pub. No. 2007/0178552, Int'l Pub. No. WO 96/027011, Int'l Pub. No. WO 98/050431 and Zhu et al., Protein Science 6:781-788, 1987.

In some aspects, the antigen binding polypeptide or antigen binding polypeptide complex comprises one, two, three, four, five, six, seven, eight, nine, ten, or more knob-into-hole modifications.

Knob-into-hole modifications are well known and can be incorporated into the antigen binding polypeptides and antigen binding polypeptide complexes of the invention using routine molecular biology and recombinant DNA techniques. See, e.g., U.S. Pub. No. 2003/0078385; Int'l Pub. No. WO 96/027011; Ridgway et al., Protein Eng., 9:617-621, 1996; and Merchant et al., Nat. Biotechnol., 16:677-681, 1998.

In some aspects, the knob-into-hole modification is an amino acid substitution. As used herein, such a substitution is described based on the EU numbering scheme of Kabat, which corresponds to the numbering in the Protein Data Bank (PDB).

In some aspects, the knob-into-hole modification is a knob substitution of S354C and/or T366W, based on the EU numbering scheme.

In some aspects, the knob-into-hole modification is a hole substitution of Y349C, T366S, L368A, Y407V, L234A, L235A, P239A, M428L, N433S, M252Y, S254T, T256E, or any combination thereof, based on the EU numbering scheme.

In some aspects, the knob-into-hole modifications are hole substitutions of Y349C, T366S, L368A and Y407V, based on the EU numbering scheme. In some aspects, the knob-into-hole modifications are a hole substitutions of L234A, L235A and P239A, based on the EU numbering scheme. In some aspects, the knob-into-hole modifications are hole substitutions of L234A and L235A, based on the EU numbering scheme. In some aspects, the knob-into-hole modifications are hole substitutions of M428L and N433S, based on the EU numbering scheme. In some aspects, the knob-into-hole modifications are hole substitutions of M252Y, S254T and T256E, based on the EU numbering scheme.

In some aspects, an antigen binding polypeptide complex is an IgG1 or IgG4 antibody and the knob-into-hole modifications are knob substitutions of S354C and T366W and hole substitutions of Y349C, T366S, L368A and Y407V.

In some aspects, the antigen binding polypeptide complex is an IgG1 or IgG4 antibody and the knob-into-hole modifications are hole substitutions of L234A, L235A and P239A.

In some aspects, the antigen binding polypeptide complex is an IgG1 or IgG4 antibody and the knob-into-hole modifications are hole substitutions of L234A and L235A.

In some aspects, the antigen binding polypeptide complex is an IgG1 or IgG4 antibody and the knob-into-hole modifications are hole substitutions of M428L and N433S.

In some aspects, the antigen binding polypeptide complex is an IgG1 or IgG4 antibody and the knob-into-hole modifications are hole substitutions of M252Y, S254T and T256E.

### Chimeric Antigen Receptors

In some aspects of the invention, the antigen binding polypeptides and antigen binding polypeptide complexes can be used in chimeric antigen receptor (CAR) therapy. In some aspects, the invention is directed to a CAR comprised of an antigen binding polypeptide or antigen binding polypeptide complex of the invention. In some aspects, a CAR of the invention comprises an antigen binding polypeptide or antigen binding polypeptide complex of the invention and a transmembrane region. In some aspects, a CAR of the invention comprises an antigen binding polypeptide or antigen binding polypeptide complex of the invention, a transmembrane region, and an intracellular region. In some aspects, the intracellular region is comprised of a costimulatory region and/or an intracellular signal transduction region. In some aspects, the intracellular region is a T cell activation domain. In yet another aspect, the antigen binding polypeptide or antigen binding polypeptide complex of the invention is joined to the transmembrane region by an immunoglobulin hinge.

### Polypeptides, Polynucleotides, Vectors, Cells, and Protein Production Methods

In some aspects, the invention is directed to a polypeptide encoding an antigen binding polypeptide or antigen binding polypeptide complex (e.g., an antibody or antigen binding fragment thereof) described herein.

In other aspects, the invention is directed to a polypeptide comprising an amino acid sequence of one or more of SEQ ID NOs:32-47, 84, 86, 88, 90, 92, 94, 96 and 98, or an amino acid sequence having at least 90% identity or at least 95% identity to one or more of SEQ ID NOs:32-47, 84, 86, 88, 90, 92, 94, 96 and 98.

In other aspects, the invention is directed to a polypeptide comprising an amino acid sequence encoded by one or more of SEQ ID NOs:48-59, 85, 87, 89, 91, 93, 95, 97 and 99, or encoded by a polynucleotide having at least 90% identity or at least 95% identity to one or more of SEQ ID NOs:48-59, 85, 87, 89, 91, 93, 95, 97 and 99.

In some aspects, the invention is directed to a polynucleotide encoding an antigen binding polypeptide or antigen binding polypeptide complex (e.g., an antibody or antigen binding fragment thereof) described herein. In other aspects, the invention is directed to a polynucleotide encoding a CAR described herein. As used herein, a “polynucleotide” includes DNA and RNA (e.g., mRNA).

In other aspects, the invention is directed to a polynucleotide having a sequence of one or more of SEQ ID NOs:48-59, 85, 87, 89, 91, 93, 95, 97 and 99, or a polynucleotide having at least 90% identity or at least 95% identity to one or more of SEQ ID NOs:48-59, 85, 87, 89, 91, 93, 95, 97 and 99.

In other aspects, the invention is directed to a polynucleotide encoding a polypeptide of one or more of SEQ ID NOs:32-47, 84, 86, 88, 90, 92, 94, 96 and 98, or a polynucleotide encoding a polypeptide having at least 90% identity or at least 95% identity to one or more of SEQ ID NOs:32-47, 84, 86, 88, 90, 92, 94, 96 and 98.

In other aspects, the invention is directed to a vector comprising a polynucleotide described herein.

In yet other aspects, the invention is directed to a host cell comprising a polynucleotide or vector described herein.

As used herein, the term “host cell” can be any type of cell, e.g., a primary cell, a cell in culture, or a cell from a cell line. In some aspects, the term “host cell” refers to a cell containing a foreign gene [e.g., a cell subjected to gene delivery or transfected with a polynucleotide (e.g., DNA or mRNA) encoding the gene] and the progeny or potential progeny of such a cell. Progeny of such a cell may not be identical to the parent cell transfected with the nucleic acid molecule, e.g., due to mutations or environmental influences that may occur in succeeding generations or integration of the nucleic acid molecule into the host cell genome.

In other aspects, the invention is directed to an immune cell expressing a CAR of the invention or expressing a polynucleotide or vector encoding a CAR of the invention. In some aspects, the immune cell is a neutrophil, eosinophil, basophil, mast cell, monocyte, macrophage, dendritic cell, natural killer cell, or lymphocyte (B cell or T cell).

Methods which are well known to those skilled in the art can be used to construct vectors encoding antigen binding polypeptides and antigen binding polypeptide complexes (e.g., CDR, VH, VL, heavy chain and/or light chain coding sequences and appropriate transcriptional and translational control signals). These methods include, for example, in vitro recombinant DNA techniques, synthetic techniques, and in vivo genetic recombination.

A vector can be transferred to a host cell by conventional techniques and the resulting cells can then be cultured by conventional techniques to produce an antigen binding polypeptide or antigen binding polypeptide complex comprising, e.g., six CDRs, VH, VL, VH and VL, heavy chain, light chain, or heavy and light chain, or a domain thereof (e.g., one or more CDRs, VH, VL, VH and VL, heavy chain, or light chain). Thus, provided herein are host cells containing a polynucleotide encoding an antigen binding polypeptide or antigen binding polypeptide complex comprising, e.g., comprising six CDRs, VH, VL, VH and VL, heavy chain, light chain, or heavy and light chain, or a domain thereof (e.g., one or more CDRs, VH, VL, VH and VL, heavy chain, or light chain), operably linked to a promoter for expression of such sequences in the host cell. In some aspects, vectors encoding both heavy and light chains, or a domain thereof, individually, can be co-expressed in the host cell for expression. In some aspects, a host cell contains a vector comprising a polynucleotide encoding both a heavy chain and light chain, or a domain thereof. In some aspects, a host cell contains two different vectors, a first vector comprising a polynucleotide encoding a heavy chain or a domain thereof, and a second vector comprising a polynucleotide encoding a light chain or a domain thereof. In some aspects, a first host cell comprises a first vector comprising a polynucleotide encoding a heavy chain or a domain thereof, and a second host cell comprises a second vector comprising a polynucleotide encoding a light chain or a domain thereof. In some aspects, provided herein is a population of host cells comprising such a first host cell and such a second host cell.

In some aspects, provided herein is a population of vectors comprising a first vector comprising a polynucleotide encoding a light chain or domain thereof, and a second vector comprising a polynucleotide encoding a heavy chain or domain thereof. Alternatively, a single vector can be used which encodes, and is capable of expressing, both heavy and light chain polypeptides or a domain thereof.

A variety of host-vector systems can be utilized to express the polypeptides and polypeptide complexes described herein. Such host-vector systems represent vehicles by which the coding sequences of interest can be produced and subsequently purified, but also represent cells which can, when transformed or transfected with the appropriate nucleotide coding sequences, express a polypeptide or polypeptide complex described herein in situ. These include but are not limited to microorganisms such as bacteria (e.g., E. coli and B. subtilis) transformed with recombinant bacteriophage DNA, plasmid DNA or cosmid DNA expression vectors containing antibody coding sequences; yeast (e.g., Saccharomyces pichia) transformed with recombinant yeast expression vectors containing antibody coding sequences; insect cell systems infected with recombinant virus expression vectors (e.g., baculovirus) containing antibody coding sequences; plant cell systems (e.g., green algae such as Chlamydomonas reinhardtii) infected with recombinant virus expression vectors (e.g., cauliflower mosaic virus, CaMV; tobacco mosaic virus, TMV) or transformed with recombinant plasmid expression vectors (e.g., Ti plasmid) containing antibody coding sequences; or mammalian cell systems (e.g., COS (e.g., COS1 or COS), CHO, BHK, MDCK, HEK 293, NS0, PER.C6, VERO, CRL7030, HsS78Bst, HeLa, and NIH 3T3, HEK-293T, HepG2, SP210, R1.1, B-W, L-M, BSC1, BSC40, YB/20, and BMT10 cells) harboring recombinant expression constructs containing promoters derived from the genome of mammalian cells (e.g., metallothionein promoter) or from mammalian viruses (e.g., the adenovirus late promoter; the vaccinia virus 7.5K promoter). In some aspects, cells for expressing polypeptide or polypeptide complexes described herein are CHO cells, for example CHO cells from the CHO GS System™ (Lonza). In some aspects, cells for expressing polypeptides or polypeptide complexes of the invention are human cells, e.g., human cell lines. In some aspects, a mammalian expression vector is pOptiVEC™ or pcDNA3.3. In some aspects, bacterial cells such as Escherichia coli, or eukaryotic cells (e.g., mammalian cells) are used for the expression of recombinant polypeptides. For example, mammalian cells such as Chinese hamster ovary (CHO) cells in conjunction with a vector such as the major intermediate early gene promoter element from human cytomegalovirus is an effective expression system for polypeptides (Foecking M K & Hofstetter H (1986) Gene 45: 101-105; and Cockett M I et al., (1990) Biotechnology 8: 662-667). In some aspects, polypeptides or polypeptide complexes described herein are produced by HEK-293T cells.

In addition, a host cell strain can be chosen which modulates the expression of the inserted sequences, or modifies and processes the gene product in the specific fashion desired. Such modifications (e.g., glycosylation) and processing (e.g., cleavage) of protein products can contribute to the function of the protein. To this end, eukaryotic host cells which possess the cellular machinery for proper processing of the primary transcript, glycosylation, and phosphorylation of the gene product can be used. Such mammalian host cells include but are not limited to CHO, VERO, BHK, Hela, MDCK, HEK 293, NIH 3T3, W138, BT483, Hs578T, HTB2, BT20 and T47D, NS0 (a murine myeloma cell line that does not endogenously produce any immunoglobulin chains), CRL7030, COS (e.g., COS1 or COS), PER.C6, VERO, HsS78Bst, HEK-293T, HepG2, SP210, R1.1, B-W, L-M, BSC1, BSC40, YB/20, BMT10 and HsS78Bst cells.

Once a polypeptide or polypeptide complex described herein has been produced by recombinant expression, it can be purified by any method known in the art for purification of a protein or immunoglobulin molecule, for example, by chromatography (e.g., ion exchange, affinity, particularly by affinity for the specific antigen after Protein A, and size exclusion chromatography), centrifugation, differential solubility, or by any other standard technique for the purification of proteins. Further, the polypeptides or polypeptide complexes described herein can be fused to heterologous polypeptide sequences described herein (e.g., tags) or otherwise known in the art to facilitate purification.

In some aspects, a polypeptide or polypeptide complex described herein is isolated or purified. Generally, an isolated polypeptide or polypeptide complex is one that is substantially free of other polypeptides or polypeptide complexes with different antigenic specificities. For example, in some aspects, a preparation of a polypeptide or polypeptide complex described herein is substantially free of cellular material and/or chemical precursors.

### Pharmaceutical Compositions and Kits

In some aspects, the invention is directed to a pharmaceutical composition comprising an antigen binding polypeptide, antigen binding polypeptide complex (e.g., antibody or antigen binding fragment thereof), polypeptide, polynucleotide, vector, CAR or cell described herein.

In some aspects, the invention is directed to a pharmaceutical composition comprising (1) an antigen binding polypeptide, antigen binding polypeptide complex (e.g., antibody or antigen binding fragment thereof), polynucleotide, vector, CAR or cell described herein, and (2) a pharmaceutically acceptable carrier. The term “pharmaceutically acceptable carrier” includes any and all solvents, co-solvents, complexing agents, dispersion media, coatings, antibacterial and antifungal agents, isotonic and absorption delaying agents, and the like, which are not biologically or otherwise undesirable. The use of such media and agents for pharmaceutically active substances is known in the art. Except insofar as any conventional media or agent is incompatible with the active ingredient, its use in the therapeutic formulations is contemplated. Supplementary active ingredients can also be incorporated into the pharmaceutical compositions of the invention. In addition, various excipients, such as are commonly used in the art, can be included. These and other such compounds are described in the literature, e.g., in the Merck Index, Merck & Company, Rahway, NJ. Considerations for the inclusion of various components in pharmaceutical compositions are described, e.g., in Gilman et al. (Eds.) (2010); Goodman and Gilman's: The Pharmacological Basis of Therapeutics, 12th Ed., The McGraw-Hill Companies. In some aspects, the pharmaceutical composition is for parenteral, intravenous or subcutaneous administration.

In other aspects, the invention is directed to a kit comprising an antigen binding polypeptide, antigen binding polypeptide complex (e.g., antibody or antigen binding fragment thereof), polypeptide, polynucleotide, vector, cell, CAR or pharmaceutical composition described herein, or a combination thereof. Once a pharmaceutical composition has been formulated, it can be stored in sterile vials as a solution, suspension, gel, emulsion, solid, crystal, or as a dehydrated or lyophilized powder. Such formulations may be stored either in a ready-to-use form or in a form (e.g., lyophilized) that is reconstituted prior to administration. In some aspects, the invention provides kits for producing a single-dose administration unit. In some aspects, the kits of the invention can contain both a first container having a dried protein and a second container having an aqueous formulation. In some aspects, kits containing single and multi-chambered pre-filled syringes (e.g., liquid syringes and lyosyringes) are also provided. In some aspects, the kit contains components for intravenous or subcutaneous administration.

### Methods of Use

In some aspects, the invention is directed to certain methods of use of an antigen binding polypeptide, antigen binding polypeptide complex (e.g., an antibody or antigen binding fragment thereof), polypeptide, polynucleotide, vector, cell, CAR or pharmaceutical composition described herein, or a combination thereof.

In some aspects, the invention is directed to a method of neutralizing HIV infection, comprising administering to a subject in need thereof an antigen binding polypeptide, antigen binding polypeptide complex (e.g., antibody or antigen binding fragment thereof), polypeptide, polynucleotide, vector, CAR, cell or pharmaceutical composition described herein, or a combination thereof. In some aspects, the invention is directed to a method of neutralizing HIV infection, comprising administering to a subject in need thereof a therapeutically effective amount of an antigen binding polypeptide, antigen binding polypeptide complex (e.g., antibody or antigen binding fragment thereof), polypeptide, polynucleotide, vector, CAR, cell or pharmaceutical composition described herein, or a combination thereof.

In some aspects, the invention is directed to a method of treating or preventing HIV infection, comprising administering to a subject in need thereof an antigen binding polypeptide, antigen binding polypeptide complex (e.g., antibody or antigen binding fragment thereof), polypeptide, polynucleotide, vector, CAR, cell or pharmaceutical composition described herein, or a combination thereof. In some aspects, the invention is directed to a method of treating or preventing HIV infection, comprising administering to a subject in need thereof a therapeutically effective amount of an antigen binding polypeptide, antigen binding polypeptide complex (e.g., antibody or antigen binding fragment thereof), polypeptide, polynucleotide, vector, CAR, cell or pharmaceutical composition described herein, or a combination thereof.

In some aspects, the invention is directed to a method of treating or preventing acquired immunodeficiency syndrome (AIDS), comprising administering to a subject in need thereof an antigen binding polypeptide, antigen binding polypeptide complex (e.g., antibody or antigen binding fragment thereof), polypeptide, polynucleotide, vector, CAR, cell or pharmaceutical composition described herein, or a combination thereof. In some aspects, the invention is directed to a method of treating or preventing AIDS, comprising administering to a subject in need thereof a therapeutically effective amount of an antigen binding polypeptide, antigen binding polypeptide complex (e.g., antibody or antigen binding fragment thereof), polypeptide, polynucleotide, vector, CAR, cell or pharmaceutical composition described herein, or a combination thereof.

In some aspects, the invention is directed to a method of treating or preventing AIDS-related complex (ARC), comprising administering to a subject in need thereof an antigen binding polypeptide, antigen binding polypeptide complex (e.g., antibody or antigen binding fragment thereof), polypeptide, polynucleotide, vector, CAR, cell or pharmaceutical composition described herein, or a combination thereof. In some aspects, the invention is directed to a method of treating or preventing an ARC, comprising administering to a subject in need thereof a therapeutically effective amount of an antigen binding polypeptide, antigen binding polypeptide complex (e.g., antibody or antigen binding fragment thereof), polypeptide, polynucleotide, vector, CAR, cell or pharmaceutical composition described herein, or a combination thereof.

AIDS-related complex (ARC) is prodromal phase of HIV infection that presents certain symptoms that include, but are not limited to, low grade fever, unexplained weight loss, diarrhea, HIV-related opportunistic infections and generalized lymphadenopathy.

In some aspects, the invention is directed to a method of treating or preventing an HIV-related opportunistic infection, comprising administering to a subject in need thereof an antigen binding polypeptide, antigen binding polypeptide complex (e.g., antibody or antigen binding fragment thereof), polypeptide, polynucleotide, vector, CAR, cell or pharmaceutical composition described herein, or a combination thereof. In some aspects, the invention is directed to a method of treating or preventing an HIV-related opportunistic infection, comprising administering to a subject in need thereof a therapeutically effective amount of an antigen binding polypeptide, antigen binding polypeptide complex (e.g., antibody or antigen binding fragment thereof), polypeptide, polynucleotide, vector, CAR, cell or pharmaceutical composition described herein, or a combination thereof.

HIV-related opportunistic infections are illnesses that occur more frequently and/or more severely in subjects infected with HIV, due to their compromised immune systems. Examples of HIV-related opportunistic infections include, but are not limited to, candidiasis, invasive cervical cancer, coccidioidomycosis, cryptococcosis, cryptosporidiosis (Crypto), cystoisosporiasis, cytomegalovirus (CMV) infection, encephalopathy, herpes simplex virus (HSV) infection, histoplasmosis, Kaposi's sarcoma (KS), lymphoma, tuberculosis, Mycobacterium avium complex (MAC), Pneumocystis pneumonia (PCP), pneumonia, progressive multifocal leukoencephalopathy, Salmonella septicemia, toxoplasmosis, or wasting syndrome.

In some aspects, HIV is HIV-1 or HIV-2.

As used herein, the term “subject” means a human or a non-human mammal, e.g., a dog, a cat, a mouse, a rat, a cow, a sheep, a pig, a goat, a non-human primate or a bird, e.g., a chicken, as well as any other vertebrate or invertebrate. In some aspects, the subject is a human. In some aspects, the subject is a veterinary animal. In some aspects, the subject is a mammal.

As used herein, the terms “treat” or “treatment” refer to therapeutic or palliative measures. Beneficial or desired clinical results include, but are not limited to, alleviation, in whole or in part, of symptoms associated with a disease or disorder or condition, diminishment of the extent of disease, stabilized (i.e., not worsening) state of disease, delay or slowing of disease progression, amelioration or palliation of the disease state (e.g., one or more symptoms of the disease), and remission (whether partial or total), whether detectable or undetectable. “Treatment” can also mean prolonging survival as compared to expected survival if not receiving treatment.

As used herein, the terms “prevent” or “preventing” refer to the prevention of the onset, recurrence or spread, in whole or in part, of a disease or condition described herein, or a symptom thereof.

As used herein, a “therapeutically effective amount” is an amount of an antigen binding polypeptide or antigen binding polypeptide complex (e.g., an antibody or antigen binding fragment thereof) that is sufficient to achieve the desired effect and can vary according to the nature and severity of the disease condition, and the potency of the polypeptide or polypeptide complex. In some aspects, an antigen binding polypeptide or antigen binding polypeptide complex of the invention can be delivered by administering a polynucleotide, vector, CAR or cell that encodes the antigen binding polypeptide or antigen binding polypeptide complex. In some aspects, an antigen binding polypeptide or antigen binding polypeptide complex thereof can be delivered by administering a pharmaceutical composition containing the polypeptide or polypeptide complex. A therapeutic effect is the relief, to some extent, of one or more of the symptoms of the disease, and can include curing a disease. “Curing” means that the symptoms of active disease are eliminated. However, certain long-term or permanent effects of the disease can exist even after a cure is obtained.

### EXAMPLES

The following examples are provided to further illustrate aspects of the disclosure, and are not meant to constrain the disclosure to any particular application or theory of operation.

### Example 1

### Design of Antibody Constructs

Antibody heavy chain variable domains (VH) and light chain variable domains (VL) from anti-HIV antibodies are selected from publicly available databases (e.g., GenBank) or patents to illustrate the feasibility of constructing various formats of trispecific antibodies. Linkers in various length and sequence connecting VH and VL regions in different orders and orientations are tested, with and without different motifs of the constant domains (e.g., CL, CH1, CH2, CH3). “Knob” and “hole” mutations are integrated into respective halves of the antibody Fc region when Fc heterodimerization is needed. Effector function or half-life extension mutations are also incorporated into the Fc sequences when needed. Once the amino acid sequences for each multispecific antibody molecule are assembled, DNA encoding these sequences is codon optimized, synthesized (Cambridge Biologics, LLC, Brookline, MA), and cloned into a eukaryotic expression vector.

### Example 2

### Multispecific Antibody Expression and Purification

Multispecific antibodies are produced by transient transfection of expression plasmids into Expi293F cells at density of 2.5-3.0×10⁶/ml using polyethylenimine (PEI; Polyscience). Plasmid DNA and PEI were diluted in OPTi-MEM (LifeTech) separately and mixed later. The plasmid/PEI mixture, at a ratio of 1:3 (w:w), is added to the cell culture 10 minutes after mixing. Valproic acid and sodium propionate are added to final concentrations of 0.5 mM and 5 mM, respectively, 16-20 hours post transfection. Supernatant is harvested 5 days post transfection, and filtered through a 0.45 um filter. Multispecific antibodies are then purified first by affinity chromatography using Protein A resins in batch mode according to the manufacture's standard procedures. After antibodies are eluted using IgG elusion buffer (Thermo Fischer Scientific) from protein A, they are dialyzed into 10 mM Histidine (pH6.0)+25 mM NaCl overnight. Antibodies are further purified by size exclusion chromatography using Hiload 16/600 Superdex 200 PG or Superdex 200 Increase 10/300 GL (Cytiva Lifesciences). Fractions with the correct elusion profile are collected and concentrated for further characterization.

### Example 3

### Multispecific Antibody ELISA Binding Analysis

An ELISA binding assay is used to test binding of multispecific antibodies to their target antigens. Target protein for each binding site of the multispecific antibodies is coated in the wells of 96-well Immuno Plates (Thermo Fisher Scientific) overnight at 4° C. Coated plates are blocked using 5% skim milk+2% bovine serum albumin (BSA) in phosphate buffered saline (PBS)+0.25% Tween for one hour at room temperature, then washed three times with PBS+0.25% Tween 20. Serial diluted multispecific antibodies and control molecules are added to the plate and incubated at room temperature for 1 hr. Plates are washed three times with PBS+0.25% Tween 20, incubated with horseradish peroxidase (HPR) conjugated detection antibody for one hour at room temperature, washed again, and then developed with Peroxidase Substrate (KPL, Gaithersburg, MD, USA). After the reaction is terminated by adding 100 μl of KPL TMB BlueSTOP solution, the plates are read at OD₆₅₀ using a plate reader and data analyzed in GraphPad Prism.

### Example 4

### Luciferase Reporter Assay

A luciferase reporter assay is used to test binding of multispecific antibodies to their target antigens. More specifically, NFkB Luciferase Reporter Jurkat Stable Cell Line (Signosis, CA, USA) and Jurkat-Lucia™ NFAT Cells (InvivoGen, CA, USA) are prepared according to the manufacturer's protocol. Briefly, cells are thawed for 2 min in a 37° C. water bath and gently transferred to a 15 mL conical centrifuge tube containing 10 mL pre-warmed R10 media. Cells are pelleted at 300 g for 5 min at room temperature. After removing the supernatant, cells are resuspended in 20 mL pre-warmed culture media and transferred to a 75 cm² culture flask, followed by incubation in a mammalian tissue culture incubator until cells are growing and stable (^{˜}3-4 days). Cells are maintained in culture media+selective antibiotics and normally used 7 days after thawing.

For antibody stimulation, multispecific or control antibodies are serially diluted in PBS and coated onto 96-well flat-bottom culture plates by incubating 2-4 hours in a 37° C. tissue culture incubator. NFkB Luciferase Reporter Jurkat Stable Cells are resuspended to 2×10⁶ cells/mL, with 100 μl of cells added to each well containing the antibodies, and incubated in a mammalian incubator for 24 hours. Assay plates are then taken out and allowed to equilibrate to ambient temperature (10-15 min). Bio-Glo™ Reagent (Promega Cat #G7941) (ambient temperature) was added at 50 μl for each well of assay plates. After 5 minutes, luminescence activity is measured using Varioskan microplate reader (Thermo Fisher). Data is plotted using GraphPad Prism software.

In addition, Jurkat-Lucia™ NFAT Cells are resuspended to 7.5×10⁵ cells/mL, with 200 μl of cells added to each well containing the antibodies and incubated in a mammalian incubator for 24 hours. 20 μL of the cell culture supernatant is pipetted into a new 96-well white-walled microtiter plate. 50 μL of Quanti-Luc solution (InvivoGen) is then added to each well before luminescence activity is measured using Varioskan microplate reader (Thermo Fisher). Data are plotted using GraphPad Prism software.

### Example 5

### BLI Analysis of Antibody Constructs

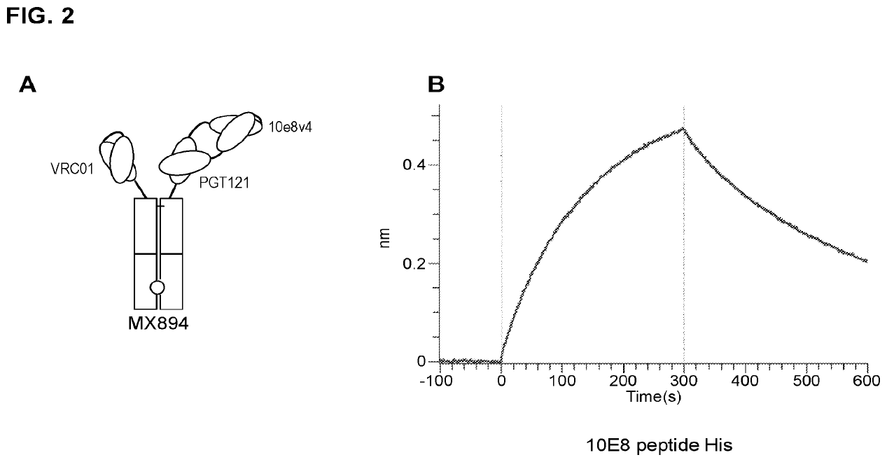

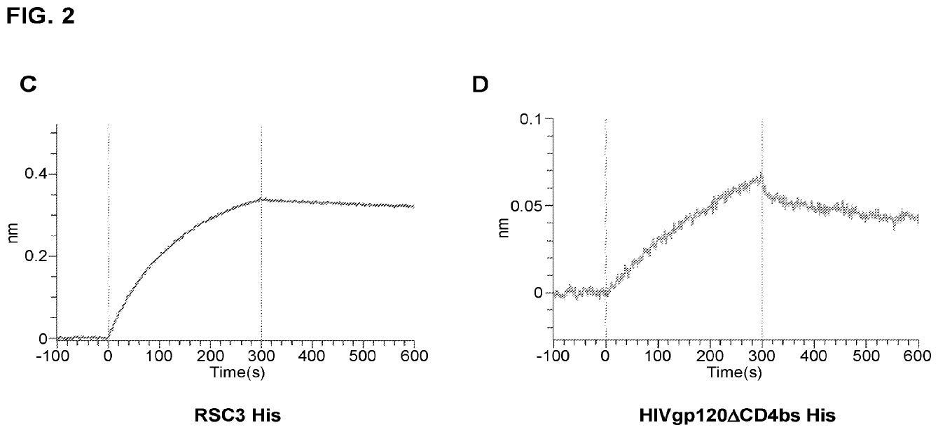

A non-limiting example of a trispecific antibody configuration is shown in FIG. 2A, called MX894 (SEQ ID NOs:84-87; VRC01scFv/PGT121×10e8v4L1IgG1LS). MX894 was analyzed for binding to 10e8 fusion peptide, and CD4 site-dependent and CD4 site-independent HIV spike protein by biolayer interferometry (BLI) as follows.

### Binding Kinetic Analyses by Biolayer Interferometry

On the Octet® R8 (Sartorius), recombinant His-tagged HIV RCS3, HIV gp140ΔCD4, or HIV 10e8 peptide was loaded by His-tag capture onto HIS1K biosensors (100 nM ligand, 300 seconds, 1000 RPM). After baseline step (100 seconds, 1000 RPM), association with each test molecule (100 nM analyte) was monitored (300 seconds, 1000 RPM). Dissociation was then monitored (300 seconds, 1000 RPM).

All assay steps occurred in 1× kinetic buffer (1×PBS pH 7.4; 0.1% BSA; 0.02% Tween-20) at 24 degrees C. Prior to each kinetic cycle, the HIS1K biosensors were regenerated in 1.5 pH glycine (5 seconds, 1000 RPM) and neutralized in 1× kinetic buffer (5 seconds, 1000 RPM) 5 consecutive times and then equilibrated back to 1× kinetic buffer (100 seconds; 1000 RPM).

Binding model fit assumed a 1:1 binding model and fit the association and dissociation together. Baseline was determined by mean of last five seconds of baseline step.

The results in FIGS. 2B-2D show that MX894 bound to 10e8 fusion peptide (FIG. 2B), and CD4 site-dependent (FIG. 2C) and CD4 site-independent (FIG. 2D) HIV spike protein.

### Example 6

### BLI Analysis of Additional Antibody Constructs

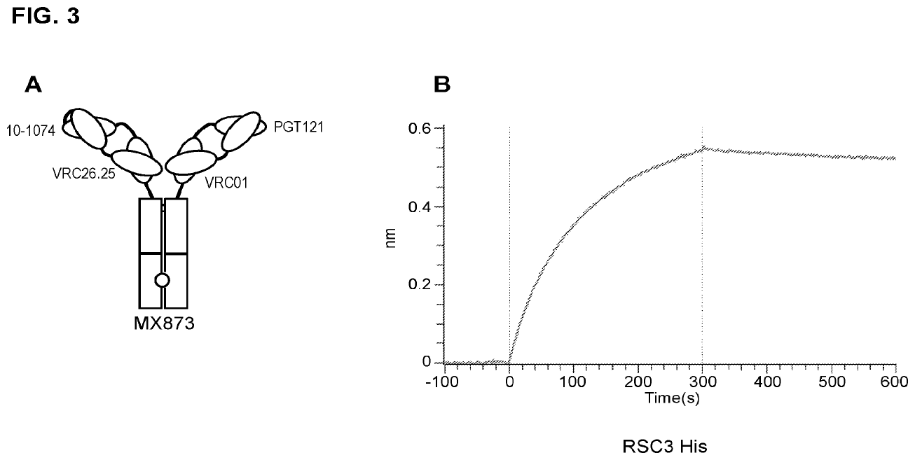

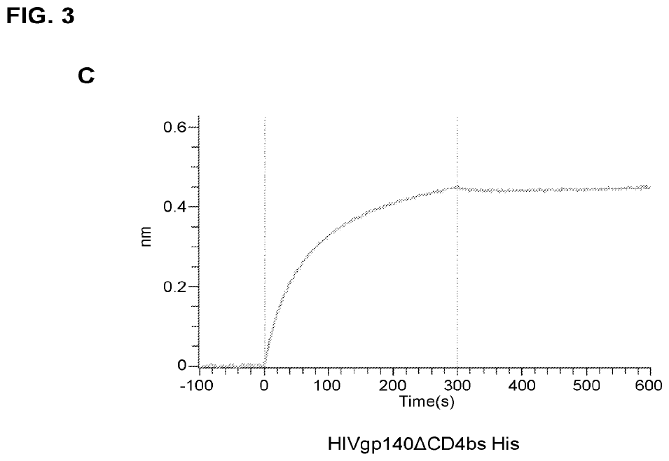

A further non-limiting example of a tetraspecific antibody configuration is shown in FIG. 3A, called MX873 (SEQ ID NOs:88-91; VRC26.25×10-1074L9/VRC01×PGT121L1 IgG1LS). MX873 was analyzed for binding to CD4 site-dependent and CD4 site-independent HIV spike protein by biolayer interferometry (BLI) as described above.

The results in FIGS. 3B-3C show that MX873 bound to CD4 site-dependent (FIG. 3B) and CD4 site-independent (FIG. 3C) HIV spike protein.

### Example 7

### BLI Analysis of Additional Antibody Constructs

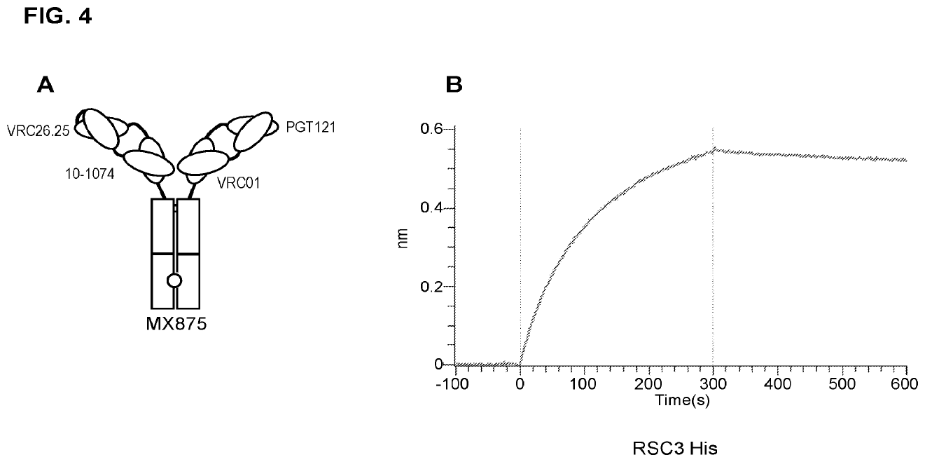

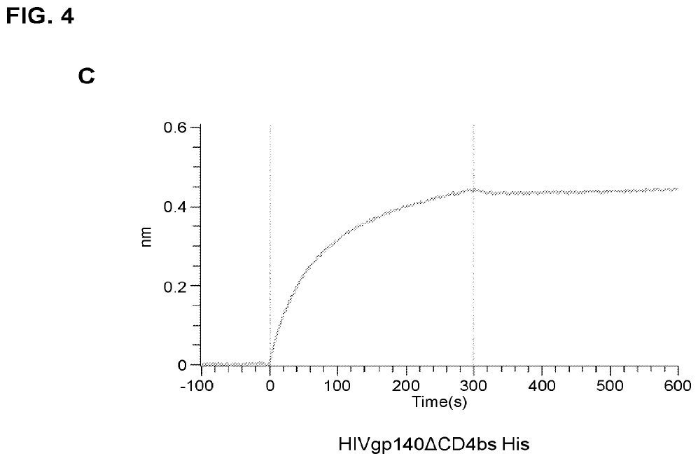

A further non-limiting example of a tetraspecific antibody configuration is shown in FIG. 4A, called MX875 (SEQ ID NOs:92-95; 10-1074×VRC26.25L9/VRC01×PGT121L1 IgG1LS). MX875 was analyzed for binding to CD4 site-dependent and CD4 site-independent HIV spike protein by biolayer interferometry (BLI) as described above.

The results in FIGS. 4B-4C show that MX875 bound to CD4 site-dependent (FIG. 4B) and CD4 site-independent (FIG. 4C) HIV spike protein.

### Example 8

### BLI Analysis of Additional Antibody Constructs

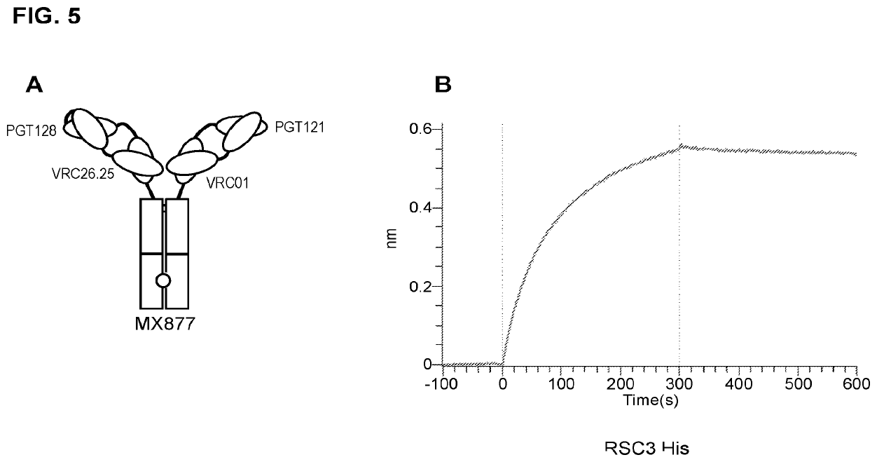

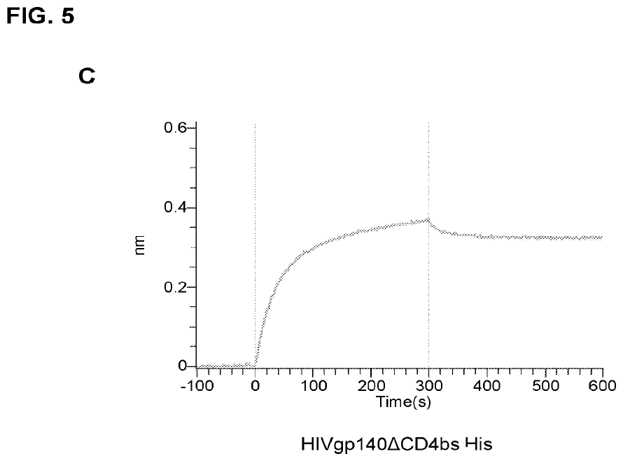

A further non-limiting example of a tetraspecific antibody configuration is shown in FIG. 5A, called MX877 (SEQ ID NOs:96-99; STAR_VRC26.25×PGT128L9/STAR_VRC01×PGT121L1 IgG1LS). MX877 was analyzed for binding to CD4 site-dependent and CD4 site-independent HIV spike protein by biolayer interferometry (BLI) as described above.

The results in FIGS. 5B-5C show that MX877 bound to CD4 site-dependent (FIG. 5B) and CD4 site-independent (FIG. 5C) HIV spike protein.

All publications, patents and patent applications mentioned in this application are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1-83. (canceled)

84. An antigen binding polypeptide complex comprising a first polypeptide and a second polypeptide;
wherein the first polypeptide has a structure represented by:
VL1-VL2-VH2-VH1-Fc;
VH1-VH2-VL2-VL1-Fc;
VL1-L1-VL2-L2-VH2-L3-VH1-Fc;
VH1-L1-VH2-L2-VL2-L3-VL1-Fc;
VL1-L1-VL2-L2-VH2-L3-VH1-L4-Fc; or
VH1-L1-VH2-L2-VL2-L3-VL1-L4-Fc;
wherein the second polypeptide has a structure represented by:
VL1-VL2-VH2-VH1-Fc;
VH1-VH2-VL2-VL1-Fc;
VL1-L1-VL2-L2-VH2-L3-VH1-Fc;
VH1-L1-VH2-L2-VL2-L3-VL1-Fc;
VL1-L1-VL2-L2-VH2-L3-VH1-L4-Fc;
VH1-L1-VH2-L2-VL2-L3-VL1-L4-Fc;
VL3-VL4-VH4-VH3-Fc;
VH3-VH4-VL4-VL3-Fc;
VL3-L5-VL4-L6-VH4-L7-VH3-Fc;
VH3-L5-VH4-L6-VL4-L7-VL3-Fc;
VL3-L5-VL4-L6-VH4-L7-VH3-L8-Fc; or
VH3-L5-VH4-L6-VL4-L7-VL3-L8-Fc; and
wherein:
VL1 is a first immunoglobulin light chain variable region that specifically binds to an HIV protein;
VL2 is a second immunoglobulin light chain variable region that specifically binds to an HIV protein;
VL3 is a third immunoglobulin light chain variable region that specifically binds to an HIV protein;
VL4 is a fourth immunoglobulin light chain variable region that specifically binds to an HIV protein;
VH1 is a first immunoglobulin heavy chain variable region that specifically binds to an HIV protein;
VH2 is a second immunoglobulin heavy chain variable region that specifically binds to an HIV protein;
VH3 is a third immunoglobulin heavy chain variable region that specifically binds to an HIV protein;
VH4 is a fourth immunoglobulin heavy chain variable region that specifically binds to an HIV protein;
Fc is a region comprising an immunoglobulin heavy chain constant region 2 (CH2), an immunoglobulin heavy chain constant region 3 (CH3), and optionally, an immunoglobulin hinge; and
L1, L2, L3, L4, L5, L6, L7, and L8 are amino acid linkers.

85. The antigen binding polypeptide complex of claim 84,
wherein the first polypeptide has a structure represented by:
VL1-VL2-VH2-VH1-CH1-Fc;
VH1-VH2-VL2-VL1-CH1-Fc;
VL1-VL2-VH2-VH1-CL-Fc;
VH1-VH2-VL2-VL1-CL-Fc;
VL1-VL2-VH2-VH1-CH1-CL-Fc;
VH1-VH2-VL2-VL1-CH1-CL-Fc;
VL1-VL2-VH2-VH1-CL-CH1-Fc;
VH1-VH2-VL2-VL1-CL-CH1-Fc;
VL1-L1-VL2-L2-VH2-L3-VH1-L4-CH1-Fc;
VH1-L1-VH2-L2-VL2-L3-VL1-L4-CH1-Fc;
VL1-L1-VL2-L2-VH2-L3-VH1-L4-CL-Fc;
VH1-L1-VH2-L2-VL2-L3-VL1-L4-CL-Fc;
VL1-L1-VL2-L2-VH2-L3-VH1-L4-CH1-L5-CL-Fc;
VH1-L1-VH2-L2-VL2-L3-VL1-L4-CH1-L5-CL-Fc;
VL1-L1-VL2-L2-VH2-L3-VH1-L4-CL-L5-CH1-Fc;
VH1-L1-VH2-L2-VL2-L3-VL1-L4-CL-L5-CH1-Fc;
VL1-L1-VL2-L2-VH2-L3-VH1-L4-CH1-L5-Fc;
VH1-L1-VH2-L2-VL2-L3-VL1-L4-CH1-L5-Fc;
VL1-L1-VL2-L2-VH2-L3-VH1-L4-CL-L5-Fc;
VH1-L1-VH2-L2-VL2-L3-VL1-L4-CL-L5-Fc;
VL1-L1-VL2-L2-VH2-L3-VH1-L4-CH1-L5-CL-L6-Fc;
VH1-L1-VH2-L2-VL2-L3-VL1-L4-CH1-L5-CL-L6-Fc;
VL1-L1-VL2-L2-VH2-L3-VH1-L4-CL-L5-CH1-L6-Fc; or
VH1-L1-VH2-L2-VL2-L3-VL1-L4-CL-L5-CH1-L6-Fc;
wherein the second polypeptide has a structure represented by:
VL1-VL2-VH2-VH1-CH1-Fc;
VH1-VH2-VL2-VL1-CH1-Fc;
VL1-VL2-VH2-VH1-CL-Fc;
VH1-VH2-VL2-VL1-CL-Fc;
VL1-VL2-VH2-VH1-CH1-CL-Fc;
VH1-VH2-VL2-VL1-CH1-CL-Fc;
VL1-VL2-VH2-VH1-CL-CH1-Fc;
VH1-VH2-VL2-VL1-CL-CH1-Fc;
VL1-L1-VL2-L2-VH2-L3-VH1-L4-CH1-Fc;
VH1-L1-VH2-L2-VL2-L3-VL1-L4-CH1-Fc;
VL1-L1-VL2-L2-VH2-L3-VH1-L4-CL-Fc;
VH1-L1-VH2-L2-VL2-L3-VL1-L4-CL-Fc;
VL1-L1-VL2-L2-VH2-L3-VH1-L4-CH1-L5-CL-Fc;
VH1-L1-VH2-L2-VL2-L3-VL1-L4-CH1-L5-CL-Fc;
VL1-L1-VL2-L2-VH2-L3-VH1-L4-CL-L5-CH1-Fc;
VH1-L1-VH2-L2-VL2-L3-VL1-L4-CL-L5-CH1-Fc;
VL1-L1-VL2-L2-VH2-L3-VH1-L4-CH1-L5-Fc;
VH1-L1-VH2-L2-VL2-L3-VL1-L4-CH1-L5-Fc;
VL1-L1-VL2-L2-VH2-L3-VH1-L4-CL-L5-Fc;
VH1-L1-VH2-L2-VL2-L3-VL1-L4-CL-L5-Fc;
VL1-L1-VL2-L2-VH2-L3-VH1-L4-CH1-L5-CL-L6-Fc;
VH1-L1-VH2-L2-VL2-L3-VL1-L4-CH1-L5-CL-L6-Fc;
VL1-L1-VL2-L2-VH2-L3-VH1-L4-CL-L5-CH1-L6-Fc;
VH1-L1-VH2-L2-VL2-L3-VL1-L4-CL-L5-CH1-L6-Fc;
VL3-VL4-VH4-VH3-CH1-Fc;
VH3-VH4-VL4-VL3-CH1-Fc;
VL3-VL4-VH4-VH3-CL-Fc;
VH3-VH4-VL4-VL3-CL-Fc;
VL3-VL4-VH4-VH3-CH1-CL-Fc;
VH3-VH4-VL4-VL3-CH1-CL-Fc;
VL3-VL4-VH4-VH3-CL-CH1-Fc;
VH3-VH4-VL4-VL3-CL-CH1-Fc;
VL3-L7-VL4-L8-VH4-L9-VH3-L10-CH1-Fc;
VH3-L7-VH4-L8-VL4-L9-VL3-L10-CH1-Fc;
VL3-L7-VL4-L8-VH4-L9-VH3-L10-CL-Fc;
VH3-L7-VH4-L8-VL4-L9-VL3-L10-CL-Fc;
VL3-L7-VL4-L8-VH4-L9-VH3-L10-CH1-L11-CL-Fc;
VH3-L7-VH4-L8-VL4-L9-VL3-L10-CH1-L11-CL-Fc;
VL3-L7-VL4-L8-VH4-L9-VH3-L10-CL-L11-CH1-Fc;
VH3-L7-VH4-L8-VL4-L9-VL3-L10-CL-L11-CH1-Fc;
VL3-L7-VL4-L8-VH4-L9-VH3-L10-CH1-L11-Fc;
VH3-L7-VH4-L8-VL4-L9-VL3-L10-CH1-L11-Fc;
VL3-L7-VL4-L8-VH4-L9-VH3-L10-CL-L11-Fc;
VH3-L7-VH4-L8-VL4-L9-VL3-L10-CL-L11-Fc;
VL3-L7-VL4-L8-VH4-L9-VH3-L10-CH1-L11-CL-L12-Fc;
VH3-L7-VH4-L8-VL4-L9-VL3-L10-CH1-L11-CL-L12-Fc;
VL3-L7-VL4-L8-VH4-L9-VH3-L10-CL-L11-CH1-L12-Fc; or
VH3-L7-VH4-L8-VL4-L9-VL3-L10-CL-L11-CH1-L12-Fc; and
wherein:
VL1 is a first immunoglobulin light chain variable region that specifically binds to an HIV protein;
VL2 is a second immunoglobulin light chain variable region that specifically binds to an HIV protein;
VL3 is a third immunoglobulin light chain variable region that specifically binds to an HIV protein;
VL4 is a fourth immunoglobulin light chain variable region that specifically binds to an HIV protein;
VH1 is a first immunoglobulin heavy chain variable region that specifically binds to an HIV protein;
VH2 is a second immunoglobulin heavy chain variable region that specifically binds to an HIV protein;
VH3 is a third immunoglobulin heavy chain variable region that specifically binds to an HIV protein;
VH4 is a fourth immunoglobulin heavy chain variable region that specifically binds to an HIV protein;
CH1 is an immunoglobulin heavy chain constant region 1;
CL is an immunoglobulin light chain constant region;
Fc is a region comprising an immunoglobulin heavy chain constant region 2 (CH2), an immunoglobulin heavy chain constant region 3 (CH3), and optionally, an immunoglobulin hinge; and
L1, L2, L3, L4, L5, L6, L7, L8, L9, L10, L11, and L12 are amino acid linkers.

86. An antigen binding polypeptide complex comprising a first polypeptide and a second polypeptide;
wherein the first polypeptide has a structure represented by:
VL1-VL2-VH2-VH1-Fc;
VH1-VH2-VL2-VL1-Fc;
VL1-L1-VL2-L2-VH2-L3-VH1-Fc;
VH1-L1-VH2-L2-VL2-L3-VL1-Fc;
VL1-L1-VL2-L2-VH2-L3-VH1-L4-Fc; or
VH1-L1-VH2-L2-VL2-L3-VL1-L4-Fc;
wherein the second polypeptide has a structure represented by:
VL3-VH3-Fc;
VH3-VL3-Fc;
VL3-L5-VH3-Fc;
VH3-L5-VL3-Fc;
VL3-L5-VH3-L6-Fc; or
VH3-L5-VL3-L6-Fc; and
wherein:
VL1 is a first immunoglobulin light chain variable region that specifically binds to an HIV protein;
VL2 is a second immunoglobulin light chain variable region that specifically binds to an HIV protein;
VL3 is a third immunoglobulin light chain variable region that specifically binds to an HIV protein;
VH1 is a first immunoglobulin heavy chain variable region that specifically binds to an HIV protein;
VH2 is a second immunoglobulin heavy chain variable region that specifically binds to an HIV protein;
VH3 is a third immunoglobulin heavy chain variable region that specifically binds to an HIV protein;
Fc is a region comprising an immunoglobulin heavy chain constant region 2 (CH2), an immunoglobulin heavy chain constant region 3 (CH3), and optionally, an immunoglobulin hinge; and
L1, L2, L3, L4, L5, and L6 are amino acid linkers.

87. The antigen binding polypeptide complex of claim 86, wherein the first polypeptide has a structure represented by:
VL1-VL2-VH2-VH1-CH1-Fc;
VH1-VH2-VL2-VL1-CH1-Fc;
VL1-VL2-VH2-VH1-CL-Fc;
VH1-VH2-VL2-VL1-CL-Fc;
VL1-VL2-VH2-VH1-CH1-CL-Fc;
VH1-VH2-VL2-VL1-CH1-CL-Fc;
VL1-VL2-VH2-VH1-CL-CH1-Fc;
VH1-VH2-VL2-VL1-CL-CH1-Fc;
VL1-L1-VL2-L2-VH2-L3-VH1-L4-CH1-Fc;
VH1-L1-VH2-L2-VL2-L3-VL1-L4-CH1-Fc;
VL1-L1-VL2-L2-VH2-L3-VH1-L4-CL-Fc;
VH1-L1-VH2-L2-VL2-L3-VL1-L4-CL-Fc;
VL1-L1-VL2-L2-VH2-L3-VH1-L4-CH1-L5-CL-Fc;
VH1-L1-VH2-L2-VL2-L3-VL1-L4-CH1-L5-CL-Fc;
VL1-L1-VL2-L2-VH2-L3-VH1-L4-CL-L5-CH1-Fc;
VH1-L1-VH2-L2-VL2-L3-VL1-L4-CL-L5-CH1-Fc;
VL1-L1-VL2-L2-VH2-L3-VH1-L4-CH1-L5-Fc;
VH1-L1-VH2-L2-VL2-L3-VL1-L4-CH1-L5-Fc;
VL1-L1-VL2-L2-VH2-L3-VH1-L4-CL-L5-Fc;
VH1-L1-VH2-L2-VL2-L3-VL1-L4-CL-L5-Fc;
VL1-L1-VL2-L2-VH2-L3-VH1-L4-CH1-L5-CL-L6-Fc;
VH1-L1-VH2-L2-VL2-L3-VL1-L4-CH1-L5-CL-L6-Fc;
VL1-L1-VL2-L2-VH2-L3-VH1-L4-CL-L5-CH1-L6-Fc; or
VH1-L1-VH2-L2-VL2-L3-VL1-L4-CL-L5-CH1-L6-Fc;
wherein the second polypeptide has a structure represented by:
VL3-VH3-CH1-Fc;
VH3-VL3-CH1-Fc;
VL3-VH3-CL-Fc;
VH3-VL3-CL-Fc;
VL3-VH3-CH1-CL-Fc;
VH3-VL3-CH1-CL-Fc;
VL3-VH3-CL-CH1-Fc;
VH3-VL3-CL-CH1-Fc;
VL3-CL-VH3-CH1-Fc;
VL3-CH1-VH3-CL-Fc;
VH3-CH1-VL3-CL-Fc;
VH3-CL-VL3-CH1-Fc;
VL3-L7-VH3-L8-CH1-Fc;
VH3-L7-VL3-L8-CH1-Fc;
VL3-L7-VH3-L8-CL-Fc;
VH3-L7-VL3-L8-CL-Fc;
VL3-L7-VH3-L8-CH1-L9-CL-Fc;
VH3-L7-VL3-L8-CH1-L9-CL-Fc;
VL3-L7-VH3-L8-CL-L9-CH1-Fc;
VH3-L7-VL3-L8-CL-L9-CH1-Fc;
VL3-L7-CL-L8-VH3-L9-CH1-Fc;
VL3-L7-CH1-L8-VH3-L9-CL-Fc;
VH3-L7-CH1-L8-VL3-L9-CL-Fc;
VH3-L7-CL-L8-VL3-L9-CH1-Fc;
VL3-L7-VH3-L8-CH1-L9-CL-L10-Fc;
VH3-L7-VL3-L8-CH1-L9-CL-L10-Fc;
VL3-L7-VH3-L8-CL-L9-CH1-L10-Fc;
VH3-L7-VL3-L8-CL-L9-CH1-L10-Fc;
VL3-L7-CL-L8-VH3-L9-CH1-L10-Fc;
VL3-L7-CH1-L8-VH3-L9-CL-L10-Fc;
VH3-L7-CH1-L8-VL3-L9-CL-L10-Fc;
VH3-L7-CL-L8-VL3-L9-CH1-L10-Fc;
VL3-VH3-L7-CH1-CL-Fc;
VH3-VL3-L7-CH1-CL-Fc;
VL3-VH3-L7-CL-CH1-Fc;
VH3-VL3-L7-CL-CH1-Fc;
VL3-CL-L7-VH3-CH1-Fc;
VL3-CH1-L7-VH3-CL-Fc;
VH3-CH1-L7-VL3-CL-Fc; or
VH3-CL-L7-VL3-CH1-Fc; and
wherein:
VL1 is a first immunoglobulin light chain variable region that specifically binds to an HIV protein;
VL2 is a second immunoglobulin light chain variable region that specifically binds to an HIV protein;
VL3 is a third immunoglobulin light chain variable region that specifically binds to an HIV protein;
VH1 is a first immunoglobulin heavy chain variable region that specifically binds to an HIV protein;
VH2 is a second immunoglobulin heavy chain variable region that specifically binds to an HIV protein;
VH3 is a third immunoglobulin heavy chain variable region that specifically binds to an HIV protein;
Fc is a region comprising an immunoglobulin heavy chain constant region 2 (CH2), an immunoglobulin heavy chain constant region 3 (CH3), and optionally, an immunoglobulin hinge;
CH1 is an immunoglobulin heavy chain constant region 1;
CL is an immunoglobulin light chain constant region; and
L1, L2, L3, L4, L5, L6, L7, L8, L9, and L10 are amino acid linkers.

88. An antigen binding polypeptide complex comprising a first polypeptide, a second polypeptide, and a third polypeptide;
wherein the first polypeptide has a structure represented by:
VL1-VL2-VH2-VH1-Fc;
VH1-VH2-VL2-VL1-Fc;
VL1-L1-VL2-L2-VH2-L3-VH1-Fc;
VH1-L1-VH2-L2-VL2-L3-VL1-Fc;
VL1-L1-VL2-L2-VH2-L3-VH1-L4-Fc; or
VH1-L1-VH2-L2-VL2-L3-VL1-L4-Fc;
wherein the second polypeptide has a structure represented by:
VL3; or
VL3-L5;
wherein the third polypeptide has a structure represented by:
VH3-Fc; or
VH3-L6-Fc; and
wherein:
VL1 is a first immunoglobulin light chain variable region that specifically binds to an HIV protein;
VL2 is a second immunoglobulin light chain variable region that specifically binds to an HIV protein;
VL3 is a third immunoglobulin light chain variable region that specifically binds to an HIV protein;
VH1 is a first immunoglobulin heavy chain variable region that specifically binds to an HIV protein;
VH2 is a second immunoglobulin heavy chain variable region that specifically binds to an HIV protein;
VH3 is a third immunoglobulin heavy chain variable region that specifically binds to an HIV protein;
Fc is a region comprising an immunoglobulin heavy chain constant region 2 (CH2), an immunoglobulin heavy chain constant region 3 (CH3), and optionally, an immunoglobulin hinge; and
L1, L2, L3, L4, L5, and L6 are amino acid linkers.

89. The antigen binding polypeptide complex of claim 88, wherein the first polypeptide has a structure represented by:
VL1-VL2-VH2-VH1-CH1-Fc;
VH1-VH2-VL2-VL1-CH1-Fc;
VL1-VL2-VH2-VH1-CL-Fc;
VH1-VH2-VL2-VL1-CL-Fc;
VL1-VL2-VH2-VH1-CH1-CL-Fc;
VH1-VH2-VL2-VL1-CH1-CL-Fc;
VL1-VL2-VH2-VH1-CL-CH1-Fc;
VH1-VH2-VL2-VL1-CL-CH1-Fc;
VL1-L1-VL2-L2-VH2-L3-VH1-L4-CH1-L5-Fc;
VH1-L1-VH2-L2-VL2-L3-VL1-L4-CH1-L5-Fc;
VL1-L1-VL2-L2-VH2-L3-VH1-L4-CL-L5-Fc;
VH1-L1-VH2-L2-VL2-L3-VL1-L4-CL-L5-Fc;
VL1-L1-VL2-L2-VH2-L3-VH1-L4-CH1-L5-CL-L6-Fc;
VH1-L1-VH2-L2-VL2-L3-VL1-L4-CH1-L5-CL-L6-Fc;
VL1-L1-VL2-L2-VH2-L3-VH1-L4-CL-L5-CH1-L6-Fc; or
VH1-L1-VH2-L2-VL2-L3-VL1-L4-CL-L5-CH1-L6-Fc;
wherein the second polypeptide has a structure represented by:
VL3-CH1;
VL3-CL;
VL3-L7-CH1; or
VL3-L7-CL;
wherein the third polypeptide has a structure represented by:
VH3-CH1-Fc;
VH3-CL-Fc;
VH3-L8-CH1-L9-Fc; or
VH3-L8-CL-L9-Fc; and
wherein:
VL1 is a first immunoglobulin light chain variable region that specifically binds to an HIV protein;
VL2 is a second immunoglobulin light chain variable region that specifically binds to an HIV protein;
VL3 is a third immunoglobulin light chain variable region that specifically binds to an HIV protein;
VH1 is a first immunoglobulin heavy chain variable region that specifically binds to an HIV protein;
VH2 is a second immunoglobulin heavy chain variable region that specifically binds to an HIV protein;
VH3 is a third immunoglobulin heavy chain variable region that specifically binds to an HIV protein;
CH1 is an immunoglobulin heavy chain constant region 1;
CL is an immunoglobulin light chain constant region; and
L1, L2, L3, L4, L5, L6, L7, L8, and L9 are amino acid linkers.

90. An antigen binding polypeptide complex comprising a first polypeptide, a second polypeptide, and a third polypeptide;
wherein the first polypeptide has a structure represented by:
VL1-VL2-VH2-VH1-Fc;
VH1-VH2-VL2-VL1-Fc;
VL1-L1-VL2-L2-VH2-L3-VH1-Fc;
VH1-L1-VH2-L2-VL2-L3-VL1-Fc;
VL1-L1-VL2-L2-VH2-L3-VH1-L4-Fc; or
VH1-L1-VH2-L2-VL2-L3-VL1-L4-Fc;
wherein the second polypeptide has a structure represented by:
VL3-Fc; or
VL3-L5-Fc;
wherein the third polypeptide has a structure represented by:
VH3; or
VH3-L6; and
wherein:
VL1 is a first immunoglobulin light chain variable region that specifically binds to an HIV protein;
VL2 is a second immunoglobulin light chain variable region that specifically binds to an HIV protein;
VL3 is a third immunoglobulin light chain variable region that specifically binds to an HIV protein;
VH1 is a first immunoglobulin heavy chain variable region that specifically binds to an HIV protein;
VH2 is a second immunoglobulin heavy chain variable region that specifically binds to an HIV protein;
VH3 is a third immunoglobulin heavy chain variable region that specifically binds to an HIV protein;
Fc is a region comprising an immunoglobulin heavy chain constant region 2 (CH2), an immunoglobulin heavy chain constant region 3 (CH3), and optionally, an immunoglobulin hinge; and
L1, L2, L3, L4, L5, and L6 are amino acid linkers.

91. The antigen binding polypeptide complex of claim 90, wherein the first polypeptide has a structure represented by:
VL1-VL2-VH2-VH1-CH1-Fc;
VH1-VH2-VL2-VL1-CH1-Fc;
VL1-VL2-VH2-VH1-CL-Fc;
VH1-VH2-VL2-VL1-CL-Fc;
VL1-VL2-VH2-VH1-CH1-CL-Fc;
VH1-VH2-VL2-VL1-CH1-CL-Fc;
VL1-VL2-VH2-VH1-CL-CH1-Fc;
VH1-VH2-VL2-VL1-CL-CH1-Fc;
VL1-L1-VL2-L2-VH2-L3-VH1-L4-CH1-L5-Fc;
VH1-L1-VH2-L2-VL2-L3-VL1-L4-CH1-L5-Fc;
VL1-L1-VL2-L2-VH2-L3-VH1-L4-CL-L5-Fc;
VH1-L1-VH2-L2-VL2-L3-VL1-L4-CL-L5-Fc;
VL1-L1-VL2-L2-VH2-L3-VH1-L4-CH1-L5-CL-L6-Fc;
VH1-L1-VH2-L2-VL2-L3-VL1-L4-CH1-L5-CL-L6-Fc;
VL1-L1-VL2-L2-VH2-L3-VH1-L4-CL-L5-CH1-L6-Fc; or
VH1-L1-VH2-L2-VL2-L3-VL1-L4-CL-L5-CH1-L6-Fc;
wherein the second polypeptide has a structure represented by:
VL3-CH1-Fc;
VL3-CL-Fc;
VL3-L7-CH1-L8-Fc; or
VL3-L7-CL-L8-Fc;
wherein the third polypeptide has a structure represented by:
VH3-CH1;
VH3-CL;
VH3-L9-CH1; or
VH3-L9-CL; and
wherein:
VL1 is a first immunoglobulin light chain variable region that specifically binds to an HIV protein;
VL2 is a second immunoglobulin light chain variable region that specifically binds to an HIV protein;
VL3 is a third immunoglobulin light chain variable region that specifically binds to an HIV protein;
VH1 is a first immunoglobulin heavy chain variable region that specifically binds to an HIV protein;
VH2 is a second immunoglobulin heavy chain variable region that specifically binds to an HIV protein;
VH3 is a third immunoglobulin heavy chain variable region that specifically binds to an HIV protein;
CH1 is an immunoglobulin heavy chain constant region 1;
CL is an immunoglobulin light chain constant region; and
L1, L2, L3, L4, L5, L6, L7, L8, and L9 are amino acid linkers.

92. The antigen binding polypeptide complex of claim 84, wherein the HIV protein is an HIV envelope protein, an HIV structural protein, an HIV functional protein, or an HIV accessory protein.

93. The antigen binding polypeptide complex of claim 84, wherein the antigen binding polypeptide complex comprises VH and VL sequences from the N6, PGT145, PG9, PG16, PGT128, PGT121, 10-1074, 3BNC117, VRC01, PGT151, 4E10, or 10E8 antibody.

94. The antigen binding polypeptide complex of claim 93, wherein the 10E8 antibody is the 10E8v4 antibody.

95. The antigen binding polypeptide complex of claim 84, wherein VH1, VH2, VH3, and VH4 each comprise a heavy chain variable region from the PGT121, VRC01, 10E8v4, or PG16 antibody; and/or VL1, VL2, VL3, and VL4 each comprise a light chain variable region from the PGT121, VRC01, 10E8v4, or PG16 antibody.

96. The antigen binding polypeptide complex of claim 84, wherein the Fc region comprises at least one knob-into-hole modification.

97. The antigen binding polypeptide complex of claim 96, wherein the antigen binding polypeptide complex is an IgG1 or IgG4 antibody and the knob-into-hole modification comprises:
(i) knob substitutions of S354C and T366W and hole substitutions of Y349C, T366S, L368A and Y407V;
(ii) hole substitutions of L234A, L235A and P239A;
(iii) hole substitutions of L234A and L235A;
(iv) hole substitutions of M428L and N433S;
(v) hole substitutions of M252Y, S254T and T256E; or
(vi) a combination thereof;
based on the EU numbering scheme.

98. A chimeric antigen receptor (CAR) comprising the antigen binding polypeptide complex of claim 84.

99. A pharmaceutical composition comprising (i) the antigen binding polypeptide complex of claim 84, and (ii) a pharmaceutically acceptable carrier.

100. A method of treating or preventing human immunodeficiency virus (HIV) infection, comprising administering to a subject in need thereof the antigen binding polypeptide complex of claim 84.

101. A method of treating or preventing acquired immune deficiency syndrome (AIDS), comprising administering to a subject in need thereof the antigen binding polypeptide complex of claim 84.

102. A method of treating or preventing AIDS-related complex (ARC), comprising administering to a subject in need thereof the antigen binding polypeptide complex of claim 84.

103. A method of treating or preventing an HIV-related opportunistic infection, comprising administering to a subject in need thereof the antigen binding polypeptide complex of claim 84.


SEQUENCE LISTING (107 sequences)

SEQ ID NO: 1 - protein, 5 aa, synthetic construct
  REGION 1..5: note = Linker
GGSSG

SEQ ID NO: 2 - protein, 5 aa, synthetic construct
  REGION 1..5: note = Linker
GSSGS

SEQ ID NO: 3 - protein, 5 aa, synthetic construct
  REGION 1..5: note = Linker
GTVAA

SEQ ID NO: 4 - protein, 5 aa, synthetic construct
  REGION 1..5: note = Linker
ASGGS

SEQ ID NO: 5 - protein, 5 aa, synthetic construct
  REGION 1..5: note = Linker
ASTGG

SEQ ID NO: 6 - protein, 6 aa, synthetic construct
  REGION 1..6: note = Linker
ASGGSG

SEQ ID NO: 7 - protein, 10 aa, synthetic construct
  REGION 1..10: note = Linker
GGSGGSSGSS

SEQ ID NO: 8 - protein, 10 aa, synthetic construct
  REGION 1..10: note = Linker
SGGSGSSGGS

SEQ ID NO: 9 - protein, 17 aa, synthetic construct
  REGION 1..17: note = Linker
GGSGGSGSGGGSASGSG

SEQ ID NO: 10 - protein, 18 aa, synthetic construct
  REGION 1..18: note = Linker
GGSGGSGSGGGGSASGSG

SEQ ID NO: 11 - protein, 20 aa, synthetic construct
  REGION 1..20: note = Linker
GGGSSGGGGSGGSGSGGSGS

SEQ ID NO: 12 - protein, 20 aa, synthetic construct
  REGION 1..20: note = Linker
GGGGSGGSGSGGGGSASGSG

SEQ ID NO: 13 - protein, 20 aa, synthetic construct
  REGION 1..20: note = Linker
GGGSSGGSGSGGSGSGGSGS

SEQ ID NO: 14 - protein, 22 aa, synthetic construct
  REGION 1..22: note = Linker
SGGSSGGSGSGGSGSGGSGSSG

SEQ ID NO: 15 - protein, 22 aa, synthetic construct
  REGION 1..22: note = Linker
GSGSSGGGGSGGSGSGGSGSSG

SEQ ID NO: 16 - protein, 40 aa, synthetic construct
  REGION 1..40: note = Linker
GGGGSGSGGSGGGSSGGGGSGGGGSGGGGSGGGGSGGGGS

SEQ ID NO: 17 - protein, 40 aa, synthetic construct
  REGION 1..40: note = Linker
GGGGSGGGGSGGGGSGGGGSGGGGSGGGGSGGGGSGGGGS

SEQ ID NO: 18 - protein, 43 aa, synthetic construct
  REGION 1..43: note = Linker
GGGGSGSGGSGGGSSGGGGSGGGGSGGGGSGGGGSGGGGSSSS

SEQ ID NO: 19 - protein, 45 aa, synthetic construct
  REGION 1..45: note = Linker
GGGGSGSGGSGGGSSGGGGSGGGGSGGGGSGGGGSGGGGSSSSGS

SEQ ID NO: 20 - protein, 132 aa, synthetic construct
  REGION 1..132: note = PGT121 VH
QMQLQESGPGLVKPSETLSLTCSVSGASISDSYWSWIRRSPGKGLEWIGYVHKSGDTNYSPSLKSRVNLSLDTSKNQVSLSLVAATAADSGKYYCARTLHGRRIYGIVAFNEWFTYFYMDVWGNGTQVTVSS

SEQ ID NO: 21 - protein, 121 aa, synthetic construct
  REGION 1..121: note = VRC01 VH
QVQLVQSGGQMKKPGESMRISCRASGYEFIDCTLNWIRLAPGKRPEWMGWLKPRGGAVNYARPLQGRVTMTRDVYSDTAFLELRSLTVDDTAVYFCTRGKNCDYNWDFEHWGRGTPVIVSS

SEQ ID NO: 22 - protein, 131 aa, synthetic construct
  REGION 1..131: note = 10E8v4 VH
EVRLVESGGGLVKPGGSLRLSCSASGFDFDNAWMTWVRQPPGKGLEWVGRITGPGEGWSVDYAESVKGRFTISRDNTKNTLYLEMNNVRTEDTGYYFCARTGKYYDFWSGYPPGEEYFQDWGQGTLVIVSS

SEQ ID NO: 23 - protein, 137 aa, synthetic construct
  REGION 1..137: note = PG16 VH
QEQLVESGGGVVQPGGSLRLSCLASGFTFHKYGMHWVRQAPGKGLEWVALISDDGMRKYHSDSMWGRVTISRDNSKNTLYLQFSSLKVEDTAMFFCAREAGGPIWHDDVKYYDFNDGYYNYHYMDVWGKGTTVTVSS

SEQ ID NO: 24 - protein, 105 aa, synthetic construct
  REGION 1..105: note = PGT121 VL
SDISVAPGETARISCGEKSLGSRAVQWYQHRAGQAPSLIIYNNQDRPSGIPERFSGSPDSPFGTTATLTITSVEAGDEADYYCHIWDSRVPTKWVFGGGTTLTVL

SEQ ID NO: 25 - protein, 100 aa, synthetic construct
  REGION 1..100: note = VRC01 VL
LTQSPGTLSLSPGETAIISCRTSQYGSLAWYQQRPGQAPRLVIYSGSTRAAGIPDRFSGSRWGPDYNLTISNLESGDFGVYYCQQYEFFGQGTKVQVDIK

SEQ ID NO: 26 - protein, 108 aa, synthetic construct
  REGION 1..108: note = 10E8v4 VL
SELTQDPAVSVALKQTVTITCRGDSLRSHYASWYQKKPGQAPVLLFYGKNNRPSGIPDRFSGSASGNRASLTITGAQAEDEADYYCSSRDKSGSRLSVFGGGTKLTVL

SEQ ID NO: 27 - protein, 110 aa, synthetic construct
  REGION 1..110: note = PG16 VL
QSALTQPASVSGSPGQTITISCNGTSSDVGGFDSVSWYQQSPGKAPKVMVFDVSHRPSGISNRFSGSKSGNTASLTISGLHIEDEGDYFCSSLTDRSHRIFGGGTKVTVL

SEQ ID NO: 28 - DNA, 1383 bp, synthetic construct
  misc_feature 1..1383: note = PGT121 VH
  source 1..1383: mol_type = other DNA
cagatgcagctccaagagtctggccctggcctggtcaagcctagcgaaacactgagcctgacctgttccgtgtccggcgccagcatcagcgatagctactggtcctggatcagaagaagccccggcaaaggcctggaatggatcggctacgtgcacaagagcggcgacaccaactatagccccagcctgaagtccagagtgaacctgagcctggacaccagcaagaaccaggtgtccctgtctctggtggccgctacagctgccgatagcggcaagtactactgcgccagaactctgcacggcagacggatctatggcatcgtggccttcaacgagtggttcacctacttctacatggacgtgtggggcaacggcacccaagtgacagtctcttctgcttcgaccaagggcccatccgtgtttcctctggctcccagcagcaagtctacaagcggaggaacagccgctctgggctgcctggtcaaggattactttcccgagcctgtgaccgtgtcctggaattctggcgctctgacaagcggcgtgcacacctttccagctgtgctgcaaagcagcggcctgtactctctgagcagcgtggtcacagtgcctagctctagcctgggcacccagacctacatctgcaatgtgaaccacaagcctagcaacaccaaggtggacaagaaggtggaacccaagagctgcgataagacccacacatgtcctccatgtcctgctccagaactgctgggcggaccttccgtgtttctgttccctccaaagcctaaggacaccctgatgatcagcaggacccctgaagtgacctgcgtggtggtggatgtgtctcacgaggacccagaagtgaagttcaattggtacgtggacggcgtggaagtgcacaacgccaagaccaagcctagagaggaacagtacaacagcacctacagagtggtgtccgtgctgacagtgctgcaccaggattggctgaacggcaaagagtacaagtgcaaggtgtccaacaaggccctgcccgcccctatcgagaaaaccatcagcaaggccaagggccagcctagggaaccccaggtttacacccttcctccatgccgggatgagctgaccaagaaccaggtgtccctgtggtgtctggtcaagggcttctaccccagcgacattgccgtggaatgggagagcaatggccagcctgagaacaactacaagaccacacctcctgtgctggactccgacggatcattcttcctgtacagcaagctgactgtggacaagagcagatggcagcagggcaacgtgttcagctgcagcgtgatgcacgaggccctgcacaaccactacacacagaagtccctgtctctgagccctggc

SEQ ID NO: 29 - DNA, 1398 bp, synthetic construct
  misc_feature 1..1398: note = PG16 VH
  source 1..1398: mol_type = other DNA
caagaacagctggtggaatctggtggcggagtggttcaacctggcggctctctgagactgagctgtctggcctccggcttcaccttccacaaatacggcatgcactgggtccgacaggcccctggaaaaggccttgaatgggtcgccctgatcagcgacgacggcatgagaaagtaccacagcgacagcatgtggggcagagtgaccatcagccgggacaacagcaagaacaccctgtacctgcagttcagctccctgaaggtggaagataccgccatgttcttttgcgccagagaagccggcggacccatctggcatgacgacgtgaagtactacgacttcaacgacggctactacaactaccactacatggacgtgtggggcaagggcaccaccgtgacagtttcttctgcttcgaccaagggcccatccgtgtttcctctggctcccagcagcaagtctacaagcggaggaacagccgctctgggctgcctggtcaaggattactttcccgagcctgtgaccgtgtcctggaattctggcgctctgacaagcggcgtgcacacctttccagctgtgctgcaaagcagcggcctgtactctctgagcagcgtggtcacagtgcctagctctagcctgggcacccagacctacatctgcaatgtgaaccacaagcctagcaacaccaaggtggacaagaaggtggaacccaagagctgcgataagacccacacatgtcctccatgtcctgctccagaactgctgggcggaccttccgtgtttctgttccctccaaagcctaaggacaccctgatgatcagcaggacccctgaagtgacctgcgtggtggtggatgtgtctcacgaggacccagaagtgaagttcaattggtacgtggacggcgtggaagtgcacaacgccaagaccaagcctagagaggaacagtacaacagcacctacagagtggtgtccgtgctgacagtgctgcaccaggattggctgaacggcaaagagtacaagtgcaaggtgtccaacaaggccctgcccgcccctatcgagaaaaccatcagcaaggccaagggccagcctagggaaccccaggtttacacccttcctccatgccgggatgagctgaccaagaaccaggtgtccctgtggtgtctggtcaagggcttctaccccagcgacattgccgtggaatgggagagcaatggccagcctgagaacaactacaagaccacacctcctgtgctggactccgacggatcattcttcctgtacagcaagctgactgtggacaagagcagatggcagcagggcaacgtgttcagctgcagcgtgatgcacgaggccctgcacaaccactacacacagaagtccctgtctctgagccctggc

SEQ ID NO: 30 - DNA, 633 bp, synthetic construct
  misc_feature 1..633: note = PGT121 VL
  source 1..633: mol_type = other DNA
agcgatatctctgtggcccctggcgagacagccagaatcagctgtggcgagaagtccctgggaagcagagccgtgcagtggtatcagcatagagccggacaggcccctagcctgatcatctacaacaaccaggacagacccagcggcatccccgagagattttctggcagccctgatagccctttcggcaccacagccacactgaccatcacatctgtggaagccggcgacgaggccgactactactgtcacatctgggacagcagggtgcccaccaaatgggttttcggcggaggcacaaccctgaccgttctgggacagcctaaggccaatcctaccgtgacactgttccctccaagcagcgaagaactgcaggccaacaaggccacactcgtgtgcctgatcagcgacttttatcctggcgccgtgaccgtggcctggaaggctgatggatctcctgtgaaagccggcgtggaaaccaccaagcctagcaagcagagcaacaacaaatacgccgccagcagctacctgagcctgacacctgagcagtggaagtcccacagatcctacagctgccaagtgacccacgagggcagcaccgtggaaaaaacagtggcccctaccgagtgcagc

SEQ ID NO: 31 - DNA, 648 bp, synthetic construct
  misc_feature 1..648: note = PG16 VL
  source 1..648: mol_type = other DNA
cagtctgctctgacacagcctgcctctgtgtctggctctcctggccagaccatcaccatcagctgtaacggcaccagctctgacgtcggcggctttgatagcgtgtcctggtatcagcagagccccggcaaagcccctaaagtgatggtgttcgatgtgtcccaccggcctagcggcatcagcaatagattcagcggcagcaagagcggcaacaccgccagcctgacaatctctggactgcacatcgaggacgagggcgactacttctgtagcagcctgaccgacagaagccaccggatttttggcggaggcaccaaagtgaccgtgctgggacagcctaaggccaatcctaccgtgacactgttccctccaagcagcgaagaactgcaggccaacaaggccacactcgtgtgcctgatcagcgacttttatcctggcgccgtgaccgtggcctggaaggctgatggatctcctgtgaaagccggcgtggaaaccaccaagcctagcaagcagagcaacaacaaatacgccgccagcagctacctgagcctgacacctgagcagtggaagtcccacagatcctacagctgccaagtgacccacgagggcagcaccgtggaaaaaacagtggcccctaccgagtgcagc

SEQ ID NO: 32 - protein, 241 aa, synthetic construct
  REGION 1..241: note = VRC01scFv
LTQSPGTLSLSPGETAIISCRTSQYGSLAWYQQRPGQAPRLVIYSGSTRAAGIPDRFSGSRWGPDYNLTISNLESGDFGVYYCQQYEFFGQGTKVQVDIKGGGGSGGSGSGGGGSASGSGQVQLVQSGGQMKKPGESMRISCRASGYEFIDCTLNWIRLAPGKRPEWMGWLKPRGGAVNYARPLQGRVTMTRDVYSDTAFLELRSLTVDDTAVYFCTRGKNCDYNWDFEHWGRGTPVIVSS

SEQ ID NO: 33 - DNA, 1428 bp, synthetic construct
  misc_feature 1..1428: note = VRC01scFv
  source 1..1428: mol_type = other DNA
gagatcgtgctgacacagagccctggcaccctgagcctgtctccaggcgagacagccatcatcagctgccggacaagccagtacggcagcctggcctggtatcagcagaggcctggacaggcccccagactcgtgatctacagcggcagcacaagagccgccggaatccccgatagattcagcggctccagatggggccctgactacaacctgaccatcagcaacctggaaagcggcgacttcggcgtgtactactgccagcagtacgagttcttcggccagggcaccaaggtgcaggtggacatcaagggcggcggaggaagcggcggatctggatctggtggcggaggatctgcttctggctctggacaggttcagctggttcagtctggcggacagatgaagaaacccggcgagagcatgcggatcagctgtagagccagcggctacgagttcatcgactgcaccctgaactggatcagactggcccctggcaaaagacccgagtggatgggatggctgaagcctagaggcggagccgtgaattacgccagacctctgcagggcagagtgaccatgaccagggacgtgtacagcgataccgccttcctggaactgagaagcctgaccgtggatgataccgccgtgtacttctgcacccggggcaagaactgcgactacaactgggacttcgagcactggggcagaggcacacctgtgatcgttagctctgctagcggaggttccggagataagacccacacatgtcctccatgtcctgctccagaactgctgggcggaccttccgtgtttctgttccctccaaagcctaaggacaccctgatgatcagcaggacccctgaagtgacctgcgtggtggtggatgtgtctcacgaggacccagaagtgaagttcaattggtacgtggacggcgtggaagtgcacaacgccaagaccaagcctagagaggaacagtacaacagcacctacagagtggtgtccgtgctgacagtgctgcaccaggattggctgaacggcaaagagtacaagtgcaaggtgtccaacaaggccctgcccgcccctatcgagaaaaccatcagcaaggccaagggccagcctagggaaccccaggtttacacccttcctccatgccgggatgagctgaccaagaaccaggtgtccctgtggtgtctggtcaagggcttctaccccagcgacattgccgtggaatgggagagcaatggccagcctgagaacaactacaagaccacacctcctgtgctggactccgacggatcattcttcctgtacagcaagctgactgtggacaagagcagatggcagcagggcaacgtgttcagctgcagcgtgatgcacgaggccctgcacaaccactacacacagaagtccctgtctctgagccctggc

SEQ ID NO: 34 - protein, 259 aa, synthetic construct
  REGION 1..259: note = 10e8v4scFv
SELTQDPAVSVALKQTVTITCRGDSLRSHYASWYQKKPGQAPVLLFYGKNNRPSGIPDRFSGSASGNRASLTITGAQAEDEADYYCSSRDKSGSRLSVFGGGTKLTVLGGGGSGGSGSGGGGSASGSGEVRLVESGGGLVKPGGSLRLSCSASGFDFDNAWMTWVRQPPGKGLEWVGRITGPGEGWSVDYAESVKGRFTISRDNTKNTLYLEMNNVRTEDTGYYFCARTGKYYDFWSGYPPGEEYFQDWGQGTLVIVSS

SEQ ID NO: 35 - DNA, 1473 bp, synthetic construct
  misc_feature 1..1473: note = 10e8v4scFv
  source 1..1473: mol_type = other DNA
agcgaactgacccaagatcctgccgtgtctgtggccctgaagcagaccgtgaccatcacctgtagaggcgacagcctgagaagccactacgccagctggtatcagaagaagcctggacaggctcccgtgctgctgttctacggcaagaacaacagacccagcggcatccccgatagattttctggcagcgccagcggcaatagagccagcctgacaattactggcgcccaggccgaagatgaggccgactactactgcagcagcagagacaagagcggcagcagactgtccgtttttggcggaggcaccaagctgaccgtgctgggcggcggaggaagcggcggatctggatctggtggcggaggatctgcttctggctctggagaagttcggctggtggaatctggcggcggacttgtgaaacctggcggctctctgagactgagctgtagcgcctccggcttcgacttcgacaatgcctggatgacctgggtccgacagcctcctggaaaaggcctggaatgggtcggaagaatcacaggccctggcgaaggctggagtgtggattatgccgagagcgtgaagggcagattcaccatcagccgggacaacaccaagaacaccctgtacctggaaatgaacaacgtgcggaccgaggataccggctactacttctgtgcccggaccggcaagtactacgacttttggagcggctaccctcctggcgaggaatactttcaggattggggccagggcaccctggtcatcgtttcttctgctagcggaggttccggagataagacccacacatgtcctccatgtcctgctccagaactgctgggcggaccttccgtgtttctgttccctccaaagcctaaggacaccctgatgatcagcaggacccctgaagtgacctgcgtggtggtggatgtgtctcacgaggacccagaagtgaagttcaattggtacgtggacggcgtggaagtgcacaacgccaagaccaagcctagagaggaacagtacaacagcacctacagagtggtgtccgtgctgacagtgctgcaccaggattggctgaacggcaaagagtacaagtgcaaggtgtccaacaaggccctgcccgcccctatcgagaaaaccatcagcaaggccaagggccagcctagggaaccccaggtttacacccttcctccatgccgggatgagctgaccaagaaccaggtgtccctgtggtgtctggtcaagggcttctaccccagcgacattgccgtggaatgggagagcaatggccagcctgagaacaactacaagaccacacctcctgtgctggactccgacggatcattcttcctgtacagcaagctgactgtggacaagagcagatggcagcagggcaacgtgttcagctgcagcgtgatgcacgaggccctgcacaaccactacacacagaagtccctgtctctgagccctggc

SEQ ID NO: 36 - protein, 720 aa, synthetic construct
  REGION 1..720: note = PGT121VRC01L1holeFc
SDISVAPGETARISCGEKSLGSRAVQWYQHRAGQAPSLIIYNNQDRPSGIPERFSGSPDSPFGTTATLTITSVEAGDEADYYCHIWDSRVPTKWVFGGGTTLTVLGGSSGLTQSPGTLSLSPGETAIISCRTSQYGSLAWYQQRPGQAPRLVIYSGSTRAAGIPDRFSGSRWGPDYNLTISNLESGDFGVYYCQQYEFFGQGTKVQVDIKGGGGSGGSGSGGGGSASGSGQVQLVQSGGQMKKPGESMRISCRASGYEFIDCTLNWIRLAPGKRPEWMGWLKPRGGAVNYARPLQGRVTMTRDVYSDTAFLELRSLTVDDTAVYFCTRGKNCDYNWDFEHWGRGTPVIVSSGGSSGQMQLQESGPGLVKPSETLSLTCSVSGASISDSYWSWIRRSPGKGLEWIGYVHKSGDTNYSPSLKSRVNLSLDTSKNQVSLSLVAATAADSGKYYCARTLHGRRIYGIVAFNEWFTYFYMDVWGNGTQVTVSSASGGSGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 37 - protein, 720 aa, synthetic construct
  REGION 1..720: note = VRC01PGT121L1holeFc
LTQSPGTLSLSPGETAIISCRTSQYGSLAWYQQRPGQAPRLVIYSGSTRAAGIPDRFSGSRWGPDYNLTISNLESGDFGVYYCQQYEFFGQGTKVQVDIKGGSSGSDISVAPGETARISCGEKSLGSRAVQWYQHRAGQAPSLIIYNNQDRPSGIPERFSGSPDSPFGTTATLTITSVEAGDEADYYCHIWDSRVPTKWVFGGGTTLTVLGGGGSGGSGSGGGGSASGSGQMQLQESGPGLVKPSETLSLTCSVSGASISDSYWSWIRRSPGKGLEWIGYVHKSGDTNYSPSLKSRVNLSLDTSKNQVSLSLVAATAADSGKYYCARTLHGRRIYGIVAFNEWFTYFYMDVWGNGTQVTVSSGGSSGQVQLVQSGGQMKKPGESMRISCRASGYEFIDCTLNWIRLAPGKRPEWMGWLKPRGGAVNYARPLQGRVTMTRDVYSDTAFLELRSLTVDDTAVYFCTRGKNCDYNWDFEHWGRGTPVIVSSASGGSGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 38 - protein, 748 aa, synthetic construct
  REGION 1..748: note = 10e8v4PG16L1knob
SELTQDPAVSVALKQTVTITCRGDSLRSHYASWYQKKPGQAPVLLFYGKNNRPSGIPDRFSGSASGNRASLTITGAQAEDEADYYCSSRDKSGSRLSVFGGGTKLTVLGGSSGQSALTQPASVSGSPGQTITISCNGTSSDVGGFDSVSWYQQSPGKAPKVMVFDVSHRPSGISNRFSGSKSGNTASLTISGLHIEDEGDYFCSSLTDRSHRIFGGGTKVTVLGGGGSGGSGSGGGGSASGSGQEQLVESGGGVVQPGGSLRLSCLASGFTFHKYGMHWVRQAPGKGLEWVALISDDGMRKYHSDSMWGRVTISRDNSKNTLYLQFSSLKVEDTAMFFCAREAGGPIWHDDVKYYDFNDGYYNYHYMDVWGKGTTVTVSSGGSSGEVRLVESGGGLVKPGGSLRLSCSASGFDFDNAWMTWVRQPPGKGLEWVGRITGPGEGWSVDYAESVKGRFTISRDNTKNTLYLEMNNVRTEDTGYYFCARTGKYYDFWSGYPPGEEYFQDWGQGTLVIVSSASGGSGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 39 - protein, 748 aa, synthetic construct
  REGION 1..748: note = PG1610e8v4L1knob
QSALTQPASVSGSPGQTITISCNGTSSDVGGFDSVSWYQQSPGKAPKVMVFDVSHRPSGISNRFSGSKSGNTASLTISGLHIEDEGDYFCSSLTDRSHRIFGGGTKVTVLGGSSGSELTQDPAVSVALKQTVTITCRGDSLRSHYASWYQKKPGQAPVLLFYGKNNRPSGIPDRFSGSASGNRASLTITGAQAEDEADYYCSSRDKSGSRLSVFGGGTKLTVLGGGGSGGSGSGGGGSASGSGEVRLVESGGGLVKPGGSLRLSCSASGFDFDNAWMTWVRQPPGKGLEWVGRITGPGEGWSVDYAESVKGRFTISRDNTKNTLYLEMNNVRTEDTGYYFCARTGKYYDFWSGYPPGEEYFQDWGQGTLVIVSSGGSSGQEQLVESGGGVVQPGGSLRLSCLASGFTFHKYGMHWVRQAPGKGLEWVALISDDGMRKYHSDSMWGRVTISRDNSKNTLYLQFSSLKVEDTAMFFCAREAGGPIWHDDVKYYDFNDGYYNYHYMDVWGKGTTVTVSSASGGSGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 40 - protein, 738 aa, synthetic construct
  REGION 1..738: note = PGT12110e8v4L1holeFc
SDISVAPGETARISCGEKSLGSRAVQWYQHRAGQAPSLIIYNNQDRPSGIPERFSGSPDSPFGTTATLTITSVEAGDEADYYCHIWDSRVPTKWVFGGGTTLTVLGGSSGSELTQDPAVSVALKQTVTITCRGDSLRSHYASWYQKKPGQAPVLLFYGKNNRPSGIPDRFSGSASGNRASLTITGAQAEDEADYYCSSRDKSGSRLSVFGGGTKLTVLGGGGSGGSGSGGGGSASGSGEVRLVESGGGLVKPGGSLRLSCSASGFDFDNAWMTWVRQPPGKGLEWVGRITGPGEGWSVDYAESVKGRFTISRDNTKNTLYLEMNNVRTEDTGYYFCARTGKYYDFWSGYPPGEEYFQDWGQGTLVIVSSGGSSGQMQLQESGPGLVKPSETLSLTCSVSGASISDSYWSWIRRSPGKGLEWIGYVHKSGDTNYSPSLKSRVNLSLDTSKNQVSLSLVAATAADSGKYYCARTLHGRRIYGIVAFNEWFTYFYMDVWGNGTQVTVSSASGGSGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 41 - protein, 738 aa, synthetic construct
  REGION 1..738: note = 10e8v4PGT121L1holeFc
SELTQDPAVSVALKQTVTITCRGDSLRSHYASWYQKKPGQAPVLLFYGKNNRPSGIPDRFSGSASGNRASLTITGAQAEDEADYYCSSRDKSGSRLSVFGGGTKLTVLGGSSGSDISVAPGETARISCGEKSLGSRAVQWYQHRAGQAPSLIIYNNQDRPSGIPERFSGSPDSPFGTTATLTITSVEAGDEADYYCHIWDSRVPTKWVFGGGTTLTVLGGGGSGGSGSGGGGSASGSGQMQLQESGPGLVKPSETLSLTCSVSGASISDSYWSWIRRSPGKGLEWIGYVHKSGDTNYSPSLKSRVNLSLDTSKNQVSLSLVAATAADSGKYYCARTLHGRRIYGIVAFNEWFTYFYMDVWGNGTQVTVSSGGSSGEVRLVESGGGLVKPGGSLRLSCSASGFDFDNAWMTWVRQPPGKGLEWVGRITGPGEGWSVDYAESVKGRFTISRDNTKNTLYLEMNNVRTEDTGYYFCARTGKYYDFWSGYPPGEEYFQDWGQGTLVIVSSASGGSGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 42 - protein, 730 aa, synthetic construct
  REGION 1..730: note = VRC01PG16L1knob
LTQSPGTLSLSPGETAIISCRTSQYGSLAWYQQRPGQAPRLVIYSGSTRAAGIPDRFSGSRWGPDYNLTISNLESGDFGVYYCQQYEFFGQGTKVQVDIKGGSSGQSALTQPASVSGSPGQTITISCNGTSSDVGGFDSVSWYQQSPGKAPKVMVFDVSHRPSGISNRFSGSKSGNTASLTISGLHIEDEGDYFCSSLTDRSHRIFGGGTKVTVLGGGGSGGSGSGGGGSASGSGQEQLVESGGGVVQPGGSLRLSCLASGFTFHKYGMHWVRQAPGKGLEWVALISDDGMRKYHSDSMWGRVTISRDNSKNTLYLQFSSLKVEDTAMFFCAREAGGPIWHDDVKYYDFNDGYYNYHYMDVWGKGTTVTVSSGGSSGQVQLVQSGGQMKKPGESMRISCRASGYEFIDCTLNWIRLAPGKRPEWMGWLKPRGGAVNYARPLQGRVTMTRDVYSDTAFLELRSLTVDDTAVYFCTRGKNCDYNWDFEHWGRGTPVIVSSASGGSGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 43 - protein, 730 aa, synthetic construct
  REGION 1..730: note = PG16VRC01L1knob
QSALTQPASVSGSPGQTITISCNGTSSDVGGFDSVSWYQQSPGKAPKVMVFDVSHRPSGISNRFSGSKSGNTASLTISGLHIEDEGDYFCSSLTDRSHRIFGGGTKVTVLGGSSGLTQSPGTLSLSPGETAIISCRTSQYGSLAWYQQRPGQAPRLVIYSGSTRAAGIPDRFSGSRWGPDYNLTISNLESGDFGVYYCQQYEFFGQGTKVQVDIKGGGGSGGSGSGGGGSASGSGQVQLVQSGGQMKKPGESMRISCRASGYEFIDCTLNWIRLAPGKRPEWMGWLKPRGGAVNYARPLQGRVTMTRDVYSDTAFLELRSLTVDDTAVYFCTRGKNCDYNWDFEHWGRGTPVIVSSGGSSGQEQLVESGGGVVQPGGSLRLSCLASGFTFHKYGMHWVRQAPGKGLEWVALISDDGMRKYHSDSMWGRVTISRDNSKNTLYLQFSSLKVEDTAMFFCAREAGGPIWHDDVKYYDFNDGYYNYHYMDVWGKGTTVTVSSASGGSGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 44 - protein, 746 aa, synthetic construct
  REGION 1..746: note = PGT121PG16L1holeFc
SDISVAPGETARISCGEKSLGSRAVQWYQHRAGQAPSLIIYNNQDRPSGIPERFSGSPDSPFGTTATLTITSVEAGDEADYYCHIWDSRVPTKWVFGGGTTLTVLGGSSGQSALTQPASVSGSPGQTITISCNGTSSDVGGFDSVSWYQQSPGKAPKVMVFDVSHRPSGISNRFSGSKSGNTASLTISGLHIEDEGDYFCSSLTDRSHRIFGGGTKVTVLGGGGSGGSGSGGGGSASGSGQEQLVESGGGVVQPGGSLRLSCLASGFTFHKYGMHWVRQAPGKGLEWVALISDDGMRKYHSDSMWGRVTISRDNSKNTLYLQFSSLKVEDTAMFFCAREAGGPIWHDDVKYYDFNDGYYNYHYMDVWGKGTTVTVSSGGSSGQMQLQESGPGLVKPSETLSLTCSVSGASISDSYWSWIRRSPGKGLEWIGYVHKSGDTNYSPSLKSRVNLSLDTSKNQVSLSLVAATAADSGKYYCARTLHGRRIYGIVAFNEWFTYFYMDVWGNGTQVTVSSASGGSGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 45 - protein, 746 aa, synthetic construct
  REGION 1..746: note = PG16PGT121L1holeFc
QSALTQPASVSGSPGQTITISCNGTSSDVGGFDSVSWYQQSPGKAPKVMVFDVSHRPSGISNRFSGSKSGNTASLTISGLHIEDEGDYFCSSLTDRSHRIFGGGTKVTVLGGSSGSDISVAPGETARISCGEKSLGSRAVQWYQHRAGQAPSLIIYNNQDRPSGIPERFSGSPDSPFGTTATLTITSVEAGDEADYYCHIWDSRVPTKWVFGGGTTLTVLGGGGSGGSGSGGGGSASGSGQMQLQESGPGLVKPSETLSLTCSVSGASISDSYWSWIRRSPGKGLEWIGYVHKSGDTNYSPSLKSRVNLSLDTSKNQVSLSLVAATAADSGKYYCARTLHGRRIYGIVAFNEWFTYFYMDVWGNGTQVTVSSGGSSGQEQLVESGGGVVQPGGSLRLSCLASGFTFHKYGMHWVRQAPGKGLEWVALISDDGMRKYHSDSMWGRVTISRDNSKNTLYLQFSSLKVEDTAMFFCAREAGGPIWHDDVKYYDFNDGYYNYHYMDVWGKGTTVTVSSASGGSGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 46 - protein, 722 aa, synthetic construct
  REGION 1..722: note = VRC0110e8v4L1knob
LTQSPGTLSLSPGETAIISCRTSQYGSLAWYQQRPGQAPRLVIYSGSTRAAGIPDRFSGSRWGPDYNLTISNLESGDFGVYYCQQYEFFGQGTKVQVDIKGGSSGSELTQDPAVSVALKQTVTITCRGDSLRSHYASWYQKKPGQAPVLLFYGKNNRPSGIPDRFSGSASGNRASLTITGAQAEDEADYYCSSRDKSGSRLSVFGGGTKLTVLGGGGSGGSGSGGGGSASGSGEVRLVESGGGLVKPGGSLRLSCSASGFDFDNAWMTWVRQPPGKGLEWVGRITGPGEGWSVDYAESVKGRFTISRDNTKNTLYLEMNNVRTEDTGYYFCARTGKYYDFWSGYPPGEEYFQDWGQGTLVIVSSGGSSGQVQLVQSGGQMKKPGESMRISCRASGYEFIDCTLNWIRLAPGKRPEWMGWLKPRGGAVNYARPLQGRVTMTRDVYSDTAFLELRSLTVDDTAVYFCTRGKNCDYNWDFEHWGRGTPVIVSSASGGSGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 47 - protein, 722 aa, synthetic construct
  REGION 1..722: note = 10e8v4VRC01L1knob
SELTQDPAVSVALKQTVTITCRGDSLRSHYASWYQKKPGQAPVLLFYGKNNRPSGIPDRFSGSASGNRASLTITGAQAEDEADYYCSSRDKSGSRLSVFGGGTKLTVLGGSSGLTQSPGTLSLSPGETAIISCRTSQYGSLAWYQQRPGQAPRLVIYSGSTRAAGIPDRFSGSRWGPDYNLTISNLESGDFGVYYCQQYEFFGQGTKVQVDIKGGGGSGGSGSGGGGSASGSGQVQLVQSGGQMKKPGESMRISCRASGYEFIDCTLNWIRLAPGKRPEWMGWLKPRGGAVNYARPLQGRVTMTRDVYSDTAFLELRSLTVDDTAVYFCTRGKNCDYNWDFEHWGRGTPVIVSSGGSSGEVRLVESGGGLVKPGGSLRLSCSASGFDFDNAWMTWVRQPPGKGLEWVGRITGPGEGWSVDYAESVKGRFTISRDNTKNTLYLEMNNVRTEDTGYYFCARTGKYYDFWSGYPPGEEYFQDWGQGTLVIVSSASGGSGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 48 - DNA, 2169 bp, synthetic construct
  misc_feature 1..2169: note = PGT121VRC01L1holeFc
  source 1..2169: mol_type = other DNA
agcgatatctctgtggcccctggcgagacagccagaatcagctgtggcgagaagtccctgggaagcagagccgtgcagtggtatcagcatagagccggacaggcccctagcctgatcatctacaacaaccaggacagacccagcggcatccccgagagattttctggcagccctgatagccctttcggcaccacagccacactgaccatcacatctgtggaagccggcgacgaggccgactactactgtcacatctgggacagcagggtgcccaccaaatgggttttcggcggaggcacaaccctgaccgttctgggcggatcttctggagagatcgtgctgacacagagccctggcaccctgagcctgtctccaggcgagacagccatcatcagctgccggacaagccagtacggcagcctggcctggtatcagcagaggcctggacaggcccccagactcgtgatctacagcggcagcacaagagccgccggaatccccgatagattcagcggctccagatggggccctgactacaacctgaccatcagcaacctggaaagcggcgacttcggcgtgtactactgccagcagtacgagttcttcggccagggcaccaaggtgcaggtggacatcaagggaggcggaggatctggcggatctggaagtggcggcggtggatctgcttctggctctggccaggttcagctggttcagtctggcggacagatgaagaaacccggcgagagcatgcggatcagctgtagagccagcggctacgagttcatcgactgcaccctgaactggatcagactggcccctggcaaaagacccgagtggatgggatggctgaagcctagaggcggagccgtgaattacgccagacctctgcagggcagagtgaccatgaccagggacgtgtacagcgataccgccttcctggaactgagaagcctgaccgtggatgataccgccgtgtacttctgcacccggggcaagaactgcgactacaactgggacttcgagcactggggcagaggcacacctgtgatcgttagctctggcggatcttctggacagatgcagctccaagagtctggccctggcctggtcaagcctagcgaaacactgagcctgacctgttccgtgtccggcgccagcatcagcgatagctactggtcctggatcagaagaagccccggcaaaggcctggaatggatcggctacgtgcacaagagcggcgacaccaactatagccccagcctgaagtccagagtgaacctgagcctggacaccagcaagaaccaggtgtccctgtctctggtggccgctacagctgccgatagcggcaagtactactgcgccagaactctgcacggcagacggatctatggcatcgtggccttcaacgagtggttcacctacttctacatggacgtgtggggcaacggcacccaagtgacagtctcttctgcaagcggaggctccggagataagacccacacatgtcctccatgtcctgctccagagctgctgggtggcccttccgtgtttctgttccctccaaagcctaaggacaccctgatgatcagcaggacccctgaagtgacctgcgtggtggtggatgtgtctcacgaggacccagaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctgcccgctcctatcgagaaaaccatcagcaaggccaagggccagcctcgcgaacctcaagtctgtacactgcctcctagccgggacgagctgaccaagaatcaggtgtcactgagctgcgccgtgaagggcttttaccccagcgacattgccgtggaatgggagagcaatggccagccagagaacaactacaagacaacccctcctgtgctggacagcgacggctcatttttcctggtgtccaagctgacagtggacaagagcagatggcagcagggcaacgtgttcagctgcagcgtgatgcacgaggccctgcacaaccactacacccagaaaagcctgtctctgagccctggc

SEQ ID NO: 49 - DNA, 2169 bp, synthetic construct
  misc_feature 1..2169: note = VRC01PGT121L1holeFc
  source 1..2169: mol_type = other DNA
gagatcgtgctgacacagagccctggcaccctgagcctgtctccaggcgagacagccatcatcagctgccggacaagccagtacggcagcctggcctggtatcagcagaggcctggacaggcccccagactcgtgatctacagcggcagcacaagagccgccggaatccccgatagattcagcggctccagatggggccctgactacaacctgaccatcagcaacctggaaagcggcgacttcggcgtgtactactgccagcagtacgagttcttcggccagggcaccaaggtgcaggtggacatcaagggcggatcttctggaagcgatatctctgtggcccctggcgagacagccagaatcagctgtggcgagaagtccctgggaagcagagccgtgcagtggtatcagcatagagccggacaggcccctagcctgatcatctacaacaaccaggacagacccagcggcatccccgagagattttctggcagccctgatagccctttcggcaccacagccacactgaccatcacatctgtggaagccggcgacgaggccgactactactgtcacatctgggacagcagggtgcccaccaaatgggttttcggcggaggcacaaccctgaccgttctgggaggcggaggatctggcggatctggaagtggcggcggtggatctgcttctggctctggccagatgcagctccaagagtctggccctggcctggtcaagcctagcgaaacactgagcctgacctgttccgtgtccggcgccagcatcagcgatagctactggtcctggatcagaagaagccccggcaaaggcctggaatggatcggctacgtgcacaagagcggcgacaccaactatagccccagcctgaagtccagagtgaacctgagcctggacaccagcaagaaccaggtgtccctgtctctggtggccgctacagctgccgatagcggcaagtactactgcgccagaactctgcacggcagacggatctatggcatcgtggccttcaacgagtggttcacctacttctacatggacgtgtggggcaacggcacccaagtgacagtctcttctggcggatcttctggacaggttcagctggttcagtctggcggacagatgaagaaacccggcgagagcatgcggatcagctgtagagccagcggctacgagttcatcgactgcaccctgaactggatcagactggcccctggcaaaagacccgagtggatgggatggctgaagcctagaggcggagccgtgaattacgccagacctctgcagggcagagtgaccatgaccagggacgtgtacagcgataccgccttcctggaactgagaagcctgaccgtggatgataccgccgtgtacttctgcacccggggcaagaactgcgactacaactgggacttcgagcactggggcagaggcacacctgtgatcgttagctctgcaagcggaggctccggagataagacccacacatgtcctccatgtcctgctccagagctgctgggtggcccttccgtgtttctgttccctccaaagcctaaggacaccctgatgatcagcaggacccctgaagtgacctgcgtggtggtggatgtgtctcacgaggacccagaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctgcccgctcctatcgagaaaaccatcagcaaggccaagggccagcctcgcgaacctcaagtctgtacactgcctcctagccgggacgagctgaccaagaatcaggtgtcactgagctgcgccgtgaagggcttttaccccagcgacattgccgtggaatgggagagcaatggccagccagagaacaactacaagacaacccctcctgtgctggacagcgacggctcatttttcctggtgtccaagctgacagtggacaagagcagatggcagcagggcaacgtgttcagctgcagcgtgatgcacgaggccctgcacaaccactacacccagaaaagcctgtctctgagccctggc

SEQ ID NO: 50 - DNA, 2244 bp, synthetic construct
  misc_feature 1..2244: note = 10e8v4PG16L1knob
  source 1..2244: mol_type = other DNA
agcgaactgacccaagatcctgccgtgtctgtggccctgaagcagaccgtgaccatcacctgtagaggcgacagcctgagaagccactacgccagctggtatcagaagaagcctggacaggctcccgtgctgctgttctacggcaagaacaacagacccagcggcatccccgatagattttctggcagcgccagcggcaatagagccagcctgacaattactggcgcccaggccgaagatgaggccgactactactgcagcagcagagacaagagcggcagcagactgtccgtttttggcggaggcaccaagctgaccgtgctgggcggatcttctggacagtctgctctgacacagcctgcctctgtgtctggctctcctggccagaccatcaccatcagctgtaacggcaccagctctgacgtcggcggctttgatagcgtgtcctggtatcagcagagccccggcaaagcccctaaagtgatggtgttcgatgtgtcccaccggcctagcggcatcagcaatagattcagcggcagcaagagcggcaacaccgccagcctgacaatctctggactgcacatcgaggacgagggcgactacttctgtagcagcctgaccgacagaagccaccggatttttggcggaggcaccaaagtgaccgtgctgggaggcggaggatctggcggatctggaagtggcggcggtggatctgcttctggctctggccaagaacagctggtggaatctggtggcggagtggttcaacctggcggctctctgagactgagctgtctggcctccggcttcaccttccacaaatacggcatgcactgggtccgacaggcccctggaaaaggccttgaatgggtcgccctgatcagcgacgacggcatgagaaagtaccacagcgacagcatgtggggcagagtgaccatcagccgggacaacagcaagaacaccctgtacctgcagttcagctccctgaaggtggaagataccgccatgttcttttgcgccagagaagccggcggacccatctggcatgacgacgtgaagtactacgacttcaacgacggctactacaactaccactacatggacgtgtggggcaagggcaccaccgtgacagtttcttctggcggatcttctggagaagttcggctggtggaatctggcggcggacttgtgaaacctggcggctctctgagactgagctgtagcgcctccggcttcgacttcgacaatgcctggatgacctgggtccgacagcctcctggaaaaggcctggaatgggtcggaagaatcacaggccctggcgaaggctggagtgtggattatgccgagagcgtgaagggcagattcaccatcagccgggacaacaccaagaacaccctgtacctggaaatgaacaacgtgcggaccgaggataccggctactacttctgtgcccggaccggcaagtactacgacttttggagcggctaccctcctggcgaggaatactttcaggattggggccagggcaccctggtcatcgtttcttctgcaagcggaggctccggagataagacccacacatgtcctccatgtcctgctccagaactgctgggcggaccttccgtgtttctgttccctccaaagcctaaggacaccctgatgatcagcaggacccctgaagtgacctgcgtggtggtggatgtgtctcacgaggacccagaagtgaagttcaattggtacgtggacggcgtggaagtgcacaacgccaagaccaagcctagagaggaacagtacaacagcacctacagagtggtgtccgtgctgacagtgctgcaccaggattggctgaacggcaaagagtacaagtgcaaggtgtccaacaaggccctgcccgcccctatcgagaaaaccatcagcaaggccaagggccagcctagggaaccccaggtttacacccttcctccatgccgggatgagctgaccaagaaccaggtgtccctgtggtgtctggtcaagggcttctaccccagcgacattgccgtggaatgggagagcaatggccagcctgagaacaactacaagaccacacctcctgtgctggactccgacggatcattcttcctgtacagcaagctgactgtggacaagagcagatggcagcagggcaacgtgttcagctgcagcgtgatgcacgaggccctgcacaaccactacacacagaagtccctgtctctgagccctggc

SEQ ID NO: 51 - DNA, 2244 bp, synthetic construct
  misc_feature 1..2244: note = PG1610e8v4L1knob
  source 1..2244: mol_type = other DNA
cagtctgctctgacacagcctgcctctgtgtctggctctcctggccagaccatcaccatcagctgtaacggcaccagctctgacgtcggcggctttgatagcgtgtcctggtatcagcagagccccggcaaagcccctaaagtgatggtgttcgatgtgtcccaccggcctagcggcatcagcaatagattcagcggcagcaagagcggcaacaccgccagcctgacaatctctggactgcacatcgaggacgagggcgactacttctgtagcagcctgaccgacagaagccaccggatttttggcggaggcaccaaagtgaccgtgctgggcggatcttctggaagcgaactgacccaagatcctgccgtgtctgtggccctgaagcagaccgtgaccatcacctgtagaggcgacagcctgagaagccactacgccagctggtatcagaagaagcctggacaggctcccgtgctgctgttctacggcaagaacaacagacccagcggcatccccgatagattttctggcagcgccagcggcaatagagccagcctgacaattactggcgcccaggccgaagatgaggccgactactactgcagcagcagagacaagagcggcagcagactgtccgtttttggcggaggcaccaagctgaccgtgctgggaggcggaggatctggcggatctggaagtggcggcggtggatctgcttctggctctggcgaagttcggctggtggaatctggcggcggacttgtgaaacctggcggctctctgagactgagctgtagcgcctccggcttcgacttcgacaatgcctggatgacctgggtccgacagcctcctggaaaaggcctggaatgggtcggaagaatcacaggccctggcgaaggctggagtgtggattatgccgagagcgtgaagggcagattcaccatcagccgggacaacaccaagaacaccctgtacctggaaatgaacaacgtgcggaccgaggataccggctactacttctgtgcccggaccggcaagtactacgacttttggagcggctaccctcctggcgaggaatactttcaggattggggccagggcaccctggtcatcgtttcttctggcggatcttctggacaagaacagctggtggaatctggtggcggagtggttcaacctggcggctctctgagactgagctgtctggcctccggcttcaccttccacaaatacggcatgcactgggtccgacaggcccctggaaaaggccttgaatgggtcgccctgatcagcgacgacggcatgagaaagtaccacagcgacagcatgtggggcagagtgaccatcagccgggacaacagcaagaacaccctgtacctgcagttcagctccctgaaggtggaagataccgccatgttcttttgcgccagagaagccggcggacccatctggcatgacgacgtgaagtactacgacttcaacgacggctactacaactaccactacatggacgtgtggggcaagggcaccaccgtgacagtttcttctgcaagcggaggctccggagataagacccacacatgtcctccatgtcctgctccagaactgctgggcggaccttccgtgtttctgttccctccaaagcctaaggacaccctgatgatcagcaggacccctgaagtgacctgcgtggtggtggatgtgtctcacgaggacccagaagtgaagttcaattggtacgtggacggcgtggaagtgcacaacgccaagaccaagcctagagaggaacagtacaacagcacctacagagtggtgtccgtgctgacagtgctgcaccaggattggctgaacggcaaagagtacaagtgcaaggtgtccaacaaggccctgcccgcccctatcgagaaaaccatcagcaaggccaagggccagcctagggaaccccaggtttacacccttcctccatgccgggatgagctgaccaagaaccaggtgtccctgtggtgtctggtcaagggcttctaccccagcgacattgccgtggaatgggagagcaatggccagcctgagaacaactacaagaccacacctcctgtgctggactccgacggatcattcttcctgtacagcaagctgactgtggacaagagcagatggcagcagggcaacgtgttcagctgcagcgtgatgcacgaggccctgcacaaccactacacacagaagtccctgtctctgagccctggc

SEQ ID NO: 52 - DNA, 2214 bp, synthetic construct
  misc_feature 1..2214: note = PGT12110e8v4L1holeFc
  source 1..2214: mol_type = other DNA
agcgatatctctgtggcccctggcgagacagccagaatcagctgtggcgagaagtccctgggaagcagagccgtgcagtggtatcagcatagagccggacaggcccctagcctgatcatctacaacaaccaggacagacccagcggcatccccgagagattttctggcagccctgatagccctttcggcaccacagccacactgaccatcacatctgtggaagccggcgacgaggccgactactactgtcacatctgggacagcagggtgcccaccaaatgggttttcggcggaggcacaaccctgaccgttctgggcggatcttctggaagcgaactgacccaagatcctgccgtgtctgtggccctgaagcagaccgtgaccatcacctgtagaggcgacagcctgagaagccactacgccagctggtatcagaagaagcctggacaggctcccgtgctgctgttctacggcaagaacaacagacccagcggcatccccgatagattttctggcagcgccagcggcaatagagccagcctgacaattactggcgcccaggccgaagatgaggccgactactactgcagcagcagagacaagagcggcagcagactgtccgtttttggcggaggcaccaagctgaccgtgctgggaggcggaggatctggcggatctggaagtggcggcggtggatctgcttctggctctggcgaagttcggctggtggaatctggcggcggacttgtgaaacctggcggctctctgagactgagctgtagcgcctccggcttcgacttcgacaatgcctggatgacctgggtccgacagcctcctggaaaaggcctggaatgggtcggaagaatcacaggccctggcgaaggctggagtgtggattatgccgagagcgtgaagggcagattcaccatcagccgggacaacaccaagaacaccctgtacctggaaatgaacaacgtgcggaccgaggataccggctactacttctgtgcccggaccggcaagtactacgacttttggagcggctaccctcctggcgaggaatactttcaggattggggccagggcaccctggtcatcgtttcttctggcggatcttctggacagatgcagctccaagagtctggccctggcctggtcaagcctagcgaaacactgagcctgacctgttccgtgtccggcgccagcatcagcgatagctactggtcctggatcagaagaagccccggcaaaggcctggaatggatcggctacgtgcacaagagcggcgacaccaactatagccccagcctgaagtccagagtgaacctgagcctggacaccagcaagaaccaggtgtccctgtctctggtggccgctacagctgccgatagcggcaagtactactgcgccagaactctgcacggcagacggatctatggcatcgtggccttcaacgagtggttcacctacttctacatggacgtgtggggcaacggcacccaagtgacagtctcttctgcaagcggaggctccggagataagacccacacatgtcctccatgtcctgctccagagctgctgggtggcccttccgtgtttctgttccctccaaagcctaaggacaccctgatgatcagcaggacccctgaagtgacctgcgtggtggtggatgtgtctcacgaggacccagaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctgcccgctcctatcgagaaaaccatcagcaaggccaagggccagcctcgcgaacctcaagtctgtacactgcctcctagccgggacgagctgaccaagaatcaggtgtcactgagctgcgccgtgaagggcttttaccccagcgacattgccgtggaatgggagagcaatggccagccagagaacaactacaagacaacccctcctgtgctggacagcgacggctcatttttcctggtgtccaagctgacagtggacaagagcagatggcagcagggcaacgtgttcagctgcagcgtgatgcacgaggccctgcacaaccactacacccagaaaagcctgtctctgagccctggc

SEQ ID NO: 53 - DNA, 2214 bp, synthetic construct
  misc_feature 1..2214: note = 10e8v4PGT121L1holeFc
  source 1..2214: mol_type = other DNA
agcgaactgacccaagatcctgccgtgtctgtggccctgaagcagaccgtgaccatcacctgtagaggcgacagcctgagaagccactacgccagctggtatcagaagaagcctggacaggctcccgtgctgctgttctacggcaagaacaacagacccagcggcatccccgatagattttctggcagcgccagcggcaatagagccagcctgacaattactggcgcccaggccgaagatgaggccgactactactgcagcagcagagacaagagcggcagcagactgtccgtttttggcggaggcaccaagctgaccgtgctgggcggatcttctggaagcgatatctctgtggcccctggcgagacagccagaatcagctgtggcgagaagtccctgggaagcagagccgtgcagtggtatcagcatagagccggacaggcccctagcctgatcatctacaacaaccaggacagacccagcggcatccccgagagattttctggcagccctgatagccctttcggcaccacagccacactgaccatcacatctgtggaagccggcgacgaggccgactactactgtcacatctgggacagcagggtgcccaccaaatgggttttcggcggaggcacaaccctgaccgttctgggaggcggaggatctggcggatctggaagtggcggcggtggatctgcttctggctctggccagatgcagctccaagagtctggccctggcctggtcaagcctagcgaaacactgagcctgacctgttccgtgtccggcgccagcatcagcgatagctactggtcctggatcagaagaagccccggcaaaggcctggaatggatcggctacgtgcacaagagcggcgacaccaactatagccccagcctgaagtccagagtgaacctgagcctggacaccagcaagaaccaggtgtccctgtctctggtggccgctacagctgccgatagcggcaagtactactgcgccagaactctgcacggcagacggatctatggcatcgtggccttcaacgagtggttcacctacttctacatggacgtgtggggcaacggcacccaagtgacagtctcttctggcggatcttctggagaagttcggctggtggaatctggcggcggacttgtgaaacctggcggctctctgagactgagctgtagcgcctccggcttcgacttcgacaatgcctggatgacctgggtccgacagcctcctggaaaaggcctggaatgggtcggaagaatcacaggccctggcgaaggctggagtgtggattatgccgagagcgtgaagggcagattcaccatcagccgggacaacaccaagaacaccctgtacctggaaatgaacaacgtgcggaccgaggataccggctactacttctgtgcccggaccggcaagtactacgacttttggagcggctaccctcctggcgaggaatactttcaggattggggccagggcaccctggtcatcgtttcttctgcaagcggaggctccggagataagacccacacatgtcctccatgtcctgctccagagctgctgggtggcccttccgtgtttctgttccctccaaagcctaaggacaccctgatgatcagcaggacccctgaagtgacctgcgtggtggtggatgtgtctcacgaggacccagaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctgcccgctcctatcgagaaaaccatcagcaaggccaagggccagcctcgcgaacctcaagtctgtacactgcctcctagccgggacgagctgaccaagaatcaggtgtcactgagctgcgccgtgaagggcttttaccccagcgacattgccgtggaatgggagagcaatggccagccagagaacaactacaagacaacccctcctgtgctggacagcgacggctcatttttcctggtgtccaagctgacagtggacaagagcagatggcagcagggcaacgtgttcagctgcagcgtgatgcacgaggccctgcacaaccactacacccagaaaagcctgtctctgagccctggc

SEQ ID NO: 54 - DNA, 2199 bp, synthetic construct
  misc_feature 1..2199: note = VRC01PG16L1knob
  source 1..2199: mol_type = other DNA
gagatcgtgctgacacagagccctggcaccctgagcctgtctccaggcgagacagccatcatcagctgccggacaagccagtacggcagcctggcctggtatcagcagaggcctggacaggcccccagactcgtgatctacagcggcagcacaagagccgccggaatccccgatagattcagcggctccagatggggccctgactacaacctgaccatcagcaacctggaaagcggcgacttcggcgtgtactactgccagcagtacgagttcttcggccagggcaccaaggtgcaggtggacatcaagggcggatcttctggacagtctgctctgacacagcctgcctctgtgtctggctctcctggccagaccatcaccatcagctgtaacggcaccagctctgacgtcggcggctttgatagcgtgtcctggtatcagcagagccccggcaaagcccctaaagtgatggtgttcgatgtgtcccaccggcctagcggcatcagcaatagattcagcggcagcaagagcggcaacaccgccagcctgacaatctctggactgcacatcgaggacgagggcgactacttctgtagcagcctgaccgacagaagccaccggatttttggcggaggcaccaaagtgaccgtgctgggaggcggaggatctggcggatctggaagtggcggcggtggatctgcttctggctctggccaagaacagctggtggaatctggtggcggagtggttcaacctggcggctctctgagactgagctgtctggcctccggcttcaccttccacaaatacggcatgcactgggtccgacaggcccctggaaaaggccttgaatgggtcgccctgatcagcgacgacggcatgagaaagtaccacagcgacagcatgtggggcagagtgaccatcagccgggacaacagcaagaacaccctgtacctgcagttcagctccctgaaggtggaagataccgccatgttcttttgcgccagagaagccggcggacccatctggcatgacgacgtgaagtactacgacttcaacgacggctactacaactaccactacatggacgtgtggggcaagggcaccaccgtgacagtttcttctggcggatcttctggacaggttcagctggttcagtctggcggacagatgaagaaacccggcgagagcatgcggatcagctgtagagccagcggctacgagttcatcgactgcaccctgaactggatcagactggcccctggcaaaagacccgagtggatgggatggctgaagcctagaggcggagccgtgaattacgccagacctctgcagggcagagtgaccatgaccagggacgtgtacagcgataccgccttcctggaactgagaagcctgaccgtggatgataccgccgtgtacttctgcacccggggcaagaactgcgactacaactgggacttcgagcactggggcagaggcacacctgtgatcgttagctctgcaagcggaggctccggagataagacccacacatgtcctccatgtcctgctccagaactgctgggcggaccttccgtgtttctgttccctccaaagcctaaggacaccctgatgatcagcaggacccctgaagtgacctgcgtggtggtggatgtgtctcacgaggacccagaagtgaagttcaattggtacgtggacggcgtggaagtgcacaacgccaagaccaagcctagagaggaacagtacaacagcacctacagagtggtgtccgtgctgacagtgctgcaccaggattggctgaacggcaaagagtacaagtgcaaggtgtccaacaaggccctgcccgcccctatcgagaaaaccatcagcaaggccaagggccagcctagggaaccccaggtttacacccttcctccatgccgggatgagctgaccaagaaccaggtgtccctgtggtgtctggtcaagggcttctaccccagcgacattgccgtggaatgggagagcaatggccagcctgagaacaactacaagaccacacctcctgtgctggactccgacggatcattcttcctgtacagcaagctgactgtggacaagagcagatggcagcagggcaacgtgttcagctgcagcgtgatgcacgaggccctgcacaaccactacacacagaagtccctgtctctgagccctggc

SEQ ID NO: 55 - DNA, 2199 bp, synthetic construct
  misc_feature 1..2199: note = PG16VRC01L1knob
  source 1..2199: mol_type = other DNA
cagtctgctctgacacagcctgcctctgtgtctggctctcctggccagaccatcaccatcagctgtaacggcaccagctctgacgtcggcggctttgatagcgtgtcctggtatcagcagagccccggcaaagcccctaaagtgatggtgttcgatgtgtcccaccggcctagcggcatcagcaatagattcagcggcagcaagagcggcaacaccgccagcctgacaatctctggactgcacatcgaggacgagggcgactacttctgtagcagcctgaccgacagaagccaccggatttttggcggaggcaccaaagtgaccgtgctgggcggatcttctggagagatcgtgctgacacagagccctggcaccctgagcctgtctccaggcgagacagccatcatcagctgccggacaagccagtacggcagcctggcctggtatcagcagaggcctggacaggcccccagactcgtgatctacagcggcagcacaagagccgccggaatccccgatagattcagcggctccagatggggccctgactacaacctgaccatcagcaacctggaaagcggcgacttcggcgtgtactactgccagcagtacgagttcttcggccagggcaccaaggtgcaggtggacatcaagggaggcggaggatctggcggatctggaagtggcggcggtggatctgcttctggctctggccaggttcagctggttcagtctggcggacagatgaagaaacccggcgagagcatgcggatcagctgtagagccagcggctacgagttcatcgactgcaccctgaactggatcagactggcccctggcaaaagacccgagtggatgggatggctgaagcctagaggcggagccgtgaattacgccagacctctgcagggcagagtgaccatgaccagggacgtgtacagcgataccgccttcctggaactgagaagcctgaccgtggatgataccgccgtgtacttctgcacccggggcaagaactgcgactacaactgggacttcgagcactggggcagaggcacacctgtgatcgttagctctggcggatcttctggacaagaacagctggtggaatctggtggcggagtggttcaacctggcggctctctgagactgagctgtctggcctccggcttcaccttccacaaatacggcatgcactgggtccgacaggcccctggaaaaggccttgaatgggtcgccctgatcagcgacgacggcatgagaaagtaccacagcgacagcatgtggggcagagtgaccatcagccgggacaacagcaagaacaccctgtacctgcagttcagctccctgaaggtggaagataccgccatgttcttttgcgccagagaagccggcggacccatctggcatgacgacgtgaagtactacgacttcaacgacggctactacaactaccactacatggacgtgtggggcaagggcaccaccgtgacagtttcttctgcaagcggaggctccggagataagacccacacatgtcctccatgtcctgctccagaactgctgggcggaccttccgtgtttctgttccctccaaagcctaaggacaccctgatgatcagcaggacccctgaagtgacctgcgtggtggtggatgtgtctcacgaggacccagaagtgaagttcaattggtacgtggacggcgtggaagtgcacaacgccaagaccaagcctagagaggaacagtacaacagcacctacagagtggtgtccgtgctgacagtgctgcaccaggattggctgaacggcaaagagtacaagtgcaaggtgtccaacaaggccctgcccgcccctatcgagaaaaccatcagcaaggccaagggccagcctagggaaccccaggtttacacccttcctccatgccgggatgagctgaccaagaaccaggtgtccctgtggtgtctggtcaagggcttctaccccagcgacattgccgtggaatgggagagcaatggccagcctgagaacaactacaagaccacacctcctgtgctggactccgacggatcattcttcctgtacagcaagctgactgtggacaagagcagatggcagcagggcaacgtgttcagctgcagcgtgatgcacgaggccctgcacaaccactacacacagaagtccctgtctctgagccctggc

SEQ ID NO: 56 - DNA, 2238 bp, synthetic construct
  misc_feature 1..2238: note = PGT121PG16L1holeFc
  source 1..2238: mol_type = other DNA
agcgatatctctgtggcccctggcgagacagccagaatcagctgtggcgagaagtccctgggaagcagagccgtgcagtggtatcagcatagagccggacaggcccctagcctgatcatctacaacaaccaggacagacccagcggcatccccgagagattttctggcagccctgatagccctttcggcaccacagccacactgaccatcacatctgtggaagccggcgacgaggccgactactactgtcacatctgggacagcagggtgcccaccaaatgggttttcggcggaggcacaaccctgaccgttctgggcggatcttctggacagtctgctctgacacagcctgcctctgtgtctggctctcctggccagaccatcaccatcagctgtaacggcaccagctctgacgtcggcggctttgatagcgtgtcctggtatcagcagagccccggcaaagcccctaaagtgatggtgttcgatgtgtcccaccggcctagcggcatcagcaatagattcagcggcagcaagagcggcaacaccgccagcctgacaatctctggactgcacatcgaggacgagggcgactacttctgtagcagcctgaccgacagaagccaccggatttttggcggaggcaccaaagtgaccgtgctgggaggcggaggatctggcggatctggaagtggcggcggtggatctgcttctggctctggccaagaacagctggtggaatctggtggcggagtggttcaacctggcggctctctgagactgagctgtctggcctccggcttcaccttccacaaatacggcatgcactgggtccgacaggcccctggaaaaggccttgaatgggtcgccctgatcagcgacgacggcatgagaaagtaccacagcgacagcatgtggggcagagtgaccatcagccgggacaacagcaagaacaccctgtacctgcagttcagctccctgaaggtggaagataccgccatgttcttttgcgccagagaagccggcggacccatctggcatgacgacgtgaagtactacgacttcaacgacggctactacaactaccactacatggacgtgtggggcaagggcaccaccgtgacagtttcttctggcggatcttctggacagatgcagctccaagagtctggccctggcctggtcaagcctagcgaaacactgagcctgacctgttccgtgtccggcgccagcatcagcgatagctactggtcctggatcagaagaagccccggcaaaggcctggaatggatcggctacgtgcacaagagcggcgacaccaactatagccccagcctgaagtccagagtgaacctgagcctggacaccagcaagaaccaggtgtccctgtctctggtggccgctacagctgccgatagcggcaagtactactgcgccagaactctgcacggcagacggatctatggcatcgtggccttcaacgagtggttcacctacttctacatggacgtgtggggcaacggcacccaagtgacagtctcttctgcaagcggaggctccggagataagacccacacatgtcctccatgtcctgctccagagctgctgggtggcccttccgtgtttctgttccctccaaagcctaaggacaccctgatgatcagcaggacccctgaagtgacctgcgtggtggtggatgtgtctcacgaggacccagaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctgcccgctcctatcgagaaaaccatcagcaaggccaagggccagcctcgcgaacctcaagtctgtacactgcctcctagccgggacgagctgaccaagaatcaggtgtcactgagctgcgccgtgaagggcttttaccccagcgacattgccgtggaatgggagagcaatggccagccagagaacaactacaagacaacccctcctgtgctggacagcgacggctcatttttcctggtgtccaagctgacagtggacaagagcagatggcagcagggcaacgtgttcagctgcagcgtgatgcacgaggccctgcacaaccactacacccagaaaagcctgtctctgagccctggc

SEQ ID NO: 57 - DNA, 2238 bp, synthetic construct
  misc_feature 1..2238: note = PG16PGT121L1holeFc
  source 1..2238: mol_type = other DNA
cagtctgctctgacacagcctgcctctgtgtctggctctcctggccagaccatcaccatcagctgtaacggcaccagctctgacgtcggcggctttgatagcgtgtcctggtatcagcagagccccggcaaagcccctaaagtgatggtgttcgatgtgtcccaccggcctagcggcatcagcaatagattcagcggcagcaagagcggcaacaccgccagcctgacaatctctggactgcacatcgaggacgagggcgactacttctgtagcagcctgaccgacagaagccaccggatttttggcggaggcaccaaagtgaccgtgctgggcggatcttctggaagcgatatctctgtggcccctggcgagacagccagaatcagctgtggcgagaagtccctgggaagcagagccgtgcagtggtatcagcatagagccggacaggcccctagcctgatcatctacaacaaccaggacagacccagcggcatccccgagagattttctggcagccctgatagccctttcggcaccacagccacactgaccatcacatctgtggaagccggcgacgaggccgactactactgtcacatctgggacagcagggtgcccaccaaatgggttttcggcggaggcacaaccctgaccgttctgggaggcggaggatctggcggatctggaagtggcggcggtggatctgcttctggctctggccagatgcagctccaagagtctggccctggcctggtcaagcctagcgaaacactgagcctgacctgttccgtgtccggcgccagcatcagcgatagctactggtcctggatcagaagaagccccggcaaaggcctggaatggatcggctacgtgcacaagagcggcgacaccaactatagccccagcctgaagtccagagtgaacctgagcctggacaccagcaagaaccaggtgtccctgtctctggtggccgctacagctgccgatagcggcaagtactactgcgccagaactctgcacggcagacggatctatggcatcgtggccttcaacgagtggttcacctacttctacatggacgtgtggggcaacggcacccaagtgacagtctcttctggcggatcttctggacaagaacagctggtggaatctggtggcggagtggttcaacctggcggctctctgagactgagctgtctggcctccggcttcaccttccacaaatacggcatgcactgggtccgacaggcccctggaaaaggccttgaatgggtcgccctgatcagcgacgacggcatgagaaagtaccacagcgacagcatgtggggcagagtgaccatcagccgggacaacagcaagaacaccctgtacctgcagttcagctccctgaaggtggaagataccgccatgttcttttgcgccagagaagccggcggacccatctggcatgacgacgtgaagtactacgacttcaacgacggctactacaactaccactacatggacgtgtggggcaagggcaccaccgtgacagtttcttctgcaagcggaggctccggagataagacccacacatgtcctccatgtcctgctccagagctgctgggtggcccttccgtgtttctgttccctccaaagcctaaggacaccctgatgatcagcaggacccctgaagtgacctgcgtggtggtggatgtgtctcacgaggacccagaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctgcccgctcctatcgagaaaaccatcagcaaggccaagggccagcctcgcgaacctcaagtctgtacactgcctcctagccgggacgagctgaccaagaatcaggtgtcactgagctgcgccgtgaagggcttttaccccagcgacattgccgtggaatgggagagcaatggccagccagagaacaactacaagacaacccctcctgtgctggacagcgacggctcatttttcctggtgtccaagctgacagtggacaagagcagatggcagcagggcaacgtgttcagctgcagcgtgatgcacgaggccctgcacaaccactacacccagaaaagcctgtctctgagccctggc

SEQ ID NO: 58 - DNA, 2175 bp, synthetic construct
  misc_feature 1..2175: note = VRC0110e8v4L1knob
  source 1..2175: mol_type = other DNA
gagatcgtgctgacacagagccctggcaccctgagcctgtctccaggcgagacagccatcatcagctgccggacaagccagtacggcagcctggcctggtatcagcagaggcctggacaggcccccagactcgtgatctacagcggcagcacaagagccgccggaatccccgatagattcagcggctccagatggggccctgactacaacctgaccatcagcaacctggaaagcggcgacttcggcgtgtactactgccagcagtacgagttcttcggccagggcaccaaggtgcaggtggacatcaagggcggatcttctggaagcgaactgacccaagatcctgccgtgtctgtggccctgaagcagaccgtgaccatcacctgtagaggcgacagcctgagaagccactacgccagctggtatcagaagaagcctggacaggctcccgtgctgctgttctacggcaagaacaacagacccagcggcatccccgatagattttctggcagcgccagcggcaatagagccagcctgacaattactggcgcccaggccgaagatgaggccgactactactgcagcagcagagacaagagcggcagcagactgtccgtttttggcggaggcaccaagctgaccgtgctgggaggcggaggatctggcggatctggaagtggcggcggtggatctgcttctggctctggcgaagttcggctggtggaatctggcggcggacttgtgaaacctggcggctctctgagactgagctgtagcgcctccggcttcgacttcgacaatgcctggatgacctgggtccgacagcctcctggaaaaggcctggaatgggtcggaagaatcacaggccctggcgaaggctggagtgtggattatgccgagagcgtgaagggcagattcaccatcagccgggacaacaccaagaacaccctgtacctggaaatgaacaacgtgcggaccgaggataccggctactacttctgtgcccggaccggcaagtactacgacttttggagcggctaccctcctggcgaggaatactttcaggattggggccagggcaccctggtcatcgtttcttctggcggatcttctggacaggttcagctggttcagtctggcggacagatgaagaaacccggcgagagcatgcggatcagctgtagagccagcggctacgagttcatcgactgcaccctgaactggatcagactggcccctggcaaaagacccgagtggatgggatggctgaagcctagaggcggagccgtgaattacgccagacctctgcagggcagagtgaccatgaccagggacgtgtacagcgataccgccttcctggaactgagaagcctgaccgtggatgataccgccgtgtacttctgcacccggggcaagaactgcgactacaactgggacttcgagcactggggcagaggcacacctgtgatcgttagctctgcaagcggaggctccggagataagacccacacatgtcctccatgtcctgctccagaactgctgggcggaccttccgtgtttctgttccctccaaagcctaaggacaccctgatgatcagcaggacccctgaagtgacctgcgtggtggtggatgtgtctcacgaggacccagaagtgaagttcaattggtacgtggacggcgtggaagtgcacaacgccaagaccaagcctagagaggaacagtacaacagcacctacagagtggtgtccgtgctgacagtgctgcaccaggattggctgaacggcaaagagtacaagtgcaaggtgtccaacaaggccctgcccgcccctatcgagaaaaccatcagcaaggccaagggccagcctagggaaccccaggtttacacccttcctccatgccgggatgagctgaccaagaaccaggtgtccctgtggtgtctggtcaagggcttctaccccagcgacattgccgtggaatgggagagcaatggccagcctgagaacaactacaagaccacacctcctgtgctggactccgacggatcattcttcctgtacagcaagctgactgtggacaagagcagatggcagcagggcaacgtgttcagctgcagcgtgatgcacgaggccctgcacaaccactacacacagaagtccctgtctctgagccctggc

SEQ ID NO: 59 - DNA, 2175 bp, synthetic construct
  misc_feature 1..2175: note = 10e8v4VRC01L1knob
  source 1..2175: mol_type = other DNA
agcgaactgacccaagatcctgccgtgtctgtggccctgaagcagaccgtgaccatcacctgtagaggcgacagcctgagaagccactacgccagctggtatcagaagaagcctggacaggctcccgtgctgctgttctacggcaagaacaacagacccagcggcatccccgatagattttctggcagcgccagcggcaatagagccagcctgacaattactggcgcccaggccgaagatgaggccgactactactgcagcagcagagacaagagcggcagcagactgtccgtttttggcggaggcaccaagctgaccgtgctgggcggatcttctggagagatcgtgctgacacagagccctggcaccctgagcctgtctccaggcgagacagccatcatcagctgccggacaagccagtacggcagcctggcctggtatcagcagaggcctggacaggcccccagactcgtgatctacagcggcagcacaagagccgccggaatccccgatagattcagcggctccagatggggccctgactacaacctgaccatcagcaacctggaaagcggcgacttcggcgtgtactactgccagcagtacgagttcttcggccagggcaccaaggtgcaggtggacatcaagggaggcggaggatctggcggatctggaagtggcggcggtggatctgcttctggctctggccaggttcagctggttcagtctggcggacagatgaagaaacccggcgagagcatgcggatcagctgtagagccagcggctacgagttcatcgactgcaccctgaactggatcagactggcccctggcaaaagacccgagtggatgggatggctgaagcctagaggcggagccgtgaattacgccagacctctgcagggcagagtgaccatgaccagggacgtgtacagcgataccgccttcctggaactgagaagcctgaccgtggatgataccgccgtgtacttctgcacccggggcaagaactgcgactacaactgggacttcgagcactggggcagaggcacacctgtgatcgttagctctggcggatcttctggagaagttcggctggtggaatctggcggcggacttgtgaaacctggcggctctctgagactgagctgtagcgcctccggcttcgacttcgacaatgcctggatgacctgggtccgacagcctcctggaaaaggcctggaatgggtcggaagaatcacaggccctggcgaaggctggagtgtggattatgccgagagcgtgaagggcagattcaccatcagccgggacaacaccaagaacaccctgtacctggaaatgaacaacgtgcggaccgaggataccggctactacttctgtgcccggaccggcaagtactacgacttttggagcggctaccctcctggcgaggaatactttcaggattggggccagggcaccctggtcatcgtttcttctgcaagcggaggctccggagataagacccacacatgtcctccatgtcctgctccagaactgctgggcggaccttccgtgtttctgttccctccaaagcctaaggacaccctgatgatcagcaggacccctgaagtgacctgcgtggtggtggatgtgtctcacgaggacccagaagtgaagttcaattggtacgtggacggcgtggaagtgcacaacgccaagaccaagcctagagaggaacagtacaacagcacctacagagtggtgtccgtgctgacagtgctgcaccaggattggctgaacggcaaagagtacaagtgcaaggtgtccaacaaggccctgcccgcccctatcgagaaaaccatcagcaaggccaagggccagcctagggaaccccaggtttacacccttcctccatgccgggatgagctgaccaagaaccaggtgtccctgtggtgtctggtcaagggcttctaccccagcgacattgccgtggaatgggagagcaatggccagcctgagaacaactacaagaccacacctcctgtgctggactccgacggatcattcttcctgtacagcaagctgactgtggacaagagcagatggcagcagggcaacgtgttcagctgcagcgtgatgcacgaggccctgcacaaccactacacacagaagtccctgtctctgagccctggc

SEQ ID NO: 60 - protein, 5 aa, synthetic construct
  REGION 1..5: note = PGT121 heavy CDR1
DSYWS

SEQ ID NO: 61 - protein, 16 aa, synthetic construct
  REGION 1..16: note = PGT121 heavy CDR2
YVHKSGDTNYSPSLKS

SEQ ID NO: 62 - protein, 24 aa, synthetic construct
  REGION 1..24: note = PGT121 heavy CDR3
TLHGRRIYGIVAFNEWFTYFYMDV

SEQ ID NO: 63 - protein, 5 aa, synthetic construct
  REGION 1..5: note = VRC01 heavy CDR1
DCTLN

SEQ ID NO: 64 - protein, 17 aa, synthetic construct
  REGION 1..17: note = VRC01 heavy CDR2
WLKPRGGAVNYARPLQG

SEQ ID NO: 65 - protein, 12 aa, synthetic construct
  REGION 1..12: note = VRC01 heavy CDR3
GKNCDYNWDFEH

SEQ ID NO: 66 - protein, 5 aa, synthetic construct
  REGION 1..5: note = 10E8v4 heavy CDR1
NAWMT

SEQ ID NO: 67 - protein, 19 aa, synthetic construct
  REGION 1..19: note = 10E8v4 heavy CDR2
RITGPGEGWSVDYAESVKG

SEQ ID NO: 68 - protein, 20 aa, synthetic construct
  REGION 1..20: note = 10E8v4 heavy CDR3
TGKYYDFWSGYPPGEEYFQD

SEQ ID NO: 69 - protein, 5 aa, synthetic construct
  REGION 1..5: note = PG16 heavy CDR1
KYGMH

SEQ ID NO: 70 - protein, 17 aa, synthetic construct
  REGION 1..17: note = PG16 heavy CDR2
LISDDGMRKYHSDSMWG

SEQ ID NO: 71 - protein, 28 aa, synthetic construct
  REGION 1..28: note = PG16 heavy CDR3
EAGGPIWHDDVKYYDFNDGYYNYHYMDV

SEQ ID NO: 72 - protein, 14 aa, synthetic construct
  REGION 1..14: note = PG16 light CDR1
NGTSSDVGGFDSVS

SEQ ID NO: 73 - protein, 7 aa, synthetic construct
  REGION 1..7: note = PG16 light CDR2
DVSHRPS

SEQ ID NO: 74 - protein, 10 aa, synthetic construct
  REGION 1..10: note = PG16 light CDR3
SSLTDRSHRI

SEQ ID NO: 75 - protein, 11 aa, synthetic construct
  REGION 1..11: note = PGT121 light CDR1
GEKSLGSRAVQ

SEQ ID NO: 76 - protein, 7 aa, synthetic construct
  REGION 1..7: note = PGT121 light CDR2
NNQDRPS

SEQ ID NO: 77 - protein, 9 aa, synthetic construct
  REGION 1..9: note = PGT121 light CDR3
DSRVPTKWV

SEQ ID NO: 78 - protein, 9 aa, synthetic construct
  REGION 1..9: note = VRC01 light CDR1
RTSQYGSLA

SEQ ID NO: 79 - protein, 7 aa, synthetic construct
  REGION 1..7: note = VRC01 light CDR2
SGSTRAA

SEQ ID NO: 80 - protein, 5 aa, synthetic construct
  REGION 1..5: note = VRC01 light CDR3
QQYEF

SEQ ID NO: 81 - protein, 11 aa, synthetic construct
  REGION 1..11: note = 10E8v4 light CDR1
RGDSLRSHYAS

SEQ ID NO: 82 - protein, 7 aa, synthetic construct
  REGION 1..7: note = 10E8v4 light CDR2
GKNNRPS

SEQ ID NO: 83 - protein, 12 aa, synthetic construct
  REGION 1..12: note = 10E8v4 light CDR3
SSRDKSGSRLSV

SEQ ID NO: 84 - protein, 476 aa, synthetic construct
  REGION 1..476: note = MX894 - chain 1
EIVLTQSPGTLSLSPGETAIISCRTSQYGSLAWYQQRPGQAPRLVIYSGSTRAAGIPDRFSGSRWGPDYNLTISNLESGDFGVYYCQQYEFFGQGTKVQVDIKGGGGSGGSGSGGGGSASGSGQVQLVQSGGQMKKPGESMRISCRASGYEFIDCTLNWIRLAPGKRPEWMGWLKPRGGAVNYARPLQGRVTMTRDVYSDTAFLELRSLTVDDTAVYFCTRGKNCDYNWDFEHWGRGTPVIVSSASGGSGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 85 - DNA, 1428 bp, synthetic construct; identical to SEQ ID NO: 33
  misc_feature 1..1428: note = MX894 - chain 1
  source 1..1428: mol_type = other DNA

SEQ ID NO: 86 - protein, 738 aa, synthetic construct; identical to SEQ ID NO: 40
  REGION 1..738: note = MX894 - chain 2

SEQ ID NO: 87 - DNA, 2214 bp, synthetic construct; identical to SEQ ID NO: 52
  misc_feature 1..2214: note = MX894 - chain 2
  source 1..2214: mol_type = other DNA

SEQ ID NO: 88 - protein, 758 aa, synthetic construct
  REGION 1..758: note = MX873 - chain 1
QSVLTQPPSVSAAPGQKVTISCSGNTSNIGNNFVSWYQQRPGRAPQLLIYETDKRPSGIPDRFSASKSGTSGTLAITGLQTGDEADYYCATWAASLSSARVFGTGTKVIVLGGGGSSYVRPLSVALGETARISCGRQALGSRAVQWYQHRPGQAPILLIYNNQDRPSGIPERFSGTPDINFGTRATLTISGVEAGDEADYYCHMWDSRSGFSWSFGGATRLTVLGSGGSGGSGSGGGGSASGSGQVQLQESGPGLVKPSETLSVTCSVSGDSMNNYYWTWIRQSPGKGLEWIGYISDRESATYNPSLNSRVVISRDTSKNQLSLKLNSVTPADTAVYYCATARRGQRIYGVVSFGEFFYYYSMDVWGKGTTVTVSSGGGGSQVQLVESGGGVVQPGTSLRLSCAASQFRFDGYGMHWVRQAPGKGLEWVASISHDGIKKYHAEKVWGRFTISRDNSKNTLYLQMNSLRPEDTALYYCAKDLREDECEEWWSDYYDFGKQLPCAKSRGGLVGIADNWGQGTMVTVSSASGGSGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVLHEALHSHYTQKSLSLSPG

SEQ ID NO: 89 - DNA, 2274 bp, synthetic construct
  misc_feature 1..2274: note = MX873 - chain 1
  source 1..2274: mol_type = other DNA
cagtctgtgctgacacagcctccatccgtgtctgctgcccctggccagaaagtgaccatcagctgtagcggcaacaccagcaacatcggcaacaacttcgtgtcctggtatcagcagaggcccggcagagcacctcagctgctgatctacgagacagacaagaggcccagcggcatccccgatagattttccgccagcaagagcggcacctctggcacactggctatcacaggactgcagacaggcgacgaggccgattactactgtgctacatgggccgccagcctgtctagcgccagagtttttggcaccggcaccaaagtgatcgtgctgggcggaggcggttctagctacgtcagacctctgtctgtggccctgggagagacagccagaatcagctgtggcagacaggctctgggaagcagagccgtgcagtggtatcagcacagacctggacaggcccctatcctgctgatctacaacaaccaggacagacccagcggcatccccgagagattttctggcacccctgacatcaacttcggcaccagagccacactgaccatctctggcgttgaagccggcgacgaggccgattactactgtcacatgtgggacagcagaagcggcttcagctggtcttttggcggagccaccagactgacagtgctgggttctggcggaagcggaggatctggatctggtggtggtggatctgcttctggcagtggacaggttcagctgcaagagtctggccctggcctggtcaagcctagcgaaacactgagcgtgacctgtagcgtgtccggcgacagcatgaacaactactactggacctggatcagacagagccccggcaaaggcctggaatggatcggctacatcagcgacagagagagcgccacctacaatcccagcctgaacagcagagtggtcatcagccgggacaccagcaagaaccagctgagcctgaagctgaacagcgtgacccctgccgataccgccgtgtactattgtgccaccgccagaagaggccagcggatctatggcgtggtgtccttcggcgagttcttctactactacagcatggacgtgtggggcaagggcaccaccgtgacagtgtctagcggaggcggaggtagtcaggttcagctggtggaatctggtggcggagtggtgcagcctggcacatctctgagactgtcttgtgccgcctctcagttcagattcgacggctacggaatgcactgggtccgacaggcacctggcaaaggccttgaatgggtcgccagcatcagccacgacggcatcaagaagtaccacgccgagaaagtgtggggcagattcaccatcagccgggacaacagcaagaacaccctgtacctgcagatgaacagcctgcggcctgaggacacagccctgtactattgcgccaaggacctgcgcgaggacgagtgtgaagaatggtggagcgactactacgacttcggcaagcagctgccctgcgccaaaagtagaggtggccttgtgggaatcgccgacaactggggacagggcacaatggtcacagtgtcctctgcttctggcggctccggagataagacccacacatgtcctccatgtcctgctccagaactgctcggcggcccttctgtgttcctgtttcctccaaagcctaaggacaccctgatgatcagcagaacccctgaagtgacctgcgtggtggtggatgtgtctcacgaggaccccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctgcctgctcctatcgagaaaaccatcagcaaggccaagggccagcctagggaaccccaggtttacacccttcctccatgccgggatgagctgaccaagaaccaggtgtccctgtggtgtctcgtgaagggcttctaccccagcgacattgccgtggaatgggagagcaatggccagcctgagaacaactacaagacaacccctcctgtgctggacagcgacggctcattcttcctgtacagcaagctgacagtcgacaagtccagatggcagcagggcaacgtgttcagttgcagcgtgctgcacgaggccctgcacagtcactacacccagaagtccctgagcctgtctcctgga

SEQ ID NO: 90 - protein, 723 aa, synthetic construct
  REGION 1..723: note = MX873 - chain 2
EIVLTQSPGTLSLSPGETAIISCRTSQYGSLAWYQQRPGQAPRLVIYSGSTRAAGIPDRFSGSRWGPDYNLTISNLESGDFGVYYCQQYEFFGQGTKVQVDIKGGSSGSDISVAPGETARISCGEKSLGSRAVQWYQHRAGQAPSLIIYNNQDRPSGIPERFSGSPDSPFGTTATLTITSVEAGDEADYYCHIWDSRVPTKWVFGGGTTLTVLGGGGSGGSGSGGGGSASGSGQMQLQESGPGLVKPSETLSLTCSVSGASISDSYWSWIRRSPGKGLEWIGYVHKSGDTNYSPSLKSRVNLSLDTSKNQVSLSLVAATAADSGKYYCARTLHGRRIYGIVAFNEWFTYFYMDVWGNGTQVTVSSGGSSGQVQLVQSGGQMKKPGESMRISCRASGYEFIDCTLNWIRLAPGKRPEWMGWLKPRGGAVNYARPLQGRVTMTRDVYSDTAFLELRSLTVDDTAVYFCTRGKNCDYNWDFEHWGRGTPVIVSSASGGSGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 91 - DNA, 2169 bp, synthetic construct; identical to SEQ ID NO: 49
  misc_feature 1..2169: note = MX873 - chain 2
  source 1..2169: mol_type = other DNA

SEQ ID NO: 92 - protein, 758 aa, synthetic construct
  REGION 1..758: note = MX875 - chain 1
SYVRPLSVALGETARISCGRQALGSRAVQWYQHRPGQAPILLIYNNQDRPSGIPERFSGTPDINFGTRATLTISGVEAGDEADYYCHMWDSRSGFSWSFGGATRLTVLGGGGSQSVLTQPPSVSAAPGQKVTISCSGNTSNIGNNFVSWYQQRPGRAPQLLIYETDKRPSGIPDRFSASKSGTSGTLAITGLQTGDEADYYCATWAASLSSARVFGTGTKVIVLGSGGSGGSGSGGGGSASGSGQVQLVESGGGVVQPGTSLRLSCAASQFRFDGYGMHWVRQAPGKGLEWVASISHDGIKKYHAEKVWGRFTISRDNSKNTLYLQMNSLRPEDTALYYCAKDLREDECEEWWSDYYDFGKQLPCAKSRGGLVGIADNWGQGTMVTVSSGGGGSQVQLQESGPGLVKPSETLSVTCSVSGDSMNNYYWTWIRQSPGKGLEWIGYISDRESATYNPSLNSRVVISRDTSKNQLSLKLNSVTPADTAVYYCATARRGQRIYGVVSFGEFFYYYSMDVWGKGTTVTVSSASGGSGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVLHEALHSHYTQKSLSLSPG

SEQ ID NO: 93 - DNA, 2274 bp, synthetic construct
  misc_feature 1..2274: note = MX875 - chain 1
  source 1..2274: mol_type = other DNA
agctacgtcagacctctgtctgtggccctgggagagacagccagaatcagctgtggcagacaggctctgggaagcagagccgtgcagtggtatcagcacagacctggacaggcccctatcctgctgatctacaacaaccaggacagacccagcggcatccccgagagattttctggcacccctgacatcaacttcggcaccagagccacactgaccatctctggcgttgaagccggcgacgaggccgattactactgtcacatgtgggacagcagaagcggcttcagctggtcttttggcggagccaccagactgacagtgctgggcggaggcggttctcagtctgtgctgacacagcctccatccgtgtctgctgcccctggccagaaagtgaccatcagctgtagcggcaacaccagcaacatcggcaacaacttcgtgtcctggtatcagcagaggcccggcagagcacctcagctgctgatctacgagacagacaagaggcccagcggcatccccgatagattttccgccagcaagagcggcacctctggcacactggctatcacaggactgcagacaggcgacgaggccgattactactgtgctacatgggccgccagcctgtctagcgccagagtttttggcaccggcaccaaagtgatcgtgctgggttctggcggaagcggaggatctggatctggtggtggtggatctgcttctggcagtggacaggttcagctggtggaatctggtggcggagtggtgcagcctggcacatctctgagactgtcttgtgccgcctctcagttcagattcgacggctacggaatgcactgggtccgacaggcacctggcaaaggccttgaatgggtcgccagcatcagccacgacggcatcaagaagtaccacgccgagaaagtgtggggcagattcaccatcagccgggacaacagcaagaacaccctgtacctgcagatgaacagcctgcggcctgaggacacagccctgtactattgcgccaaggacctgcgcgaggacgagtgtgaagaatggtggagcgactactacgacttcggcaagcagctgccctgcgccaaaagtagaggtggccttgtgggaatcgccgacaactggggacagggcacaatggtcacagtgtcctctggaggcggaggtagtcaggttcagctgcaagagtctggccctggcctggtcaagcctagcgaaacactgagcgtgacctgtagcgtgtccggcgacagcatgaacaactactactggacctggatcagacagagccccggcaaaggcctggaatggatcggctacatcagcgacagagagagcgccacctacaatcccagcctgaacagcagagtggtcatcagccgggacaccagcaagaaccagctgagcctgaagctgaacagcgtgacccctgccgataccgccgtgtactattgtgccaccgccagaagaggccagcggatctatggcgtggtgtccttcggcgagttcttctactactacagcatggacgtgtggggcaagggcaccaccgtgacagtgtctagcgcttctggcggctccggagataagacccacacatgtcctccatgtcctgctccagaactgctcggcggcccttctgtgttcctgtttcctccaaagcctaaggacaccctgatgatcagcagaacccctgaagtgacctgcgtggtggtggatgtgtctcacgaggaccccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctgcctgctcctatcgagaaaaccatcagcaaggccaagggccagcctagggaaccccaggtttacacccttcctccatgccgggatgagctgaccaagaaccaggtgtccctgtggtgtctcgtgaagggcttctaccccagcgacattgccgtggaatgggagagcaatggccagcctgagaacaactacaagacaacccctcctgtgctggacagcgacggctcattcttcctgtacagcaagctgacagtcgacaagtccagatggcagcagggcaacgtgttcagttgcagcgtgctgcacgaggccctgcacagtcactacacccagaagtccctgagcctgtctcctgga

SEQ ID NO: 94 - protein, 723 aa, synthetic construct; identical to SEQ ID NO: 90
  REGION 1..723: note = MX875 - chain 2

SEQ ID NO: 95 - DNA, 2169 bp, synthetic construct; identical to SEQ ID NO: 49
  misc_feature 1..2169: note = MX875 - chain 2
  source 1..2169: mol_type = other DNA

SEQ ID NO: 96 - protein, 758 aa, synthetic construct
  REGION 1..758: note = MX877 - chain 1
QSVLTQPPSVSAAPGQKVTISCSGNTSNIGNNFVSWYQQRPGRAPQLLIYETDKRPSGIPDRFSASKSGTSGTLAITGLQTGDEADYYCATWAASLSSARVFGTGTKVIVLGGGGSESALTQPPSASGSPGQSITISCTGTSNNFVSWYQQHAGKAPKLVIYDVNKRPSGVPDRFSGSKSGNTASLTVSGLQTDDEAVYYCGSLVGNWDVIFGGGTKLTVLGSGGSGGSGSGGGGSASGSGEPQLQESGPTLVEASETLSLTCAVSGDSTAACNSFWGWVRQPPGKGLEWVGSLSHCASYWNRGWTYHNPSLKSRLTLALDTPKNLVFLKLNSVTAADTATYYCARFGGEVLRYTDWPKPAWVDLWGRGTLVTVSSGGGGSQVQLVESGGGVVQPGTSLRLSCAASQFRFDGYGMHWVRQAPGKGLEWVASISHDGIKKYHAEKVWGRFTISRDNSKNTLYLQMNSLRPEDTALYYCAKDLREDECEEWWSDYYDFGKQLPCAKSRGGLVGIADNWGQGTMVTVSSASGGSGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVLHEALHSHYTQKSLSLSPG

SEQ ID NO: 97 - DNA, 2274 bp, synthetic construct
  misc_feature 1..2274: note = MX877 - chain 1
  source 1..2274: mol_type = other DNA
cagtctgtgctgacacagcctccatccgtgtctgctgcccctggccagaaagtgaccatcagctgtagcggcaacaccagcaacatcggcaacaacttcgtgtcctggtatcagcagaggcccggcagagcacctcagctgctgatctacgagacagacaagaggcccagcggcatccccgatagattttccgccagcaagagcggcacctctggcacactggctatcacaggactgcagacaggcgacgaggccgattactactgtgctacatgggccgccagcctgtctagcgccagagtttttggcaccggcaccaaagtgatcgtgctgggcggaggcggttctgaaagcgctctgacccagcctccatctgcatctggaagccctggccagagcatcaccatcagctgtaccggcaccagcaacaacttcgtgtcctggtatcaacagcacgccggcaaggcccctaagctggtcatctacgacgtgaacaagaggccctccggcgtgcccgatagattcagcggctctaagtctggcaacaccgccagcctgactgtgtctggcctgcagacagatgatgaggccgtgtattactgcggcagcctcgtcggaaactgggacgtgatatttggaggcggcaccaagctgaccgtgctgggttctggcggaagcggaggatctggatctggtggtggtggatctgcttctggcagtggagaacctcagctccaagaaagcggccccacactggtggaagcctctgaaaccctgtctctgacctgtgccgtgtctggggattctaccgccgcctgcaatagcttctggggctgggttcgacagcctcctggaaagggactcgagtgggtcggaagcctgtctcactgtgccagctactggaacagaggctggacctatcacaaccctagcctgaagtccaggctgaccctggctctggacacccctaagaacctggtgttcctgaaactgaactccgtgacagccgccgacacagccacctactactgcgctagatttggcggcgaggtgctgagatacaccgactggcctaaacctgcctgggttgacctgtggggcagaggcacactggttaccgtgtcctctggaggcggaggtagtcaggttcagctggtggaatctggtggcggagtggtgcagcctggcacatctctgagactgtcttgtgccgcctctcagttcagattcgacggctacggaatgcactgggtccgacaggcacctggcaaaggccttgaatgggtcgccagcatcagccacgacggcatcaagaagtaccacgccgagaaagtgtggggcagattcaccatcagccgggacaacagcaagaacaccctgtacctgcagatgaacagcctgcggcctgaggacacagccctgtactattgcgccaaggacctgcgcgaggacgagtgtgaagaatggtggagcgactactacgacttcggcaagcagctgccctgcgccaaaagtagaggtggccttgtgggaatcgccgacaactggggacagggcacaatggtcacagtgtcctctgcttctggcggctccggagataagacccacacatgtcctccatgtcctgctccagaactgctcggcggcccttctgtgttcctgtttcctccaaagcctaaggacaccctgatgatcagcagaacccctgaagtgacctgcgtggtggtggatgtgtctcacgaggaccccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctgcctgctcctatcgagaaaaccatcagcaaggccaagggccagcctagggaaccccaggtttacacccttcctccatgccgggatgagctgaccaagaaccaggtgtccctgtggtgtctcgtgaagggcttctaccccagcgacattgccgtggaatgggagagcaatggccagcctgagaacaactacaagacaacccctcctgtgctggacagcgacggctcattcttcctgtacagcaagctgacagtcgacaagtccagatggcagcagggcaacgtgttcagttgcagcgtgctgcacgaggccctgcacagtcactacacccagaagtccctgagcctgtctcctgga

SEQ ID NO: 98 - protein, 723 aa, synthetic construct; identical to SEQ ID NO: 90
  REGION 1..723: note = MX877 - chain 2

SEQ ID NO: 99 - DNA, 2169 bp, synthetic construct; identical to SEQ ID NO: 49
  misc_feature 1..2169: note = MX877 - chain 2
  source 1..2169: mol_type = other DNA

SEQ ID NO: 100-103 - skipped (4 entries)

SEQ ID NO: 104 - protein, 5 aa, synthetic construct
  REGION 1..5: note = Amino acid linker
GGSGG

SEQ ID NO: 105 - protein, 20 aa, synthetic construct
  REGION 1..20: note = Amino acid linker
GSGGSAGSGSGGGGSASGSG

SEQ ID NO: 106 - protein, 5 aa, synthetic construct
  REGION 1..5: note = Amino acid linker
GGGGS

SEQ ID NO: 107 - protein, 20 aa, synthetic construct
  REGION 1..20: note = Amino acid linker
GSGGSGGSGSGGGGSASGSG